(19) United States
(12) Patent Application Publication
(10) Pub. No.: US 2016/0052898 A1
(43) Pub. Date: Feb. 25, 2016

(54) NOVEL COMPOUNDS

(71) Applicant: BAYER PHARMA AKTIENGESELLSCHAFT, Berlin (DE)

(72) Inventors: Kai THEDE, Berlin (DE); William Johnston SCOTT, Guilford, CT (US); Eckhard BENDER, Langenfeld (DE); Stefan GOLZ, Mülheim an der Ruhr (DE); Andrea HÄGEBARTH, Berlin (DE); Philip LIENAU, Berlin (DE); Florian PUEHLER, Massachusettes (DE); Daniel BASTING, Köln (DE); Dirk SCHNEIDER, Wuppertal (DE); Manfred MÖWES, Berlin (DE); Anja RICHTER, Berlin (DE); Ludwig ZORN, Berlin (DE); Ningshu LIU, Berlin (DE); Ursula MÖNNING, Woltersdorf (DE); Franziska SIEGEL, Berlin (DE)

(21) Appl. No.: 14/778,603

(22) PCT Filed: Mar. 20, 2014
(86) PCT No.: PCT/EP2014/055616

§ 371 Date: Sep. 20, 2015

Related U.S. Application Data
(60) Provisional application No. 61/803,552, filed on Mar. 20, 2013.

(51) Int. Cl. C07D 295/15; C07D 491/08; C07D 498/08; C07D 333/24; C07D 307/54; C07D 491/107; C07D 267/10; C07D 211/42; C07D 211/46; C07C 237/40; C07D 207/12; C07D 231/10; C07D 265/30

(52) U.S. Cl. CPC C07D 295/15; C07D 231/10; C07D 491/08; C07D 498/08; C07D 333/24; C07D 307/54; C07D 265/30; C07D 267/10; C07D 211/42; C07D 211/46; C07C 237/40; C07D 207/12; C07D 491/107

(57) ABSTRACT

The present invention relates to substituted N-biphenyl-3-acetylamino-benzamides and N-[3-(acetylamino)phenyl]-biphenyl-carboxamides of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyperproliferative disorder, as a sole agent or in combination with other active ingredients.

The present invention relates substituted N-biphenyl-3-acetylamino-benzamides and N-[3-(acetylamino)phenyl]-biphenyl-carboxamides of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative disorder, as a sole agent or in combination with other active ingredients.

### BACKGROUND OF THE INVENTION

The Wnt signaling pathways are a group of signal transduction pathways made of proteins that pass signals from outside of a cell through cell surface receptors to the inside of the cell.

Wnt proteins are secreted glycoproteins with a molecular weight in the range of 39-46 kD, whereby in total 19 different members of the Wnt protein family are known (McMahon et al., Trends Genet. 8, 1992, 236-242). They are the ligands of so-called Frizzled receptors, which form a family of seven-transmembrane spanning receptors comprising 10 distinct subtypes. A certain Wnt ligand can thereby activate several different Frizzled receptor subtypes and vice versa a particular Frizzled receptor can be activated by different Wnt protein subtypes (Huang et al., Genome Biol. 5, 2004, 234.1-234.8).

Binding of a Wnt to its receptor can activate two different signaling cascades, one is called the non-canonical pathway, which involves CamK II and PKC (Kuhl et al., Trends Genet. 16 (7), 2000, 279 283). The other, the so-called canonical pathway (Tamai et al., Mol. Cell 13, 2004, 149-156) regulates the concentration of the transcription factor β-catenin.

In the case of non-stimulated canonical Wnt signaling, β-catenin is captured by a destruction complex consisting of adenomatous polyposis coli (APC), glycogen synthase kinase 3-β (GSK-3β), Axin-1 or -2 and Casein Kinase 1α. Captured β-catenin is then phosphorylated, ubiquitinated and subsequently degraded by the proteasome.

However, when a canonical Wnt activates the membrane complex of a Frizzled receptor and its Lipoprotein 5 or 6 (LRP 5/6) co-receptor, this leads to the recruitment of dishevelled (Dvl) by the receptors and subsequent phosphorylation of LRP 5/6, followed by binding of Axin-1 or Axin-2 to the membrane complex as well. The deprivation of Axin from the β-catenin destruction complex leads to the disassembly of the latter and β-catenin can reach the nucleus, where it together with TCF and LEF transcription factors and other transcriptional coregulators like Pygopus, BCL9/Legless, CDK8 module of Mediator and TRRAP initiates transcription of genes with promoters containing TCF elements (Najdi, J. Carcinogenesis 2011; 10:5).

The Wnt signaling cascade can be constitutively activated by mutations in genes involved in this pathway. This is especially well documented for mutations of the APC and axin genes, and also for mutations of the β-catenin phosphorylation sites, all of which are important for the development of colorectal and hepatocellular carcinomas (Polakis, EMBO J., 31, 2012, 2737-2746).

The Wnt signaling cascade has important physiological roles in embryonal development and tissue homeostasis the latter especially for hair follicles, bones and the gastrointestinal tract. Deregulation of the Wnt pathway can activate in a cell and tissue specific manner a number of genes known to be important in carcinogenesis. Among them are c-myc, cyclin D1, Axin-2 and metalloproteases (He et al., Science 281, 1998, 1509-1512).

Deregulated Wnt activity can drive cancer formation, increased Wnt signaling can thereby be caused through autocrine Wnt signaling, as shown for different breast, ovarian, prostate and lung carcinomas as well as for various cancer cell lines (Bafico, Cancer Cell 6, 2004, 497-506; Yee, Mol. Cancer 9, 2010, 162-176; Nguyen, Cell 138, 2009, 51-62).

For cancer stem cells (CSCs) it was shown that they have increased Wnt signaling activity and that its inhibition can reduce the formation of metastases (Vermeulen et al., Nature Cell Biol. 12 (5), 2010, 468-476; Polakis, EMBO J. 31, 2012, 2737-2746; Reya, Nature, 434, 2005, 843-850).

Furthermore, there is a lot of evidence supporting an important role of Wnt signaling in cardiovascular diseases. One aspect thereby is heart failure and cardiac hypertrophy where deletion of Dapper-1, an activator of the canonical β-catenin Wnt pathway has been shown to reduce functional impairement and hypertrophy (Hagenmueller, M. et al.: Dapper-1 induces myocardial remodeling through activation of canonical wnt signaling in cardiomyocytes; Hypertension, 61 (6), 2013, 1177-1183).

Additional support for a role of Wnt signaling in heart failure comes from animal experimental models and clinical studies with patients, in which it was shown, that the level of secreted frizzled related protein 3 (sFRP3) is associated with the progression of heart failure (Askevold, E. T. et al.: The cardiokine secreted Frizzled-related protein 3, a modulator of Wnt signaling in clinical and experimental heart failure; J. Intern Med., 2014 (doi:10.1111/joim.12175)). For cardiac remodeling and infarct healing the expression of Fzd2 receptors on myofibroblasts migrating into the infarct area has been demonstrated (Blankesteijn, W. M. et al.: A homologue of Drosophila tissue polarity gene frizzled is expressed in migrating myofibroblasts in the infarcted rat heart; Nat. Med. 3, 1997, 541-544). The manifold effects of Wnt signaling in heart failure, fibrosis and arrhythmias have been recently reviewed by Dawson et al. (Dawson, K. et al.: Role of the Wnt-Frizzled system in cardiac pathophysiology: a rapidly developing, poorly understood area with enormous potential; J. Physiol. 591 (6), 2013, 1409-1432).

For the vasculature, effects of Wnt signaling could be shown as well, mainly in respect to restenosis via enhancement of vascular smooth muscle cell proliferation (Tsaousi, A. et al.: Wnt4/b-catenin signaling induces VSMC proliferation and is associated with initmal thickening; Circ. Res. 108, 2011, 427-436).

Besides the effects on heart and vasculature, dysregulated Wnt signaling is also an important component in chronic kidney disease as could be shown for upregulated Wnt activity in immune cells from corresponding patients (Al-Chaqmaqchi, H. A. et al.: Activation of Wnt/b-catenin pathway in monocytes derived from chronic kidney disease patients; PLoS One, 8 (7), 2013, doi: 10.1371) and altered levels of secreted Wnt inhibitor in patient sera (de Oliveira, R. B. et al.: Disturbances of Wnt/b-catenin pathway and energy metabolism in early CKD: effect of phosphate binders; Nephrol. Dial. Transplant. (2013) 28 (10): 2510-2517).

In adults, mis-regulation of the Wnt pathway also leads to a variety of abnormalities and degenerative diseases. An LRP mutation has been identified that causes increased bone density at defined locations such as the jaw and palate (Boyden L M et al.: High bone density due to a mutation in LDL-receptor-related protein 5; N Engl J Med. 2002 May 16; 346(20):1513-21, Gong Y, et al.: LDL receptor-related protein 5 (LRP5) affects bone accrual and eye development; Cell 2001; 107:513-23). The mutation is a single amino-acid substitution that makes LRP5 insensitive to Dkk-mediated Wnt pathway inhibition, indicating that the phenotype results from overactive Wnt signaling in the bone. Recent reports have suggested that WNT signaling is an important regulator for adipogenesis or insulin secretion and might be involved in the pathogenesis of type 2 diabetes. It has been shown that expression of the WNTSB gene was detectable in several tissues, including adipose, pancreas, and liver. Subsequent in vitro experiments identified the fact that expression of the Wnt5b gene was increased at an early phase of adipocyte differentiation in mouse 3T3-L1 cells. Furthermore, overexpression of the Wnt5b gene in preadipocytes resulted in the promotion of adipogenesis and the enhancement of adipocytokine-gene expression. These results indicate that the WNTSB gene may contribute to conferring susceptibility to type 2 diabetes and may be involved in the pathogenesis of this disease through the regulation of adipocyte function (Kanazawa A, et al.: Association of the gene encoding wingless-type mammary tumor virus integration-site family member 58 (WNT58) with type 2 diabetes; Am J Hum Genet. 2004 November; 75(5):832-43)

Accordingly, identification of methods and compounds that modulate the WNT-dependent cellular responses may offer an avenue for regulating physiological functions and therapeutic treatment of diseases associated with aberrant activity of the pathways.

Inhibitors of the WNT signalling pathway are disclosed e.g. in US2008-0075714(A1), US2011-0189097(A1), US2012-0322717(A9), WO2010/014948(A1), WO2012/088712(A1), WO2012/140274(A2,A3) and WO2013/093508(A2).

WO 2005/084368(A2) discloses heteroalkyl-substituted biphenyl-4-carboxylic acid arylamide analogues and the use of such compounds for treating conditions related to capsaicin receptor activation, for identifying other agents that bind to capsaicin receptor, and as probes for the detection and localization of capsaicin receptors. The structural scope of the compounds claimed in claim 1 is huge, whereas the structural space spanned by the few examples is much smaller. There is no specific example which is covered by the formula (I) as described and defined herein.

WO 2000/55120(A1) and WO 2000/07991 (A1) disclose amide derivatives and their use for the treatment of cytokine mediated diseases. The few specific examples disclosed in WO 2000/55120(A1) and WO 2000/07991 (A1) are not covered by the formula (I) as described and defined herein.

WO 1998/28282 (A2) discloses oxygen or sulfur containing heteroaromatics as factor Xa inhibitors. The specific examples disclosed in WO 1998/28282 (A2) are not covered by the formula (I) as described and defined herein.

WO 2011/035321 (A1) discloses methods of treating Wnt/Frizzled-related diseases, comprising administering niclosamide compounds. According to the specification of WO 2011/035321 (A1) libraries of FDA-approved drugs were examined for their utility as Frizzled internalization modulators, employing a primary imaged-based GFP-fluorescence assay that used Frizzled1 endocytosis as the readout. It was discovered that the antihelminthic niclosamide, a drug used for the treatment of tapeworms, promotes Frizzled1 internalization (endocytosis), down regulates Dishevelled-2 protein, and inhibits Wnt3A-stimulated β-catenin stabilization and LEF/TCF reporter activity. The specific examples disclosed in WO 2011/035321 (A1) are not covered by the formula (I) as described and defined herein. Additionally, WO 2011/035321 (A1) does neither teach nor suggest the compounds of formula (I) as described and defined herein. The same is true for the related publication WO 2004/006906 (A2) which discloses a method for treating a patient having a cancer or other neoplasm by administering to the patient a niclosamide.

JP 2010-138079 (A) relates to amide derivatives exhibiting insecticidal effects. The specific examples disclosed in JP 2010-138079 (A) are not covered by the formula (I) as described and defined herein. WO 2004/022536 (A1) relates to heterocyclic compounds that inhibit phosphodiesterase type 4 (PDE 4) and their use for treating inflammatory conditions, diseases of the central nervous system and insulin resistant diabetes. The specific examples disclosed in WO 2004/022536 (A1) are not covered by the formula (I) as described and defined herein.

### SUMMARY OF THE INVENTION

The present invention relates to compounds of general formula (I):

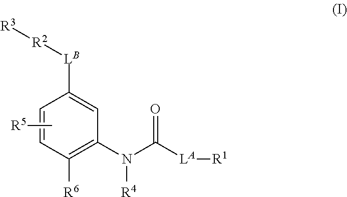

in which:
L^{A} represents a methylene or ethylene group, said methylene or ethylene group being optionally substituted, one or more times, identically or differently, with a substituent selected from:
   hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-, 3- to 10-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
   C₃-C₇-cycloalkyl-, C₄-C₇-cycloalkenyl-, 3- to 10-membered heterocycloalkyl-,
   4- to 10-membered heterocycloalkenyl-, aryl-, heteroaryl-, —N(R⁷)—(C₁-C₆-alkyl),
   —N(R⁷)—C(═O)—O—(C₁-C₆-alkyl), —N(R⁷)R⁷;
   wherein said C₃-C₇-cycloalkyl-, C₄-C₇-cycloalkenyl-, 3- to 10-membered heterocycloalkyl-,
   4- to 10-membered heterocycloalkenyl-, aryl-, heteroaryl-, and —N(R⁷)—(C₁-C₆-alkyl) group is optionally substituted, one or more times, identically or differently, with a substituent selected from: halo-, hydroxy-, cyano-, C₁-C₃-alkyl-, C₁-C₃-alkoxy-,
   halo-C₁-C₃-alkyl-, hydroxy-C₁-C₃-alkyl-, halo-C₁-C₃-alkoxy-,
   C₃-C₇-cycloalkyl-, 3- to 10-membered heterocycloalkyl-,
   —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰,
   —S(═O)₂N(H)R⁹, —S(═O)₂NR¹⁰R⁹, —S(═O)(═NR¹⁰)R⁹, —S(═O)(═NR¹⁰)R⁹, —N═S(═O)(R¹⁰)R⁹;
R² represents:

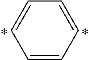

wherein “*” represents the point of attachment to R³ or L^{B}, respectively; wherein said group is optionally substituted, one or more times, identically or differently, with halo- or a C₁-C₃-alkyl-group;
R³ represents a phenyl-group,
   said phenyl-group being optionally substituted, one or more times, identically or differently, with a substituent selected from: halo-, hydroxy-, cyano-, C₁-C₃-alkyl-, C₁-C₃-alkoxy-,
   halo-C₁-C₃-alkyl-, hydroxy-C₁-C₃-alkyl-, NH₂—C₁-C₃-alkyl-, halo-C₁-C₃-alkoxy-,
   C₃-C₇-cycloalkyl-, 3- to 10-membered heterocycloalkyl-,
   —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰,
   —S(═O)₂N(H)R⁹, —S(═O)₂NR¹⁰R⁹, —S(═O)(═NR¹⁰)R⁹, —S(═O)(═NR¹⁰)R⁹, —N═S(═O)(R¹⁰)R⁹;
   or, when two substituents are present ortho to each other on the phenyl-group, said two substituents together form a bridge: *O(CH₂)₂O*, *O(CH₂)O*, *O—C(H)₂—C(H)₂*, *NH(C(═O))NH*, wherein * represent the points of attachment to the phenyl-group;
R⁴ represents a hydrogen atom or a group selected from:
   C₁-C₆-alkyl-, C₃-C₄-alkenyl-, C₃-C₄-alkynyl-,
   —(CH₂)ₘ—C₃-C₇-cycloalkyl, —(CH₂)ₘ—C₄-C₇-cycloalkenyl,
   —(CH₂)ₘ-(3- to 10-membered heterocycloalkyl),
   —(CH₂)ₘ-(4- to 10-membered heterocycloalkenyl),
   —(CH₂)ₘ-aryl, —(CH₂)ₘ-heteroaryl;
R⁵ represents a hydrogen atom or a halogen atom or a group selected from:
   cyano-, C₁-C₃-alkyl-, C₁-C₃-alkoxy-;
R⁶ represents a group selected from:
   C₁-C₆-alkyl-, C₂-C₆-alkenyl-, C₂-C₆-alkynyl-
   C₁-C₆-alkoxy-, C₃-C₆-cycloalkoxy-, halo-, hydroxy-, cyano-, aryl-,
   heteroaryl-, —N(R⁹)(R¹⁰), —C(═O)—O—R⁹, —C(═O)—N(R⁹)(R¹⁰), R⁹—S—, R⁹—S(═O)—, R⁹—S(═O)₂—;
   said C₁-C₆-alkyl-, C₂-C₆-alkenyl-, C₂-C₆-alkynyl-, aryl-, heteroaryl- or C₁-C₆-alkoxy-group being optionally substituted, one or more times, identically or differently, with
   halo-, cyano-, nitro-, hydroxy-, C₁-C₃-alkyl-, C₁-C₃-alkoxy-, halo-C₁-C₃-alkoxy-,
   hydroxy-C₁-C₃-alkoxy-, C₁-C₃-alkoxy-C₁-C₃-alkoxy-,
   C₃-C₇-cycloalkyl-, C₄-C₇-cycloalkenyl-,
   3- to 10-membered heterocycloalkyl-, 4- to 10-membered heterocycloalkenyl-,
   aryl-, heteroaryl-, —C(═O)R⁹, —C(═O)O—R⁹, —OC(═O)—R⁹, —N(H)C(═O)R⁹, —N(R¹⁰)C(═O)R⁹,
   —N(H)C(═O)NR¹⁰R⁹, —N(R¹¹)C(═O)NR¹⁰R⁹, —N(H)R⁹, —NR¹⁰R⁹,
   —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹;
R⁷ represents —H or C₁-C₃-alkyl-;
R⁹, R¹⁰, R¹¹
   represent, independently from each other, —H, C₁-C₃-alkyl- or C₃-C₆-cycloalkyl-;
   said C₁-C₃-alkyl-group being optionally substituted with C₁-C₃-alkoxy- or —N(R¹²)R¹³;
or
R⁹R¹⁰ together with the atom or the group of atoms they are attached to, form a 3- to 10-membered heterocycloalkyl- or 4- to 10-membered heterocycloalkenyl-group;
R¹², R¹³
   represent, independently from each other, —H or C₁-C₃-alkyl-;
or
R¹², R¹³ together with the atom they are attached to, form a 3- to 10-membered heterocycloalkyl- or 4- to 10-membered heterocycloalkenyl-group;
m represents 0, 1, or 2;
or a tautomer, an N-oxide, a hydrate, a solvate, or a salt thereof, or a mixture of same.

The present invention further relates to a pharmaceutical composition comprising a compound of formula (I), supra.

The present invention further relates to the use of a compound of formula (I), supra, for the prophylaxis or treatment of a disease.

The present invention further relates to the use of a compound of formula (I), supra, for the preparation of a medicament for the prophylaxis or treatment of a disease.

### DETAILED DESCRIPTION OF THE INVENTION

The terms as mentioned in the present text have preferably the following meanings: The term “halogen atom” or “halo-” is to be understood as meaning a fluorine, chlorine, bromine or iodine atom.

The term “C₁-C₆-alkyl” is to be understood as preferably meaning a linear or branched, saturated, monovalent hydrocarbon group having 1, 2, 3, 4, 5 or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methyl butyl, 1-ethyl propyl, 1,2-dimethylpropyl, neo-pentyl, 1,1-dimethylpropyl, 4-methyl pentyl, 3-methyl pentyl, 2-methylpentyl, 1-methylpentyl, 2-ethyl butyl, 1-ethyl butyl, 3,3-dimethyl butyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “halo-C₁-C₆-alkyl” is to be understood as preferably meaning a linear or branched, saturated, monovalent hydrocarbon group in which the term “C₁-C₆-alkyl” is defined supra, and in which one or more of the hydrogen atoms is replaced, identically or differently, by a halogen atom. Particularly, said halogen atom is F. Said halo-C₁-C₆-alkyl group is, for example, —CF₃, —CHF2, —CH₂F, —CF₂CF₃, or —CH₂CF₃.

The term “C₁-C₆-alkoxy” is to be understood as preferably meaning a linear or branched, saturated, monovalent group of formula O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, identically or differently, by a halogen atom. Particularly, said halogen atom is F. Said halo-C₁-C₆-alkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, identically or differently, by a halogen atom. Particularly, said halogen atom is F. Said halo-C₁-C₆-alkoxy-C₁-C₆-alkyl group is, for example, —CH₂CH₂OCF₃, —CH₂CH₂OCHF₂, —CH₂CH₂OCH₂F, —CH₂CH₂OCF₂CF₃, or —CH₂CH₂OCH₂CF₃.

The term “C₁-C₆-alkoxy-C₂-C₆-alkoxy” is to be understood as preferably meaning a saturated, monovalent C₂-C₆-alkoxy group, as defined supra, in which one of the hydrogen atoms is replaced by a C₁-C₆-alkoxy group, as defined supra, e.g. methoxyalkoxy, ethoxyalkoxy, pentoxyalkoxy, hexoxyalkoxy group or methoxyethoxy, ethoxyethoxy, iso-propoxyhexoxy group, in which the term “alkoxy” is defined supra, or an isomer thereof.

The term “C₂-C₆-alkenyl” is to be understood as preferably meaning a linear or branched, monovalent hydrocarbon group, which contains one or more double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methyl prop-2-enyl, 1-methyl prop-2-enyl, 2-methyl prop-1-enyl, (E)-1-methyl prop-1-enyl, (Z)-1-methyl prop-1-enyl, 3-methyl but-3-enyl, 2-methyl but-3-enyl, 1-methyl but-3-enyl, 3-methyl but-2-enyl, (E)-2-methyl but-2-enyl, (Z)-2-methyl but-2-enyl, (E)-1-methyl but-2-enyl, (Z)-1-methyl but-2-enyl, (E)-3-methyl but-1-enyl, (Z)-3-methyl but-1-enyl, (E)-2-methyl but-1-enyl, (Z)-2-methyl but-1-enyl, (E)-1-methyl but-1-enyl, (Z)-1-methyl but-1-enyl, 1,1-dimethylprop-2-enyl, 1-ethyl prop-1-enyl, 1-propylvinyl, 1-isopropylvinyl, 4-methyl pent-4-enyl, 3-methyl pent-4-enyl, 2-methyl pent-4-enyl, 1-methyl pent-4-enyl, 4-methyl pent-3-enyl, (E)-3-methyl pent-3-enyl, (Z)-3-methyl pent-3-enyl, (E)-2-methyl pent-3-enyl, (Z)-2-methyl pent-3-enyl, (E)-1-methyl pent-3-enyl, (Z)-1-methyl pent-3-enyl, (E)-4-methyl pent-2-enyl, (Z)-4-methyl pent-2-enyl, (E)-3-methyl pent-2-enyl, (Z)-3-methyl pent-2-enyl, (E)-2-methyl pent-2-enyl, (Z)-2-methyl pent-2-enyl, (E)-1-methyl pent-2-enyl, (Z)-1-methyl pent-2-enyl, (E)-4-methyl pent-1-enyl, (Z)-4-methyl pent-1-enyl, (E)-3-methyl pent-1-enyl, (Z)-3-methyl pent-1-enyl, (E)-2-methyl pent-1-enyl, (Z)-2-methyl pent-1-enyl, (E)-1-methyl pent-1-enyl, (Z)-1-methyl pent-1-enyl, 3-ethyl but-3-enyl, 2-ethyl but-3-enyl, 1-ethyl but-3-enyl, (E)-3-ethyl but-2-enyl, (Z)-3-ethyl but-2-enyl, (E)-2-ethyl but-2-enyl, (Z)-2-ethyl but-2-enyl, (E)-1-ethyl but-2-enyl, (Z)-1-ethyl but-2-enyl, (E)-3-ethyl but-1-enyl, (Z)-3-ethyl but-1-enyl, 2-ethyl but-1-enyl, (E)-1-ethyl but-1-enyl, (Z)-1-ethyl but-1-enyl, 2-propyl prop-2-enyl, 1-propyl prop-2-enyl, 2-isopropyl prop-2-enyl, 1-isopropyl prop-2-enyl, (E)-2-propyl prop-1-enyl, (Z)-2-propyl prop-1-enyl, (E)-1-propyl prop-1-enyl, (Z)-1-propyl prop-1-enyl, (E)-2-isopropyl prop-1-enyl, (Z)-2-isopropylprop-1-enyl, (E)-1-isopropylprop-1-enyl, (Z)-1-isopropyl prop-1-enyl, (E)-3,3-dimethylprop-1-enyl, (Z)-3,3-dimethyl prop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₆-alkynyl” is to be understood as preferably meaning a linear or branched, monovalent hydrocarbon group which contains one or more triple bonds, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methyl prop-2-ynyl, 2-methyl but-3-ynyl, 1-methyl but-3-ynyl, 1-methyl but-2-ynyl, 3-methyl but-1-ynyl, 1-ethyl prop-2-ynyl, 3-methylpent-4-ynyl, 2-methyl pent-4-ynyl, 1-methylpent-4-ynyl, 2-methyl pent-3-ynyl, 1-methylpent-3-ynyl, 4-methyl pent-2-ynyl, 1-methylpent-2-ynyl, 4-methyl pent-1-ynyl, 3-methyl pent-1-ynyl, 2-ethyl but-3-ynyl, 1-ethyl but-3-ynyl, 1-ethyl but-2-ynyl, 1-propylprop-2-ynyl, 1-isopropyl prop-2-ynyl, 2,2-dimethyl but-3-ynyl, 1,1-dimethyl but-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₇-cycloalkyl” is to be understood as meaning a saturated, monovalent, monocyclic hydrocarbon ring which contains 3, 4, 5, 6 or 7 carbon atoms. Said C₃-C₇-cycloalkyl group is for example a cyclopropyl, cyclobutyl, cyclopentyl, cyclohexyl or cycloheptyl ring. Particularly, said ring contains 3, 4, 5 or 6 carbon atoms (“C₃-C₆-cycloalkyl”).

The term “C₄-C₈-cycloalkenyl” is to be understood as preferably meaning a monovalent, monocyclic hydrocarbon ring which contains 4, 5, 6, 7 or 8 carbon atoms and one or two double bonds, in conjugation or not, as the size of said cycloalkenyl ring allows. Particularly, said ring contains 4, 5 or 6 carbon atoms (“C₄-C₆-cycloalkenyl”). Said C₄-C₈-cycloalkenyl group is for example a cyclobutenyl, cyclopentenyl, or cyclohexenyl group.

The term “C₃-C₆-cycloalkoxy” is to be understood as meaning a saturated, monovalent, monocyclic group of formula —O—(C₃-C₆-cycloalkyl), in which the term “C₃-C₆-cycloalkyl” is defined supra, e.g. a cyclopropyloxy, cyclobutyloxy, cyclopentyloxy or cyclohexyloxy group.

The term “3- to 10-membered heterocycloalkyl”, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), 0, S, S(═O), S(═O)₂, NR^{a}, in which R^{a} represents a hydrogen atom, or a C₁-C₆-alkyl-group; it being possible for said heterocycloalkyl group to be attached to the rest of the molecule via any one of the carbon atoms or, if present, a nitrogen atom.

Particularly, said 3- to 10-membered heterocycloalkyl can contain 2, 3, 4, 5 or 6 carbon atoms, and one or more of the above-mentioned heteroatom-containing groups (a “3- to 7-membered heterocycloalkyl”), more particularly said heterocycloalkyl can contain 4, 5 or 6 carbon atoms, and one or more of the above-mentioned heteroatom-containing groups (a “4- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

The term “4- to 10-membered heterocycloalkenyl”, is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), 0, S, S(═O), S(═O)₂, NR^{a}, in which R^{a} represents a hydrogen atom or a C₁-C₆-alkyl-group; it being possible for said heterocycloalkenyl group to be attached to the rest of the molecule via any one of the carbon atoms or, if present, a nitrogen atom. Examples of said heterocycloalkenyl may contain one or more double bonds, e.g. 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group.

The term “aryl” is to be understood as preferably meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a biphenyl group (a “C₁₂-aryl” group), or a ring having 13 carbon atoms, (a “C₁₃-aryl” group), e.g. a fluorenyl group, or a ring having 14 carbon atoms, (a “C₁₄-aryl” group), e.g. an anthracenyl group. Preferably, the aryl group is a phenyl group.

The term “heteroaryl” is understood as preferably meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridinyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridyl includes pyridin-2-yl, pyridin-3-yl, and pyridin-4-yl; or the term thienyl includes thien-2-yl and thien-3-yl. Preferably, the heteroaryl group is a pyridinyl group.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆, particularly C₁-C₂, C₁-C₃, C₁-C₄, C₁-C₅, C₁-C₆, more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅, particularly C₂-C₃.

Further, as used herein, the term “C₃-C₇”, as used throughout this text, e.g. in the context of the definition of “C₃-C₇-cycloalkyl”, is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

Where the plural form of the word compounds, salts, polymorphs, hydrates, solvates and the like, is used herein, this is taken to mean also a single compound, salt, polymorph, isomer, hydrate, solvate or the like.

The compounds of this invention contain one or more asymmetric centres, depending upon the location and nature of the various substituents desired. Asymmetric carbon atoms may be present in the (R) or (S) configuration. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

The present invention includes all possible stereoisomers of the compounds of the present invention as single stereoisomers, or as any mixture of said stereoisomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, viz.:

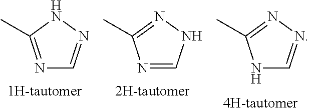

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example. Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula (I):

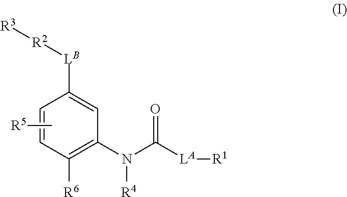

in which:
L^{A} represents a methylene or ethylene group, said methylene or ethylene group being optionally substituted, one or more times, identically or differently, with a substituent selected from:
   hydroxy-, cyano-, C₁-C₃-alkyl-, C₁-C₃-alkoxy-, halo-C₁-C₃-alkyl-, hydroxy-C₁-C₃-alkyl-,
   halo-C₁-C₃-alkoxy-, C₃-C₇-cycloalkyl-, 3- to 10-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from:
   halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
   C₃-C₇-cycloalkyl-, C₄-C₇-cycloalkenyl-, 3- to 10-membered heterocycloalkyl-,
   4- to 10-membered heterocycloalkenyl-, aryl-, heteroaryl-, —N(R⁷)—(C₁-C₆-alkyl),
   —N(R⁷)—C(═O)—O—(C₁-C₆-alkyl), —N(R⁷)R⁷;
   wherein said C₃-C₇-cycloalkyl-, C₄-C₇-cycloalkenyl-, 3- to 10-membered heterocycloalkyl-, 4- to 10-membered heterocycloalkenyl-, aryl-, heteroaryl-, and —N(R⁷)—(C₁-C₆-alkyl) group is optionally substituted, one or more times, identically or differently, with a substituent selected from: halo-, hydroxy-, cyano-, C₁-C₃-alkyl-, C₁-C₃-alkoxy-,
   halo-C₁-C₃-alkyl-, hydroxy-C₁-C₃-alkyl-, halo-C₁-C₃-alkoxy-,
   C₃-C₇-cycloalkyl-, 3- to 10-membered heterocycloalkyl-,
   —C(═O)R⁹, —C(═O)O—R⁹, —OC(═O)—R⁹,
   —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
   —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹;
R² represents:

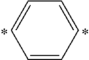

wherein “*” represents the point of attachment to R³ or L^{B}, respectively; wherein said group is optionally substituted, one or more times, identically or differently, with halo- or a C₁-C₃-alkyl-group;
R³ represents a phenyl-group,
   said phenyl-group being optionally substituted, one or more times, identically or differently, with a substituent selected from: halo-, hydroxy-, cyano-, C₁-C₃-alkyl-, C₁-C₃-alkoxy-,
   halo-C₁-C₃-alkyl-, hydroxy-C₁-C₃-alkyl-, NH₂—C₁-C₃-alkyl-, halo-C₁-C₃-alkoxy-,
   C₃-C₇-cycloalkyl-, 3- to 10-membered heterocycloalkyl-,
   —C(═O)R⁹, —C(═O)O—R⁹, —OC(═O)—R⁹,
   —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
   —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹;
   or, when two substituents are present ortho to each other on the phenyl-group, said two substituents together form a bridge: *O(CH₂)₂O*, *O(CH₂)O*, *O—C(H)₂—C(H)₂*, *NH(C(═O))NH*, wherein * represent the points of attachment to the phenyl-group;
R⁴ represents a hydrogen atom or a group selected from:
   C₁-C₆-alkyl-, C₃-C₄-alkenyl-, C₃-C₄-alkynyl-,
   —(CH₂)ₘ—C₃-C₇-cycloalkyl, —(CH₂)ₘ—C₄-C₇-cycloalkenyl,
   —(CH₂)ₘ-(3- to 10-membered heterocycloalkyl),
   —(CH₂)ₘ-(4- to 10-membered heterocycloalkenyl),
   —(CH₂)ₘ-aryl, —(CH₂)ₘ-heteroaryl;
R⁵ represents a hydrogen atom or a halogen atom or a group selected from:
   cyano-, C₁-C₃-alkyl-, C₁-C₃-alkoxy-;
R⁶ represents a group selected from:
   C₁-C₆-alkyl-, C₂-C₆-alkenyl-, C₂-C₆-alkynyl-
   C₁-C₆-alkoxy-, C₃-C₆-cycloalkoxy-, halo-, hydroxy-, cyano-, aryl-,
   heteroaryl-, —N(R⁹)(R¹⁰), —C(═O)—O—R⁹, —C(═O)—N(R⁹)(R¹⁰), R⁹—S—, R⁹—S(═O)—, R⁹—S(═O)₂—;
   said C₁-C₆-alkyl-, C₂-C₆-alkenyl-, C₂-C₆-alkynyl-, aryl-, heteroaryl- or C₁-C₆-alkoxy-group being optionally substituted, one or more times, identically or differently, with
   halo-, cyano-, nitro-, hydroxy-, C₁-C₃-alkyl-, C₁-C₃-alkoxy-, halo-C₁-C₃-alkoxy-,
   hydroxy-C₁-C₃-alkoxy-, C₁-C₃-alkoxy-C₁-C₃-alkoxy-,
   C₃-C₇-cycloalkyl-, C₄-C₇-cycloalkenyl-,
   3- to 10-membered heterocycloalkyl-, 4- to 10-membered heterocycloalkenyl-,
   aryl-, heteroaryl-, —C(═O)R⁹, —C(═O)O—R⁹, —OC(═O)—R⁹, —N(H)C(═O)R⁹, —N(R¹⁰)C(═O)R⁹,
   —N(H)C(═O)NR¹⁰R⁹, —N(R¹¹)C(═O)NR¹⁰R⁹, —N(H)R⁹, —NR¹⁰R⁹,
   —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹;
R⁷ represents —H or C₁-C₃-alkyl-;
R⁹, R¹⁰, R¹¹
   represent, independently from each other, —H, C₁-C₃-alkyl- or C₃-C₆-cycloalkyl-;
   said C₁-C₃-alkyl-group being optionally substituted with C₁-C₃-alkoxy- or —N(R¹²)R¹³;
or
R⁹R¹⁰ together with the atom or the group of atoms they are attached to, form a 3- to 10-membered heterocycloalkyl- or 4- to 10-membered heterocycloalkenyl-group;
R¹², R¹³
   represent, independently from each other, —H or C₁-C₃-alkyl-;
or
R¹², R¹³ together with the atom they are attached to, form a 3- to 10-membered heterocycloalkyl- or 4- to 10-membered heterocycloalkenyl-group;
m represents 0, 1, or 2;
or a tautomer, an N-oxide, a hydrate, a solvate, or a salt thereof, or a mixture of same.

In a embodiment, the present invention relates to compounds of the general formula (I), supra, in which:
L^{A} represents a methylene or ethylene group, said methylene or ethylene group being optionally substituted, one or more times, identically or differently, with a substituent selected from:
   hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-, 3- to 10-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-.

In another embodiment, the present invention relates to compounds of the general formula (I), supra, in which:
L^{A} represents a methylene or ethylene group, said methylene or ethylene group being optionally substituted, one or more times, identically or differently, with a substituent selected from:
   hydroxy-, cyano-, C₁-C₃-alkyl-, C₁-C₃-alkoxy-, fluoro-C₁-C₃-alkyl-,
   hydroxy-C₁-C₃-alkyl-, fluoro-C₁-C₃-alkoxy-, C₃-C₇-cycloalkyl-,
   3- to 10-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from:
   halo-, hydroxy-, cyano-, C₁-C₃-alkyl-, C₁-C₃-alkoxy-.

In another embodiment, the present invention relates to compounds of the general formula (I), supra, in which:
L^{A} represents a methylene or ethylene group, said methylene or ethylene group being optionally substituted, one or more times, identically or differently, with a substituent selected from: C₁-C₃-alkyl- and halo-C₁-C₃-alkyl-;
   or, when two substituents are present at the same carbon atom, the two substituents, together with the carbon atom they are attached to, may form a
   C₃-C₆-cycloalkyl-ring; wherein said ring is optionally substituted one or more times, identically or differently, with a substituent selected from: halo-, hydroxy-, cyano-, C₁-C₃-alkyl-, and C₁-C₃-alkoxy-.

In another embodiment, the present invention relates to compounds of the general formula (I), supra, in which:
L^{A} represents a methylene group, said methylene group being optionally substituted, one or more times, identically or differently, with a substituent selected from:
   cyano-, hydroxy-, C₁-C₃-alkyl-, C₁-C₃-alkoxy-, fluoro-C₁-C₃-alkyl-,
   hydroxy-C₁-C₃-alkyl-, C₃-C₅-cycloalkyl-, 3- to 6-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from:
   halo-, hydroxy-, cyano-, C₁-C₃-alkyl-, C₁-C₃-alkoxy-.

In another embodiment, the present invention relates to compounds of general formula (I), supra, in which:
L^{A} represents a methylene group, said methylene group being optionally substituted, one or more times, identically or differently, with a substituent selected from:
   hydroxy-, C₁-C₃-alkyl-, C₁-C₃-alkoxy-, hydroxy-C₁-C₃-alkyl-,
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from:
   halo-, hydroxy-, cyano-, C₁-C₃-alkyl-, C₁-C₃-alkoxy-.

In a preferred embodiment, the present invention relates to compounds of general formula (I), supra, in which:
L^{A} represents methylene, said methylene group being optionally substituted one or two times, identically or differently, with C₁-C₃-alkyl-,
   wherein, if said methylene is substituted with two C₁-C₃-alkyl-groups, these may, together with the carbon atom they are attached to, form a C₃-C₆-cycloalkyl-ring.

In a preferred embodiment, the present invention relates to compounds of general formula (I), supra, in which:
L^{A} represents a methylene group, said methylene group being optionally substituted, one or more times, identically or differently, with a substituent selected from:
   C₁-C₃-alkyl- and halo-C₁-C₃-alkyl-;
   or, when two substituents are present at the same carbon atom, the two substituents, together with the carbon atom they are attached to, may form a
   C₃-C₄-cycloalkyl-ring.

In a particularly preferred embodiment, the present invention relates to compounds of general formula (I), supra, in which:
L^{A} represents —CH₂—, —CH(CH₃)—, —C(CH₃)₂— or

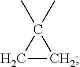

wherein the cycloproypl-ring is optionally substituted one or more times, identically or differently, with a substituent selected from: halo-, hydroxy-, cyano-, C₁-C₃-alkyl-, C₁-C₃-alkoxy-.

In another particularly preferred embodiment, the present invention relates to compounds of general formula (I), supra, in which:
L^{A} represents —C(CH₃)₂—.

In another particularly preferred embodiment, the present invention relates to compounds of general formula (I), supra, in which: L^{A} represents —CH₂— or —CH(CH₃)—.

In another particularly preferred embodiment, the present invention relates to compounds of general formula (I), supra, in which: L^{A} represents —CH₂—.

In another particularly preferred embodiment, the present invention relates to compounds of general formula (I), supra, in which: L^{A} represents —CH(CH₃)—.

In another particularly preferred embodiment, the present invention relates to compounds of general formula (I), supra, in which:
L^{A} represents

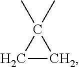

wherein the cycloproypl-ring is optionally substituted one or more times, identically or differently, with a substituent selected from: halo-, hydroxy-, cyano-, C₁-C₃-alkyl-, C₁-C₃-alkoxy-.

In another particularly preferred embodiment, the present invention relates to compounds of general formula (I), supra, in which:
L^{A} represents

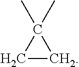

In another embodiment, the present invention relates to compounds of the general formula (I), supra, in which: L^{B} represents —N(H)—C(═O)— or —C(═O)—N(H)—.

In a preferred embodiment, the present invention relates to compounds of the general formula (I), supra, in which:
L^{B} represents *N(H)—C(═O)**;
   wherein “*” indicates the point of attachment to R², and “**” indicates the point of attachment to the phenyl group.

In another embodiment, the present invention relates to compounds of the general formula (I), supra, in which:
R¹ represents a group selected from:
   C₃-C₇-cycloalkyl-, C₄-C₇-cycloalkenyl-, 3- to 10-membered heterocycloalkyl-,
   4- to 10-membered heterocycloalkenyl-, aryl-, heteroaryl-, —N(R⁷)—(C₁-C₆-alkyl); wherein each group is optionally substituted, one or more times, identically or differently, with a substituent selected from: halo-, hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-,
   3- to 10-membered heterocycloalkyl-, —C(═O)R⁹, —C(═O)O—R⁹, —OC(═O)—R⁹,
   —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
   —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹.

In another embodiment, the present invention relates to compounds of the general formula (I), supra, in which:
R¹ represents a group selected from:
   C₃-C₇-cycloalkyl-, 3- to 10-membered heterocycloalkyl-, aryl-, heteroaryl-, —N(R⁷)—(C₁-C₆-alkyl);
   wherein each group is optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl, fluoro-C₁-C₃-alkoxy-,
   C₃-C₇-cycloalkyl-, 3- to 10-membered heterocycloalkyl-,
   —C(═O)R⁹, —C(═O)O—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹, R⁹—S(═O)₂—.

In another embodiment, the present invention relates to compounds of the general formula (I), supra, in which:
R¹ represents a group selected from:
   C₃-C₇-cycloalkyl-, 3- to 10-membered heterocycloalkyl-,
   aryl-, heteroaryl-, —N(R⁷)—(C₁-C₆-alkyl), —N(R⁷)—C(═O)—O—(C₁-C₆-alkyl), —N(R⁷)R⁷;
   wherein said C₃-C₇-cycloalkyl-, 3- to 10-membered heterocycloalkyl-,
   aryl-, heteroaryl-, and —N(R⁷)—(C₁-C₆-alkyl) group is optionally substituted, one or more times,
   identically or differently, with a substituent selected from: halo-, hydroxy-, C₁-C₃-alkyl-, C₁-C₃-alkoxy-, hydroxy-C₁-C₃-alkyl-, C₃-C₇-cycloalkyl-, R⁹—S(═O)₂—.

In another embodiment, the present invention relates to compounds of the general formula (I), supra, in which:
R¹ represents a group selected from:
   3- to 10-membered heterocycloalkyl-, N(R⁷)—(C₁-C₆-alkyl), —N(R⁷)—C(═O)—O—(C₁-C₆-alkyl),
   —N(R⁷)R⁷; wherein said 3- to 10-membered heterocycloalkyl-, and —N(R⁷)—(C₁-C₆-alkyl) group is optionally substituted, one or more times, identically or differently, with a substituent selected from: halo-, hydroxy-, C₁-C₃-alkyl-, C₁-C₃-alkoxy-, hydroxy-C₁-C₃-alkyl-, C₃-C₇-cycloalkyl-, R⁹—S(═O)₂—.

In another embodiment, the present invention relates to compounds of the general formula (I), supra, in which:
R¹ represents a group selected from:
   3- to 10-membered heterocycloalkyl-, or 5- to 6-membered heteroaryl-,
   wherein each group is optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
   C₃-C₅-cycloalkyl-, 3- to 6-membered heterocycloalkyl-,
   —C(═O)R⁹, —C(═O)O—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹, R⁹—S(═O)₂—.

In another embodiment, the present invention relates to compounds of the general formula (I), supra, in which:
R¹ represents a morpholino group, which is attached to L^{A} via its nitrogen atom, and which may be optionally substituted one or two times, identically or differently, with C₁-C₃-alkyl-, or two of said C₁-C₃-alkyl groups together may form a C₁-C₃-alkylene group (forming a bridge between two different ring carbon atoms of said morpholino group),
or
R¹ represents thiomorpholino, 4-cycloproylpiperazino, 4-methylpiperazino, piperidino or pyrazol-1-yl group, said groups being attached to L^{A} via their ring nitrogen atom.

In another embodiment, the present invention relates to compounds of the general formula (I), supra, in which: R¹ represents a

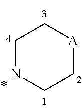

group;
wherein * indicates the point of attachment to L^{A}; wherein A represents a group selected from: —O—, —S—, —S(O)₂—, —NR⁹—; wherein the carbon atoms 1 and 4, 1 and 3, 2 and 3, or 2 and 4 are optionally bridged via a methylene or ethylene group.

In a preferred embodiment, the present invention relates to compounds of the general formula (I), supra, in which:
R¹ represents a morpholino group, which is attached to L^{A} via its nitrogen atom, and which may be optionally substituted one or two times, identically or differently, with C₁-C₃-alkyl-, or two of said C₁-C₃-alkyl-groups together may form a C₁-C₃-alkylene group (forming a bridge between two different ring carbon atoms of said morpholino group).

In a particularly preferred embodiment, the present invention relates to compounds of the general formula (I), supra, in which:
R¹ represents a group selected from:

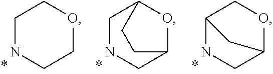

wherein “*” indicates the point of attachment to L^{A}.

In another particularly preferred embodiment, the present invention relates to compounds of the general formula (I), supra, in which:
R¹ represents a group selected from:

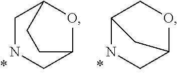

wherein “*” indicates the point of attachment to L^{A}.

In another particularly preferred embodiment, the present invention relates to compounds of the general formula (I), supra, in which:
R¹ represents a group selected from:

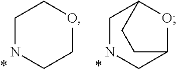

wherein “*” indicates the point of attachment to L^{A}.

In another particularly preferred embodiment, the present invention relates to compounds of the general formula (I), supra, in which:
R¹ represents

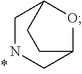

wherein “*” indicates the point of attachment to L^{A}.

In another particularly preferred embodiment, the present invention relates to compounds of the general formula (I), supra, in which:
R¹ represents

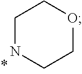

wherein “*” indicates the point of attachment to L^{A}.

In a particularly preferred embodiment, the present invention relates to compounds of the general formula (I), supra, in which:
R¹ represents

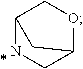

wherein “*” indicates the point of attachment to L^{A}.

In another preferred embodiment, the present invention relates to compounds of the general formula (I), supra, in which:
R² represents:

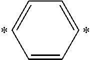

wherein “*” represents the point of attachment to R³ or L^{B}, respectively; wherein said group is optionally substituted, one or more times, identically or differently, with halo- or a C₁-C₃-alkyl-group.

In another preferred embodiment, the present invention relates to compounds of the general formula (I), supra, in which:
R² represents:

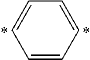

wherein “*” represents the point of attachment to R³ or L^{B}, respectively; wherein said group is optionally substituted, one or more times, identically or differently, with halo-;

In another preferred embodiment, the present invention relates to compounds of the general formula (I), supra, in which:
R² represents:

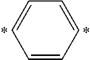

wherein “*” represents the point of attachment to R³ or L^{B}, respectively.

In another embodiment, the present invention relates to compounds of the general formula (I), supra, in which:
R³ represents a phenyl-group,
   said phenyl-group being optionally substituted, one or more times, identically or differently, with a substituent selected from: halo-, hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-,
   C₃-C₇-cycloalkyl-, 3- to 10-membered heterocycloalkyl-,
   —C(═O)R⁹, —C(═O)O—R⁹, —OC(═O)—R⁹,
   —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
   —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹.

In another embodiment, the present invention relates to compounds of the general formula (I), supra, in which:
R³ represents a phenyl-group,
   said phenyl-group being optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
   C₃-C₅-cycloalkyl-, 3- to 6-membered heterocycloalkyl-,
   —C(═O)R⁹, —C(═O)O—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹.

In another embodiment, the present invention relates to compounds of the general formula (I), supra, in which:
R³ represents a phenyl-group;
   said phenyl-group being optionally substituted, one or more times, identically or differently, with a substituent selected from: halo-, hydroxy-, cyano-, C₁-C₃-alkyl-, NH₂—C₁-C₃-alkyl-,
   halo-C₁-C₃-alkyl-, —C(═O)O—R⁹, —N(H)C(═O)R⁹, —N(H)R⁹, —NR¹⁰R⁹;
   or, when two substituents are present ortho to each other on the phenyl-group, said two substituents together form a bridge: *O(CH₂)₂O*, *O(CH₂)O*, *O—C(H)₂—C(H)₂*, *NH(C(═O))NH*, wherein * represent the points of attachment to the phenyl-group.

In another embodiment, the present invention relates to compounds of the general formula (I), supra, in which:
R³ represents a phenyl-group,
   said phenyl-group being optionally substituted, one or more times, identically or differently, with a substituent selected from: halo-, hydroxy-, cyano-, C₁-C₃-alkyl-, NH₂—C₁-C₃-alkyl-, halo-C₁-C₃-alkyl-, —C(═O)O—R⁹, —N(H)C(═O)R⁹, —N(H)R⁹, —NR¹⁰R⁹;
   or, when two substituents are present ortho to each other on the phenyl-group, said two substituents together form a bridge: *O—C(H)₂—C(H)₂*; wherein * represent the points of attachment to the phenyl-group.

In another embodiment, the present invention relates to compounds of the general formula (I), supra, in which:
R³ represents a phenyl-group,
   said phenyl-group being optionally substituted, one or more times, identically or differently, with a substituent selected from: halo-, hydroxy-, cyano-, C₁-C₂-alkyl-, C₁-C₂-alkoxy-, fluoro-C₁-C₂-alkyl-, hydroxy-C₁-C₂-alkyl-, fluoro-C₁-C₂-alkoxy-,
   —C(═O)R⁹, —C(═O)O—R⁹, —N(H)C(═O)R⁹, —N(H)R⁹, —NR¹⁰R⁹, —C(═O)N(H)R⁹, —C(═O)NR¹⁰R⁹.

In another embodiment, the present invention relates to compounds of the general formula (I), supra, in which:
R³ represents a phenyl-group,
   said phenyl-group being optionally substituted, one or two times, identically or differently, with fluoro, chloro, —NH₂ or methoxy.

In a preferred embodiment, the present invention relates to compounds of the general formula (I), supra, in which:
R³ represents a phenyl-group,
   said phenyl-group being optionally substituted one or two times, identically or differently, with fluoro or methoxy.

In another preferred embodiment, the present invention relates to compounds of the general formula (I), supra, in which: R³ represents a para-fluorophenyl-group.

In another preferred embodiment, the present invention relates to compounds of the general formula (I), supra, in which: R³ represents a para-methoxyphenyl-group.

In a particularly preferred embodiment, the present invention relates to compounds of the general formula (I), supra, in which: R³ represents a phenyl-group, said phenyl-group being optionally substituted, one or two times, with fluoro.

In another particularly preferred embodiment, the present invention relates to compounds of the general formula (I), supra, in which: R³ represents an unsubstituted phenyl-group.

In another particularly preferred embodiment, the present invention relates to compounds of the general formula (I), supra, in which: R³ represents an ortho-fluorophenyl-group.

In another particularly preferred embodiment, the present invention relates to compounds of the general formula (I), supra, in which: R³ represents a meta-fluorophenyl-group.

In another particularly preferred embodiment, the present invention relates to compounds of the general formula (I), supra, in which: R³ represents a 2,3-difluorophenyl-group.

In another particularly preferred embodiment, the present invention relates to compounds of the general formula (I), supra, in which: R³ represents a 3,5-difluorophenyl-group.

In another particularly preferred embodiment, the present invention relates to compounds of the general formula (I), supra, in which: R³ represents a 2,6-difluorophenyl-group.

In another embodiment, the present invention relates to compounds of the general formula (I), supra, in which: R⁴ represents a hydrogen atom or a group selected from: C₁-C₆-alkyl-, C₃-C₄-alkenyl-,
C₃-C₄-alkynyl-, —(CH₂)ₘ—C₃-C₇-cycloalkyl, —(CH₂)ₘ—C₄-C₇-cycloalkenyl,
—(CH₂)ₘ-(3 to 10 membered heterocycloalkyl), —(CH₂)ₘ-(4 to 10 membered heterocycloalkenyl), —(CH₂)ₘ-aryl, —(CH₂)ₘ-heteroaryl.

In another embodiment, the present invention relates to compounds of the general formula (I), supra, in which:
R⁴ represents a hydrogen atom or a group selected from:
   C₁-C₆-alkyl-, —(CH₂)ₘ—C₃-C₇-cycloalkyl,
   —(CH₂)ₘ-(3 to 10 membered heterocycloalkyl),
   —(CH₂)ₘ-aryl, —(CH₂)ₘ-heteroaryl.

In another embodiment, the present invention relates to compounds of the general formula (I), supra, in which:
R⁴ represents a hydrogen atom or a group selected from:
   C₁-C₆-alkyl-, —(CH₂)ₘ—C₃-C₇-cycloalkyl, —(CH₂)ₘ-aryl.

In another embodiment, the present invention relates to compounds of the general formula (I), supra, in which: R⁴ represents C₁-C₆-alkyl-.

In another embodiment, the present invention relates to compounds of the general formula (I), supra, in which: R⁴ represents —(CH₂)ₘ—C₃-C₇-cycloalkyl.

In another embodiment, the present invention relates to compounds of the general formula (I), supra, in which: R⁴ represents —(CH₂)ₘ-aryl.

In another embodiment, the present invention relates to compounds of the general formula (I), supra, in which: R⁴ represents —H, C₁-C₃-alkyl- or benzyl-.

In another embodiment, the present invention relates to compounds of the general formula (I), supra, in which: R⁴ represents C₁-C₃-alkyl-.

In a preferred embodiment, the present invention relates to compounds of the general formula (I), supra, in which: R⁴ represents hydrogen.

In another embodiment, the present invention relates to compounds of the general formula (I), supra, in which: R⁵ represents a hydrogen atom or a halogen atom or a group selected from: cyano-, C₁-C₃-alkyl-, C₁-C₃-alkoxy-.

In another embodiment, the present invention relates to compounds of the general formula (I), supra, in which: R⁵ represents a group selected from: cyano-, C₁-C₃-alkyl-, C₁-C₃-alkoxy-.

In another embodiment, the present invention relates to compounds of the general formula (I), supra, in which: R⁵ represents a hydrogen atom or a halogen atom.

In another embodiment, the present invention relates to compounds of the general formula (I), supra, in which: R⁵ represents hydrogen, fluoro or chloro.

In another embodiment, the present invention relates to compounds of the general formula (I), supra, in which: R⁵ represents fluoro or chloro.

In a preferred embodiment, the present invention relates to compounds of the general formula (I), supra, in which: R⁵ represents hydrogen.

In another embodiment, the present invention relates to compounds of the general formula (I), supra, in which: R⁶ represents a group selected from: C₁-C₆-alkyl-, C₂-C₆-alkenyl-, C₂-C₆-alkynyl-C₁-C₆-alkoxy-, halo-, hydroxy-, halo-C₁-C₆-alkyl-, halo-C₁-C₆-alkoxy-, cyano-, -aryl,

-heteroaryl, —N(R⁹)(R¹⁰), —C(═O)—O—R⁹, —C(═O)—N(R⁹)(R¹⁰);
said C₁-C₆-alkyl-, C₂-C₆-alkenyl-, C₂-C₆-alkynyl-, aryl-, heteroaryl- or C₁-C₆-alkoxy-group being optionally substituted, one or more times, identically or differently, with halo-, cyano-, nitro-, hydroxy-, C₁-C₃-alkyl-, C₁-C₃-alkoxy-, halo-C₁-C₃-alkoxy-, hydroxy-C₁-C₃-alkoxy-,
C₁-C₃-alkoxy-C₁-C₃-alkoxy-, C₃-C₇-cycloalkyl-, C₄-C₇-cycloalkenyl-,
3- to 10-membered heterocycloalkyl-, 4- to 10-membered heterocycloalkenyl-, aryl-, heteroaryl-, —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹,
—N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—,
—N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰,
—S(═O)₂N(H)R⁹, —S(═O)₂NR¹⁰R⁹, —S(═O)(═NR¹⁰)R⁹, —S(═O)(═NR¹⁰)R⁹, —N═S(═O)(R¹⁰)R⁹.

In another embodiment, the present invention relates to compounds of the general formula (I), supra, in which: R⁶ represents a group selected from: C₁-C₆-alkyl-, C₁-C₆-alkoxy-, halo-, hydroxy-, fluoro-C₁-C₆-alkyl-, fluoro-C₁-C₆-alkoxy-, phenyl-, 5- to 6-membered heteroaryl-, cyano-, —C(═O)—O—R⁹, —C(═O)—N(R⁹)(R¹⁰); said C₁-C₆-alkyl- or C₁-C₆-alkoxy-group being optionally substituted, one or more times, identically or differently, with C₁-C₃-alkyl-, C₁-C₃-alkoxy-, halo-C₁-C₃-alkoxy-, hydroxy-C₁-C₃-alkoxy-, C₁-C₃-alkoxy-C₁-C₃-alkoxy-, C₃-C₇-cycloalkyl-,

3- to 10-membered heterocycloalkyl-, aryl-, heteroaryl-, —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹, —NR¹⁰R⁹, —C(═O)N(H)R⁹, —C(═O)NR¹⁰R⁹.

In another embodiment, the present invention relates to compounds of the general formula (I), supra, in which: R⁶ represents a group selected from: C₁-C₆-alkyl-, C₁-C₆-alkoxy-, halo-, hydroxy-, fluoro-C₁-C₆-alkyl-, fluoro-C₁-C₆-alkoxy-, cyano-, —C(═O)—O—R⁹, —C(═O)—N(R⁹)(R¹⁰);

said C₁-C₆-alkyl- or C₁-C₆-alkoxy-group being optionally substituted, one or more times, identically or differently, with C₃-C₇-cycloalkyl-, 3- to 10-membered heterocycloalkyl-, aryl-, heteroaryl-, —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹, —NR¹⁰R⁹, —C(═O)N(H)R⁹, —C(═O)NR¹⁰R⁹.

In another embodiment, the present invention relates to compounds of the general formula (I), supra, in which: R⁶ represents a group selected from:

C₁-C₆-alkyl-, C₁-C₆-alkoxy-, C₃-C₆-cycloalkoxy-, halo-, hydroxy-, halo-C₁-C₆-alkyl-,
halo-C₁-C₆-alkoxy-, cyano-, -heteroaryl, —C(═O)—O—R⁹, —C(═O)—N(R⁹)(R¹⁰), R⁹—S—, R⁹—S(═O)—, R⁹—S(═O)₂—;
said C₁-C₆-alkyl- and C₁-C₆-alkoxy-group being optionally substituted, one or more times, identically or differently, with hydroxy-, C₁-C₃-alkoxy-, C₁-C₃-alkoxy-C₁-C₃-alkoxy-, phenyl, —N(H)C(═O)R⁹, —N(H)R⁹, —C(═O)N(H)R⁹, —C(═O)NR¹⁰R⁹, R⁹—S(═O)₂—.

In another embodiment, the present invention relates to compounds of the general formula (I), supra, in which: R⁶ represents a group selected from: C₁-C₆-alkyl-, C₁-C₆-alkoxy-, C₃-C₆-cycloalkoxy-, halo-, hydroxy-, halo-C₁-C₆-alkyl-, halo-C₁-C₆-alkoxy-, cyano-, -heteroaryl, —C(═O)—O—R⁹, —C(═O)—N(R⁹)(R¹⁰), R⁹—S—, R⁹—S(═O)—, R⁹—S(═O)₂—; said C₁-C₆-alkyl- and C₁-C₆-alkoxy-group being optionally substituted, one or more times, identically or differently, with hydroxy-, C₁-C₃-alkoxy-, C₁-C₃-alkoxy-C₁-C₃-alkoxy-, —N(H)C(═O)R⁹, —N(H)R⁹, —C(═O)N(H)R⁹, —C(═O)NR¹⁰R⁹, R⁹—S(═O)₂—.

In another embodiment, the present invention relates to compounds of the general formula (I), supra, in which: R⁶ represents halo-, cyano-, C₁-C₄-alkyl-, fluoro-C₁-C₃-alkyl-,

C₁-C₄-alkoxy- or fluoro-C₁-C₃-alkoxy-, —C(O)NR⁹R¹⁰ or a 5-membered heteroaryl-; wherein said C₁-C₄-alkyl- and C₁-C₄-alkoxy-group may be optionally substituted by one phenyl-group.

In a preferred embodiment, the present invention relates to compounds of the general formula (I), supra, in which: R⁶ represents halogen, C₁-C₄-alkyl-, fluoro-C₁-C₃-alkyl-, C₁-C₄-alkoxy- or fluoro-C₁-C₃-alkoxy-.

In another preferred embodiment, the present invention relates to compounds of the general formula (I), supra, in which: R⁶ represents halogen.

In another preferred embodiment, the present invention relates to compounds of the general formula (I), supra, in which: R⁶ represents fluoro-C₁-C₃-alkyl-.

In another preferred embodiment, the present invention relates to compounds of the general formula (I), supra, in which: R⁶ represents fluoro-C₁-C₃-alkoxy-.

In another preferred embodiment, the present invention relates to compounds of the general formula (I), supra, in which: R⁶ represents C₁-C₄-alkoxy-.

In a particularly preferred embodiment, the present invention relates to compounds of the general formula (I), supra, in which: R⁶ represents chloro, C₁-C₄-alkyl-, methoxy-, trifluoromethoxy- or trifluoromethyl-.

In another particularly preferred embodiment, the present invention relates to compounds of the general formula (I), supra, in which: R⁶ represents chloro.

In another particularly preferred embodiment, the present invention relates to compounds of the general formula (I), supra, in which: R⁶ represents C₁-C₄-alkyl-.

In another particularly preferred embodiment, the present invention relates to compounds of the general formula (I), supra, in which: R⁶ represents methoxy.

In another particularly preferred embodiment, the present invention relates to compounds of the general formula (I), supra, in which: R⁶ represents trifluoromethyl.

In another particularly preferred embodiment, the present invention relates to compounds of the general formula (I), supra, in which: R⁶ represents trifluoromethoxy or tert-butyl;

In another particularly preferred embodiment, the present invention relates to compounds of the general formula (I), supra, in which: R⁶ represents tert-butyl.

In another particularly preferred embodiment, the present invention relates to compounds of the general formula (I), supra, in which: R⁶ represents trifluoromethoxy.

In another particularly preferred embodiment, the present invention relates to compounds of the general formula (I), supra, in which: R⁶ represents —C(═O)—N(R⁹)(R¹⁰).

In another embodiment, the present invention relates to compounds of the general formula (I), supra, in which: R⁷ represents —H or C₁-C₃-alkyl-.

In another embodiment, the present invention relates to compounds of the general formula (I), supra, in which: R⁹ represents —H or C₁-C₃-alkyl-.

In another embodiment, the present invention relates to compounds of the general formula (I), supra, in which: R¹⁰ represents —H or C₁-C₃-alkyl-.

In another embodiment, the present invention relates to compounds of the general formula (I), supra, in which: R¹⁰ represents C₃-C₆-cycloalkyl-; said C₁-C₃-alkyl-group being optionally substituted with C₁-C₃-alkoxy- or —N(R¹²)R¹³.

In another embodiment, the present invention relates to compounds of the general formula (I), supra, in which: R¹¹ represents —H or C₁-C₃-alkyl-.

In another embodiment, the present invention relates to compounds of the general formula (I), supra, in which: R¹², R¹³ represent, independently from each other, —H or C₁-C₃-alkyl-.

In another embodiment, the present invention relates to compounds of the general formula (I), supra, in which: R¹², R¹³ together with the atom they are attached to, form a 3- to 10-membered heterocycloalkyl- or 4- to 10-membered heterocycloalkenyl-group.

In another embodiment, the present invention relates to compounds of the general formula (I), supra, in which: m represents 0, 1, or 2.

In another embodiment, the present invention relates to compounds of the general formula (I), supra, in which: m represents 0 or 1.

In another embodiment, the present invention relates to compounds of the general formula (I), supra, in which: m represents 0.

In another embodiment, the present invention relates to compounds of the general formula (I), supra, in which: m represents 1.

In another embodiment, the present invention relates to compounds of the general formula (Ia):

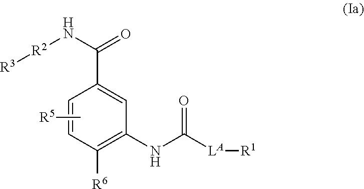

in which R¹, R², R³, R⁵, R⁶ and L^{A} are as defined for general formula (I), supra.

In another embodiment, the present invention relates to compounds of the general formula (Ib):

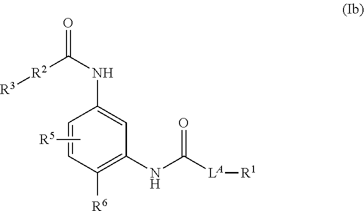

in which R¹, R², R³, R⁵, R⁶ and L^{A} are as defined for general formula (I), supra.

In another embodiment, the present invention relates to compounds of the general formula (Ic):

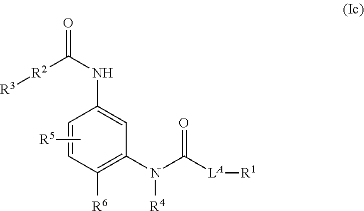

in which R¹, R², R³, R⁴, R⁵, R⁶ and L^{A} are as defined for general formula (I), supra.

It is to be understood that the present invention relates also to any combination of the preferred embodiments described above.

Some examples of combinations are given hereinafter. However, the invention is not limited to these combinations.

In a preferred embodiment, the present invention relates to compounds of general formula (I):

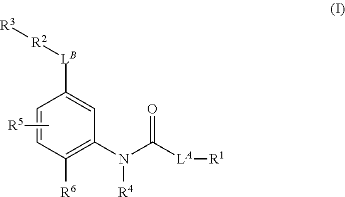

in which:
L^{A} represents a methylene or ethylene group, said methylene or ethylene group being optionally substituted, one or more times, identically or differently, with a substituent selected from:
   hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-, 3- to 10-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
   C₃-C₇-cycloalkyl-, C₄-C₇-cycloalkenyl-, 3- to 10-membered heterocycloalkyl-,
   4- to 10-membered heterocycloalkenyl-, aryl-, heteroaryl-, —N(R⁷)—(C₁-C₆-alkyl);
   wherein each group is optionally substituted, one or more times, identically or differently, with a substituent selected from: halo-, hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-,
   C₃-C₇-cycloalkyl-, 3- to 10-membered heterocycloalkyl-,
   —C(═O)R⁹, —C(═O)O—R⁹, —OC(═O)—R⁹,
   —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
   —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹;
R² represents:

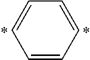

wherein “*” represents the point of attachment to R³ or L^{B}, respectively; wherein said group is optionally substituted, one or more times, identically or differently, with a C₁-C₃-alkyl-group;
R³ represents a phenyl-group,
   said phenyl-group being optionally substituted, one or more times, identically or differently, with a substituent selected from: halo-, hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-,
   C₃-C₇-cycloalkyl-, 3- to 10-membered heterocycloalkyl-,
   —C(═O)R⁹, —C(═O)O—R⁹, —OC(═O)—R⁹,
   —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
   —NR¹⁰R⁹, —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹;
R⁴ represents a hydrogen atom or a group selected from:
   C₁-C₆-alkyl-, C₃-C₄-alkenyl-, C₃-C₄-alkynyl-,
   —(CH₂)ₘ—C₃-C₇-cycloalkyl, —(CH₂)ₘ—C₄-C₇-cycloalkenyl,
   —(CH₂)ₘ-(3- to 10-membered heterocycloalkyl),
   —(CH₂)ₘ-(4- to 10-membered heterocycloalkenyl),
   —(CH₂)ₘ-aryl, —(CH₂)ₘ-heteroaryl;
R⁵ represents a hydrogen atom or a halogen atom or a group selected from:
   cyano-, C₁-C₃-alkyl-, C₁-C₃-alkoxy-;
R⁶ represents a group selected from:
   C₁-C₆-alkyl-, C₂-C₆-alkenyl-, C₂-C₆-alkynyl-
   C₁-C₆-alkoxy-, halo-, hydroxy-, cyano-, aryl-,
   heteroaryl-, —N(R⁹)(R¹⁰), —C(═O)—O—R⁹, —C(═O)—N(R⁹)(R¹⁰);
   said C₁-C₆-alkyl-, C₂-C₆-alkenyl-, C₂-C₆-alkynyl-, aryl-, heteroaryl- or C₁-C₆-alkoxy-group being optionally substituted, one or more times, identically or differently, with halo-, cyano-, nitro-, hydroxy-, C₁-C₃-alkyl-, C₁-C₃-alkoxy-, halo-C₁-C₃-alkoxy-, hydroxy-C₁-C₃-alkoxy-, C₁-C₃-alkoxy-C₁-C₃-alkoxy-,
   C₃-C₇-cycloalkyl-, C₄-C₇-cycloalkenyl-, 3- to 10-membered heterocycloalkyl-,
   4- to 10-membered heterocycloalkenyl-,
   aryl-, heteroaryl-, —C(═O)R⁹, —C(═O)O—R⁹, —OC(═O)—R⁹, —N(H)C(═O)R⁹, —N(R¹⁰)C(═O)R⁹,
   —N(H)C(═O)NR¹⁰R⁹, —N(R¹¹)C(═O)NR¹⁰R⁹, —N(H)R⁹, —NR¹⁰R⁹,
   —C(═O)N(H)R⁹, —C(═O)NR¹⁰R⁹, R⁹—S—, R⁹—S(═O)—, R⁹—S(═O)₂—,
   —N(H)S(═O)R⁹, —N(R¹⁰)S(═O)R⁹, —S(═O)N(H)R⁹, —S(═O)NR¹⁰R⁹,
   —N(H)S(═O)₂R⁹, —N(R⁹)S(═O)₂R¹⁰, —S(═O)₂N(H)R⁹, —S(═O)₂NR¹⁰R⁹,
   —S(═O)(═NR¹⁰)R⁹, —S(═O)(═NR¹⁰)R⁹, —N═S(═O)(R¹⁰)R⁹;
R⁷ represents —H or C₁-C₃-alkyl-;
R⁹, R¹⁰, R¹¹
   represent, independently from each other, —H or C₁-C₃-alkyl-;
or
R⁹R¹⁹ together with the atom or the group of atoms they are attached to, form a 3- to 10-membered heterocycloalkyl- or 4- to 10-membered heterocycloalkenyl-group;
m represents 0, 1, or 2;
or a tautomer, an N-oxide, a hydrate, a solvate, or a salt thereof, or a mixture of same.

In a preferred embodiment, the present invention relates to compounds of general formula (I), supra, in which:
L^{A} represents a methylene or ethylene group, said methylene or ethylene group being optionally substituted, one or more times, identically or differently, with a substituent selected from:
   hydroxy-, cyano-, C₁-C₃-alkyl-, C₁-C₃-alkoxy-, fluoro-C₁-C₃-alkyl-,
   hydroxy-C₁-C₃-alkyl-, fluoro-C₁-C₃-alkoxy-, C₃-C₇-cycloalkyl-,
   3- to 10-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
   C₃-C₇-cycloalkyl-, 3- to 10-membered heterocycloalkyl-, aryl-, heteroaryl-, —N(R⁷)—(C₁-C₆-alkyl);
   wherein each group is optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
   C₃-C₇-cycloalkyl-, 3- to 10-membered heterocycloalkyl-,
   —C(═O)R⁹, —C(═O)O—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹, R⁹—S(═O)₂—;
R² represents:

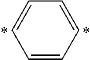

wherein “*” represents the point of attachment to R³ or L^{B}, respectively; wherein said group is optionally substituted, one or more times, identically or differently, with a C₁-C₃-alkyl-group;
R³ represents a phenyl-group,
   said phenyl-group being optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
   C₃-C₅-cycloalkyl-, 3- to 6-membered heterocycloalkyl-,
   —C(═O)R⁹, —C(═O)O—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹;
R⁴ represents a hydrogen atom or a group selected from:
   C₁-C₆-alkyl-, —(CH₂)ₘ—C₃-C₇-cycloalkyl,
   —(CH₂)ₘ-(3- to 10-membered heterocycloalkyl),
   —(CH₂)ₘ-aryl, —(CH₂)ₘ-heteroaryl;
R⁵ represents a hydrogen atom or a halogen atom or a group selected from:
   cyano-, C₁-C₃-alkyl-, C₁-C₃-alkoxy-;
R⁶ represents a group selected from:

C₁-C₆-alkyl-, C₁-C₆-alkoxy-, halo-, hydroxy-, fluoro-C₁-C₆-alkyl-, fluoro-C₁-C₆-alkoxy-, phenyl-, 5- to 6-membered heteroaryl-, cyano-, —C(═O)—O—R⁹, —C(═O)—N(R⁹)(R¹⁰);
said C₁-C₆-alkyl- or C₁-C₆-alkoxy-group being optionally substituted, one or more times, identically or differently, with
hydroxy-, C₁-C₃-alkyl-, C₁-C₃-alkoxy-, halo-C₁-C₃-alkoxy-,
hydroxy-C₁-C₃-alkoxy-, C₁-C₃-alkoxy-C₁-C₃-alkoxy-,
C₃-C₇-cycloalkyl-, 3- to 10-membered heterocycloalkyl-, aryl-, heteroaryl-,
—C(═O)R⁹, —C(═O)O—R⁹, —OC(═O)—R⁹, —N(H)C(═O)R⁹, —N(R¹⁰)C(═O)R⁹,
—N(H)C(═O)NR¹⁰R⁹, —N(R¹¹)C(═O)NR¹⁰R⁹, —N(H)R⁹, —NR¹⁰R⁹,
—C(═O)N(H)R⁹, —C(═O)NR¹⁰R⁹;
R⁷ represents —H or C₁-C₃-alkyl-;
R⁹, R¹⁰, R¹¹
   represent, independently from each other, —H or C₁-C₃-alkyl-;
m represents 0, 1, or 2;
or a tautomer, an N-oxide, a hydrate, a solvate, or a salt thereof, or a mixture of same.

In another preferred embodiment, the present invention relates to compounds of general formula (I), supra, in which:
L^{A} represents a methylene group, said methylene group being optionally substituted, one or more times, identically or differently, with a substituent selected from:
   cyano-, hydroxy-, C₁-C₃-alkyl-, C₁-C₃-alkoxy-, fluoro-C₁-C₃-alkyl-,
   hydroxy-C₁-C₃-alkyl-, C₃-C₅-cycloalkyl-, 3- to 6-membered heterocycloalkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
   3- to 10-membered heterocycloalkyl-, 5- to 6-membered heteroaryl-;
   wherein each group is optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
   C₃-C₅-cycloalkyl-, 3- to 6-membered heterocycloalkyl-,
   —C(═O)R⁹, —C(═O)O—R⁹, —N(H)C(═O)R⁹, —N(R¹⁰)C(═O)R⁹, —N(H)C(═O)NR¹⁰R⁹,
   —N(R¹¹)C(═O)NR¹⁰R⁹, —N(H)R⁹, —NR¹⁰R⁹, —C(═O)N(H)R⁹, —C(═O)NR¹⁰R⁹, R⁹—S(═O)₂—;
R² represents:

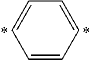

wherein “*” represents the point of attachment to R³ or L^{B}, respectively; wherein said group is optionally substituted, one or more times, identically or differently, with a C₁-C₃-alkyl-group;
R³ represents a phenyl-group,
   said phenyl-group being optionally substituted, one or more times, identically or differently, with a substituent selected from: halo-, hydroxy-, cyano-, C₁-C₂-alkyl-, C₁-C₂-alkoxy-, fluoro-C₁-C₂-alkyl-, hydroxy-C₁-C₂-alkyl, fluoro-C₁-C₂-alkoxy-,
   —C(═O)R⁹, —C(═O)O—R⁹, —N(H)C(═O)R⁹, —N(H)R⁹, —NR¹⁰R⁹, —C(═O)N(H)R⁹, —C(═O)NR¹⁰R⁹;
R⁴ represents a hydrogen atom or a group selected from:
   C₁-C₆-alkyl-, —(CH₂)ₘ—C₃-C₇-cycloalkyl, —(CH₂)ₘ-aryl;
R⁵ represents a hydrogen atom or a halogen atom or a group selected from:
   cyano-, C₁-C₃-alkyl-, C₁-C₃-alkoxy-;
R⁶ represents a group selected from:
   C₁-C₆-alkyl-, C₁-C₆-alkoxy-, halo-, hydroxy-, fluoro-C₁-C₆-alkyl-, fluoro-C₁-C₆-alkoxy-, cyano-, —C(═O)—O—R⁹, —C(═O)—N(R⁹)(R¹⁰);
   said C₁-C₆-alkyl-, or C₁-C₆-alkoxy-group being optionally substituted, one or more times, identically or differently, with
   C₃-C₇-cycloalkyl-, 3- to 10-membered heterocycloalkyl-, aryl-, heteroaryl-,
   —C(═O)R⁹, —C(═O)O—R⁹, —OC(═O)—R⁹, —N(H)C(═O)R⁹, —N(R¹⁰)C(═O)R⁹,
   —N(H)C(═O)NR¹⁰R⁹, —N(R¹¹)C(═O)NR¹⁰R⁹, —N(H)R⁹, —NR¹⁰R⁹,
   —C(═O)N(H)R⁹, —C(═O)NR¹⁰R⁹;
R⁹, R¹⁰, R¹¹
   represent, independently from each other, —H or C₁-C₃-alkyl-;
m represents 0 or 1;
or a tautomer, an N-oxide, a hydrate, a solvate, or a salt thereof, or a mixture of same.

In another preferred embodiment, the present invention relates to compounds of general formula (I), supra, in which:
L^{A} represents a methylene group, said methylene group being optionally substituted, one or more times, identically or differently, with a substituent selected from:
   hydroxy-, C₁-C₃-alkyl-, C₁-C₃-alkoxy-, hydroxy-C₁-C₃-alkyl-;
   or, when two substituents are present at the same carbon atom, the two substituents, together with the carbon atom they are attached to, may form a
   C₃-C₆-cycloalkyl- or 3- to 6-membered heterocycloalkyl-ring; wherein said ring is optionally substituted one or more times, identically or differently, with a substituent selected from:
   halo-, hydroxy-, cyano-, C₁-C₃-alkyl-, C₁-C₃-alkoxy-;
L^{B} represents —N(H)—C(═O)— or —C(═O)—N(H)—;
R¹ represents a morpholino group, which is attached to L^{A} via its nitrogen atom, and which may be optionally substituted one or two times, identically or differently, with C₁-C₃-alkyl-, or two of said C₁-C₃-alkyl-groups together may form a C₁-C₃-alkylene group (forming a bridge between two different ring carbon atoms of said morpholino group);
or
R¹ represents thiomorpholino, 4-cyclopropylpiperazino, 4-methylpiperazino, piperidino or pyrazol-1-yl group, said groups being attached to L^{A} via their ring nitrogen atom;
R² represents:

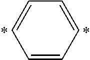

wherein “*” represents the point of attachment to R³ or L^{B}, respectively;
R³ represents a phenyl-group,
   said phenyl-group being optionally substituted, one or two times, identically or differently, with fluoro, chloro, —NH₂ or methoxy;
R⁴ represents hydrogen, C₁-C₃-alkyl- or benzyl-;
R⁵ represents hydrogen, fluoro or chloro;
R⁶ represents halo-, cyano-, C₁-C₄-alkyl-, fluoro-C₁-C₃-alkyl-, C₁-C₄-alkoxy- or fluoro-C₁-C₃-alkoxy-, —C(═O)NR⁹R¹⁰ or 5-membered heteroaryl-,
   wherein said C₁-C₄-alkyl- and C₁-C₄-alkoxy-group may be optionally substituted by one phenyl-group;
or a tautomer, an N-oxide, a hydrate, a solvate, or a salt thereof, or a mixture of same.

In another preferred embodiment, the present invention relates to compounds of general formula (I), supra, in which:
L^{A} represents methylene, said methylene group being optionally substituted one or two times, identically or differently, with C₁-C₃-alkyl-,
   wherein, if said methylene is substituted with two C₁-C₃-alkyl-groups, these may, together with the carbon atom they are attached to, form a C₃-C₆-cycloalkyl-ring;
L^{B} represents —N(H)—C(═O)— or —C(═O)—N(H)—;
R¹ represents a morpholino group, which is attached to L^{A} via its nitrogen atom, and which may be optionally substituted one or two times, identically or differently, with C₁-C₃-alkyl-,
   or two of said C₁-C₃-alkyl-groups together may form a C₁-C₃-alkylene group (forming a bridge between two different ring carbon atoms of said morpholino group);
R² represents:

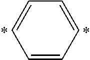

wherein “*” represents the point of attachment to R³ or L^{B}, respectively;
R³ represents a phenyl-group,
   said phenyl-group being optionally substituted one or two times, identically or differently, with fluoro or methoxy;
R⁴ represents hydrogen;
R⁵ represents hydrogen;
R⁶ represents halogen, C₁-C₄-alkyl-, fluoro-C₁-C₃-alkyl-, C₁-C₄-alkoxy- or fluoro-C₁-C₃-alkoxy-;
or a tautomer, an N-oxide, a hydrate, a solvate, or a salt thereof, or a mixture of same.

In another preferred embodiment, the present invention relates to compounds of general formula (I), supra, in which:
L^{A} represents methylene, said methylene group being optionally substituted one or two times, identically or differently, with C₁-C₃-alkyl-,
   wherein, if said methylene is substituted with two C₁-C₃-alkyl-groups, these may, together with the carbon atom they are attached to, form a C₃-C₆-cycloalkyl-ring;
L^{B} represents —N(H)—C(═O)— or —C(═O)—N(H)—;
R¹ represents a morpholino group, which is attached to L^{A} via its nitrogen atom, and which may be optionally substituted one or two times, identically or differently, with C₁-C₃-alkyl-,
   or two of said C₁-C₃-alkyl-groups together may form a C₁-C₃-alkylene group (forming a bridge between two different ring carbon atoms of said morpholino group);
R² represents:

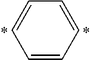

wherein “*” represents the point of attachment to R³ or L^{B}, respectively;
R³ represents a phenyl-group,
   said phenyl-group being optionally substituted one or two times, identically or differently, with fluoro or methoxy;
R⁴ represents hydrogen;
R⁵ represents hydrogen;
R⁶ represents trifluoromethoxy;
or a tautomer, an N-oxide, a hydrate, a solvate, or a salt thereof, or a mixture of same.

In a particularly preferred embodiment, the present invention relates to compounds of general formula (I), supra, in which:
L^{A} represents —CH₂—, —CH(CH₃)—, —C(CH₃)₂— or

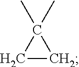

wherein the cycloproypl-ring is optionally substituted one or more times, identically or differently, with a substituent selected from: halo-, hydroxy-, cyano-, C₁-C₃-alkyl-, C₁-C₃-alkoxy-.
L^{B} represents —N(H)—C(═O)— or —C(═O)—N(H)—;
R¹ represents a group selected from:

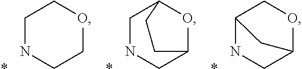

wherein “*” indicates the point of attachment to L^{A};
R² represents:

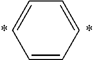

wherein “*” represents the point of attachment to R³ or L^{B}, respectively;
R³ represents a phenyl-group,
   said phenyl-group being optionally substituted, one or two times, with fluoro;
R⁴ represents hydrogen;
R⁵ represents hydrogen;
R⁶ represents chloro, C₁-C₄-alkyl-, methoxy-, trifluoromethoxy- or trifluoromethyl-;
or a tautomer, an N-oxide, a hydrate, a solvate, or a salt thereof, or a mixture of same.

In another particularly preferred embodiment, the present invention relates to compounds of general formula (I), supra, in which:
L^{A} represents —CH₂—, —CH(CH₃)—, —C(CH₃)₂— or

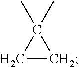

L^{B} represents —N(H)—C(═O)— or —C(═O)—N(H)—;
R¹ represents a group selected from:

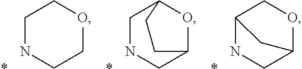

wherein “*” indicates the point of attachment to L^{A};
R² represents:

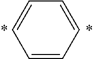

wherein “*” represents the point of attachment to R³ or L^{B}, respectively;
R³ represents a phenyl-group,
   said phenyl-group being optionally substituted, one or two times, with fluoro;
R⁴ represents hydrogen;
R⁵ represents hydrogen;
R⁶ represents trifluoromethoxy;
or a tautomer, an N-oxide, a hydrate, a solvate, or a salt thereof, or a mixture of same.

In another particularly preferred embodiment, the present invention relates to compounds of general formula (I), supra, in which:
L^{A} represents —CH₂—, —CH(CH₃)— or

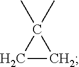

L^{B} represents —N(H)—C(═O)— or —C(═O)—N(H)—;
R¹ represents a group selected from:

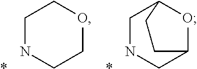

wherein “*” indicates the point of attachment to L^{A};
R² represents:

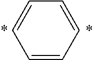

wherein “*” represents the point of attachment to R³ or L^{B}, respectively;
R³ represents a phenyl-group,
   said phenyl-group being optionally substituted, one or two times, with fluoro;
R⁴ represents hydrogen;
R⁵ represents hydrogen;
R⁶ represents trifluoromethoxy or tert-butyl;
or a tautomer, an N-oxide, a hydrate, a solvate, or a salt thereof, or a mixture of same.

In another particularly preferred embodiment, the present invention relates to compounds of general formula (I), supra, in which:
L^{A} represents —CH₂— or —CH(CH₃)—;
L^{B} represents —N(H)—C(═O)— or —C(═O)—N(H)—;
R¹ represents a group selected from:

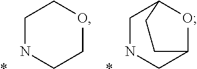

wherein “*” indicates the point of attachment to L^{A};
R² represents:

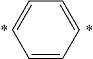

wherein “*” represents the point of attachment to R³ or L^{B}, respectively;
R³ represents a phenyl-group,
   said phenyl-group being optionally substituted, one or two times, with fluoro;
R⁴ represents hydrogen;
R⁵ represents hydrogen;
R⁶ represents trifluoromethoxy;
or a tautomer, an N-oxide, a hydrate, a solvate, or a salt thereof, or a mixture of same.

In another preferred embodiment, the present invention relates to compounds of general formula

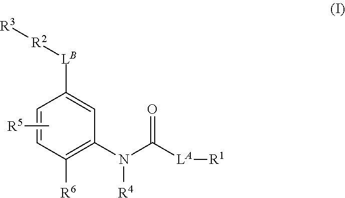

in which:
L^{A} represents a methylene or ethylene group, said methylene or ethylene group being optionally substituted, one or more times, identically or differently, with a substituent selected from: C₁-C₃-alkyl- and halo-C₁-C₃-alkyl-;
   or, when two substituents are present at the same carbon atom, the two substituents, together with the carbon atom they are attached to, may form a
   C₃-C₆-cycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
   C₃-C₇-cycloalkyl-, 3- to 10-membered heterocycloalkyl-,
   aryl-, heteroaryl-, —N(R⁷)—(C₁-C₆-alkyl), —N(R⁷)—C(═O)—O—(C₁-C₆-alkyl), —N(R⁷)R⁷;
   wherein said C₃-C₇-cycloalkyl-, 3- to 10-membered heterocycloalkyl-,
   aryl-, heteroaryl-, and —N(R⁷)—(C₁-C₆-alkyl) group is optionally substituted, one or more times, identically or differently, with a substituent selected from: halo-, hydroxy-, C₁-C₃-alkyl-, C₁-C₃-alkoxy-, hydroxy-C₁-C₃-alkyl-, C₃-C₇-cycloalkyl-, R⁹—S(═O)₂—;
R² represents:

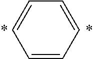

wherein “*” represents the point of attachment to R³ or L^{B}, respectively; wherein said group is optionally substituted, one or more times, identically or differently, with halo- or a C₁-C₃-alkyl-group;
R³ represents a phenyl-group;
   said phenyl-group being optionally substituted, one or more times, identically or differently, with a substituent selected from: halo-, hydroxy-, cyano-, C₁-C₃-alkyl-, NH₂—C₁-C₃-alkyl-, halo-C₁-C₃-alkyl-, —C(═O)O—R⁹, —N(H)C(═O)R⁹, —N(H)R⁹, —NR¹⁰R⁹;
   or, when two substituents are present ortho to each other on the phenyl-group, said two substituents together form a bridge: *O(CH₂)₂O*, *O(CH₂)O*, *O—C(H)₂—C(H)₂*, *NH(C(═O))NH*, wherein * represent the points of attachment to the phenyl-group;
R⁴ represents a hydrogen atom or a group selected from:
   C₁-C₃-alkyl, —(CH₂)-phenyl;
R⁵ represents a hydrogen atom or a halogen atom;
R⁶ represents a group selected from:
   C₁-C₆-alkyl-, C₁-C₆-alkoxy-, C₃-C₆-cycloalkoxy-, halo-, hydroxy-, halo-C₁-C₆-alkyl-, halo-C₁-C₆-alkoxy-, cyano-, -heteroaryl, —C(═O)—O—R⁹, —C(═O)—N(R⁹)(R¹⁰), R⁹—S—, R⁹—S(═O)—, R⁹—S(═O)₂—;
   said C₁-C₆-alkyl- and C₁-C₆-alkoxy-group being optionally substituted, one or more times, identically or differently, with
   hydroxy-, C₁-C₃-alkoxy-, C₁-C₃-alkoxy-C₁-C₃-alkoxy-, phenyl,
   —N(H)C(═O)R⁹, —N(H)R⁹, —C(═O)N(H)R⁹, —C(═O)NR¹⁰R⁹, R⁹—S(═O)₂—;
R⁷ represents —H or C₁-C₃-alkyl-;
R⁹, R¹⁰, R¹¹
   represent, independently from each other, —H, C₁-C₃-alkyl- or C₃-C₆-cycloalkyl-;
   said C₁-C₃-alkyl-group being optionally substituted with C₁-C₃-alkoxy- or —N(R¹²)R¹³;
or
R⁹R¹⁰ together with the atom or the group of atoms they are attached to, form a 3- to 10-membered heterocycloalkyl- or 4- to 10-membered heterocycloalkenyl-group;
R¹², R¹³
   represent, independently from each other, —H or C₁-C₃-alkyl-;
or
R¹², R¹³ together with the atom they are attached to, form a 3- to 10-membered heterocycloalkyl- or 4- to 10-membered heterocycloalkenyl-group;
or a tautomer, an N-oxide, a hydrate, a solvate, or a salt thereof, or a mixture of same.

In another preferred embodiment, the present invention relates to compounds of general formula (I):

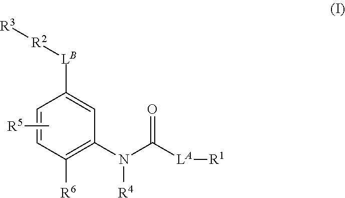

in which:
L^{A} represents a methylene group, said methylene group being optionally substituted, one or more times, identically or differently, with a substituent selected from:
   C₁-C₃-alkyl- and halo-C₁-C₃-alkyl-;
   or, when two substituents are present at the same carbon atom, the two substituents, together with the carbon atom they are attached to, may form a
   C₃-C₄-cycloalkyl-ring;
L^{B} represents —N(H)—C(═O)— or —C(═O)—N(H)—;
R¹ represents a group selected from:
   3- to 10-membered heterocycloalkyl-, N(R⁷)—(C₁-C₆-alkyl), —N(R⁷)—C(═O)—O—(C₁-C₆-alkyl), —N(R⁷)R⁷; wherein said 3- to 10-membered heterocycloalkyl-, and —N(R⁷)—(C₁-C₆-alkyl) group is optionally substituted, one or more times, identically or differently, with a substituent selected from: halo-, hydroxy-, C₁-C₃-alkyl-, C₁-C₃-alkoxy-, hydroxy-C₁-C₃-alkyl-, C₃-C₇-cycloalkyl-, R⁹—S(═O)₂—;
R² represents:

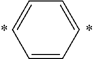

wherein “*” represents the point of attachment to R³ or L^{B}, respectively; wherein said group is optionally substituted, one or more times, identically or differently, with halo-;
R³ represents a phenyl-group,
   said phenyl-group being optionally substituted, one or more times, identically or differently, with a substituent selected from: halo-, hydroxy-, cyano-, C₁-C₃-alkyl-, NH₂—C₁-C₃-alkyl-, halo-C₁-C₃-alkyl-, —C(═O)O—R⁹, —N(H)C(═O)R⁹, —N(H)R⁹, —NR¹⁰R⁹;
   or, when two substituents are present ortho to each other on the phenyl-group, said two substituents together form a bridge: *O—C(H)₂—C(H)₂*; wherein * represent the points of attachment to the phenyl-group;
R⁴ represents a hydrogen atom;
R⁵ represents a hydrogen atom or a halogen atom;
R⁶ represents a group selected from:
   C₁-C₆-alkyl-, C₁-C₆-alkoxy-, C₃-C₆-cycloalkoxy-, halo-, hydroxy-, halo-C₁-C₆-alkyl-, halo-C₁-C₆-alkoxy-, cyano-, -heteroaryl, —C(═O)—O—R⁹, —C(═O)—N(R⁹)(R¹⁰), R⁹—S—, R⁹—S(═O)—, R⁹—S(═O)₂—;
   said C₁-C₆-alkyl- and C₁-C₆-alkoxy-group being optionally substituted, one or more times, identically or differently, with hydroxy-, C₁-C₃-alkoxy-, C₁-C₃-alkoxy-C₁-C₃-alkoxy-, —N(H)C(═O)R⁹, —N(H)R⁹, —C(═O)N(H)R⁹, —C(═O)NR¹⁰R⁹, R⁹—S(═O)₂—;
R⁷ represents —H or C₁-C₃-alkyl-;
R⁹, R¹⁰, R¹¹
   represent, independently from each other, —H, C₁-C₃-alkyl- or C₃-C₆-cycloalkyl-;
   said C₁-C₃-alkyl-group being optionally substituted with C₁-C₃-alkoxy- or —N(R¹²)R¹³;
or
R⁹R¹⁰ together with the atom or the group of atoms they are attached to, form a 3- to 10-membered heterocycloalkyl- or 4- to 10-membered heterocycloalkenyl-group;
R¹², R¹³
   represent, independently from each other, —H or C₁-C₃-alkyl-;
or
R¹², R¹³ together with the atom they are attached to, form a 3- to 10-membered heterocycloalkyl- or 4- to 10-membered heterocycloalkenyl-group;
or a tautomer, an N-oxide, a hydrate, a solvate, or a salt thereof, or a mixture of same.

It is to be understood that the present invention relates also to any combination of the preferred embodiments described above.

More particularly still, the present invention covers compounds of general formula (I) which are disclosed in the Examples section of this text, infra.

In accordance with another aspect, the present invention covers methods of preparing compounds of the present invention, said methods comprising the steps as described in the Experimental Section herein.

In a preferred embodiment, the present invention relates to a method of preparing a compound of general formula (I), supra, said method comprising the step of allowing an intermediate compound of general formula (VI):

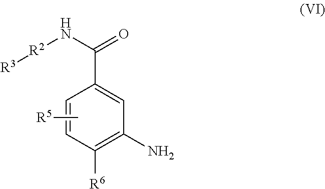

in which R², R³, R⁵, and R⁶ are as defined for general formula (I), supra;
to react with a carboxylic acid HO₂C-L^{A}-R¹ or the corresponding acyl chloride Cl—C(═O)-L^{A}-R¹, wherein L^{A} and R¹ are as defined for the compounds of general formula (I), supra; or alternatively
to react with suitable reagents, such as Cl—C(═O)-L^{A}-LG, in which L^{A} is as defined for the compounds of general formula (I), and LG stands for a leaving group, preferably chloro or bromo, and subsequently with agents suitable for the introduction of R¹, exemplified by but not limited to cyclic secondary amines;
thereby giving, upon optional deprotection, a compound of general formula (Ia):

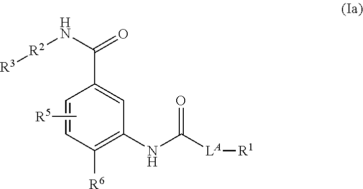

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XI):

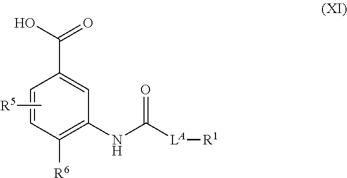

in which L^{A}, R¹, R⁵, and R⁶ are as defined for general formula (I), supra;
to react with a compound of general formula R³R²NH₂, in which R² and R³ are as defined for the compounds of general formula (I), supra;
thereby giving, upon optional deprotection, a compound of general formula (Ia):

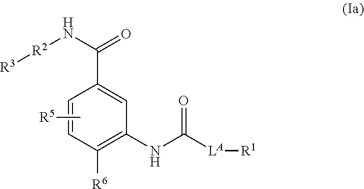

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XIa):

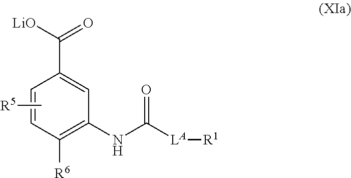

in which L^{A}, R¹, R⁵, and R⁶ are as defined for general formula (I), supra;
to react with a compound of general formula R³R²NH₂, in which R² and R³ are as defined for the compounds of general formula (I), supra;
thereby giving, upon optional deprotection, a compound of general formula (Ia):

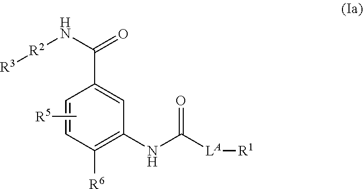

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XVII):

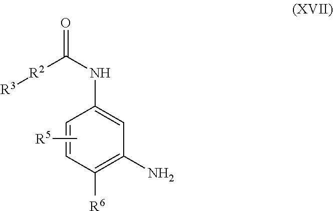

in which R², R³, R⁵, and R⁶ are as defined for general formula (I), supra;
to react with a carboxylic acid HO₂C-L^{A}-R¹ or the corresponding acyl chloride Cl—C(═O)-L^{A}-R¹, wherein L^{A} and R¹ are as defined for the compounds of general formula (I), supra; or alternatively
to react with suitable reagents, such as Cl—C(═O)-L^{A}-LG, in which L^{A} is as defined for the compounds of general formula (I), and LG stands for a leaving group, preferably chloro or bromo, and subsequently with agents suitable for the introduction of R¹, exemplified by but not limited to cyclic secondary amines;
thereby giving, upon optional deprotection, a compound of general formula (Ib):

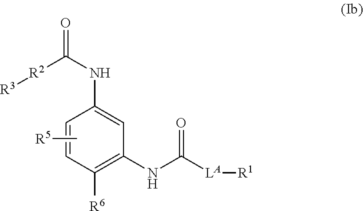

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XXII):

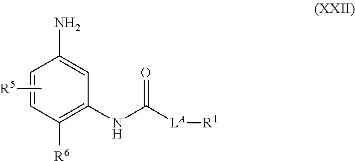

in which L^{A}, R¹, R⁵ and R⁶ are as defined for general formula (I), supra;
to react with a carboxylic acid HO₂C—R²—R³, wherein R² and R³ are as defined for the compounds of general formula (I), supra; or alternatively
to react with a carboxylic acid X—R²—CO₂H, in which R² is as defined for the compounds of general formula (I), supra, and subsequently subjected to a palladium catalysed coupling reaction, such as a Suzuki coupling, with R³—X′, in which R³ is as defined for the compounds of general formula (I), supra. In X—R²—CO₂H and R³—X′, both X and X′ represent groups enabling palladium catalysed coupling reactions, such as chloro, bromo, iodo, trifluoromethylsulfonyloxy, or a boronic acid or an ester thereof, with the proviso that if X represents a boronic ester or an ester thereof, X′ stands for bromo, iodo, or trifluoromethylsulfonyloxy and the like, or vice versa;
thereby giving, upon optional deprotection, a compound of general formula (Ib):

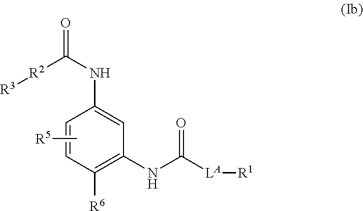

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XXIV):

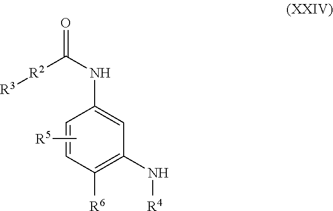

in which R², R³, R⁴, R⁵ and R⁶ are as defined for general formula (I), supra;
to react with a carboxylic acid HO₂C-L^{A}-R¹ or the corresponding acyl chloride Cl—C(═O)-L^{A}-R¹, wherein L^{A} and R¹ are as defined for the compounds of general formula (I), supra;
thereby giving, upon optional deprotection, a compound of general formula (Ic):

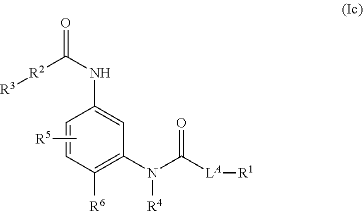

in which L^{A}, R¹, R², R³, R⁴, R⁵ and R⁶ are as defined for the compounds of general formula (I), supra.

In accordance with another embodiment, the present invention also relates to a method of preparing a compound of general formula (I), supra, said method comprising the step of allowing an intermediate compound of general formula (XXV):

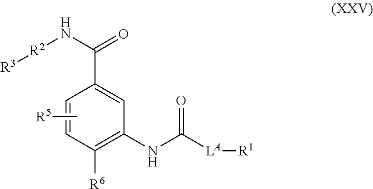

in which L^{A}, R¹, R², R⁵ and R⁶ are as defined for general formula (I), supra;
to react with a compound of general formula R³—X′, wherein R³ is as defined for the compounds of general formula (I), supra;
wherein both, X and X′ represent groups enabling palladium catalysed coupling reactions, such as chloro, bromo, iodo, trifluoromethylsulfonyloxy, or a boronic acid or an ester thereof, with the proviso that if X represents a boronic ester or an ester thereof, X′ stands for chloro, bromo, iodo, or trifluoromethylsulfonyloxy and the like, or vice versa.
thereby giving, upon optional deprotection, a compound of general formula (Ia):

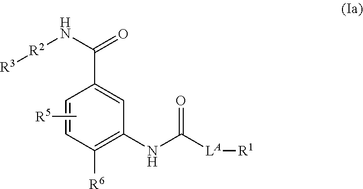

in which L^{A}, R¹, R², R³, R⁴, R⁵ and R⁶ are as defined for the compounds of general formula (I), supra.

In accordance with a further aspect, the present invention covers intermediate compounds which are useful in the preparation of compounds of the present invention of general formula (I), particularly in the method described herein. In particular, the present invention covers intermediate compounds of general formula (VI):

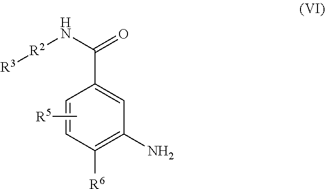

in which R², R³, R⁵, and R⁶ are as defined for general formula (I), supra.

The present invention also covers intermediate compounds of general formula (XI):

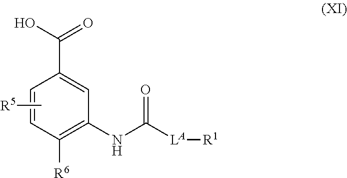

in which L^{A}, R¹, R⁵, and R⁶ are as defined for the compounds of general formula (I), supra.

The present invention also covers intermediate compounds of general formula (XIa):

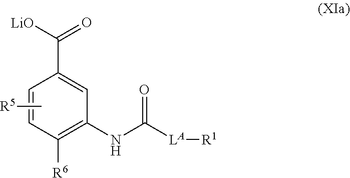

in which L^{A}, R¹, R⁵, and R⁶ are as defined for general formula (I), supra.

The present invention also covers intermediate compounds of general formula (XVII):

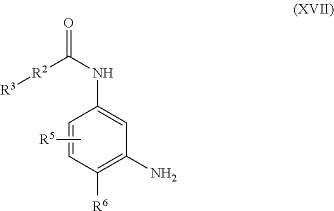

in which R², R³, R⁵, and R⁶ are as defined for general formula (I), supra.

The present invention also covers intermediate compounds of general formula (XXII):

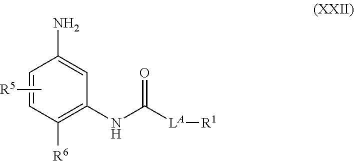

in which L^{A}, R¹, R⁵ and R⁶ are as defined for general formula (I), supra.

The present invention also covers intermediate compounds of general formula (XXIV):

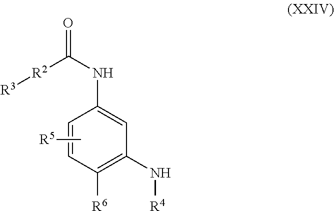

in which R², R³, R⁴, R⁵ and R⁶ are as defined for general formula (I), supra.

The present invention also covers intermediate compounds of general formula (XXV):

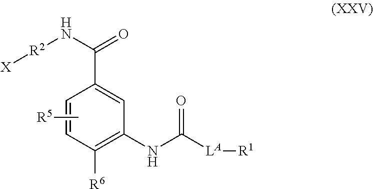

in which L^{A}, R¹, R², R⁵ and R⁶ are as defined for general formula (I), supra, and X represents a group enabling palladium catalysed coupling reactions, such as chloro, bromo, iodo, trifluoromethylsulfonyloxy, or a boronic acid or an ester thereof.

In accordance with yet another aspect, the present invention covers the use of the intermediate compounds of general formula (VI):

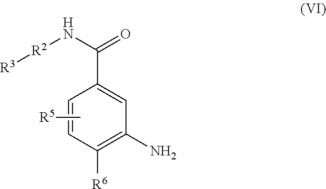

in which R², R³, R⁵, and R⁶ are as defined for general formula (I) supra,
for the preparation of a compound of general formula (I) as defined supra.

In accordance with yet another aspect, the present invention covers the use of the intermediate compounds of general formula (XI):

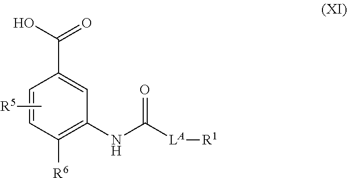

in which L^{A}, R¹, R⁵, and R⁶ are as defined for the compounds of general formula (I) supra,
for the preparation of a compound of general formula (I) as defined supra.

In accordance with yet another aspect, the present invention covers the use of the intermediate compounds of general formula (XIa):

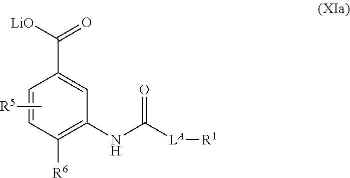

in which L^{A}, R¹, R⁵, and R⁶ are as defined for general formula (I) supra,
for the preparation of a compound of general formula (I) as defined supra.

In accordance with yet another aspect, the present invention covers the use of the intermediate compounds of general formula (XVII):

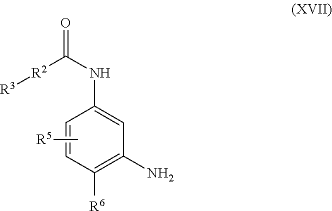

in which R², R³, R⁵, and R⁶ are as defined for general formula (I) supra,
for the preparation of a compound of general formula (I) as defined supra.

In accordance with yet another aspect, the present invention covers the use of the intermediate compounds of general formula (XXII):

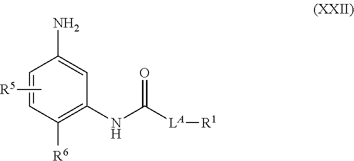

in which L^{A}, R¹, R⁵ and R⁶ are as defined for general formula (I) supra,
for the preparation of a compound of general formula (I) as defined supra.

In accordance with yet another aspect, the present invention covers the use of the intermediate compounds of general formula (XXIV):

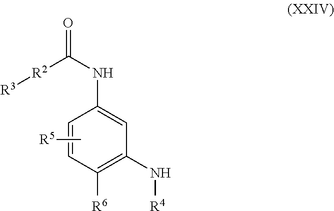

in which R², R³, R⁴, R⁵ and R⁶ are as defined for general formula (I) supra,
for the preparation of a compound of general formula (I) as defined supra.

In accordance with yet another aspect, the present invention covers the use of the intermediate compounds of general formula (XXV):

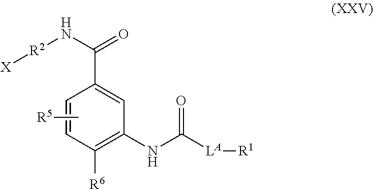

in which L^{A}, R¹, R², R⁵ and R⁶ are as defined for general formula (I), supra, and X represents a group enabling palladium catalysed coupling reactions, such as chloro, bromo, iodo, trifluoromethylsulfonyloxy, or a boronic acid or an ester thereof;
for the preparation of a compound of general formula (I) as defined supra.

### GENERAL SYNTHESIS OF THE COMPOUNDS OF THE INVENTION

The following paragraphs outline a variety of synthetic approaches suitable to prepare compounds of formulae (Ia), (Ib) and (Ic), in which L^{A}, R¹, R², R³, R⁵ and R⁶ are as defined for the compounds of general formula (I), supra. Formulae (Ia) and (Ib), in which R⁴ represents hydrogen, both constitute subsets of formula (I) in that they feature different orientations of the amide linker L^{B}, which stands for —NH—C(═O)— in formula (Ia) whilst representing —C(═O)—NH— in formula (Ib), as shown in Scheme A. In formula (Ic), L^{B} represents —C(═O)—NH—, alike formula (Ib), and R⁴ is as defined for the compounds of general formula (I), supra, but different from hydrogen.

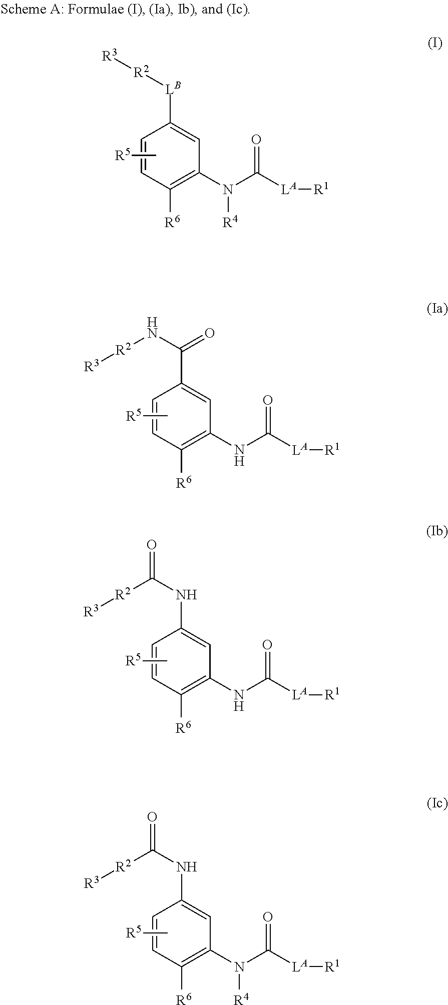

In addition to the routes described below, also other routes may be used to synthesise the target compounds, in accordance with common general knowledge of a person skilled in the art of organic synthesis. The order of transformations exemplified in the following Schemes is therefore not intended to be limiting, and suitable synthesis steps from various schemes can be combined to form additional synthesis sequences. In addition, interconversion of any of the substituents R¹, R², R³, R⁴, R⁵ and/or R⁶, can be achieved before and/or after the exemplified transformations. These modifications can be such as the introduction of protective groups, cleavage of protective groups, reduction or oxidation of functional groups, halogenation, metallation, metal catalysed coupling reactions, substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 3^{rd} edition, Wiley 1999). Specific examples are described in the subsequent paragraphs. Further, it is possible that two or more successive steps may be performed without work-up being performed between said steps, e.g. a “one-pot” reaction, as it is well-known to a person skilled in the art.

Scheme B outlines the preparation of compounds of the formula (Ia), in which L^{A}, R¹, R², R³, R⁵, and R⁶ are as defined for the compounds of general formula (I), supra, starting from meta-nitrobenzoic acid derivatives (II), in which R⁵ and R⁶ are as defined for the compounds of general formula (I), which can be converted into the corresponding benzoyl chlorides (III), by treatment with a suitable chlorinating agent, such as oxalyl chloride. Benzoic acid derivatives of the formula (II) are well known to the person skilled in the art, and are often commercially available. Said benzoyl chlorides of the formula (III) can be subsequently converted into amides of the general formula (V), e.g. directly by aminolysis with amines R³—R²—NH₂, in which R² and R³ are as defined for the compounds of general formula (I). Alternatively, amides of the formula (V) can be accomplished in two steps by aminolysis of (III) using an amine X—R²—NH₂, in which R² is as defined for the compounds of general formula (I), giving rise to amides of the formula (IV). Said amides can be subsequently coupled with R³—X′, in which R³ is as defined for the compounds of general formula (I), in a palladium catalysed coupling reaction such as a Suzuki coupling to furnish amides of general formula (V). In X—R²—NH₂ and R³—X′, both X and X′ represent groups enabling palladium catalysed coupling reactions, such as chloro, bromo, iodo, trifluoromethylsulfonyloxy, or a boronic acid or an ester thereof, with the proviso that if X represents a boronic ester or an ester thereof, X′ stands for chloro, bromo, iodo, or trifluoromethylsulfonyloxy and the like, or vice versa.

The nitro group present in said amides (V) is then reduced by treatment with a suitable reducing agent, such as titanium(III)chloride, or hydrogenation in the presence of a suitable catalyst, e.g. palladium on charcoal, to give anilines of the formula (VI). Said anilines of the formula (VI) are then elaborated into compounds of the formula (Ia). This can be accomplished directly by reacting a compound of the formula (VI) with a carboxylic acid HO₂C-L^{A}-R¹, wherein L^{A} and R¹ are as defined for the compounds of general formula (I), in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide. Alternatively, the transformation of anilines (VI) into compounds of the formula (Ia) can be performed by reaction of anilines (VI) with suitable reagents, such as Cl—C(═O)-L^{A}-LG, in which L^{A} is as defined for the compounds of general formula (I), and LG stands for a leaving group, preferably chloro or bromo, to give the corresponding compounds of formula (VII), which are subsequently reacted with agents suitable for the introduction of R¹, exemplified by but not limited to cyclic secondary amines, to give compounds of the formula (Ia).

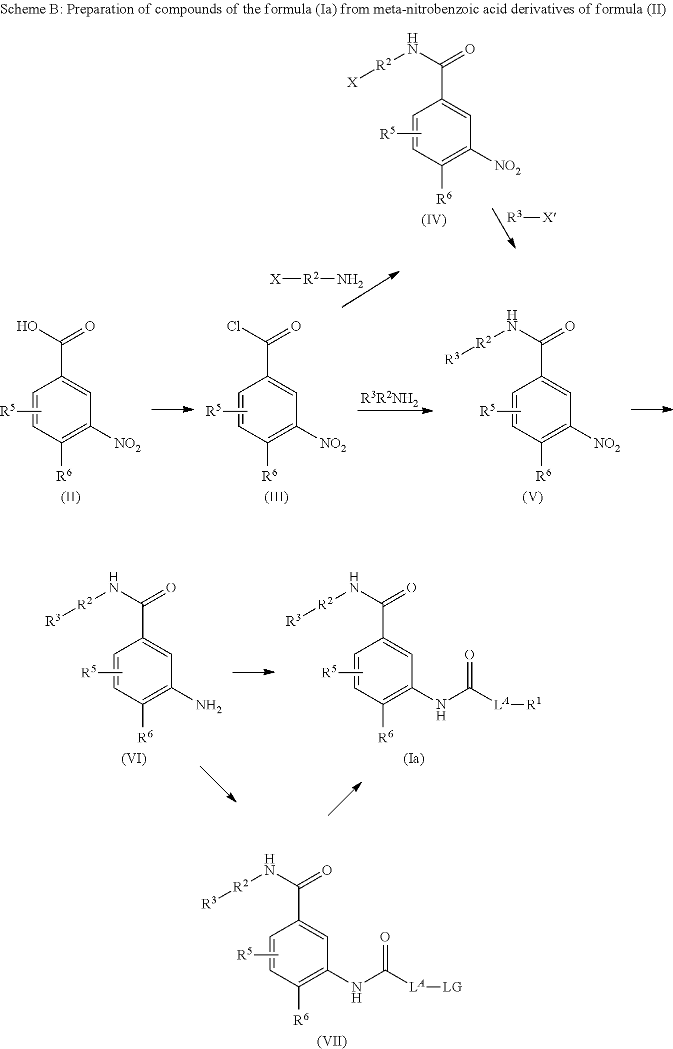

Alternatively, compounds of the formula (Ia) can be prepared starting from meta-aminobenzoic acid derivatives of formula (VIII), in which R⁵ and R⁶ are as defined for the compounds of general formula (I), supra, as outlined in Scheme C. Said meta-aminobenzoic acid derivatives of formula (VIII) are well known to the person skilled in the art and are commercially available in many cases. Compounds of formula (VIII) can be reacted with an amine R³R²NH₂, in which R² and R³ are as defined for the compounds of general formula (I), supra, in a standard amide coupling reaction, to give amide derivatives of formula (VI). Said compounds of formula (VI) can also be obtained by coupling the aformentioned acids of formula (VIII) with an amine X—R²—NH₂, in which R² is as defined for the compounds of general formula (I), supra, giving rise to amides of the formula (IX). These are subsequently subjected to a palladium catalysed coupling reaction, such as a Suzuki coupling, with R³—X′, in which R³ is as defined for the compounds of general formula (I), in order to furnish amides of general formula (VI), respectively. In X—R²—NH₂ and R³—X′, both X and X′ represent groups enabling palladium catalysed coupling reactions, such as chloro, bromo, iodo, trifluoromethylsulfonyloxy, or a boronic acid or an ester thereof, with the proviso that if X represents a boronic ester or an ester thereof, X′ stands for chloro, bromo, iodo, or trifluoromethylsulfonyloxy and the like, or vice versa. Amides of the formula (VI) are subsequently converted into compounds of formula (Ia) as described supra in context with Scheme B.

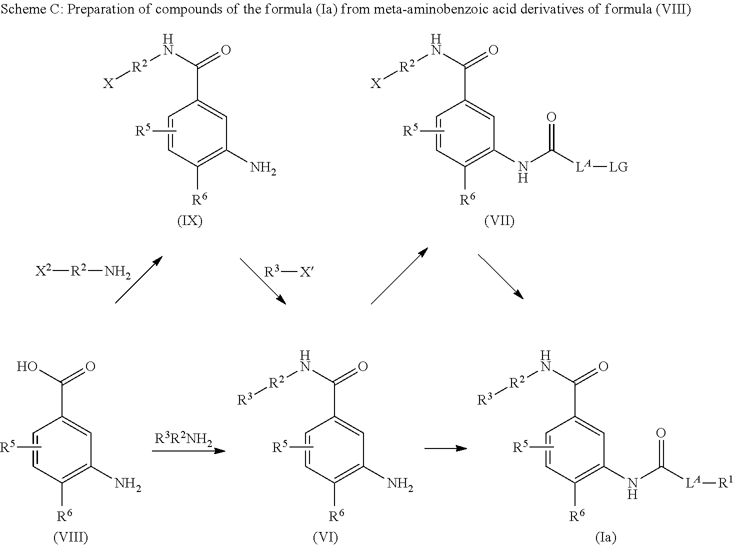

The sequence of synthetic steps can be varied as outlined in Scheme D, in order to convert meta-aminobenzoic acid derivatives of formula (VIII), in which R⁵ and R⁶ are as defined for the compounds of general formula (I), into compounds of the formula (Ia). Said benzoic acid derivatives of the formula (VIII) can be converted into compounds of the formula (X), in which LG stands for a leaving group, preferably chloro or bromo, followed e.g. by aminolysis of compounds of the formula (X) using reagents suitable for the introduction of R¹, exemplified by but not limited to suitable cyclic secondary amines, to give compounds of the formula (XI). Subsequently, the carboxy group present in compounds of the formula (XI) can be coupled with an amine R³R²NH₂, in which R² and R³ are as defined for the compounds of general formula (I), supra, in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide, to afford compounds of the formula (Ia).

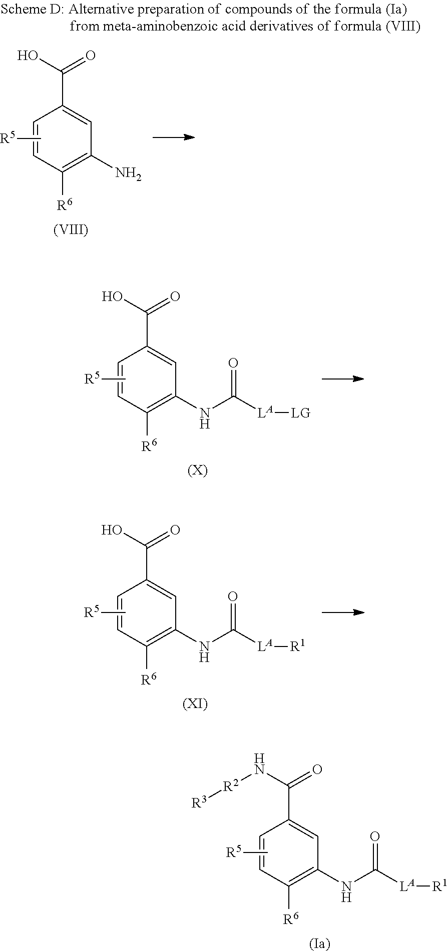

Instead of said benzoic acid derivatives of formula (VIII), also the corresponding ester analogues of formula (XII), in which R⁵ and R⁶ are as defined for the compounds of general formula (I), and in which R^{E} stands for a C₁-C₆-alkyl group, preferably methyl or ethyl, can be employed in a similar fashion in order to prepare compounds of the formula (Ia), as outlined in Scheme E. Esters of the formula (XII) are well known to the person skilled in the art, and are commercially available in many cases. Elaboration of said benzoic acid esters of formula (XII) into compounds of formula (XIV), in which R¹ is as defined for the compounds of general formula (I), supra, can proceed via compounds of formula (XIII), in which LG stands for a leaving group, preferably chloro or bromo, and can be performed analogously as described in context with Scheme D. Subsequently, the ester group present in compounds of formula (XIV) can be saponified by reaction with lithium hydroxide to yield the lithium salt of the formula (XIa). Said lithium salt of formula (XIa) is then converted into compounds of formula (Ia).

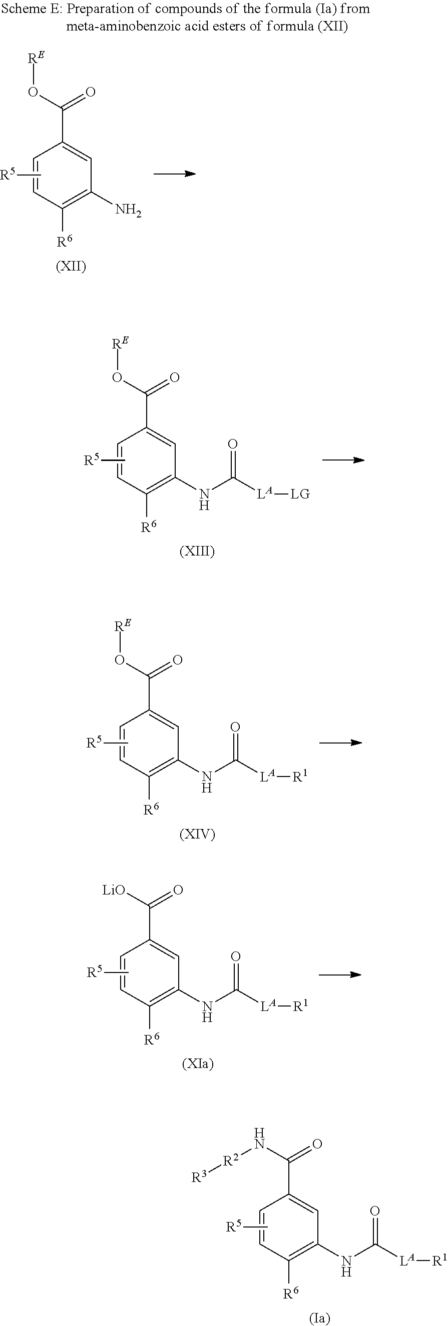

A first approach to compounds of the formula (Ib) from meta-nitroaniline derivatives of formula (XV), in which R⁵ and R⁶ are as defined for the compounds of general formula (I), supra, is outlined in Scheme F. Said meta-nitroaniline derivatives of formula (XV) are well known to the person skilled in the art, and are often commercially available. They can be converted into amide derivatives of formula (XVI) e.g. by a reacting with a carboxylic acid chloride R³—R²—C(═O)Cl, in which R² and R³ are as defined for the compounds of general formula (I), supra, in the presence of a suitable base, such as potassium carbonate, and in a suitable solvent, such as acetonitrile. Basic solvents, such as pyridine, can take over both the role of a base and of a solvent, respectively. Alternatively, conversion of (XV) into (XVI) can be performed via standard amide coupling reactions. The nitro group present in amides of the formula (XVI) can be subsequently reduced e.g. by hydrogenation in the presence of a suitable catalyst, e.g. palladium on charcoal, to give the corresponding aniline derivatives of formula (XVII). Said anilines of the formula (XVII) can then be elaborated into compounds of the formula (Ib). This can be accomplished directly by reacting a compound of the formula (XVII) with a carboxylic acid HO₂C-L^{A}-R¹, wherein L^{A} and R¹ are as defined for the compounds of general formula (I), in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide. Alternatively, the transformation of anilines (XVII) into compounds of the formula (Ia) can be performed by reaction of anilines (XVII) with suitable reagents, such as Cl—C(═O)-L^{A}-LG, in which L^{A} is as defined for the compounds of general formula (I), and LG stands for a leaving group, preferably chloro or bromo, to give the corresponding compounds of formula (XVIII), which are subsequently reacted with agents suitable for the introduction of R¹, exemplified by but not limited to cyclic secondary amines, to give compounds of the formula (Ib).

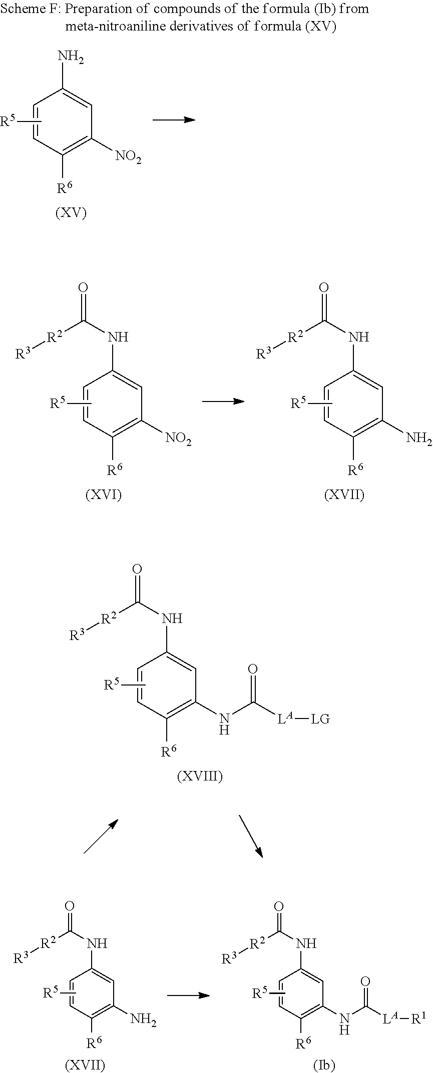

Scheme G outlines an approach complimentary to Scheme F as an alternative synthesis route for compounds of the formula (Ib), from meta-nitroaniline derivatives of formula (XIX), in which R⁵ and R⁶ are as defined for the compounds of general formula (I), supra, and which differ from the compounds of formula (XV) by the inverse arrangement of their nitro and amino groups, respectively. Said meta-nitroaniline derivatives of formula (XIX) are well known to the person skilled in the art, and are often commercially available. They can be converted into amide derivatives of formula (XX), in which L^{A} is as defined for the compounds of general formula (I), supra, and in which LG stands for a leaving group, preferably chloro or bromo, by a reacting with a carboxylic acid LG-L^{A}—CO₂H, in a standard amide coupling reaction. Said amides of the formula (XX) can be subsequently converted into compounds of the formula (XXI), in which R¹ is as defined for the compounds of general formula (I), supra, using reagents suitable for the introduction of R¹, exemplified by but not limited to cyclic secondary amines. The nitro group present in amides of the formula (XXI) is then reduced e.g. by hydrogenation in the presence of a suitable catalyst, e.g. palladium on charcoal, to give the corresponding aniline derivatives of formula (XXII). Compounds of formula (XXII) can be reacted with a carboxylic acid R³R²CO₂H, wherein R² and R³ are as defined for the compounds of general formula (I), supra, in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide, to give compounds of the formula (Ib). The compounds of formula (Ib) can also be obtained by coupling the aformentioned anilines of formula (XXII) with a carboxylic acid X—R²—CO₂H, in which R² is as defined for the compounds of general formula (I), supra, giving rise to amides of the formula (XXIII). These can be subsequently subjected to a palladium catalysed coupling reaction, such as a Suzuki coupling, with R³—X′, in which R³ is as defined for the compounds of general formula (I), in order to furnish compounds of the formula (Ib), respectively. In X—R²—CO₂H and R³—X′, both X and X′ represent groups enabling palladium catalysed coupling reactions, such as chloro, bromo, iodo, trifluoromethylsulfonyloxy, or a boronic acid or an ester thereof, with the proviso that if X represents a boronic ester or an ester thereof, X′ stands for chloro, bromo, iodo, or trifluoromethylsulfonyloxy and the like, or vice versa.

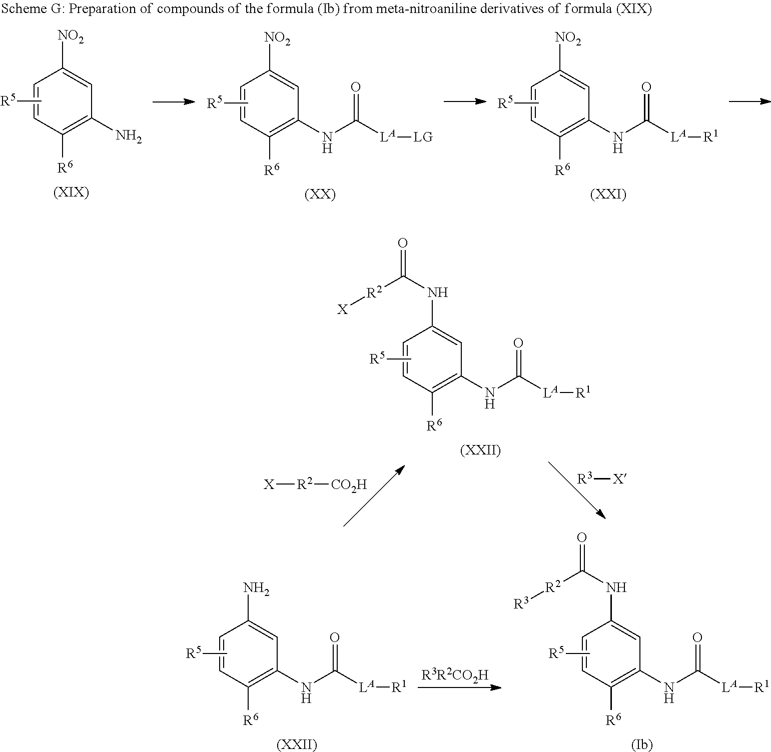

Scheme H illustrates the introduction of R⁴ groups different from hydrogen. In order so to do, primary anilines of the formula (XVII), in which L^{A}, R¹, R², R³, R⁵, and R⁶ are as defined for the compounds of general formula (I), supra, and which can be prepared according to Scheme F, can be converted into secondary anilines of the formula (XXIV), in which R⁴ is as defined for the compounds of general formula (I), supra, but different from hydrogen. This can be accomplished by various methods known to the person skilled in the art, such as a reductive amination with an aldehyde suitable to confer R⁴, e.g. benzaldehyde for R⁴=benzyl, in the presence of a suitable borohydride reagent, such as sodium triacetoxyborohydride, and in the presence of a suitable acid, such as acetic acid, in a suitable solvent, such as a chlorinated hydrocarbon, preferably dichloromethane. The resulting compounds of the formula (XXIV) are subsequently elaborated into compounds of the formula (Ic), in which L^{A}, R¹, R², R³, R⁴, R⁵ and R⁶ are as defined for the compounds of general formula (I), supra, with the proviso that R⁴ is different from hydrogen.

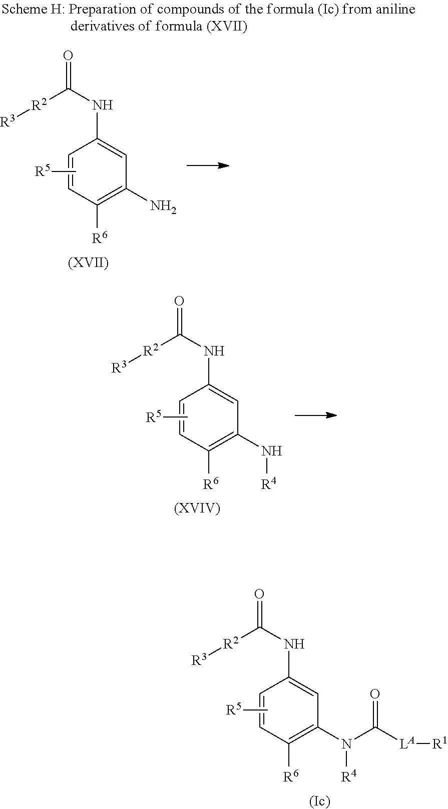

Further details (reaction conditions, suitable solvents etc.) can be obtained from the experimental section below.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

### EXPERIMENTAL SECTION

The following table lists the abbreviations used in this paragraph, and in the examples section.

| Abbreviation | Meaning |
|---|---|
| anh | anhydrous |
| br. | broad signal (in NMR data) |
| d | day(s) |
| DAD | Diode Array Detector |
| DCM | dichloromethane |
| DME | 1,2-dimethoxyethane |
| DMF | N,N-dimethylformamide |
| DMSO | dimethyl sulfoxide |
| ELSD | Evaporative Light Scattering Detector |
| ESI | electrospray ionisation |
| EtOAc | ethyl acetate |
| h | hour |
| HPLC, LC | high performance liquid chromatography |
| m/z | mass-to-charge ratio (in mass spectrum) |
| mc | multiplet centred |
| MeOH | methanol |
| min | Minute |
| MPLC | medium pressure liquid chromatography |
| MS | mass spectroscopy |
| neg | negative |
| NMR | nuclear magnetic resonance |
| PE | petroleum ether |
| pos | positive |
| ppm | Chemical shift δ in parts per million |
| PYBOP | (1H-benzotriazol-1-yloxy)(tripyrrolidin-1-yl)phosphonium |
| | hexafluorophosphate |
| Rt | retention time |
| rt | room temperature |
| THF | tetrahydrofurane |
| TLC | thin layer chromatography |

### Methods:

### Method 1:

Instrument: Waters Acquity UPLC-MS SQD; column: Acquity UPLC BEH C18 1.7 50×2.1 mm; Eluent A: water+0.05% vol. formic acid (98%), Eluent B: acetonitrile+0.05% vol. formic acid (98%); gradient: 0-1.6 min 1-99% B, 1.6-2.0 min 99% B; rate 0.8 mL/min; temperature: 60° C.; DAD scan: 210-400 nm; ELSD.

### Method 2:

Instrument: Waters Autopurificationsystem SQD; column: Waters XBrigde C18 5μ 100×30 mm; water+0.1% vol. formic acid (99%)/acetonitrile gradient; temperature: room temperature; injection: 2500 μL; DAD scan: 210-400 nm.

### Method 3:

Instrument: Waters Acquity UPLC-MS SQD; column: Acquity UPLC BEH C18 1.7 50×2.1 mm; Eluent A: water+0.2% vol. ammonia (32%), Eluent B: acetonitrile; gradient: 0-1.6 min 1-99% B, 1.6-2.0 min 99% B; rate 0.8 mL/min; temperature: 60° C.; DAD scan: 210-400 nm; ELSD.

### Method 4:

Instrument: Waters Acquity UPLC-MS SQD; column: Acquity UPLC BEH C18 1.7 50×2.1 mm; Eluent A: water+0.1% vol. formic acid (99%), Eluent B: acetonitrile; gradient: 0-1.6 min 1-99% B, 1.6-2.0 min 99% B; rate 0.8 mL/min; temperature: 60° C.; DAD scan: 210-400 nm; ELSD.

### Method 5:

Instrument: Waters Autopurificationsystem SQD; column: Waters XBrigde C18 5μ 100×30 mm; water+0.2% vol. ammonia (32%)/acetonitrile gradient; temperature: room temperature; injection: 2500 μL; DAD scan: 210-400 nm.

### Method 6:

Instrument: JASCO P2000 Polarimeter; wavelength 589 nm; temperature: 20° C.; integration time 10 s; path length 100 mm.

### Method 7:

Instrument: Acquity UPLC from Waters; mass detector: LCT from Micromass (now Waters); column: Kinetex C18 from Phenomenex, 50×2.1 mm, 2.6 μm particle, 60° C.; solvent: A: water+0.05% formic acid; B: acetonitrile+0.05% formic acid; injection: 0.5 μl; rate: 1.3 mL/min; gradient 99% A, 1% B until 1.9 min linear to 1% A, 99% B; 1.9-2.10 min unchanged; until 2.20 min back to 99% A, 1% B.

### Intermediates

### Example 1A

### 4-methoxy-3-nitrobenzoyl chloride

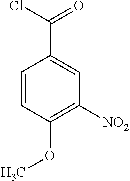

3.00 g (15.2 mmol) of 4-methoxy-3-nitrobenzoic acid were stirred in 20 mL of dichloromethane at room temperature. 59 μL (0.76 mmol) of DMF and 2.66 mL (30.4 mmol) of oxalyl chloride were added and the mixture was stirred for additional 2 h at 50° C. after the gas formation had stopped. 1.33 mL (15.2 mmol) of oxalyl chloride were added and the mixture was stirred for 6 h at 50° C. Then the solvents were evaporated and the remaining material was provided in 20 mL of dichloromethane at room temperature. 59 μL (0.76 mmol) of DMF and 2.66 mL (30.4 mmol) of oxalyl chloride were added and the mixture was stirred for additional 2 h at 50° C. after the gas formation had stopped. After concentration, 3.25 g of raw material were obtained which were used without further purification.

### Example 2A

### 3-nitro-4-(trifluoromethyl)benzoyl chloride

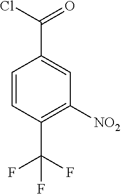 [SMILES: O=C(Cl)c1ccc(C(F)(F)F)c([N+](=O)[O-])c1]

5.00 g (21.3 mmol) of 3-nitro-4-(trifluoromethyl)benzoic acid were stirred in 28 mL of dichloromethane at room temperature. 0.08 mL (1.06 mmol) of DMF and 3.7 mL (42.5 mmol) of oxalyl chloride were added, and the mixture was stirred for additional 1.5 h at 50° C. after the gas formation had stopped. The mixture was left at room temperature over night. After concentration, 4.58 g of raw material were obtained, which were used without further purification.

### Example 3A

### 2-chloro-4-methoxy-5-nitrobenzoyl chloride

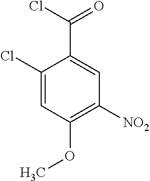

2.00 g (8.64 mmol) of 2-chloro-4-methoxy-5-nitrobenzoic acid were stirred in 15 mL of dichloromethane at room temperature. 33 μL (0.43 mmol) of DMF and 1.51 mL (17.3 mmol) of oxalyl chloride were added, and the mixture was stirred for 2 h at 50° C. 1.51 mL (17.3 mmol) of oxalyl chloride were added at room temperature, and the mixture was stirred for 1 h at 50° C. over night at room temperature. After concentration, 2.10 g of raw material were obtained, which were used without further purification.

### Example 4A

### N-(biphenyl-4-yl)-4-methoxy-3-nitrobenzamide

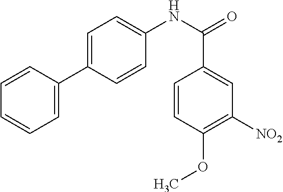 [SMILES: COc1ccc(C(=O)Nc2ccc(-c3ccccc3)cc2)cc1[N+](=O)[O-]]

In one flask 66.7 mg (394 μmol) of biphenyl-4-amine and 82 μL (591 μmol) of triethylamine in 5.1 mL of THF were stirred at room temperature. 100 mg of 85% purity (394 μmol) of the compound from example 1A were added, and the mixture was stirred for 68 h. In another flask 2.76 g (16.3 mmol) of biphenyl-4-amine and 3.4 mL (24.4 mmol) of triethylamine in 208 mL of THF were stirred at room temperature. 4.13 g of 85% purity (16.3 mmol) of the compound from example 1A were added, and the mixture was stirred for 68 h. Both mixtures were poured into water together and extracted with ethyl acetate. The combined organic phases were washed with brine, dried over sodium sulfate, filtered and concentrated. 5.67 g (96% of theory) of the title compound were obtained.

¹H-NMR (300 MHz, DMSO-d₆): δ [ppm]=4.03 (s, 3H), 7.30-7.39 (m, 1H), 7.42-7.50 (m, 2H), 7.54 (d, 1H), 7.64-7.73 (m, 4H), 7.83-7.91 (m, 2H), 8.31 (dd, 1H), 8.55 (d, 1H), 10.43 (s, 1H).

LC-MS (Method 3): Rₜ=1.31 min; MS (ESIpos): m/z=349 [M+H]⁺.

### Example 5A

### N-(biphenyl-4-yl)-3-nitro-4-(trifluoromethyl)benzamide

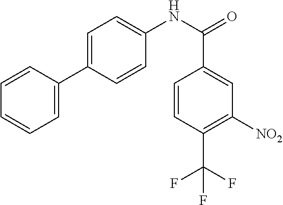 [SMILES: O=C(Nc1ccc(-c2ccccc2)cc1)c1ccc(C(F)(F)F)c([N+](=O)[O-])c1]

3.06 g (18.1 mmol) of biphenyl-4-amine and 3.8 mL (27.1 mmol) of triethylamine in 230 mL of THF were stirred at room temperature. 4.58 g (18.1 mmol) of the compound from example 2A were added, and the mixture was stirred for 68 h. The mixture was poured into 300 mL of water and extracted with ethyl acetate. The combined organic phases were washed with brine, dried over sodium sulfate, filtered and concentrated. 7.27 g (99% of theory) of the title compound were obtained.

¹H-NMR (400 MHz, DMSO-d₆): δ [ppm]=7.32-7.39 (m, 1H), 7.43-7.50 (m, 2H), 7.66-7.75 (m, 4H), 7.85-7.91 (m, 2H), 8.26 (d, 1H), 8.47 (d, 1H), 8.68-8.70 (m, 1H), 10.76 (s, 1H).

LC-MS (Method 3): Rₜ=1.43 min; MS (ESIpos): m/z=387 [M+H]⁺.

### Example 6A

### N-(biphenyl-4-yl)-2-chloro-4-methoxy-5-nitrobenzamide

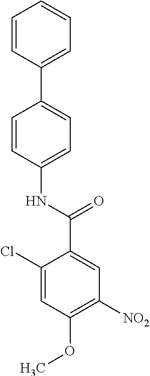 [SMILES: COc1cc(Cl)c(C(=O)Nc2ccc(-c3ccccc3)cc2)cc1[N+](=O)[O-]]

1.42 g (8.40 mmol) of biphenyl-4-amine and 1.76 mL (12.6 mmol) of triethylamine in 100 mL of THF were stirred at room temperature. 2.10 g (8.40 mmol) of the compound from example 3A were added, and the mixture was stirred at room temperature over night. The mixture was poured into water and extracted with ethyl acetate. The combined organic phases were washed with 1N aqueous hydrogen chloride solution and saturated aqueous sodium bicarbonate solution, dried over sodium sulfate, filtered and concentrated. 1.22 g (35% of theory) of the title compound were obtained, which were used without further purification.

¹H-NMR (300 MHz, DMSO-d₆): δ [ppm]=4.03 (s, 3H), 7.30-7.39 (m, 1H), 7.41-7.50 (m, 2H), 7.60-7.73 (m, 5H), 7.75-7.84 (m, 2H), 8.24 (s, 1H), 10.68 (s, 1H).

LC-MS (Method 1): Rₜ=1.35 min; MS (ESIpos): m/z=383 [M+H]⁺.

### Example 7A

### 3-amino-N-(biphenyl-4-yl)-4-methoxybenzamide

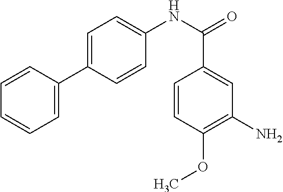 [SMILES: COc1ccc(C(=O)Nc2ccc(-c3ccccc3)cc2)cc1N]

3.32 g (9.54 mmol) of the compound from example 4A were stirred in a mixture of 100 mL of ethyl acetate and 50 mL of THF. 1.01 g (0.95 mmol) of palladium on charcoal (10%, 50% water) were added, and the mixture was stirred under a hydrogen atmosphere at room temperature for 3.25 h. After filtration, the solvents were evaporated. 3.53 g of the title compound were obtained and used without further purification.

¹H-NMR (300 MHz, DMSO-d₆): δ [ppm]=3.81 (s, 3H), 4.91 (s, 2H), 6.81-6.90 (m, 1H), 7.17-7.24 (m, 2H), 7.26-7.33 (m, 1H), 7.37-7.45 (m, 2H), 7.57-7.66 (m, 4H), 7.80-7.86 (m, 2H), 10.02 (s, 1H).

LC-MS (Method 3): Rₜ=1.19 min; MS (ESIpos): m/z=319 [M+H]⁺.

### Example 8A

### 3-amino-N-(biphenyl-4-yl)-4-(trifluoromethyl)benzamide

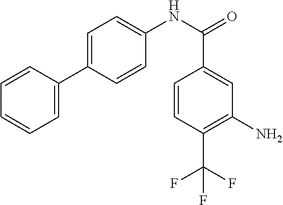 [SMILES: Nc1cc(C(=O)Nc2ccc(-c3ccccc3)cc2)ccc1C(F)(F)F]

4.52 g (11.7 mmol) of the compound from example 5A were dissolved in a mixture of 120 mL of ethyl acetate and 20 mL of THF. 1.25 g of palladium on charcoal (10%, 50% water) were added, and the mixture was stirred under a hydrogen atmosphere at room temperature for 1.5 h. After filtration, the solvents were evaporated. The remaining material was dissolved in a mixture of 120 mL of ethyl acetate and 40 mL of THF. 1.25 g of palladium on charcoal (10%, 50% water) were added, and the mixture was stirred under a hydrogen atmosphere at room temperature for 3 h. Additional 1.25 g of palladium on charcoal (10%, 50% water) were added, and the mixture was stirred under a hydrogen atmosphere at room temperature for 3 h. Afterwards another 1.25 g of palladium on charcoal (10%, 50% water) were added, and the mixture was stirred under a hydrogen atmosphere at room temperature for 6 h. After filtration, the solvents were evaporated. 3.81 g (91% of theory) of the title compound were obtained.

¹H-NMR (400 MHz, DMSO-d₆): δ [ppm]=5.85 (s, 2H), 7.14 (d, 1H), 7.31-7.37 (m, 2H), 7.42-7.50 (m, 3H), 7.63-7.71 (m, 4H), 7.83-7.89 (m, 2H), 10.36 (s, 1H).

LC-MS (Method 1): Rₜ=1.34 min; MS (ESIpos): m/z=357 [M+H]⁺.

### Example 9A

### 5-amino-N-(biphenyl-4-yl)-2-chloro-4-methoxybenzamide

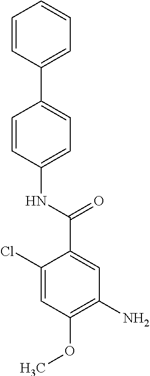 [SMILES: COc1cc(Cl)c(C(=O)Nc2ccc(-c3ccccc3)cc2)cc1N]

1.08 g of 93% purity (2.63 mmol) of the compound from example 6A were provided in 16 mL of THF and cooled to 0° C. 17.9 mL (21.0 mmol) of a 10% aqueous hydrogen chloride solution containing 15% of titanium(III) trichloride were added, and the mixture was stirred at room temperature over night. After cooling to 0° C., the mixture was neutralized by addition of sodium bicarbonate, saturated with sodium chloride and stirred for 2 h with a mixture of ethyl acetate and THF. After filtration, the solution was washed with brine, dried over sodium sulfate, filtered and concentrated. 960 mg of the title compound were obtained and used without further purification.

¹H-NMR (300 MHz, DMSO-d₆): δ [ppm]=3.83 (s, 3H), 5.10 (s, 2H), 6.81 (s, 1H), 6.92 (s, 1H), 7.29-7.37 (m, 1H), 7.40-7.50 (m, 2H), 7.61-7.70 (m, 4H), 7.76-7.85 (m, 2H), 10.36 (s, 1H).

LC-MS (Method 1): Rₜ=1.27 min; MS (ESIpos): m/z=353 [M+H]⁺.

### Example 10A

### methyl 2-amino-4-(biphenyl-4-ylcarbamoyl)benzoate

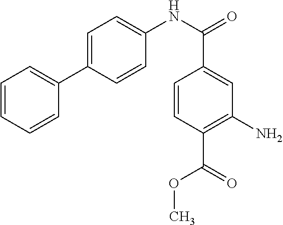 [SMILES: COC(=O)c1ccc(C(=O)Nc2ccc(-c3ccccc3)cc2)cc1N]

A mixture of 3-amino-4-(methoxycarbonyl)benzoic acid (1.00 g, 5.12 mmol) and biphenyl-4-amine (1.73 g, 10.2 mmol, 2.0 equiv) in DMF (35 mL) was treated with propanephosphonic anhydride (50%, 5.98 mL, 10.2 mmol, 2.0 equiv), followed by diisopropylethylamine (4.5 mL, 25.6 mmol, 5.0 equiv). The resulting mixture was allowed to stir at room temperature for 5 h. The resulting solution was concentrated under reduced pressure until a precipitate began to form (removal of approximately 20 mL). The resulting mixture was treated with water (25 mL). The resulting solids were separated, washed with water, and dried at 50° C. under reduced pressure to give impure methyl 2-amino-4-(biphenyl-4-ylcarbamoyl)benzoate (2.9 g). This material was used in subsequent reactions without further purification.

LC-MS (Method 3): Rₜ=1.31 min; MS (ESIpos): m/z=347 ([M+H]⁺, 100%), 693 ([2M+H]⁺, 10%); MS (ESIneg): m/z=345 [M−H]⁻, 100%).

### Example 11A

### 3-amino-N-(biphenyl-4-yl)-4-bromobenzamide

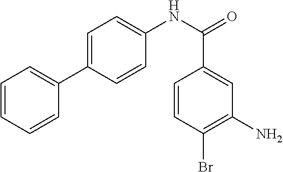 [SMILES: Nc1cc(C(=O)Nc2ccc(-c3ccccc3)cc2)ccc1Br]

A mixture of 3-amino-4-bromobenzoic acid (4.5 g, 20.8 mmol) and biphenyl-4-amine (7.1 g, 41.7 mmol, 2.0 equiv) in DMF (150 mL) was treated with propanephosphonic anhydride (50%, 24 mL, 41.7 mmol, 2.0 equiv), followed by diisopropylethylamine (18 mL, 104 mmol, 5.0 equiv). The resulting mixture was allowed to stir at room temperature for 24 h. The resulting mixture was treated with water (150 mL). The resulting solids were separated, washed with water, and dried at 50° C. under reduced pressure to give impure 3-amino-N-(biphenyl-4-yl)-4-bromobenzamide (6.0 g, 79%). This material was used in subsequent reactions without further purification.

¹H-NMR (300 MHz, DMSO-d₆): δ [ppm]=5.54 (s, 2H), 7.03 (dd, J=2.3, 8.1 Hz, 1H), 7.28-7.33 (m, 2H), 7.42 (t, J=7.7 Hz, 2H), 7.46 (d, J=8.1 Hz, 1H), 7.60-7.65 (m, 4H), 7.83 (d, J=8.6 Hz, 2H), 10.21 (s, 1H).

LC-MS (Method 3): Rₜ=1.32 min; MS (ESIpos): m/z=367 ([M+H]⁺, 100%); MS (ESIneg): m/z=365 [M−H]⁻, 90%).

### Example 12A

### N-(biphenyl-4-yl)-3-[(chloroacetyl)amino]-4-methoxybenzamide

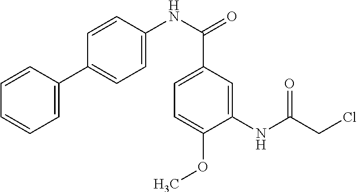 [SMILES: COc1ccc(C(=O)Nc2ccc(-c3ccccc3)cc2)cc1NC(=O)CCl]

1.00 g (3.14 mmol) of the compound from example 7A and 279 μL (3.46 mmol) of pyridine were provided in 10 mL of dichloromethane. 263 μL (3.30 mmol) of chloroacetyl chloride were added at 0° C., and the mixture was stirred at room temperature over night. Water and ethanol were added, and the solid was filtered off, washed with ethanol and dried. 968 mg (78% of theory) of the title compound were obtained.

¹H-NMR (400 MHz, DMSO-d₆): δ [ppm]=3.95 (s, 3H), 4.42 (s, 2H), 7.22 (d, 1H), 7.30-7.37 (m, 1H), 7.41-7.49 (m, 2H), 7.63-7.70 (m, 4H), 7.80-7.90 (m, 3H), 8.52-8.60 (m, 1H), 9.66 (s, 1H), 10.23 (s, 1H).

LC-MS (Method 4): Rₜ=1.29 min; MS (ESIpos): m/z=395 [M+H]⁺.

### Example 13A

### N-(biphenyl-4-yl)-3-[(chloroacetyl)amino]-4-(trifluoromethyl)benzamide

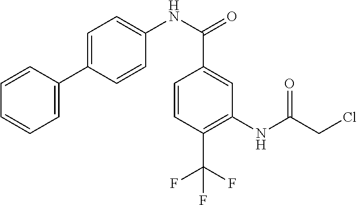 [SMILES: O=C(CCl)Nc1cc(C(=O)Nc2ccc(-c3ccccc3)cc2)ccc1C(F)(F)F]

To a solution of 3-amino-N-(biphenyl-4-yl)-4-(trifluoromethyl)benzamide (prepared in a manner analogous to that described in example 8A, 1.75 g, 4.91 mmol) and pyridine (0.42 mL, 5.16 mmol, 1.05 equiv) in CH₂Cl₂ (20 mL) at 0° C. was added chloroacetyl chloride (0.41 mL, 5.16 mmol, 1.05 equiv) dropwise. The resulting mixture was allowed to warm to room temperature and was stirred at that temperature for 5 h. The resulting mixture was concentrated under reduced pressure, then treated with EtOH (25 mL). The resulting solids were removed, washed with water followed by EtOH, then dried at 50° C. under reduced pressure to give N-(biphenyl-4-yl)-3-[(chloroacetyl)amino]-4-(trifluoromethyl)benzamide (1.11 g, 52%).

¹H-NMR (300 MHz, DMSO-d₆): δ [ppm]=4.33 (s, 2H), 7.31 (tm, J=7.3 Hz, 1H), 7.43 (t, J=7.6 Hz, 2H), 7.62-7.69 (m, 4H), 7.84 (d, J=8.7 Hz, 2H), 7.92 (d, J=8.9 Hz, 1H), 8.03-8.07 (m, 2H), 10.08 (s, 1H), 10.54 (s, 1H).

LC-MS (Method 3): Rₜ=1.32 min; MS (ESIpos): m/z=433 ([M+H]⁺, 60%), 865 ([2M+H]⁺, 20%); MS (ESIneg): m/z=431 ([M−H]⁻, 100%), 863 ([2M+H]⁻, 10%).

### Example 14A

### methyl 4-(biphenyl-4-ylcarbamoyl)-2-[(chloroacetyl)amino]benzoate

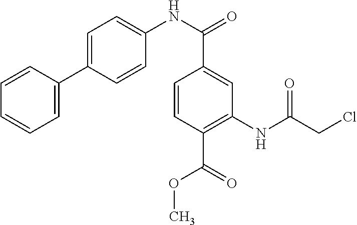 [SMILES: COC(=O)c1ccc(C(=O)Nc2ccc(-c3ccccc3)cc2)cc1NC(=O)CCl]

To a solution of methyl 2-amino-4-(biphenyl-4-ylcarbamoyl)benzoate (prepared in a manner analogous to that described in example 10A, 4.74 g, 13.7 mmol) and pyridine (2.77 mL, 34.2 mmol, 2.5 equiv) in CH₂Cl₂ (80 mL) at 0° C. was added chloroacetyl chloride (1.20 mL, 15.1 mmol, 1.1 equiv) dropwise. The resulting mixture was allowed to warm to room temperature and was stirred at that temperature for 6 h. The resulting mixture was concentrated under reduced pressure, then treated with EtOH (75 mL). The resulting solids were removed, washed with EtOH, followed by water, followed by EtOH, then dried at 50° C. under reduced pressure to give methyl 4-(biphenyl-4-ylcarbamoyl)-2-[(chloroacetyl)amino]benzoate (2.95 g, 51%).

¹H-NMR (300 MHz, DMSO-d₆): δ [ppm]=3.89 (s, 3H), 4.44 (s, 2H), 7.31 (t, J=7.3 Hz, 1H), 7.43 (t, J=7.7 Hz, 2H), 7.63-7.68 (m, 4H), 7.78 (dd, J=1.5, 8.3 Hz, 1H), 7.84 (d, J=8.8 Hz, 1H), 8.07 (d, J=8.3 Hz, 2H), 8.77 (d, J=1.5 Hz, 1H), 10.54 (s, 1H), 11.24 (s, 1H).

LC-MS (Method 3): Rₜ=1.37 min; MS (ESIpos): m/z=423 ([M+H]⁺, 100%), 845 ([2M+H]⁺, 20%); MS (ESIneg): m/z=421 ([M−H]⁻, 100%).

### Example 15A

### N-(biphenyl-4-yl)-4-bromo-3-[(chloroacetyl)amino]benzamide

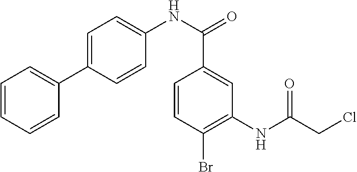 [SMILES: O=C(CCl)Nc1cc(C(=O)Nc2ccc(-c3ccccc3)cc2)ccc1Br]

To a solution of 3-amino-N-(biphenyl-4-yl)-4-bromobenzamide (prepared in a manner analogous to that described in example 11A, 6.04 g, 16.5 mmol) and pyridine (2.79 mL, 34.5 mmol, 2.1 equiv) in CH₂Cl₂ (100 mL) at 0° C. was added chloroacetyl chloride (1.38 mL, 17.3 mmol, 1.05 equiv) dropwise. The resulting mixture was allowed to warm to room temperature and was stirred at that temperature for 12 h. The resulting mixture was concentrated under reduced pressure, then treated with EtOH (75 mL). The resulting solids were removed, washed with water, followed by EtOH, then dried at 50° C. under reduced pressure to give N-(biphenyl-4-yl)-4-bromo-3-[(chloroacetyl)amino]benzamide (5.46 g, 75%).

¹H-NMR (300 MHz, DMSO-d₆): δ [ppm]=4.37 (s, 2H), 7.31 (t, J=7.3 Hz, 1H), 7.42 (t, J=7.7 Hz, 2H), 7.63-7.67 (m, 4H), 7.76 (dd, J=2.3, 8.6 Hz, 1H), 7.82-7.86 (m, 3H), 8.16 (d, J=2.0 Hz, 1H), 9.98 (s, 1H), 10.41 (s, 1H).

LC-MS (Method 3): Rₜ=1.34 min; MS (ESIpos): m/z=443 ([M+H]⁺, 80%); MS (ESIneg): m/z=441 ([M−H]⁻, 80%).

### Example 16A

### N-(biphenyl-4-yl)-3-[(2-chloropropanoyl)amino]-4-(trifluoromethyl)benzamide

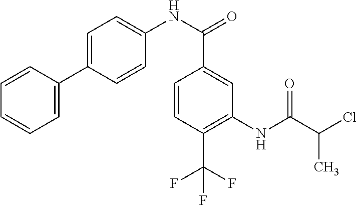 [SMILES: CC(Cl)C(=O)Nc1cc(C(=O)Nc2ccc(-c3ccccc3)cc2)ccc1C(F)(F)F]

3.00 g (8.42 mmol) of the compound from example 8A were provided in 50 mL of toluene, 1.63 mL (16.8 mmol) of 2-chloropropanoyl chloride were added, and the mixture was stirred for 90 minutes at 100° C. After concentration, 3.02 g of raw material were obtained, which were used without further purification.

¹H-NMR (400 MHz, DMSO-d₆): δ [ppm]=1.65 (d, 3H), 4.83 (q, 1H), 7.32-7.38 (m, 1H), 7.44-7.50 (m, 2H), 7.65-7.73 (m, 4H), 7.86-7.91 (m, 2H), 7.97 (d, 1H), 8.04 (s, 1H), 8.10 (d, 1H), 10.15 (s, 1H), 10.60 (s, 1H).

LC-MS (Method 1): Rₜ=1.39 min; MS (ESIpos): m/z=447 [M+H]⁺.

### Example 17A

### N-(biphenyl-4-yl)-3-[(2-bromo-2-methylpropanoyl)amino]-4-(trifluoromethyl)benzamide

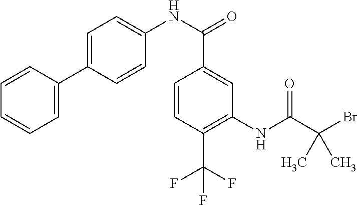 [SMILES: CC(C)(Br)C(=O)Nc1cc(C(=O)Nc2ccc(-c3ccccc3)cc2)ccc1C(F)(F)F]

500 mg (1.40 mmol) of the compound from example 8A and 125 μL (1.54 mmol) of pyridine were provided in 5 mL of dichloromethane. 339 mg (1.47 mmol) of 2-bromo-2-methylpropanoyl bromide were added at 0° C., and the mixture was stirred at room temperature over night. Water was added, and the phases were separated. The aqueous phase was extracted twice with dichloromethane, and the combined organic phases were washed with brine, dried over sodium sulfate, filtered and concentrated. 665 mg (94% of theory) of the title compound were obtained.

¹H-NMR (300 MHz, DMSO-d₆): δ [ppm]=2.01 (s, 6H), 7.30-7.41 (m, 1H), 7.42-7.52 (m, 2H), 7.63-7.75 (m, 4H), 7.85-7.92 (m, 2H), 7.93-8.01 (m, 2H), 8.09-8.17 (m, 1H), 9.91 (s, 1H), 10.62 (s, 1H).

LC-MS (Method 4): Rₜ=1.50 min; MS (ESIpos): m/z=505 [M+H]⁺.

### Example 18A

### dilithium N-(biphenyl-4-yl)-4-carboxy-3-{(2)-[2-(morpholin-4-yl)-1-oxidanidylethylidene]amino}benzenecarboximidate

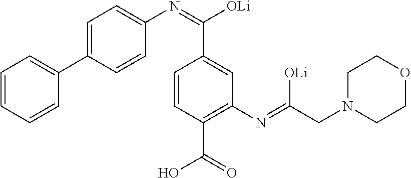

To a solution of the compound from example 12 (500 mg, 1.06 mmol) in a mixture of THF (6 mL) and methanol (1.5 mL) was added a 1M aqueous solution of lithium hydroxide (1.4 mL, 1.4 mmol, 1.3 equiv) at room temperature. The mixture was stirred for 4 h at room temperature. The resulting mixture was concentrated under reduced pressure, and washed with CH₂Cl₂ (10 mL). The resulting aqueous layer was concentrated to dryness under reduced pressure to give the title compound (510 mg), which was used without further purification.

¹H-NMR (400 MHz, DMSO-d₆): δ [ppm]=3.10 (s, 2H), 3.66-3.76 (m, 4H), 7.28-7.35 (m, 1H), 7.44 (t, 2H), 7.51 (dd, 1H), 7.58-7.70 (m, 4H), 7.78-7.87 (m, 2H), 8.03 (d, 1H), 9.04 (d, 1H), 14.27 (s, 1H).

LC-MS (Method 3): Rₜ=0.77 min; MS (ESIpos): m/z=460 [M−2Li+3H]⁺.

### Example 19A

### 3-[(chloroacetyl)amino]-4-(trifluoromethoxy)benzoic acid

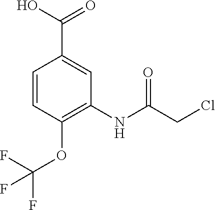 [SMILES: O=C(CCl)Nc1cc(C(=O)O)ccc1OC(F)(F)F]

To a solution of 3-amino-4-(trifluoromethoxy)benzoic acid (2.50 g, 11.3 mmol) and pyridine (1.92 mL, 23.7 mmol, 2.1 equiv) in CH₂Cl₂ (50 mL) at 0° C. was added chloroacetyl chloride (0.95 mL, 11.9 mmol, 1.05 equiv) dropwise. The resulting mixture was allowed to warm to room temperature and was stirred at that temperature for 5 h. The resulting solution was treated with a CH₂Cl₂/isopropanol mixture (4:1, 50 mL). The resulting solution was washed with an aqueous 1N HCl solution (50 mL), dried (MgSO₄ anh), and concentrated under reduced pressure to give impure 3-[(chloroacetyl)amino]-4-(trifluoromethyl)benzoic acid (3.52 g). This material was used in subsequent reactions without further purification.

¹H-NMR (300 MHz, DMSO-d₆): δ [ppm]=4.35 (s, 2H), 7.52 (ddm, J=1.5, 8.7 Hz, 1H), 7.80 (dd, J=2.1, 8.7 Hz, 1H), 8.47 (d, J=2.1 Hz, 1H), 10.17 (s, 1H), 13.28 (br. s, 1H).

LC-MS (Method 3): Rₜ=0.95 min; MS (ESIpos): m/z=298 ([M+H]⁺, 100%); MS (ESIneg): m/z=296 ([M−H]⁻, 100%), 593 ([2M−H]⁻, 100%).

### Example 20A

### 3-[(morpholin-4-ylacetyl)amino]-4-(trifluoromethoxy)benzoic acid

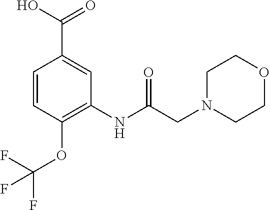 [SMILES: O=C(CN1CCOCC1)Nc1cc(C(=O)O)ccc1OC(F)(F)F]

To a solution of 3-[(chloroacetyl)amino]-4-(trifluoromethoxy)benzoic acid (prepared in a manner analogous to that described in example 19A, 3.52 g, 11.8 mmol) in DMF (50 mL) was added morpholine (2.2 mL, 24.8 mmol, 2.1 equiv), triethylamine (3.5 mL, 24.8 mmol, 2.1 equiv) and potassium iodide (0.30 g, 1.83 mmol, 0.16 equiv). The reaction mixture was stirred at room temperature for 16 h. The resulting mixture was diluted with water (75 mL). The aqueous solution was extracted with a CH₂Cl₂/isopropanol solution (4:1, 5×50 mL). The combined organic phases were washed with a saturated NaCl solution (50 mL), dried (Na₂SO₄ anh), and concentrated under reduced pressure to give impure 3-[(morpholin-4-ylacetyl)amino]-4-(trifluoromethoxy)benzoic acid (2.87 g). This material was used in subsequent reactions without further purification.

¹H-NMR (300 MHz, DMSO-d₆): δ [ppm]=2.54-2.59 (m, 4H), 3.20 (s, 2H), 3.61-3.66 (m, 4H), 7.49-7.54 (m, 1H), 7.76 (dd, J=2.1, 8.6 Hz, 1H), 8.80 (d, J=2.1 Hz, 1H), 9.81 (s, 1H).

LC-MS (Method 3): Rₜ=0.58 min; MS (ESIpos): m/z=349 ([M+H]⁺, 100%); MS (ESIneg): m/z=347 ([M−H]⁻, 100%).

### Example 21A

### methyl 4-(benzyloxy)-3-[(chloroacetyl)amino]benzoate

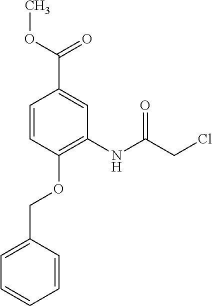 [SMILES: COC(=O)c1ccc(OCc2ccccc2)c(NC(=O)CCl)c1]

To a solution of methyl 3-amino-4-(benzyloxy)-benzoate (5.00 g, 19.4 mmol) and pyridine (3.30 mL, 40.8 mmol, 2.1 equiv) in CH₂Cl₂ (80 mL) at 0° C. was added chloroacetyl chloride (1.63 mL, 20.4 mmol, 1.05 equiv) dropwise. The resulting mixture was allowed to warm to room temperature and was stirred at that temperature for 12 h. The resulting solution was diluted with CH₂Cl₂ (75 mL). The resulting solution was washed with water (50 mL), dried (Na₂SO₄ anh) and concentrated under reduced pressure to give impure methyl 4-(benzyloxy)-3-[(chloroacetyl)amino]benzoate (7.26 g). This material was used in subsequent reactions without further purification.

LC-MS (Method 3): Rₜ=1.27 min; MS (ESIpos): m/z=334 ([M+H]⁺, 100%); MS (ESIneg): m/z=332 ([M−H]⁻, 100%).

### Example 22A

### methyl 4-(benzyloxy)-3-[(morpholin-4-ylacetyl)amino]benzoate

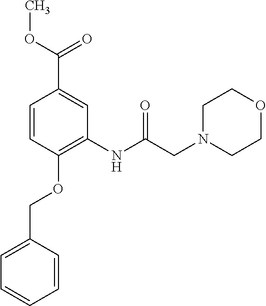 [SMILES: COC(=O)c1ccc(OCc2ccccc2)c(NC(=O)CN2CCOCC2)c1]

To a solution of methyl 4-(benzyloxy)-3-[(chloroacetyl)amino]benzoate (prepared in a manner analogous to that described in example 21A, 7.26 g, 21.8 mmol) in DMF (93 mL) was added morpholine (2.8 mL, 32.6 mmol, 1.5 equiv), triethylamine (4.5 mL, 32.6 mmol, 1.5 equiv) and potassium iodide (0.56 g, 3.37 mmol, 0.16 equiv). The reaction mixture was stirred at room temperature for 16 h. The resulting mixture was poured onto water (75 mL) to form a precipitate. The precipitate was removed by filtration, washed with water, and dried at 50° C. under reduced pressure to give methyl 4-(benzyloxy)-3-[(morpholin-4-ylacetyl)amino]benzoate (2.15 g, 26%).

¹H-NMR (300 MHz, DMSO-d₆): δ [ppm]=2.35-2.39 (m, 4H), 3.06 (s, 2H), 3.20-3.24 (m, 4H), 3.79 (s, 3H), 5.21 (s, 2H), 7.30 (d, J=8.6 Hz, 1H), 7.35-7.44 (m, 3H), 7.51-7.54 (m, 2H), 7.70 (dd, J=2.0, 8.6 Hz, 1H), 8.89 (d, J=2.1 Hz, 1H), 9.71 (s, 1H).

LC-MS (Method 3): Rₜ=1.23 min; MS (ESIpos): m/z=385 ([M+H]⁺, 100%), 769 ([2M+H]⁺, 30%); MS (ESIneg): m/z=383 ([M−H]⁻, 100%).

### Example 23A

### lithium 4-(benzyloxy)-3-[(morpholin-4-ylacetyl)amino]benzoate

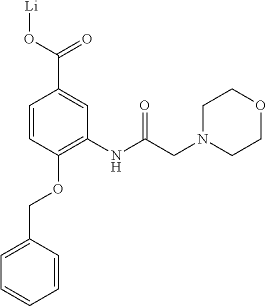

To a solution of methyl 4-(benzyloxy)-3-[(morpholin-4-ylacetyl)amino]benzoate (prepared in a manner analogous to that described in example 22A, 2.15 g, 5.59 mmol) in a mixture of THF (46 mL) and methanol (12 mL) was added an aqueous lithium hydroxide solution (1.0 N, 6.7 mL, 6.7 mmol, 1.2 equiv). The resulting solution was stirred for 12 h at room temperature. The resulting mixture was concentrated under reduced pressure to give lithium 4-(benzyloxy)-3-[(morpholin-4-ylacetyl)amino]benzoate (2.13 g, 100%). This material was used in subsequent reactions without further purification.

¹H-NMR (300 MHz, DMSO-d₆): δ [ppm]=2.35-2.40 (m, 4H), 3.02 (s, 2H), 3.22-3.27 (m, 4H), 5.10 (s, 2H), 7.02 (br. d, J=8.3 Hz, 1H), 7.33-7.42 (m, 3H), 7.48-7.52 (m, 2H), 7.56 (br. d, J=7.8 Hz, 1H), 8.70 (br. s, 1H), 9.52 (s, 1H).

LC-MS (Method 3): Rₜ=0.63 min; MS (ESIpos): m/z=371 ([M−Li+2H]⁺, 100%); MS (ESIneg): m/z=369 ([M−Li]⁻, 100%).

### Example 24A

### N-(biphenyl-4-yl)-4-hydroxy-3-[(morpholin-4-ylacetyl)amino]benzamide

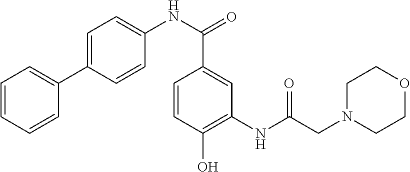 [SMILES: O=C(CN1CCOCC1)Nc1cc(C(=O)Nc2ccc(-c3ccccc3)cc2)ccc1O]

To a solution of 4-(benzyloxy)-N-(biphenyl-4-yl)-3-[(morpholin-4-ylacetyl)amino]benzamide (prepared in a manner analogous to that described in example 24, 0.21 g, 0.39 mmol) in THF (10 mL) was added 10% palladium on carbon (0.07 g). The resulting slurry was stirred under a hydrogen atmosphere at room temperature for 7 h. The resulting slurry was filtered and concentrated under reduced pressure to give N-(biphenyl-4-yl)-4-hydroxy-3-[(morpholin-4-ylacetyl)amino]benzamide (0.12 g, 68%).

¹H-NMR (300 MHz, DMSO-d₆): δ [ppm]=2.51-2.55 (m, 4H), 3.14 (s, 2H), 3.61-3.65 (m, 4H), 6.94 (d, J=8.3 Hz, 1H), 7.30 (t, J=7.3 Hz, 1H), 7.41 (t, J=7.7 Hz, 2H), 7.58 (dd, J=2.0, 8.3 Hz, 1H), 7.60-7.65 (m, 4H), 7.82 (d, J=8.6 Hz, 2H), 8.68 (d, J=2.0 Hz, 1H), 9.65 (s, 1H), 10.10 (s, 1H).

LC-MS (Method 3): Rₜ=0.74 min; MS (ESIpos): m/z=432 ([M+H]⁺, 100%), 863 ([2M+H]⁺, 10%); MS (ESIneg): m/z=430 ([M−H]⁻, 100%), 861 ([2M−H]⁻, 10%).

### Example 25A

### N-(4-methoxy-3-nitrophenyl)biphenyl-4-carboxamide

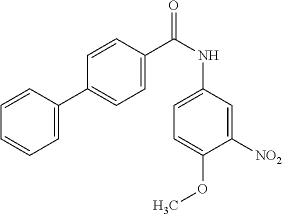 [SMILES: COc1ccc(NC(=O)c2ccc(-c3ccccc3)cc2)cc1[N+](=O)[O-]]

3.00 g (17.8 mmol) of 4-methoxy-3-nitroaniline and 8.63 g (62.4 mmol) of potassium carbonate were suspended in 120 mL of acetonitrile. 3.86 g (17.8 mmol) of biphenyl-4-carbonyl chloride were added at 0° C., and the mixture was stirred over night at room temperature. Afterwards the mixture was poured into ice-cold water and stirred for 15 minutes. The precipitate was sucked off, washed with water and dried. 5.49 g (88% of theory) of the title compound were obtained.

¹H-NMR (300 MHz, DMSO-d₆): δ [ppm]=3.93 (s, 3H), 7.38-7.47 (m, 2H), 7.48-7.56 (m, 2H), 7.74-7.80 (m, 2H), 7.83-7.89 (m, 2H), 8.00-8.11 (m, 3H), 8.45 (d, 1H), 10.52 (s, 1H).

LC-MS (Method 4): Rₜ=1.27 min; MS (ESIpos): m/z=349 [M+H]⁺.

### Example 26A

### N-(4-fluoro-3-nitrophenyl)biphenyl-4-carboxamide

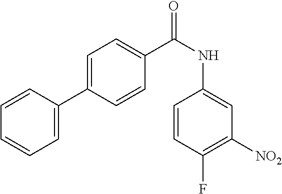 [SMILES: O=C(Nc1ccc(F)c([N+](=O)[O-])c1)c1ccc(-c2ccccc2)cc1]

4.90 g (31.4 mmol) of 4-fluoro-3-nitroaniline and 15.2 g (110 mmol) of potassium carbonate in 200 mL of acetonitrile were stirred at 0° C. 6.80 g (31.4 mmol) of biphenyl-4-carbonyl chloride were added, and the mixture was stirred for 20 h at room temperature. Afterwards the mixture was poured into ice-cold water and stirred for 15 minutes. The precipitate was filtered off, washed with water and dried. 8.39 g (79% of theory) of the title compound were obtained and used without further purification.

¹H-NMR (400 MHz, DMSO-d₆): δ [ppm]=7.39-7.47 (m, 1H), 7.49-7.55 (m, 2H), 7.62 (dd, 1H), 7.74-7.81 (m, 2H), 7.84-7.90 (m, 2H), 8.06-8.14 (m, 2H), 8.14-8.23 (m, 1H), 8.73 (dd, 1H), 10.71 (s, 1H).

LC-MS (Method 4): Rₜ=1.34 min; MS (ESIpos): m/z=337 [M+H]⁺.

### Example 27A

### N-[3-nitro-4-(trifluoromethoxy)phenyl]biphenyl-4-carboxamide

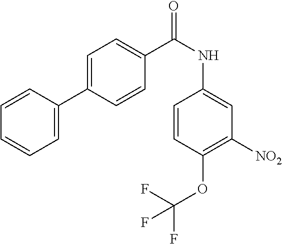 [SMILES: O=C(Nc1ccc(OC(F)(F)F)c([N+](=O)[O-])c1)c1ccc(-c2ccccc2)cc1]

500 mg (2.25 mmol) of 3-nitro-4-(trifluoromethoxy)aniline were provided in 7.5 mL of pyridine. 585 mg (2.70 mmol) of biphenyl-4-carbonyl chloride were added, and the mixture was stirred for 1 h at room temperature. Afterwards the mixture was poured into water and stirred for 15 minutes. The precipitate was filtered off, washed with water and dried. 810 mg (90% of theory) of the title compound were obtained and used without further purification.

¹H-NMR (400 MHz, DMSO-d₆): δ [ppm]=7.41-7.47 (m, 1H), 7.49-7.55 (m, 2H), 7.75-7.81 (m, 3H), 7.85-7.92 (m, 2H), 8.07-8.13 (m, 2H), 8.24 (dd, 1H), 8.74 (d, 1H), 10.87 (s, 1H).

LC-MS (Method 4): Rₜ=1.47 min; MS (ESIpos): m/z=403 [M+H]⁺.

### Example 28A

### N-(3-amino-4-methoxyphenyl)biphenyl-4-carboxamide

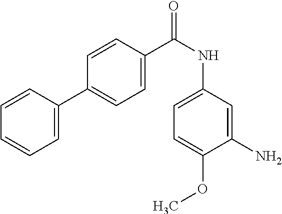 [SMILES: COc1ccc(NC(=O)c2ccc(-c3ccccc3)cc2)cc1N]

33.5 g (96.2 mmol) of the compound from example 25A were provided in a mixture of 0.4 L of ethanol and 0.6 L of THF. 5.12 g of palladium on charcoal (10%, 50% water) were added, and the mixture was stirred under a hydrogen atmosphere at room temperature for 6 h. After filtration, the solvents were evaporated. 30.2 g (99% of theory) of the title compound were obtained.

¹H-NMR (400 MHz, DMSO-d₆): δ [ppm]=3.75 (s, 3H), 4.76 (s, 2H), 6.75 (d, 1H), 6.91 (dd, 1H), 7.16 (d, 1H), 7.38-7.46 (m, 1H), 7.47-7.55 (m, 2H), 7.72-7.77 (m, 2H), 7.78-7.84 (m, 2H), 7.99-8.06 (m, 2H), 9.92 (s, 1H).

LC-MS (Method 1): Rₜ=1.11 min; MS (ESIpos): m/z=319 [M+H]⁺.

### Example 29A

### N-(3-amino-4-fluorophenyl)biphenyl-4-carboxamide

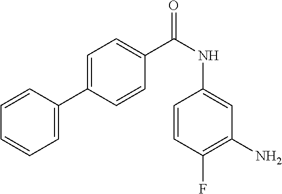 [SMILES: Nc1cc(NC(=O)c2ccc(-c3ccccc3)cc2)ccc1F]

8.39 g (24.9 mmol) of the compound from example 26A were suspended in a mixture of 100 mL of ethyl acetate and 200 mL of THF. 1.40 g (1.32 mmol) of palladium on charcoal (10%, 50% water) were added, and the mixture was stirred under a hydrogen atmosphere at room temperature for 3.25 h. After filtration, the solvents were evaporated. 7.64 g (100% of theory) of the title compound were obtained and used without further purification.

¹H-NMR (300 MHz, DMSO-d₆): δ [ppm]=5.18 (s, 2H), 6.82-6.99 (m, 2H), 7.33 (dd, 1H), 7.38-7.46 (m, 1H), 7.47-7.55 (m, 2H), 7.71-7.78 (m, 2H), 7.79-7.85 (m, 2H), 7.99-8.06 (m, 2H), 10.07 (s, 1H).

LC-MS (Method 4): Rₜ=1.18 min; MS (ESIpos): m/z=307 [M+H]⁺.

### Example 30A

### N-[3-amino-4-(trifluoromethoxy)phenyl]biphenyl-4-carboxamide

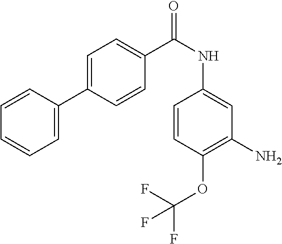 [SMILES: Nc1cc(NC(=O)c2ccc(-c3ccccc3)cc2)ccc1OC(F)(F)F]

7.10 g (17.7 mmol) of the compound from example 27A were suspended in a mixture of 74 mL of ethanol and 110 mL of THF. 0.94 g (0.88 mmol) of palladium on charcoal (10%, 50% water) were added, and the mixture was stirred under a hydrogen atmosphere at room temperature for 3 h. After filtration, the solvents were evaporated. 6.40 g (95% of theory) of the title compound were obtained and used without further purification.

¹H-NMR (300 MHz, DMSO-d₆): δ [ppm]=5.40 (s, 2H), 6.93 (dd, 1H), 7.06 (dd, 1H), 7.38-7.46 (m, 2H), 7.47-7.56 (m, 2H), 7.72-7.79 (m, 2H), 7.79-7.86 (m, 2H), 8.00-8.07 (m, 2H), 10.15 (s, 1H).

LC-MS (Method 4): Rₜ=1.36 min; MS (ESIpos): m/z=373 [M+H]⁺.

### Example 31A

### N-{3-[(chloroacetyl)amino]-4-methoxyphenyl}biphenyl-4-carboxamide

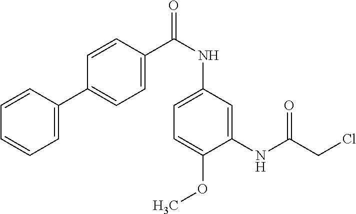 [SMILES: COc1ccc(NC(=O)c2ccc(-c3ccccc3)cc2)cc1NC(=O)CCl]

To a solution of N-(3-amino-4-methoxyphenyl)biphenyl-4-carboxamide (prepared in a manner analogous to that described in example 28A, 2.50 g, 7.85 mmol) and pyridine (0.70 mL, 8.64 mmol, 1.10 equiv) in CH₂Cl₂ (25 mL) at 0° C. was added chloroacetyl chloride (0.66 mL, 8.24 mmol, 1.05 equiv). The resulting mixture was warmed to room temperature, and stirred at that temperature for 12 h. The resulting mixture was concentrated under reduced pressure, was then triturated with ethanol (25 mL). The remaining solids were removed by filtration, washed with ethanol, followed by water, followed by ethanol, and were dried at 50° C. under reduced pressure to give N-{3-[(chloroacetyl)amino]-4-methoxyphenyl}biphenyl-4-carboxamide (2.97 g, 96%)

¹H-NMR (300 MHz, DMSO-d₆): δ [ppm]=3.82 (s, 3H), 4.37 (s, 2H), 7.03 (d, J=9.0 Hz, 1H), 7.38 (t, J=7.3 Hz, 1H), 7.48 (t, J=7.3 Hz, 2H), 7.60 (dd, J=2.5, 8.9 Hz, 1H), 7.73 (d, J=7.2 Hz, 2H), 7.79 (d, J=8.3 Hz, 2H), 8.03 (d, J=8.3 Hz, 2H), 8.36 (d, J=2.1 Hz, 1H), 9.50 (s, 1H), 10.21 (s, 1H).

LC-MS (Method 3): Rₜ=1.25 min; MS (ESIpos): m/z=395 ([M+H]⁺, 100%), 789 ([2M+H]⁺, 40%); MS (ESIneg): m/z=393 ([M−H]⁻, 100%).

### Example 32A

### N-{3-[(2-chloropropanoy)amino]-4-methoxyphenyl}biphenyl-4-carboxamide

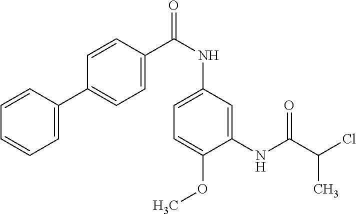 [SMILES: COc1ccc(NC(=O)c2ccc(-c3ccccc3)cc2)cc1NC(=O)C(C)Cl]

1.00 g (3.14 mmol) of the compound from example 28A were provided in 15 mL of toluene, 0.61 mL (6.28 mmol) of 2-chloropropanoyl chloride were added, and the mixture was stirred for 2 h at 100° C. After concentration, 876 mg of raw material were obtained, which were used without further purification.

¹H-NMR (400 MHz, DMSO-d₆): δ [ppm]=1.62 (d, 3H), 3.85 (s, 3H), 4.99 (q, 1H), 7.07 (d, 1H), 7.39-7.45 (m, 1H), 7.48-7.54 (m, 2H), 7.64 (dd, 1H), 7.73-7.79 (m, 2H), 7.80-7.85 (m, 2H), 8.04-8.10 (m, 2H), 8.39 (d, 1H), 9.54 (s, 1H), 10.23 (s, 1H).

LC-MS (Method 1): Rₜ=1.33 min; MS (ESIpos): m/z=409 [M+H]⁺.

### Example 33A

### N-{3-[(2-chloropropanoy)amino]-4-(trifluoromethoxy)phenyl}biphenyl-4-carboxamide

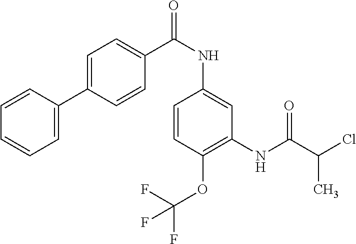 [SMILES: CC(Cl)C(=O)Nc1cc(NC(=O)c2ccc(-c3ccccc3)cc2)ccc1OC(F)(F)F]

1.00 g (2.69 mmol) of the compound from example 30A were provided in 20 mL of toluene, 0.52 mL (5.37 mmol) of 2-chloropropanoyl chloride were added, and the mixture was stirred for 2 h at 100° C. After concentration, 1.19 g of raw material were obtained, which were used without further purification.

LC-MS (Method 1): Rₜ=1.43 min; MS (ESIpos): m/z=463 [M+H]⁺.

### Example 34A

### N-[3-(benzylamino)-4-methoxyphenyl]biphenyl-4-carboxamide

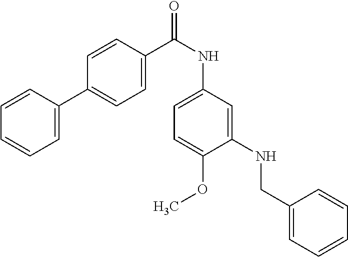 [SMILES: COc1ccc(NC(=O)c2ccc(-c3ccccc3)cc2)cc1NCc1ccccc1]

500 mg (1.57 mmol) of the compound from example 28A and 0.8 mL (7.85 mmol) benzaldehyde were dissolved in 50 mL of dichloromethane at room temperature, 333 mg (1.57 mmol) of sodium triacetoxyborohydride and 0.09 mL (1.57 mmol) of acetic acid were added, and the mixture was stirred at room temperature over night. 333 mg (1.57 mmol) of sodium triacetoxyborohydride and 0.09 mL (1.57 mmol) of acetic acid were added, and the mixture was stirred at room temperature over night. After concentration, the remaining material was taken up in ethyl acetate and was washed with a saturated aqueous sodium bicarbonate solution, dried over sodium sulfate, filtered and concentrated. Purification by HPLC (column: chromatorex C18, 10 μm, 195×51 mm, mobile phase: acetonitrile/water gradient) yielded 293 mg (46% of theory) of the title compound.

¹H-NMR (400 MHz, DMSO-d₆): δ [ppm]=3.80 (s, 3H), 4.32 (d, 2H), 5.52 (t, 1H), 6.79 (d, 1H), 6.98 (d, 1H), 7.06 (dd, 1H), 7.18-7.25 (m, 1H), 7.28-7.34 (m, 2H), 7.34-7.39 (m, 2H), 7.39-7.44 (m, 1H), 7.47-7.54 (m, 2H), 7.71-7.77 (m, 2H), 7.77-7.82 (m, 2H), 7.96-8.02 (m, 2H), 9.90 (s, 1H).

LC-MS (Method 4): Rₜ=1.44 min; MS (ESIpos): m/z=409 [M+H]⁺.

### Example 35A

### N-[4-methoxy-3-(methylamino)phenyl]biphenyl-4-carboxamide

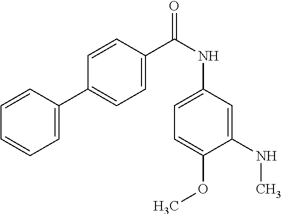 [SMILES: CNc1cc(NC(=O)c2ccc(-c3ccccc3)cc2)ccc1OC]

195 μL (2.07 mmol) of acetic anhydride were provided at 0° C., 95 μL (2.53 mmol) of formic acid were added, and the mixture was stirred at 55° C. for 2 h. After cooling to room temperature, 10 mL of THF and a solution of 250 mg (785 μmol) of the compound from example 28A in 4 mL of THF were added, and the mixture was stirred for 3 h at room temperature. After concentration, the remaining material was taken up in 10 mL of THF, 196 μL (1.96 mmol) of a 10M solution of borane dimethylsulfide complex in THF were added at 0° C., and the mixture was stirred at 0° C. for 1 h and at room temperature over night. Methanol was added, and the mixture was stirred for 1 h. A 1M aqueous solution of hydrogenchloride was added, and the mixture was stirred for 1 h. Water was added, and the mixture was set for a pH of 10 by addition of potassium carbonate and extracted twice with dichloromethane. The combined organic phase were washed with water, dried over sodium sulfate, filtered and concentrated. 285 mg (87% of theory) of the title compound were obtained and used without further purification.

¹H-NMR (300 MHz, DMSO-d₆): δ [ppm]=2.72 (d, 3H), 3.76 (s, 3H), 5.05 (q, 1H), 6.75 (d, 1H), 6.96 (d, 1H), 7.06 (dd, 1H), 7.38-7.45 (m, 1H), 7.47-7.55 (m, 2H), 7.73-7.84 (m, 4H), 8.01-8.09 (m, 2H), 9.97 (s, 1H).

LC-MS (Method 4): Rₜ=1.19 min; MS (ESIpos): m/z=333 [M+H]⁺.

### Example 36A

### 2-chloro-N-[5-nitro-2-(trifluoromethoxy)phenyl]acetamide

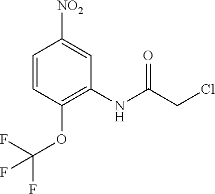 [SMILES: O=C(CCl)Nc1cc([N+](=O)[O-])ccc1OC(F)(F)F]

To a solution of 5-nitro-2-(trifluoromethoxy)aniline (17.3 g, 77.7 mmol) and pyridine (6.60 mL, 81.5 mmol, 1.05 equiv) in CH₂Cl₂ (250 mL) at 0° C. was added chloroacetyl chloride (6.50 mL, 81.5 mmol, 1.05 equiv) dropwise. The resulting mixture was warmed to room temperature and was stirred at that temperature for 12 h. The resulting mixture was diluted with CH₂Cl₂ (250 mL), washed with water (200 mL) followed by a saturated NaCl solution (250 mL), dried (MgSO₄ anh), and concentrated under reduced pressure to give impure 2-chloro-N-[5-nitro-2-(trifluoromethoxy)phenyl]acetamide (23.8 g). This material was used in subsequent reactions without further purification.

¹H-NMR (300 MHz, DMSO-d₆): δ [ppm]=4.40 (s, 2H), 7.69 (dd, J=1.7, 9.0 Hz, 1H), 8.09 (dd, J=3.0, 9.2 Hz, 1H), 8.88 (d, J=2.8 Hz, 1H), 10.41 (s, 1H).

LC-MS (Method 3): Rₜ=1.09 min; MS (ESIneg): m/z=297 ([M−H]⁻, 100%).

### Example 37A

### N-(2-tert-butyl-5-nitrophenyl)-2-chloroacetamide

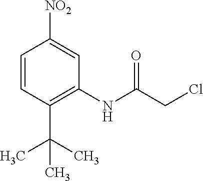 [SMILES: CC(C)(C)c1ccc([N+](=O)[O-])cc1NC(=O)CCl]

To a solution of 2-tert-butyl-5-nitroaniline (2.55 g, 13.1 mmol) and pyridine (2.20 mL, 27.6 mmol, 2.1 equiv) in CH₂Cl₂ (55 mL) at 0° C. was added chloroacetyl chloride (1.10 mL, 13.8 mmol, 1.05 equiv) dropwise. The resulting mixture was allowed to warm to room temperature and was stirred at that temperature for 12 h. The resulting solution was diluted with CH₂Cl₂ (50 mL), washed with water (50 mL), dried (Na₂SO₄ anh), and concentrated under reduced pressure to afford impure N-(2-tert-butyl-5-nitrophenyl)-2-chloroacetamide (3.94 g). This material was used in subsequent reactions without further purification.

LC-MS (Method 3): Rₜ=1.16 min; MS (ESIpos): m/z=271 ([M+H]⁺, 40%); MS (ESIneg): m/z=269 ([M−H]⁻, 100%).

### Example 38A

### N-(2-bromo-5-nitrophenyl)-2-chloroacetamide

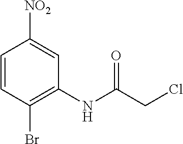 [SMILES: O=C(CCl)Nc1cc([N+](=O)[O-])ccc1Br]

To a solution of 2-bromo-5-nitroaniline (9.85 g, 45.4 mmol) and pyridine (7.34 mL, 90.8 mmol, 2.0 equiv) in CH₂Cl₂ (150 mL) at 0° C. was added chloroacetyl chloride (3.80 mL, 47.7 mmol, 1.05 equiv) dropwise. The resulting mixture was allowed to warm to room temperature and was stirred at that temperature for 12 h. The resulting solution was diluted with CH₂Cl₂ (150 mL), washed with water (100 mL), dried (Na₂SO₄ anh), and concentrated under reduced pressure to afford N-(2-bromo-5-nitrophenyl)-2-chloroacetamide (14.1 g). This material was used in subsequent reactions without further purification.

LC-MS (Method 3): Rₜ=1.05 min; MS (ESIneg): m/z=291 ([M−H]⁻, 80%).

### Example 39A

### 2-chloro-N-(2-chloro-5-nitrophenyl)acetamide

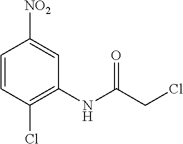 [SMILES: O=C(CCl)Nc1cc([N+](=O)[O-])ccc1Cl]

To a solution of 2-chloro-5-nitroaniline (3.00 g, 17.4 mmol) and pyridine (1.69 mL mL, 20.9 mmol, 1.2 equiv) in CH₂Cl₂ (60 mL) at 0° C. was added chloroacetyl chloride (1.66 mL, 20.9 mmol, 1.2 equiv) dropwise. The resulting mixture was allowed to warm to room temperature and was stirred at that temperature for 12 h. The resulting solution was diluted with CH₂Cl₂ (60 mL), washed with water (500 mL) followed by a saturated NaCl solution (50 mL), dried (MgSO₄ anh), and concentrated under reduced pressure to afford 2-chloro-N-(2-chloro-5-nitrophenyl)acetamide (4.4 g, 100%).

¹H-NMR (300 MHz, DMSO-d₆): δ [ppm]=4.42 (s, 2H), 7.80 (d J=8.8 Hz, 1H), 8.02 (dd, J=2.8, 8.8 Hz, 1H), 8.69 (d, J=2.5 Hz, 1H), 10.16 (s, 1H).

LC-MS (Method 3): Rₜ=0.97 min; MS (ESIneg): m/z=247 ([M−H]⁻, 100%).

### Example 40A

### 2-chloro-N-(2-methyl-5-nitrophenyl)acetamide

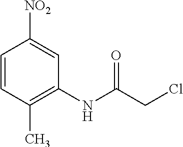

To a solution of 2-methyl-5-nitroaniline (2.00 g, 13.1 mmol) and pyridine (1.28 mL, 15.8 mmol, 1.2 equiv) in CH₂Cl₂ (30 mL) at 0° C. was added chloroacetyl chloride (1.1 mL, 13.8 mmol, 1.05 equiv) dropwise. The resulting mixture was warmed to room temperature, and was stirred at that temperature for 12 h. The resulting solution was diluted with CH₂Cl₂ (30 mL), washed with water (25 mL) followed by a saturated NaCl solution (25 mL), dried (MgSO₄ anh), and concentrated under reduced pressure to afford 2-chloro-N-(2-methyl-5-nitrophenyl)acetamide (2.2 g, 72%).

¹H-NMR (400 MHz, DMSO-d₆): δ [ppm]=2.32 (s, 3H), 4.35 (s, 2H), 7.50 (d, J=8.6 Hz, 1H), 7.94 (dd, J=2.5, 8.3 Hz, 1H), 8.39 (d, J=2.5 Hz, 1H), 9.87 (s, 1H).

LC-MS (Method 3): Rₜ=1.25 min; MS (ESIpos): m/z=229 ([M+H]⁺, 70%); MS (ESIneg): m/z=227 ([M−H]⁻, 100%).

### Example 41A

### 2-chloro-N-(2-methoxy-5-nitrophenyl)acetamide

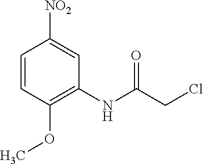 [SMILES: COc1ccc([N+](=O)[O-])cc1NC(=O)CCl]

To a solution of 2-methoxy-5-nitroaniline (10.00 g, 59.5 mmol) and pyridine (5.1 mL, 62.4 mmol, 1.05 equiv) in CH₂Cl₂ (175 mL) at 0° C. was added chloroacetyl chloride (4.97 mL, 62.4 mmol, 1.05 equiv) dropwise. The resulting mixture was warmed to room temperature, and was stirred at that temperature for 12 h. The resulting solution was concentrated under reduced pressure. The remaining solids were triturated with ethanol, filtered, washed with ethanol, followed by water, followed by ethanol, and dried at 50° C. under reduced pressure to give 2-chloro-N-(2-methoxy-5-nitrophenyl)acetamide (14.1 g, 97%).

¹H-NMR (400 MHz, DMSO-d₆): δ [ppm]=3.98 (s, 3H), 4.41 (s, 2H), 7.27 (d, J=9.1 Hz, 1H), 8.04 (dd, J=2.8, 9.1 Hz, 1H), 8.95 (d, J=2.8 Hz, 1H), 9.85 (s, 1H).

LC-MS (Method 3): Rₜ=0.95 min; MS (ESIpos): m/z=245 ([M+H]⁺, 100%); MS (ESIneg): m/z=243 ([M−H]⁻, 100%).

### Example 42A

### 2-{[tert-butyl(dimethyl)silyl]oxy}-5-nitroaniline

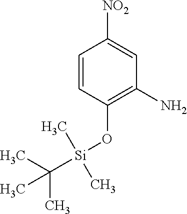 [SMILES: CC(C)(C)[Si](C)(C)Oc1ccc([N+](=O)[O-])cc1N]

To a solution of 2-amino-4-nitrophenol (5.00 g, 32.4 mmol) in CH₂Cl₂ (65 mL) at 0° C. was added tert-butyldimethylsilyl chloride (4.65 g, 30.8 mmol, 0.95 equiv) followed by triethylamine (4.97 mL, 35.7 mmol, 1.10 equiv). The resulting solution was stirred at room temperature for 5 h. The solution was then treated with tert-butyldimethylsilyl chloride (1.22 g, 8.10 mmol, 0.25 equiv) and triethylamine (1.13 mL, 8.11 mmol, 0.25 equiv) and stirred at room temperature for additional 48 h. The resulting solution was treated with diethyl ether (100 mL), then added to a saturated aqueous ammonium chloride solution (100 mL). The water phase from the diluted reaction mixture was extracted with diethyl ether (50 mL). The diethyl ether phase was combined with the organic phase from the diluted reaction mixture. The combined organic phases were washed with water (50 mL) followed by a saturated NaCl solution (50 mL), then dried (Na₂SO₄ anh), and concentrated under reduced pressure to give 2-{[tert-butyl(dimethyl)silyl]oxy}-5-nitroaniline (8.1 g, 93%).

¹H-NMR (400 MHz, DMSO-d₆): δ [ppm]=0.23 (s, 6H), 0.94 (s, 9H), 5.15 (s, 2H), 6.82 (d, J=8.9 Hz, 1H), 7.35 (dd, J=2.9, 8.7 Hz, 1H), 7.51 (d, J=2.8 Hz, 1H).

LC-MS (Method 4): Rₜ=1.51 min; MS (ESIpos): m/z=269 ([M+H]⁺, 100%).

### Example 43A

### N-(2-{[tert-butyl(dimethyl)silyl]oxy}-5-nitrophenyl)-2-chloroacetamide

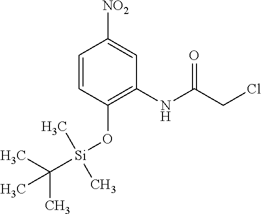 [SMILES: CC(C)(C)[Si](C)(C)Oc1ccc([N+](=O)[O-])cc1NC(=O)CCl]

To a solution of 2-{[tert-butyl(dimethyl)silyl]oxy}-5-nitroaniline (prepared in a manner analogous to that described in example 42A, 8.06 g, 30.0 mmol) and pyridine (5.10 mL, 63.1 mmol, 2.1 equiv) in CH₂Cl₂ (125 mL) at 0° C. was added chloroacetyl chloride (2.51 mL, 31.5 mmol, 1.05 equiv). The resulting mixture was warmed to room temperature and stirred at that temperature for 12 h. The resulting mixture was diluted with CH₂Cl₂ (100 mL), washed with water (75 mL), dried (Na₂SO₄ anh), and concentrated under reduced pressure to give impure N-(2-{[tert-butyl(dimethyl)silyl]oxy}-5-nitrophenyl)-2-chloroacetamide (9.6 g).

¹H-NMR (400 MHz, DMSO-d₆): δ [ppm]=−0.08 (s, 6H), 0.80 (s, 9H), 4.41 (s, 2H), 7.02 (d, J=8.8 Hz, 1H), 7.91 (dd, J=2.8, 8.8 Hz, 1H), 8.92 (d, J=2.8 Hz, 1H), 9.74 (s, 1H).

### Example 44A

### 2-(morpholin-4-yl)-N-[5-nitro-2-(trifluoromethoxy)phenyl]acetamide

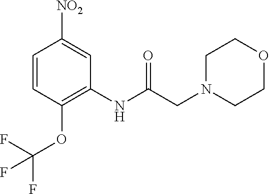 [SMILES: O=C(CN1CCOCC1)Nc1cc([N+](=O)[O-])ccc1OC(F)(F)F]

To a solution of 2-chloro-N-[5-nitro-2-(trifluoromethoxy)phenyl]acetamide (prepared in a manner analogous to that described in example 36A, 20.6 g, 69.0 mmol) in DMF (300 mL) was added morpholine (9.0 mL, 103.5 mmol, 1.5 equiv), triethylamine (14.4 mL, 103.5 mmol, 1.5 equiv) and potassium iodide (1.78 g, 10.7 mmol, 0.16 equiv). The reaction mixture was stirred at room temperature for 16 h. The resulting mixture was poured onto water (300 mL). The resulting mixture was extracted with ethyl acetate (3×100 mL). The combined organic phases were washed with half-saturated NaCl solution, dried (Na₂SO₄ anh) and concentrated under reduced pressure to give 2-(morpholin-4-yl)-N-[5-nitro-2-(trifluoromethoxy)phenyl]acetamide (20.0 g, 83%).

¹H-NMR (400 MHz, DMSO-d₆): δ [ppm]=2.53-2.56 (m, 4H), 3.22 (s, 2H), 3.59-3.62 (m, 4H), 7.72 (dq, J=1.7, 9.1 Hz, 1H), 8.05 (dd, J=2.8, 9.1 Hz, 1H), 9.11 (d, J=2.8 Hz, 1H), 10.05 (s, 1H).

LC-MS (Method 3): Rₜ=1.15 min; MS (ESIpos): m/z=350 ([M+H]⁺, 100%); MS (ESIneg): m/z=348 ([M−H]⁻, 100%).

### Example 45A

### N-(2-tert-butyl-5-nitrophenyl)-2-(morpholin-4-yl)acetamide

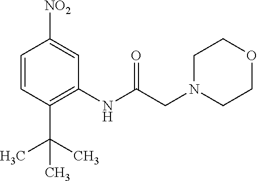 [SMILES: CC(C)(C)c1ccc([N+](=O)[O-])cc1NC(=O)CN1CCOCC1]

To a solution of N-(2-tert-butyl-5-nitrophenyl)-2-chloroacetamide (prepared in a manner analogous to that described in example 37A, 3.94 g, 14.6 mmol) in DMF (60 mL) was added morpholine (1.90 mL, 21.8 mmol, 1.5 equiv), triethylamine (3.04 mL, 21.8 mmol, 1.5 equiv) and potassium iodide (0.37 g, 2.56 mmol, 0.16 equiv). The reaction mixture was stirred at room temperature for 16 h. The resulting mixture was poured onto water (75 mL). The resulting mixture was extracted with ethyl acetate (3×50 mL). The combined organic phases were dried (Na₂SO₄ anh) and concentrated under reduced pressure to give N-(2-tert-butyl-5-nitrophenyl)-2-(morpholin-4-yl)acetamide (1.61 g, 34%).

¹H-NMR (400 MHz, DMSO-d₆): δ [ppm]=1.42 (s, 9H), 2.57-2.62 (m, 4H), 3.21 (s, 2H), 3.60-3.65 (m, 4H), 7.63 (d, J=9.0 Hz, 1H), 7.93 (dd, J=2.6, 8.9 Hz, 1H), 8.82 (d, J=2.5 Hz, 1H), 9.69 (s, 1H).

LC-MS (Method 3): Rₜ=1.19 min; MS (ESIpos): m/z=322 ([M+H]⁺, 100%); MS (ESIneg): m/z=320 ([M−H]⁻, 100%).

### Example 46A

### N-(2-bromo-5-nitrophenyl)-2-(morpholin-4-yl)acetamide

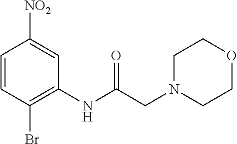 [SMILES: O=C(CN1CCOCC1)Nc1cc([N+](=O)[O-])ccc1Br]

To a solution of N-(2-bromo-5-nitrophenyl)-2-chloroacetamide (prepared in a manner analogous to that described in example 38A, 13.2 g, 45.0 mmol) in DMF (200 mL) was added morpholine (5.9 mL, 67.5 mmol, 1.5 equiv), triethylamine (9.4 mL, 67.5 mmol, 1.5 equiv) and potassium iodide (1.16 g, 6.98 mmol, 0.16 equiv). The reaction mixture was stirred at room temperature for 16 h. The resulting mixture was poured onto water (200 mL). The resulting precipitate was removed by filtration and washed with water to give N-(2-bromo-5-nitrophenyl)-2-(morpholin-4-yl)acetamide (11.1 g, 72%).

¹H-NMR (400 MHz, DMSO-d₆): δ [ppm]=2.54-2.60 (m, 4H), 3.21 (s, 2H), 3.65-3.69 (m, 4H), 7.86 (dd, J=2.6, 8.7 Hz, 1H), 7.97 (d, J=8.9 Hz, 1H), 9.13 (d, J=2.8 Hz, 1H), 10.22 (s, 1H).

LC-MS (Method 3): Rₜ=1.08 min; MS (ESIpos): m/z=344 ([M+H]⁺, 100%); MS (ESIneg): m/z=342 ([M−H]⁻, 50%).

### Example 47A

### N-(2-chloro-5-nitrophenyl)-2-(morpholin-4-yl)acetamide

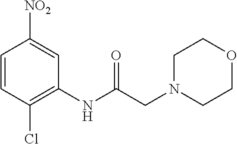 [SMILES: O=C(CN1CCOCC1)Nc1cc([N+](=O)[O-])ccc1Cl]

To a solution of 2-chloro-N-(2-chloro-5-nitrophenyl)acetamide (prepared in a manner analogous to that described in example 39A, 4.40 g, 17.7 mmol) in DMF (75 mL) was added morpholine (2.3 mL, 26.5 mmol, 1.5 equiv), triethylamine (3.7 mL, 26.5 mmol, 1.5 equiv) and potassium iodide (0.45 g, 2.74 mmol, 0.16 equiv). The reaction mixture was stirred at room temperature for 16 h. The resulting mixture was poured onto water (75 mL). The resulting precipitate was removed by filtration, washed with water followed by ethanol, and dried at 50° C. under reduced pressure to give N-(2-chloro-5-nitrophenyl)-2-(morpholin-4-yl)acetamide (4.8 g, 90%).

¹H-NMR (400 MHz, DMSO-d₆): δ [ppm]=2.54-2.58 (m, 4H), 3.22 (s, 2H), 3.63-3.66 (m, 4H), 7.82 (d, J=8.8 Hz, 1H), 7.96 (dd, J=2.8, 8.8 Hz, 1H), 9.11 (d, J=2.5 Hz, 1H), 10.17 (s, 1H).

LC-MS (Method 3): Rₜ=1.07 min; MS (ESIneg): m/z=298 ([M−H]⁻, 100%).

### Example 48A

### N-(2-methyl-5-nitrophenyl)-2-(morpholin-4-yl)acetamide

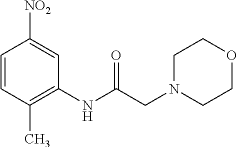 [SMILES: Cc1ccc([N+](=O)[O-])cc1NC(=O)CN1CCOCC1]

To a solution of 2-chloro-N-(2-methyl-5-nitrophenyl)acetamide (prepared in a manner analogous to that described in example 40A, 2.16 g, 9.5 mmol) in DMF (35 mL) was added morpholine (1.2 mL, 14.2 mmol, 1.5 equiv), triethylamine (2.0 mL, 14.2 mmol, 1.5 equiv) and potassium iodide (0.24 g, 1.46 mmol, 0.16 equiv). The reaction mixture was stirred at room temperature for 16 h. The resulting mixture was poured onto water (35 mL). The resulting precipitate was removed by filtration, washed with water followed by ethanol, and dried at 50° C. under reduced pressure to give N-(2-methyl-5-nitrophenyl)-2-(morpholin-4-yl)acetamide (2.1 g, 79%).

¹H-NMR (400 MHz, DMSO-d₆): δ [ppm]=2.34 (s, 3H), 2.53-2.56 (m, 4H), 3.17 (s, 2H), 3.61-3.65 (m, 4H), 7.50 (d, J=8.8 Hz, 1H), 7.90 (dd, J=2.5, 8.3 Hz, 1H), 8.71 (d, J=2.5 Hz, 1H), 9.65 (s, 1H).

LC-MS (Method 3): Rₜ=0.95 min; MS (ESIpos): m/z=280 ([M+H]⁺, 50%); MS (ESIneg): m/z=278 ([M−H]⁻, 100%).

### Example 49A

### N-(2-methoxy-5-nitrophenyl)-2-(morpholin-4-yl)acetamide

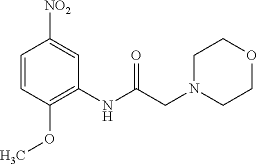 [SMILES: COc1ccc([N+](=O)[O-])cc1NC(=O)CN1CCOCC1]

To a solution of 2-chloro-N-(2-methoxy-5-nitrophenyl)acetamide (prepared in a manner analogous to that described in example 41A, 14.1 g, 57.6 mmol) in DMF (250 mL) was added morpholine (7.5 mL, 86.5 mmol, 1.5 equiv), triethylamine (12.1 mL, 86.5 mmol, 1.5 equiv) and potassium iodide (1.48 g, 8.93 mmol, 0.16 equiv). The reaction mixture was stirred at room temperature for 16 h. The resulting mixture was poured onto water (250 mL). The resulting mixture was extractecd with ethyl acetate (3×100 mL). The combined organic phases were washed with a half-saturated NaCl solution, dried (Na₂SO₄ anh), and concentrated under reduced pressure. The resulting material was triturated with ethanol to give N-(2-methoxy-5-nitrophenyl)-2-(morpholin-4-yl)acetamide as a precipitate (15.5 g, 91%).

¹H-NMR (400 MHz, DMSO-d₆): δ [ppm]=2.51-2.54 (m, 4H), 3.17 (s, 2H), 3.61-3.64 (m, 4H), 4.02 (s, 3H), 7.26 (d, J=9.1 Hz, 1H), 8.00 (dd, J=2.8, 9.1 Hz, 1H), 9.08 (d, J=3.0 Hz, 1H), 9.89 (s, 1H).

LC-MS (Method 3): Rₜ=0.96 min; MS (ESIpos): m/z=296 ([M+H]⁺, 70%); MS (ESIneg): m/z=294 ([M−H]⁻, 100%).

### Example 50A

### N-(2-{[tert-butyl(dimethyl)silyl]oxy}-5-nitrophenyl)-2-(morpholin-4-yl)acetamide

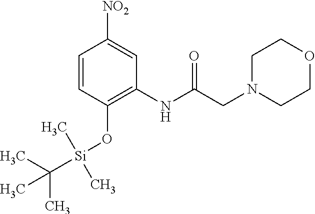 [SMILES: CC(C)(C)[Si](C)(C)Oc1ccc([N+](=O)[O-])cc1NC(=O)CN1CCOCC1]

STEP 1: To a solution of N-(2-{[tert-butyl(dimethyl)silyl]oxy}-5-nitrophenyl)-2-chloroacetamide (prepared in a manner analogous to that described in example 43A, 9.64 g, 28.0 mmol) in DMF (120 mL) was added morpholine (3.7 mL, 41.9 mmol, 1.5 equiv), triethylamine (5.8 mL, 41.9 mmol, 1.5 equiv) and potassium iodide (0.72 g, 4.33 mmol, 0.16 equiv). The reaction mixture was stirred at room temperature for 16 h. The resulting mixture was poured onto water (100 mL). The resulting precipitate was removed by filtration (0.45 g). The mother liquor was extracted with a CH₂Cl₂/isopropanol mixture (4:1, 4×100 mL). The combined organic phases were dried (Na₂SO₄ anh) and concentrated under reduced pressure to give impure N-(2-hydroxy-5-nitrophenyl)-2-(morpholin-4-yl)acetamide (3.6 g)

¹H-NMR (400 MHz, DMSO-d₆): δ [ppm]=2.50-2.54 (m, 4H), 3.17 (s, 2H), 3.60-3.63 (m, 4H), 6.97 (d, J=8.8 Hz, 1H), 7.86 (dd, J=3.0, 8.8 Hz, 1H), 9.04 (d, J=2.8 Hz, 1H), 9.78 (s, 1H).

LC-MS (Method 3): Rₜ=0.47 min; MS (ESIpos): m/z=282 ([M+H]⁺, 100%); MS (ESIneg): m/z=280 ([M−H]⁻, 100%).

STEP 2: To a solution of N-(2-hydroxy-5-nitrophenyl)-2-(morpholin-4-yl)acetamide from STEP 1 (1.50 g) in CH₂Cl₂ (30 mL) was added tert-butyldimethylsilyl chloride (0.96 g, 6.4 mmol) followed by triethylamine (1.04 mL, 7.47 mmol). The resulting mixture was stirred at room temperature for 12 h. Additional tert-butyldimethylsilyl chloride (0.48 g, 3.2 mmol) and triethylamine (1.04 mL, 7.47 mmol) was added, and the resulting mixture was stirred at room temperature for 48 h. The resulting mixture was diluted with diethyl ether (25 mL), then washed with a saturated aqueous ammonium chloride solution (25 mL). The aqueous phase was back-extracted with diethyl ether (25 mL). The combined organic phases were washed with a saturated aqueous ammonium chloride solution (25 mL), followed by water (25 mL), followed by a saturated NaCl solution (25 mL), then dried (Na₂SO₄ anh), and concentrated under reduced pressure. The resulting material was purified using MPLC (Biotage Isolera; 50 g SNAP cartridge: 100% hexane 2.0 min., gradient to 70% hexane/30% EtOAc 3.5 min., 70% hexane/30% EtOAc 2.0 min., gradient to 45% hexane/55% EtOAc 1.5 min., 45% hexane/55% EtOAc 12.0 min.) to give N-(2-{[tert-butyl(dimethyl)silyl]oxy}-5-nitrophenyl)-2-(morpholin-4-yl)acetamide (1.35 g, 9% over two steps).

¹H-NMR (400 MHz, CDCl₃): δ [ppm]=0.38 (s, 6H), 1.07 (s, 9H), 2.63 (br. s, 4H), 3.24 (br. s, 2H), 3.79 (br. s, 4H), 6.90 (d, J=9.0 Hz, 1H), 7.91 (dd, J=3.0, 8.9 Hz, 1H), 9.34 (br. s, 2H).

### Example 51A

### N-[5-amino-2-(trifluoromethoxy)phenyl]-2-(morpholin-4-yl)acetamide

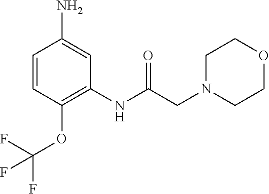 [SMILES: Nc1ccc(OC(F)(F)F)c(NC(=O)CN2CCOCC2)c1]

To a solution of 2-(morpholin-4-yl)-N-[5-nitro-2-(trifluoromethoxy)phenyl]acetamide (prepared in a manner analogous to that described in example 44A, 20.0 g, 57.1 mmol) in ethyl acetate (500 mL) was added 10% palladium on carbon (6.1 g, 5.72 mmol Pd, 10 mol % Pd). The resulting slurry was stirred under a hydrogen atmosphere for 3.25 h. The resulting slurry was filtered and concentrated under reduced pressure to afford N-[5-amino-2-(trifluoromethoxy)phenyl]-2-(morpholin-4-yl)acetamide (17.8 g, 98%).

¹H-NMR (400 MHz, DMSO-d₆): δ [ppm]=2.49-2.52 (m, 4H), 3.10 (s, 2H), 3.57-3.60 (m, 4H), 5.37 (s, 2H), 6.26 (dd, J=2.5, 8.8 Hz, 1H), 6.99 (dd, J=1.3, 8.8 Hz, 1H), 7.51 (d, J=2.5 Hz, 1H), 9.50 (s, 1H).

LC-MS (Method 5): Rₜ=0.99 min; MS (ESIpos): m/z=320 ([M+H]⁺, 90%); MS (ESIneg): m/z=318 ([M−H]⁻, 100%).

### Example 52A

### N-(5-amino-2-tert-butylphenyl)-2-(morpholin-4-yl)acetamide

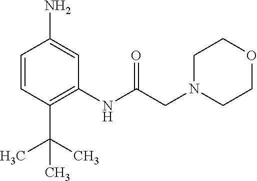 [SMILES: CC(C)(C)c1ccc(N)cc1NC(=O)CN1CCOCC1]

To a solution of N-(2-tert-butyl-5-nitrophenyl)-2-(morpholin-4-yl)acetamide (prepared in a manner analogous to that described in example 45A, 1.61 g, 5.01 mmol) in ethyl acetate (50 mL) was added 10% palladium on carbon (0.53 g, 0.50 mmol Pd, 10 mol % Pd). The resulting slurry was stirred under a hydrogen atmosphere for 4 h. The resulting slurry was filtered and concentrated under reduced pressure to afford N-(5-amino-2-tert-butylphenyl)-2-(morpholin-4-yl)acetamide (0.39 g, 27%).

¹H-NMR (400 MHz, DMSO-d₆): δ [ppm]=1.28 (s, 9H), 2.52-2.56 (m, 4H), 3.07 (s, 2H), 3.58-3.63 (m, 4H), 4.89 (s, 2H), 6.27 (dd, J=2.5, 8.5 Hz, 1H), 6.95 (d, J=8.5 Hz, 1H), 7.02 (d, J=2.5 Hz, 1H), 9.18 (s, 1H).

LC-MS (Method 4): Rₜ=0.98 min; MS (ESIpos): m/z=292 ([M+H]⁺, 100%), 583 ([2M+H]⁺, 10%); MS (ESIneg): m/z=290 ([M−H]⁻, 100%).

### Example 53A

### N-(5-amino-2-bromophenyl)-2-(morpholin-4-yl)acetamide

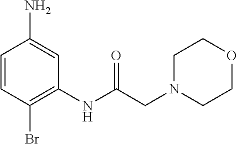 [SMILES: Nc1ccc(Br)c(NC(=O)CN2CCOCC2)c1]

To a solution of N-(2-bromo-5-nitrophenyl)-2-(morpholin-4-yl)acetamide (prepared in a manner analogous to that described in example 46A, 2.00 g, 5.81 mmol) in THF (60 mL) at 0° C. was added titanium trichloride (15% in an aqueous 10% HCl solution, 18.1 mL, 46.5 mmol, 8 equiv). The mixture was warmed to room temperature and was stirred at that temperature for 16 h. Additional titanium trichloride (15% in an aqueous 10% HCl solution, 18.1 mL, 46.5 mmol, 8 equiv) was added and the mixture was stirred at room temperature for 16 h. The resulting mixture was cooled with an ice bath and was cautiously neutralized with solid NaHCO₃. The resulting foam was extracted with ethyl acetate (4×100 mL). The combined organic phases were washed with a saturated NaCl solution (100 mL), dried (Na₂SO₄ anh), and concentrated under reduced pressure to give N-(5-amino-2-bromophenyl)-2-(morpholin-4-yl)acetamide (1.10 g, 50%).

¹H-NMR (400 MHz, DMSO-d₆): δ [ppm]=2.50-2.55 (m, 4H), 3.10 (s, 2H), 3.63-3.67 (m, 4H), 5.33 (s, 2H), 6.22 (dd, J=2.8, 8.7 Hz, 1H), 7.16 (d, J=8.7 Hz, 1H), 7.59 (d, J=2.6 Hz, 1H), 9.65 (s, 1H).

LC-MS (Method 4): Rₜ=0.92 min; MS (ESIpos): m/z=314 ([M+H]⁺, 100%).

### Example 54A

### N-(5-amino-2-chlorophenyl)-2-(morpholin-4-yl)acetamide

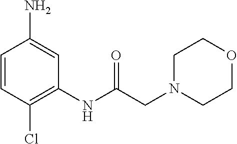 [SMILES: Nc1ccc(Cl)c(NC(=O)CN2CCOCC2)c1]

To a solution of N-(2-chloro-5-nitrophenyl)-2-(morpholin-4-yl)acetamide (prepared in a manner analogous to that described in example 47A, 1.00 g, 3.33 mmol) in methanol (10 mL) at 0° C. was added tin(II) chloride dihydrate (3.76 g, 16.7 mmol, 5.0 equiv). The resulting mixture was heated at the reflux temperature for 16 h, was then cooled to room temperature. The resulting mixture was treated with ethanol (20 mL). The resulting precipitate was removed with filtration, washed with a saturated Na₂CO₃ solution, followed by water, followed by ethanol, then dried at 50° C. under reduced pressure to give N-(5-amino-2-chlorophenyl)-2-(morpholin-4-yl)acetamide (0.45 g, 50%).

LC-MS (Method 4): Rₜ=0.87 min; MS (ESIpos): m/z=270 ([M+H]⁺, 100%); MS (ESIneg): m/z=268 ([M−H]⁻, 60%).

### Example 55A

### N-(5-amino-2-methylphenyl)-2-(morpholin-4-yl)acetamide

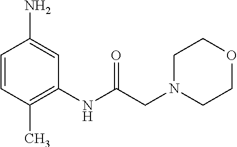 [SMILES: Cc1ccc(N)cc1NC(=O)CN1CCOCC1]

To a solution of N-(2-methyl-5-nitrophenyl)-2-(morpholin-4-yl)acetamide (prepared in a manner analogous to that described in example 48A, 2.09 g, 7.47 mmol) in ethyl acetate (80 mL) was added 10% palladium on carbon (0.80 g, 0.75 mmol Pd, 10 mol % Pd). The resulting slurry was stirred under a hydrogen atmosphere for 1.5 h. The resulting slurry was filtered and concentrated under reduced pressure to afford N-(5-amino-2-methylphenyl)-2-(morpholin-4-yl)acetamide (1.80 g, 97%).

¹H-NMR (400 MHz, DMSO-d₆): δ [ppm]=2.06 (s, 3H), 2.52-2.55 (m, 4H), 3.08 (s, 2H), 3.62-3.65 (m, 4H), 4.86 (s, 2H), 6.25 (dd, J=2.2, 7.9 Hz, 1H), 6.82 (d, J=8.2 Hz, 1H), 7.14 (d, J=2.2 Hz, 1H), 9.16 (s, 1H).

### Example 56A

### N-(5-amino-2-methoxyphenyl)-2-(morpholin-4-yl)acetamide

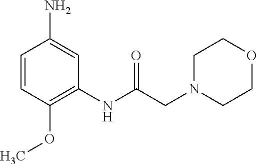 [SMILES: COc1ccc(N)cc1NC(=O)CN1CCOCC1]

To a solution of N-(2-methoxy-5-nitrophenyl)-2-(morpholin-4-yl)acetamide (prepared in a manner analogous to that described in example 49A, 15.5 g, 52.5 mmol) in ethyl acetate (500 mL) was added 10% palladium on carbon (5.59 g, 5.25 mmol Pd, 10 mol % Pd). The resulting slurry was stirred under a hydrogen atmosphere for 2 h. The resulting slurry was filtered and concentrated under reduced pressure to afford N-(5-amino-2-methoxyphenyl)-2-(morpholin-4-yl)acetamide (12.2 g, 88%).

¹H-NMR (400 MHz, DMSO-d₆): δ [ppm]=3.05 (s, 2H), 3.59-3.63 (m, 4H), 3.70 (s, 3H), 4.68 (s, 2H), 6.19 (dd, J=2.6, 8.7 Hz, 1H), 6.71 (d, J=8.5 Hz, 1H), 7.54 (d, J=2.8 Hz, 1H), 9.56 (s, 1H), protons at 2.48-2.50 ppm partially obscured by solvent.

LC-MS (Method 4): Rₜ=0.74 min; MS (ESIpos): m/z=266 ([M+H]⁺, 100%); MS (ESIneg): m/z=264 ([M−H]⁻, 90%).

### Example 57A

### N-(5-amino-2-{[tert-butyl(dimethyl)silyl]oxy}phenyl)-2-(morpholin-4-yl)acetamide

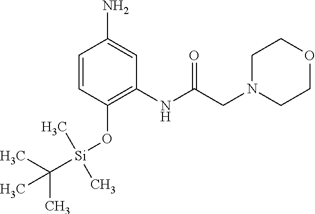 [SMILES: CC(C)(C)[Si](C)(C)Oc1ccc(N)cc1NC(=O)CN1CCOCC1]

To a solution of N-(2-{[tert-butyl(dimethyl)silyl]oxy}-5-nitrophenyl)-2-(morpholin-4-yl)acetamide (prepared in a manner analogous to that described in example 50A, 1.35 g, 3.41 mmol) in ethyl acetate (80 mL) was added 10% palladium on carbon (0.70 g, 0.66 mmol Pd, 19 mol % Pd). The resulting slurry was stirred under a hydrogen atmosphere for 7 h. The resulting slurry was filtered and concentrated under reduced pressure to afford N-(5-amino-2-{[tert-butyl(dimethyl)silyl]oxy}phenyl)-2-(morpholin-4-yl)acetamide (1.2 g, 92%).

¹H-NMR (400 MHz, DMSO-d₆): δ [ppm]=0.19 (s, 6H), 0.96 (s, 9H), 3.07 (s, 2H), 3.58-3.61 (m, 4H), 4.67 (s, 2H), 6.14 (dd, J=2.8, 8.6 Hz, 1H), 6.56 (d, J=8.6 Hz, 1H), 7.57 (d, J=2.8 Hz, 1H), 9.01 (s, 1H), protons at 2.43-2-45 ppm partially obscured by solvent.

LC-MS (Method 4): Rₜ=1.30 min; MS (ESIpos): m/z=366 ([M+H]⁺, 90%); MS (ESIneg): m/z=364 [M−H]⁻, 90%).

### Example 58A

### 4-bromo-N-{3-[(morpholin-4-ylacetyl)amino]-4-(trifluoromethoxy)phenyl}benzamide

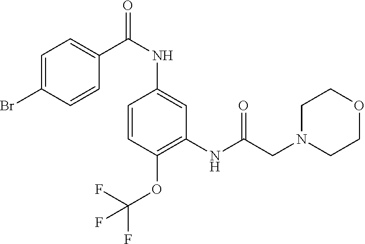 [SMILES: O=C(CN1CCOCC1)Nc1cc(NC(=O)c2ccc(Br)cc2)ccc1OC(F)(F)F]

To a solution of N-[5-amino-2-(trifluoromethoxy)phenyl]-2-(morpholin-4-yl)acetamide (prepared in a manner analogous to that described in example 51A, 0.76 g, 2.38 mmol) and 4-bromobenzoic acid (0.57 g, 2.86 mmol, 1.2 equiv) in DMF (25 mL) was added (benzotriazol-1-yloxy)tripyrrolidinophosphonium hexafluorophosphate (PYBOP, 2.97 g, 3.60 mmol, 1.20 equiv) followed by diisopropylethylamine (1.66 mL, 9.5 mmol, 4.0 equiv). The resulting mixture was stirred at room temperature for 24 h, was then diluted with water (25 mL). The resulting mixture was extracted with ethyl acetate (50 mL). The organic phase was dried (Na₂SO₄ anh) and concentrated under reduced pressure. The residue was crystallized from ethanol to give 4-bromo-N-{3-[(morpholin-4-ylacetyl)amino]-4-(trifluoromethoxy)phenyl}benzamide (0.70 g, 58%).

¹H-NMR (400 MHz, DMSO-d₆): δ [ppm]=2.51-2.55 (m, 4H), 3.16 (s, 2H), 3.58-3.62 (m, 4H), 7.40 (dd, J=1.3, 9.0 Hz, 1H), 7.68 (dd, J=2.5, 9.0 Hz, 1H), 7.72 (d, J=8.7 Hz, 2H), 7.88 (d, J=8.7 Hz, 2H), 8.65 (d, J=2.5 Hz, 1H), 9.76 (s, 1H), 10.52 (s, 1H).

### Example 59A

### 4-bromo-N-{4-methoxy-3-[(morpholin-4-ylacetyl)amino]phenyl}benzamide

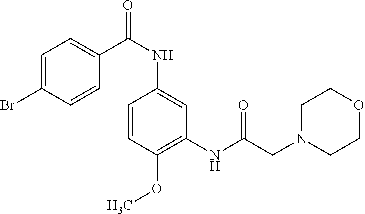 [SMILES: COc1ccc(NC(=O)c2ccc(Br)cc2)cc1NC(=O)CN1CCOCC1]

To a solution of N-(5-amino-2-methoxyphenyl)-2-(morpholin-4-yl)acetamide (prepared in a manner analogous to that described in example 56A, 1.93 g, 7.27 mmol) and 4-bromobenzoic acid (1.75 g, 8.73 mmol, 1.3 equiv) in DMF (75 mL) was added propanephosphonic acid cyclic anhydride solution (50% in ethyl acetate, 5.09 mL, 8.73 mmol, 1.2 equiv) followed by diisopropylethylamine (3.80 mL, 21.8 mmol, 3.0 equiv). The resulting mixture was stirred at room temperature for 24 h, was then treated with water (100 mL). The resulting mixture was extracted with ethyl acetate (100 mL). The organic phase was dried (Na₂SO₄ anh), and concentrated under reduced pressure. The residue was crystalized from ethanol to give 4-bromo-N-{4-methoxy-3-[(morpholin-4-ylacetyl)amino]phenyl}benzamide (1.70 g, 52%).

¹H-NMR (400 MHz, DMSO-d₆): δ [ppm]=2.50-2.54 (m, 4H), 3.11 (s, 2H), 3.61-3.66 (m, 4H), 3.85 (s, 3H), 7.01 (d, J=8.8 Hz, 1H), 7.52 (dd, J=2.6, 8.9 Hz, 1H), 7.69 (d, J=8.5 Hz, 2H), 7.88 (d, J=8.5 Hz, 2H), 8.51 (d, J=2.5 Hz, 1H), 9.70 (s, 1H), 10.21 (s, 1H).

LC-MS (Method 3): Rₜ=1.13 min; MS (ESIpos): m/z=448 ([M+H]⁺, 100%); MS (ESIneg): m/z=446 ([M−H]⁻, 100%).

### Example 60A

### tert-butyl [4′-({4-methoxy-3-[(morpholin-4-ylacetyl)amino]phenyl}carbamoyl)biphenyl-4-yl]carbamate

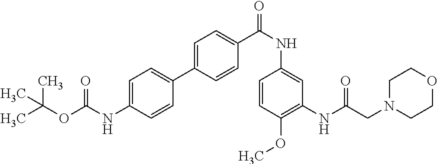 [SMILES: COc1ccc(NC(=O)c2ccc(-c3ccc(NC(=O)OC(C)(C)C)cc3)cc2)cc1NC(=O)CN1CCOCC1]

A mixture of 4-bromo-N-{4-methoxy-3-[(morpholin-4-ylacetyl)amino]phenyl}benzamide (prepared in a manner analogous to that described in example 59A, 0.075 g, 0.17 mmol) and {4-[(tert-butoxycarbonyl)amino]phenyl}boronic acid (0.079 g, 0.33 mmol, 2.0 equiv), [1,1′-bis(diphenylphosphino)ferrocene]palladium(II) chloride CH₂Cl₂ complex (Pd(dppf)Cl₂CH₂Cl₂, 0.013 g, 0.017 mmol, 10 mol %) and an aqueous potassium carbonate solution (2.0 N, 0.25 mL, 0.50 mmol, 3.0 equiv) in dioxane (2 mL) under an argon atmosphere was heated in a microwave apparatus at 105° C. for 1 h. The resulting mixture was cooled to room temperature and treated with water (2 mL). The aqueous solution was extracted with ethyl acetate (3×10 mL). The combined organic phases were dried (Na₂SO₄ anh) and concentrated under reduced pressure to give tert-butyl[4′-({4-methoxy-3-[(morpholin-4-ylacetyl)amino]phenyl}carbamoyl)biphenyl-4-yl]carbamate (0.097 g).

LC-MS (Method 3): Rₜ=1.26 min; MS (ESIpos): m/z=561 ([M+H]⁺, 100%); MS (ESIneg): m/z=559 ([M−H]⁻, 100%).

### Example 61A

### methyl 4-methoxy-3-[(morpholin-4-ylacetyl)amino]benzoate

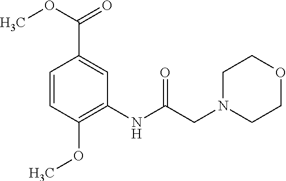 [SMILES: COC(=O)c1ccc(OC)c(NC(=O)CN2CCOCC2)c1]

To a solution of methyl 3-amino-4-methoxybenzoate (1.00 g, 5.52 mmol) and diisopropylethylamine (2.88 mL, 16.6 mmol, 3.0 equiv) in DMF (20 mL) was added morpholin-4-ylacetic acid (0.96 g, 6.62 mmol, 1.2 equiv) followed by propanephosphonic acid cyclic anhydride solution (50% in ethyl acetate, 3.87 mL, 6.62 mmol, 1.2 equiv). The resulting mixture was stirred at room temperature for 16 h, was then treated with water (25 mL). The resulting mixture was extracted with ethyl acetate (25 mL). The organic phase was dried (Na₂SO₄ anh), and concentrated under reduced pressure. The residue (1.5 g) was purified using MPLC (Biotage Isolera; 25 g SNAP cartridge: 100% hexane 2.0 min., gradient to 50% hexane/50% EtOAc 4.5 min., 50% hexane/50% EtOAc 8.5 min., gradient to 45% hexane/55% EtOAc 0.6 min., gradient to 100% EtOAc 5.8 min., 100% EtOAc 5.5 min.) to give methyl 4-methoxy-3-[(morpholin-4-ylacetyl)amino]benzoate (1.1 g, 62%).

¹H-NMR (400 MHz, DMSO-d₆): δ [ppm]=2.50-2.53 (m, 4H), 3.13 (s, 2H), 3.61-3.64 (m, 4H), 3.78 (s, 3H), 3.94 (s, 3H), 7.15 (d, J=8.6 Hz, 1H), 7.69 (dd, J=2.0, 8.6 Hz, 1H), 8.79 (d, J=2.3 Hz, 1H), 9.75 (s, 1H).

LC-MS (Method 3): Rₜ=1.02 min; MS (ESIpos): m/z=309 ([M+H]⁺, 90%); MS (ESIneg): m/z=307 ([M−H]⁻, 100%).

### Example 62A

### 4-methoxy-3-[(morpholin-4-ylacetyl)amino]benzoic acid

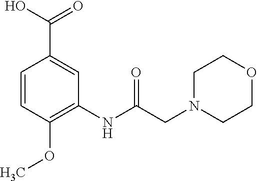 [SMILES: COc1ccc(C(=O)O)cc1NC(=O)CN1CCOCC1]

To a solution of methyl 4-methoxy-3-[(morpholin-4-ylacetyl)amino]benzoate (prepared in a manner analogous to that described in example 61A, 1.04 g, 3.37 mmol) in methanol (7 mL) was added an aqueous lithium hydroxide solution (1 N, 10.1 mL, 10.1 mmol, 3.0 equiv). The resulting solution was stirred at room temperature for 12 h, was then concentrated under reduced pressure. The residue was dissolved in water (10 mL), acidified with an aqueous 2N HCl solution (5.06 mL, 10.1 mmol, 3.0 equiv), and concentrated under reduced pressure. The residue was treated with toluene (10 mL), then concentrated under reduced pressure to give 4-methoxy-3-[(morpholin-4-ylacetyl)amino]benzoic acid (0.80 g, 81%).

LC-MS (Method 3): Rₜ=0.45 min; MS (ESIpos): m/z=295 ([M+H]⁺, 100%); MS (ESIneg): m/z=293 ([M−H]⁻, 100%).

### Example 63A

### 1-(4-methylpiperazin-1-yl)cyclopropanecarboxylic acid hydrochloride (1:1)

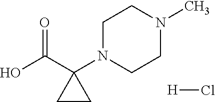

The title compound was prepared according to the following scheme:

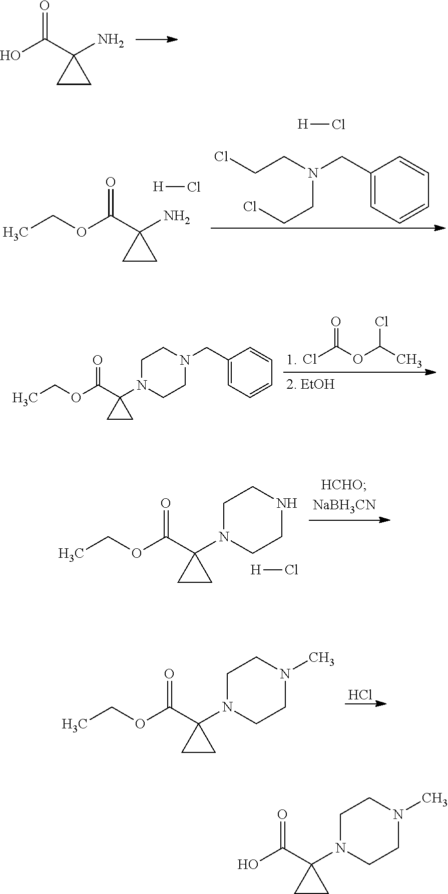

### LC-MS Methods for Examples 63A and Example 64A

MS instrument type: Agilent 1956A; HPLC instrument type: Agilent 1200 Series; UV DAD; column: Agilent TC-C₁₈, 2.1×50 mm, 5 μm; mobile phase A: 0.0375% TFA in water, mobile phase B: 0.0188% TFA in acetonitrile; gradient: 0.0 min 100% A->1.0 min 100% A->3.4 min 20% A->3.9 min 0% A->3.91 min 100% A->4.0 min 100% A->4.5 min 100% A; flow rate: 0.0 min 0.6 ml/min->1.0 min/3.4 min/3.9 min/3.91 min 0.6 ml/min->4.0 min/4.5 min 1.0 ml/min; column temp: 40° C.; UV detection: 220 nm.

### Step 1

### ethyl 1-aminocyclopropanecarboxylate hydrochloride (1:1)

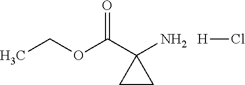

Thionyl chloride (150 mL, 2.056 mol) was added slowly below 0° C. to a suspension of 1-aminocyclopropanecarboxylic acid (100 g, 0.989 mol) in anhydrous ethanol (1 L). The mixture was stirred at 70° C. for 20 h. TLC (methanol, R_{f}=0.4) showed that most of the starting material was consumed. Then the solution was concentrated to give 210 g of crude product. The residue was dissolved in water and adjusted to a pH between 9 and 10 with potassium carbonate. The aqueous layer was extracted with dichloromethane (1 L×3). The combined organic layers were concentrated to dryness. The residue was dissolved in ethyl acetate (300 mL) and hydrochloride in ethyl acetate (250 mL, 4M) was added slowly to the solution below −30° C. It was stirred for 30 min at 0° C. A solid precipitated and it was filtered under nitrogen atmosphere to give ethyl 1-aminocyclopropanecarboxylate hydrochloride (132 g, 80.6% yield) as a white solid.

The following ¹H-NMR is from the free amine.

¹H-NMR (400 MHz, chloroform-d₁): δ [ppm]=0.91-1.02 (m, 2H), 1.15-1.30 (m, 5H), 2.17 (s, 2H), 4.10 (d, 2H).

### Step 2

### ethyl 1-(4-benzylpiperazin-1-yl)cyclopropanecarboxylate

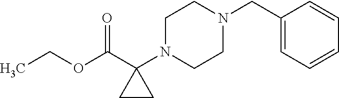 [SMILES: CCOC(=O)C1(N2CCN(Cc3ccccc3)CC2)CC1]

A mixture of ethyl 1-aminocyclopropanecarboxylate hydrochloride (120 g, 0.725 mol), N,N-diisopropylethylamine (942 g, 7.29 mol), N-benzyl-2-chloro-N-(2-chloroethyl)ethanamine hydrochloride (213 g, 0.793 mol) in anhydrous ethanol (1.6 L) was stirred under reflux for 16 h. TLC (PE:EtOAc=5:1, R_{f}=0.4) showed that most of the starting material was consumed. Then the mixture was concentrated. The residue was partitioned between dichloromethane (1 L) and water (0.5 L). The layers were separated and the aqueous layer was extracted with dichloromethane (0.5 L×2). The combined organic layers were concentrated. The residue was purified by chromatography on silica gel (PE:EtOAc=20:1 to 10:1) to give ethyl 1-(4-benzylpiperazin-1-yl)cyclopropanecarboxylate (100 g, 47.8%) as a light yellow oil.

¹H-NMR (400 MHz, chloroform-d₁): δ [ppm]=0.88-0.97 (m, 2H), 1.23-1.36 (m, 5H), 2.37 (br. S, 4H), 2.98 (br. S, 4H), 3.51 (s, 2H), 4.15 (q, 2H), 7.23-7.36 (m, 5H).

### Step 3

### ethyl 1-(piperazin-1-yl)cyclopropanecarboxylate hydrochloride (1:1)

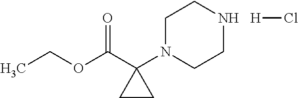

To a solution of ethyl 1-(4-benzylpiperazin-1-yl)cyclopropanecarboxylate (83 g, 0.288 mol) in anhydrous dichloromethane (700 mL) 1-chloroethyl carbonochloridate (60.4 g, 0.422 mol) was slowly added below 0° C. After the addition, the mixture was stirred at 18° C. for 1 h. TLC (PE:EtOAc=4:1, R_{f}=0.85) showed that the reaction was complete. Then it was concentrated to dryness. The residue was dissolved in ethanol (700 mL). It was stirred under reflux for 16 h. TLC (PE:EtOAc=4:1, R_{f}=0) showed the reaction was complete. Then it was concentrated to dryness. The residue was stirred with ethanol:methyl-tert-butylether=5:1 to give ethyl 1-(piperazin-1-yl)cyclopropanecarboxylate hydrochloride (1:1) (62 g, 92%) as a white solid.

¹H-NMR (400 MHz, methanol-d₄): δ [ppm]=1.27 (t, 3H), 1.50-1.65 (m, 4H), 3.50 (mc, 4H), 3.65-3.85 (m, 4H), 4.21 (q, 2H).

### Step 4

### ethyl 1-(4-methylpiperazin-1-yl)cyclopropanecarboxylate

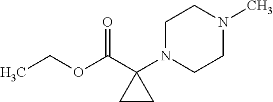

To a solution of ethyl 1-(piperazin-1-yl)cyclopropanecarboxylate hydrochloride (25 g, 0.107 mol) in water (250 mL) was added solid sodium hydrogen carbonate (10 g, 0.119 mol) so that a pH of 7-8 was reached. Then formaldehyde (13.5 g, 0.166 mol, 37% in water) and sodium cyanoborohydride (17.3 g, 0.275 mol) were added below 10° C. The mixture was stirred 18 h at 18° C. TLC (PE:EtOAc=1:1, R_{f}=0.1) showed that most of the starting material was consumed. Then it was extracted with dichloromethane (50 mL×3). The combined organic phases were concentrated to dryness. The residue was purified by chromatography on silica gel (PE:EtOAc=3:1 to dichloromethane:methanol=15:1) to give ethyl 1-(4-methylpiperazin-1-yl)cyclopropanecarboxylate (12 g, 53%).

¹H-NMR (400 MHz, methanol-d₄): δ [ppm]=0.98-1.04 (m, 2H), 1.24 (t, 3H), 1.26-1.31 (m, 2H), 2.70 (s, 3H), 2.97 (mc, 4H), 3.20 (mc, 4H), 4.11 (q, 2H).

### Step 5

### 1-(4-methylpiperazin-1-yl)cyclopropanecarboxylic acid hydrochloride (1:1)

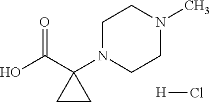

To a round bottom flask containing ethyl 1-(4-methylpiperazin-1-yl)cyclopropanecarboxylate (14 g, 65.9 mmol) was added aqueous hydrochloric acid (6M, 100 mL) slowly below 20° C. After the addition, the mixture was stirred at 100-140° C. for 24 h. TLC (dichloromethane:methanol=8:1, R_{f}=0.0) showed that the reaction was complete. Then the reaction mixture was concentrated to dryness. The residue was stirred in ethanol and the solid was filtered off to give 1-(4-methylpiperazin-1-yl)cyclopropanecarboxylic acid hydrochloride (1:1) (6.4 g, 44%) as a white solid.

¹H-NMR (400 MHz, water-d₂): δ [ppm]=1.27-1.37 (m, 2H), 1.45-1.56 (m, 2H), 2.88 (d, 3H), 3.08-3.23 (m, 2H), 3.45-3.53 (m, 2H), 3.55-3.68 (m, 2H), 3.72-3.87 (m, 2H).

ELSD: M/Z=211.1 (M+H⁺).

### Example 64A

### 1-(4-cyclopropylpiperazin-1-yl)cyclopropanecarboxylic acid hydrochloride (1:1)

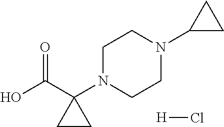

### Step 1

### ethyl 1-(4-cyclopropylpiperazin-1-yl)cyclopropanecarboxylate

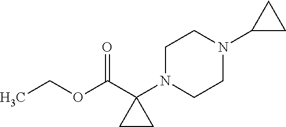 [SMILES: CCOC(=O)C1(N2CCN(C3CC3)CC2)CC1]

To a solution of ethyl 1-(piperazin-1-yl)cyclopropanecarboxylate hydrochloride (12.8 g, 54.5 mmol) in a mixture of anhydrous THF (68 mL) and methanol (68 mL) (1-ethoxycyclopropoxy)trimethylsilane (21.9 ml, 108.9 mmol) and acetic acid (10 mL) were added. Then sodium cyanoborohydride (5.14 g, 81.8 mmol) was added in portions. After the addition, the mixture was stirred at 60° C. for 16 h. TLC (dichloromethane:methanol=4:1, R_{f}=0.9) showed that the reaction was complete. It was cooled to 18° C. and quenched with water (5 mL). It was concentrated to dryness and the residue was partitioned between dichloromethane (100 mL) and aqueous saturated sodium hydrogen carbonate (20 mL). The layers were separated and the aqueous layer was extracted with dichloromethane (100 mL). The combined organic layers were washed with water (15 mL) and concentrated to dryness. The residue was purified by column chromatography on silica gel (PE:EtOAc=20:1 to 8:1) to give ethyl 1-(4-cyclopropylpiperazin-1-yl)cyclopropanecarboxylate (12 g, 92%) as a light yellow oil.

¹H-NMR (400 MHz, methanol-d₄): δ [ppm]=0.40-0.45 (m, 4H), 0.91-0.97 (m, 2H), 1.19-1.28 (m, 5H), 1.58-1.66 (m, 1H), 2.40-2.70 (m, 4H), 2.87-3.09 (m, 4H), 4.10 (q, 2H).

### Step 2

### 1-(4-cyclopropylpiperazin-1-yl)cyclopropanecarboxylic acid hydrochloride (1:1)

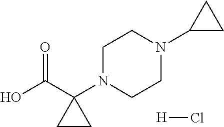

To a round bottom flask containing ethyl 1-(piperazin-1-yl)cyclopropanecarboxylate (12 g, 50.4 mmol) was added aqueous hydrochloric acid (6M, 100 mL) below 0° C. After the addition, the mixture was stirred at 100° C. for 16 h. TLC (dichloromethane:methanol=10:1, R_{f}=0.4) showed that the reaction was complete. Then the reaction mixture was concentrated under reduced pressure and the residue was stirred in ethanol (40 mL). The solid was filtered off to give 1-(4-cyclopropylpiperazin-1-yl)cyclopropanecarboxylic acid hydrochloride (1:1) (10.2 g, 82%) as a white solid.

¹H-NMR (400 MHz, water-d₂): δ [ppm]=0.87-0.98 (m, 4H), 1.25-1.33 (m, 2H), 1.45-1.53 (m, 2H), 2.77-2.85 (m, 1H), 3.28-3.78 (m, 8H).

ELSD: M/Z=211.1 (M+H⁺).

### Example 65A

### 1-(morpholin-4-yl)cyclopropanecarboxylic acid hydrochloride (1:1)

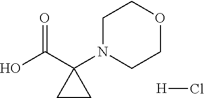

The title compound is known from WO2010/136778.

### Example 66A

### 4-[(2-methoxyethoxy)methyl]-3-nitrobenzoic acid

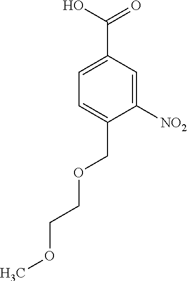 [SMILES: COCCOCc1ccc(C(=O)O)cc1[N+](=O)[O-]]

615 mg (15.4 mmol) of sodium hydride (60%) were added at 0° C. in small portions to 15 mL of 2-methoxyethanol and stirred for 10 minutes. 1.00 g (3.85 mmol) of 4-(bromomethyl)-3-nitrobenzoic acid was added. The reaction mixture was allowed to warm up to room temperature, was stirred for 1 h, was poured into water, was acidified with a 1N aqueous solution of hydrogen chloride and was extracted with ethyl acetate. The combined organic phases were washed with brine, dried over sodium sulfate and concentrated to yield 1.23 g of the title compound, which was used without further purification.

¹H-NMR (400 MHz, DMSO-d₆): δ [ppm]=3.27 (s, 3H), 3.50-3.55 (m, 2H), 3.64-3.68 (m, 2H), 4.91 (s, 2H), 7.91 (d, 1H), 8.27 (dd, 1H), 8.49 (d, 1H), 13.67 (s, 1H).

LC-MS (Method 4): Rₜ=0.87 min; MS (ESIneg): m/z=254 [M−H]⁻.

### Example 67A

### N-(biphenyl-4-yl)-4-[(2-methoxyethoxy)methyl]-3-nitrobenzamide

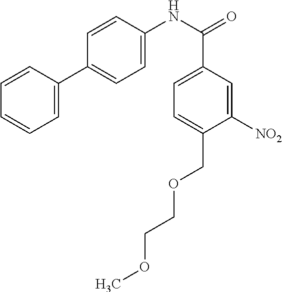 [SMILES: COCCOCc1ccc(C(=O)Nc2ccc(-c3ccccc3)cc2)cc1[N+](=O)[O-]]

917 mg (5.42 mmol) of biphenyl-4-amine and 1.9 mL (10.8 mmol) of N,N-diisopropylethylamine were provided in 10 mL of DMF. A solution of 1.23 g of the compound of example 66A in 10 mL of DMF and 4.22 mL (7.23 mmol) of a 50% solution of 2,4,6-tripropyl-1,3,5,2,4,6-trioxatriphosphinane 2,4,6-trioxide (T3P) in DMF were added, and the mixture was stirred over night at room temperature. After concentration, the residue (6.1 g) was purified using MPLC (Biotage Isolera; silica gel; hexane/EtOAc gradient) to yield 1.17 g (72% of theory) of the title compound.

¹H-NMR (300 MHz, DMSO-d₆): δ [ppm]=3.28 (s, 3H), 3.50-3.56 (m, 2H), 3.64-3.70 (m, 2H), 4.93 (s, 2H), 7.31-7.39 (m, 1H), 7.43-7.50 (m, 2H), 7.65-7.75 (m, 4H), 7.86-7.97 (m, 3H), 8.37 (dd, 1H), 8.67 (d, 1H), 10.63 (s, 1H).

LC-MS (Method 1): Rₜ=1.35 min; MS (ESIpos): m/z=407 [M+H]⁺.

### Example 68A

### 3-amino-N-(biphenyl-4-yl)-4-[(2-methoxyethoxy)methyl]benzamide

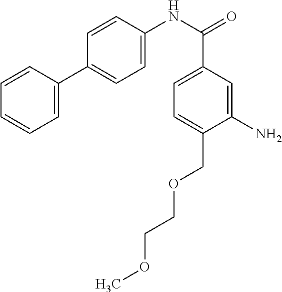 [SMILES: COCCOCc1ccc(C(=O)Nc2ccc(-c3ccccc3)cc2)cc1N]

To a solution of the compound of example 67A (1.14 g, 2.52 mmol) in 30 mL of tetrahydrofuran was added a 15% solution of titanium(III) chloride in 10% hydrogen chloride dropwise (21.5 mL, 25.2 mmol, 10 equiv) at 0° C. The reaction mixture was allowed to warm up to room temperature and was stirred over night. The pH of the mixture was adjusted under stirring with solid sodium bicarbonate to 7. The suspension was saturated with solid sodium chloride and stirred with 70 mL of a mixture of tetrahydrofuran/ethyl acetate 1:1 for 2 h. The suspension was filtered and the filtrate was washed with brine, dried over sodium sulfate and concentrated under reduced pressure. 0.95 g (100% of theory) of the title compound were obtained, which were used without further purification.

¹H-NMR (400 MHz, DMSO-d₆): δ [ppm]=3.28 (s, 3H), 3.49-3.54 (m, 2H), 3.55-3.60 (m, 2H), 4.46 (s, 2H), 5.21 (s, 2H), 7.13 (dd, 1H), 7.19 (d, 1H), 7.22 (d, 1H), 7.31-7.37 (m, 1H), 7.42-7.48 (m, 2H), 7.63-7.70 (m, 4H), 7.85-7.90 (m, 2H), 10.16 (s, 1H).

LC-MS (Method 4): Rₜ=1.22 min; MS (ESIpos): m/z=377 [M+H]⁺.

### Example 69A

### N-(biphenyl-4-yl)-3-[(chloroacetyl)amino]-4-[(2-methoxyethoxy)methyl]benzamide

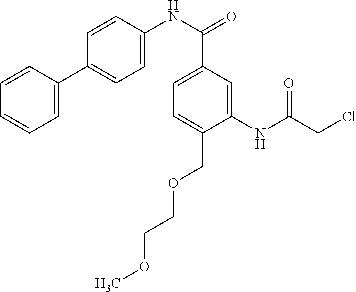 [SMILES: COCCOCc1ccc(C(=O)Nc2ccc(-c3ccccc3)cc2)cc1NC(=O)CCl]

475 mg (1.26 mmol) of the compound of example 68A were provided in 11 mL of toluene, 0.2 mL (2.52 mmol) of chloroacetyl chloride were added, and the mixture was stirred for 2 h at 100° C. After concentration, the residue (0.72 g) was purified using MPLC (Biotage Isolera; silica gel; hexane/EtOAc gradient) to yield 148 mg (26% of theory) of the title compound.

¹H-NMR (300 MHz, DMSO-d₆): δ [ppm]=3.28 (s, 3H), 3.50-3.56 (m, 2H), 3.58-3.65 (m, 2H), 4.38 (s, 2H), 4.59 (s, 2H), 7.29-7.38 (m, 1H), 7.42-7.50 (m, 2H), 7.57 (d, 1H), 7.65-7.72 (m, 4H), 7.81-7.92 (m, 3H), 8.14-8.19 (m, 1H), 9.84 (s, 1H), 10.39 (s, 1H).

LC-MS (Method 4): Rₜ=1.29 min; MS (ESIpos): m/z=453 [M+H]⁺.

### Example 70A

### 4-[(3-methoxypropoxy)methyl]-3-nitrobenzoic acid

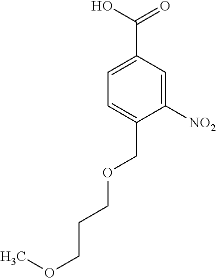 [SMILES: COCCCOCc1ccc(C(=O)O)cc1[N+](=O)[O-]]

615 mg (15.4 mmol) of sodium hydride (60%) were added at 0° C. in small portions to 10 mL of 3-methoxypropan-1-ol and stirred for 10 minutes. 1.00 g (3.85 mmol) of 4-(bromomethyl)-3-nitrobenzoic acid was added, the reaction mixture was allowed to warm up to room temperature, was stirred for 1 h, was poured into water, was acidified with a 1N aqueous solution of hydrogen chloride and was extracted with ethyl acetate. The combined organic phases were washed with brine, dried over sodium sulfate and concentrated to yield 1.16 g of the title compound, which was used without further purification.

¹H-NMR (300 MHz, DMSO-d₆): δ [ppm]=1.80 (quin, 2H), 3.23 (s, 3H), 3.40 (t, 2H), 3.56 (t, 2H), 7.87 (d, 1H), 8.26 (d, 1H), 8.47 (s, 1H), 13.60 (s, 1H).

LC-MS (Method 4): Rₜ=0.96 min; MS (ESIneg): m/z=268 [M−H]⁻.

### Example 71A

### N-(biphenyl-4-yl)-4-[(3-methoxypropoxy)methyl]-3-nitrobenzamide

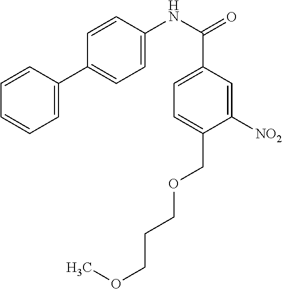 [SMILES: COCCCOCc1ccc(C(=O)Nc2ccc(-c3ccccc3)cc2)cc1[N+](=O)[O-]]

875 mg (5.17 mmol) of biphenyl-4-amine and 1.8 mL (10.3 mmol) of N,N-diisopropylethylamine were provided in 10 mL of DMF. A solution of 1.16 g of the compound of example 70A in 10 mL of DMF and 4.02 mL (6.89 mmol) of a 50% solution of 2,4,6-tripropyl-1,3,5,2,4,6-trioxatriphosphinane 2,4,6-trioxide (T3P) in DMF were added, and the mixture was stirred over night at room temperature. After concentration, the residue (5.8 g) was purified using MPLC (Biotage Isolera; silica gel; hexane/EtOAc gradient) to yield 1.00 g (69% of theory) of the title compound.

LC-MS (Method 4): Rₜ=1.42 min; MS (ESIpos): m/z=421 [M+H]⁺.

### Example 72A

### 3-amino-N-(biphenyl-4-yl)-4-[(3-methoxypropoxy)methyl]benzamide

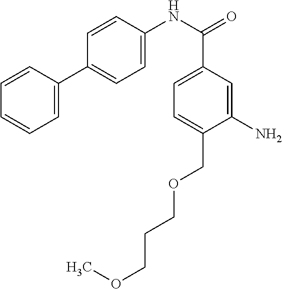 [SMILES: COCCCOCc1ccc(C(=O)Nc2ccc(-c3ccccc3)cc2)cc1N]

To a solution of the compound of example 71A (1.12 g, 2.40 mmol) in 30 mL of tetrahydrofuran was added a 15% solution of titanium(III) chloride in 10% hydrogen chloride dropwise (20.4 mL, 23.4 mmol, 10 equiv) at 0° C. The reaction mixture was allowed to warm up to room temperature and was stirred over night. The pH of the mixture was adjusted under stirring with solid sodium bicarbonate to 7. The suspension was saturated with solid sodium chloride and stirred with 70 mL of a mixture of tetrahydrofuran/ethyl acetate 1:1 for 2 h. The suspension was filtered and the filtrate was washed with brine, dried over sodium sulfate and concentrated under reduced pressure. The residue (1.03 g) was purified using MPLC (Biotage Isolera; silica gel; hexane/EtOAc gradient) to yield 566 mg (60% of theory) of the title compound.

¹H-NMR (300 MHz, DMSO-d₆): δ [ppm]=1.78 (quin, 2H), 3.22 (s, 3H), 3.39 (t, 2H), 3.48 (t, 2H), 4.42 (s, 2H), 5.20 (s, 2H), 7.10-7.16 (m, 1H), 7.19 (d, 2H), 7.30-7.37 (m, 1H), 7.41-7.50 (m, 2H), 7.62-7.71 (m, 4H), 7.84-7.91 (m, 2H), 10.18 (s, 1H).

LC-MS (Method 4): Rₜ=1.27 min; MS (ESIpos): m/z=391 [M+H]⁺.

### Example 73A

### N-(biphenyl-4-yl)-3-[(chloroacetyl)amino]-4-[(3-methoxypropoxy)methyl]benzamide

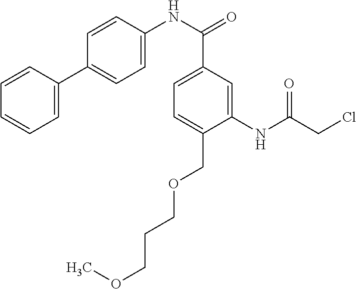 [SMILES: COCCCOCc1ccc(C(=O)Nc2ccc(-c3ccccc3)cc2)cc1NC(=O)CCl]

360 mg (0.92 mmol) of the compound of example 72A were provided in 8 mL of toluene, 0.15 mL (1.84 mmol) of chloroacetyl chloride were added, and the mixture was stirred for 2 h at 100° C. After concentration, the residue (0.52 g) was purified using MPLC (Biotage Isolera; silica gel; hexane/EtOAc gradient) to yield 237 mg (55% of theory) of the title compound.

¹H-NMR (300 MHz, DMSO-d₆): δ [ppm]=1.81 (quin, 2H), 3.22 (s, 3H), 3.40 (t, 2H), 3.52 (t, 2H), 4.39 (s, 2H), 4.55 (s, 2H), 7.31-7.38 (m, 1H), 7.42-7.50 (m, 2H), 7.56 (d, 1H), 7.65-7.72 (m, 4H), 7.82-7.91 (m, 3H), 8.13-8.18 (m, 1H), 9.86 (s, 1H), 10.39 (s, 1H).

LC-MS (Method 4): Rₜ=1.33 min; MS (ESIpos): m/z=467 [M+H]⁺.

### Example 74A

### methyl 4-(benzyloxy)-3-({[1-(morpholin-4-yl)cyclopropyl]carbonyl}amino)benzoate

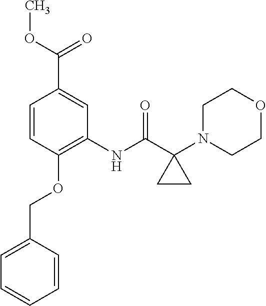 [SMILES: COC(=O)c1ccc(OCc2ccccc2)c(NC(=O)C2(N3CCOCC3)CC2)c1]

To a solution of methyl 3-amino-4-(benzyloxy)benzoate (5.00 g, 19.4 mmol) and 1-(morpholin-4-yl)cyclopropanecarboxylic acid hydrochloride (1:1) (example 65A) (4.84 g, 23.3 mmol) in DMF (50 mL) was added (benzotriazol-1-yloxy)tripyrrolidinophosphonium hexafluorophosphate (PYBOP, 20.2 g, 38.9 mmol) and diisopropylethylamine (16.9 mL, 97.2 mmol). The resulting mixture was stirred at room temperature over night, was concentrated under reduced pressure, was then dissolved in dichloromethane, was washed with 1N aqueous hydrogen chloride solution and saturated, aqueous sodium bicarbonate solution, was dried over sodium sulfate and concentrated under reduced pressure. The remaining solids were then triturated with ethanol (100 mL), and the resulting mixture was stirred for 30 minutes. The remaining solids were removed by filtration, washed with ethanol, and were dried at 50° C. under reduced pressure to give the title compound (7.98 g, 100% of theory).

LC-MS (Method 4): Rₜ=1.32 min; MS (ESIpos): m/z=411 [M+H]⁺.

### Example 75A

### 4-(benzyloxy)-3-({[1-(morpholin-4-yl)cyclopropyl]carbonyl}amino)benzoic acid

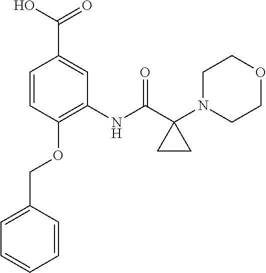 [SMILES: O=C(O)c1ccc(OCc2ccccc2)c(NC(=O)C2(N3CCOCC3)CC2)c1]

7.98 g (19.4 mmol) of the compound of intermediate 74A were provided in 80 mL of dioxane, 931 mg (38.9 mmol) of lithium hydroxide and 34 mL of water were added at room temperature and the mixture was stirred at room temperature for 22 hours. Water and a 2N aqueous hydrogen chloride solution were then added until an acidic pH of 1.5-2 was achieved. After stirring for 15 minutes, the precipitate was filtered off, washed with water and dried. 5.70 g (74% of theory) of the title compound were obtained, which were used without further purification.

¹H-NMR (400 MHz, DMSO-d₆): δ [ppm]=1.04-1.09 (m, 2H), 1.10-1.16 (m, 2H), 2.21-2.29 (m, 4H), 3.14-3.23 (m, 4H), 5.25 (s, 2H), 7.29 (d, 1H), 7.38-7.47 (m, 3H), 7.54-7.59 (m, 2H), 7.67 (dd, 1H), 8.92 (d, 1H), 10.37 (s, 1H).

LC-MS (Method 1): Rₜ=1.13 min; MS (ESIpos): m/z=397 [M+H]⁺.

### Example 76A

### N-(biphenyl-4-yl)-4-hydroxy-3-({[1-(morpholin-4-yl)cyclopropyl]carbonyl}amino)benzamide

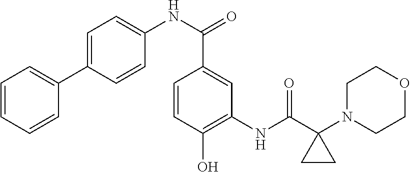 [SMILES: O=C(Nc1ccc(-c2ccccc2)cc1)c1ccc(O)c(NC(=O)C2(N3CCOCC3)CC2)c1]

To a solution of the compound of example 88 (0.50 g, 0.91 mmol) in a mixture of THF (43 mL) and methanol (16 mL) was added 10% palladium on carbon (0.16 g, 0.15 mmol Pd, 50% water). The resulting slurry was stirred under a hydrogen atmosphere at room temperature until the starting material was consumed. The resulting suspension was filtered and concentrated under reduced pressure to give the title compound (0.36 g, 87%).

¹H-NMR (300 MHz, DMSO-d₆): δ [ppm]=1.08-1.16 (m, 2H), 1.17-1.24 (m, 2H), 2.41-2.49 (m, 4H), 3.68-3.76 (m, 4H), 6.98 (d, 1H), 7.29-7.37 (m, 1H), 7.39-7.50 (m, 2H), 7.58 (dd, 1H), 7.62-7.70 (m, 4H), 7.81-7.89 (m, 2H), 8.79 (d, 1H), 10.13 (s, 1H), 10.54 (s, 1H), 11.00 (s, 1H).

LC-MS (Method 1): Rₜ=1.26 min; MS (ESIpos): m/z=458 [M+H]⁺.

### Example 77A

### tert-butyl {3-[4-(biphenyl-4-ylcarbamoyl)-2-({[1-(morpholin-4-yl)cyclopropyl]carbonyl}amino)phenoxy]propyl}carbamate

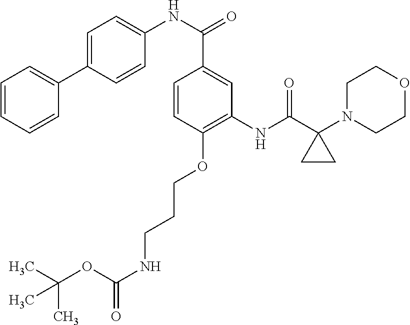 [SMILES: CC(C)(C)OC(=O)NCCCOc1ccc(C(=O)Nc2ccc(-c3ccccc3)cc2)cc1NC(=O)C1(N2CCOCC2)CC1]

To a solution of tert-butyl(3-hydroxypropyl)carbamate (0.19 mL, 1.11 mmol) and triethylamine (0.31 mL, 2.23 mmol) in dichloromethane (4 mL) was added methanesulfonyl chloride (0.13 mL, 1.67 mmol) dropwise. The resulting mixture was stirred at room temperature for 1.5 h, water was added and the mixture was extracted with dichloromethane. The combined organic phases were washed with a saturated, aqueous sodium bicarbonate solution and brine, were dried over sodium sulfate and concentrated under reduced pressure. A solution of the remaining material in DMF (2.5 mL) was added to a mixture of the compound of example 76A (364 mg, 0.80 mmol) and cesium carbonate (518 mg, 1.59 mmol) in DMF (2.5 mL). The resulting mixture was stirred for 1 h at 70° C., water was added and the mixture was extracted with dichloromethane. The combined organic phases were washed with water, were dried over sodium sulfate and concentrated under reduced pressure to give the title compound (560 mg), which was used without further purification.

¹H-NMR (300 MHz, DMSO-d₆): δ [ppm]=1.09-1.18 (m, 2H), 1.18-1.27 (m, 2H), 1.38 (s, 9H), 1.93-2.09 (m, 2H), 2.39-2.48 (m, 4H), 3.10-3.22 (m, 2H), 3.64-3.78 (m, 4H), 4.21 (t, 2H), 6.94-7.06 (m, 1H), 7.19 (d, 1H), 7.28-7.38 (m, 1H), 7.41-7.51 (m, 2H), 7.61-7.78 (m, 5H), 7.82-7.89 (m, 2H), 8.89 (d, 1H), 10.23 (s, 1H), 10.40 (s, 1H).

LC-MS (Method 4): Rₜ=1.43 min; MS (ESIpos): m/z=615 [M+H]⁺.

### Example 78A

### 3-amino-N-(biphenyl-4-yl)-4-(trifluoromethoxy)benzamide

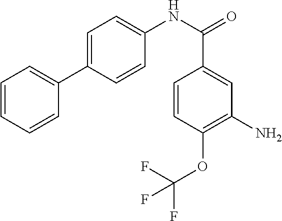 [SMILES: Nc1cc(C(=O)Nc2ccc(-c3ccccc3)cc2)ccc1OC(F)(F)F]

To a solution of biphenyl-4-amine (765 mg, 4.52 mmol) and 3-amino-4-(trifluoromethoxy)benzoic acid (known from WO2007/31791, 500 mg, 2.26 mmol) in DMF (5 mL) was added (benzotriazol-1-yloxy)tripyrrolidinophosphonium hexafluorophosphate (PYBOP, 2.35 g, 4.52 mmol) followed by diisopropylethylamine (2.0 mL, 11.3 mmol). The resulting mixture was stirred at room temperature for 3 days, was then treated with water and stirred for 15 minutes. The precipitate was collected by filtration, washed with water and dried. The residue (1.94 g) was purified using MPLC (Biotage Isolera; silica gel; hexane/EtOAc gradient) and preparative HPLC (column: chromatorex C18, 10 μm, 195×51 mm, mobile phase: acetonitrile/water gradient with the addition of 0.1% formic acid) to give the title compound (433 mg, 53%).

¹H-NMR (300 MHz, DMSO-d₆): δ [ppm]=5.67 (s, 2H), 7.12 (dd, 1H), 7.25 (dd, 1H), 7.29-7.38 (m, 2H), 7.41-7.50 (m, 2H), 7.63-7.71 (m, 4H), 7.82-7.89 (m, 2H), 10.29 (s, 1H).

LC-MS (Method 1): Rₜ=1.35 min; MS (ESIpos): m/z=373 [M+H]⁺.

### Example 79A

### 4-(methoxymethyl)-3-nitrobenzoic acid

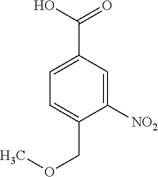

To a solution of 10.0 g (38.5 mmol) of 4-(bromomethyl)-3-nitrobenzoic acid in 200 mL of methanol were added 231 mL (115 mmol, 3 equiv) of a 0.5M solution of sodium methanolate in methanol. The resulting mixture was stirred at 60° C. for 1 h. After cooling to room temperature, the reaction mixture was poured into water and the organic solvents were evaporated under reduced pressure. A 1N aqueous hydrogen chloride solution was then added until an acidic pH was achieved. After stirring for 5 minutes, the precipitate was filtered off, washed with water and dried. 5.96 g (73% of theory) of the title compound were obtained.

¹H-NMR (400 MHz, DMSO-d₆): δ [ppm]=3.39 (s, 3H), 4.82 (s, 2H), 7.87 (d, 1H), 8.26 (dd, 1H), 8.48 (d, 1H).

LC-MS (Method 4): Rₜ=0.87 min; MS (ESIneg): m/z=210 [M−H]⁻.

### Example 80A

### N-(biphenyl-4-yl)-4-(methoxymethyl)-3-nitrobenzamide

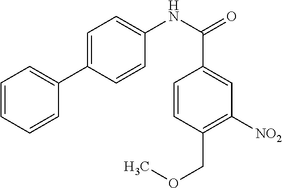 [SMILES: COCc1ccc(C(=O)Nc2ccc(-c3ccccc3)cc2)cc1[N+](=O)[O-]]

3.49 g (20.6 mmol, 1.5 equiv) of biphenyl-4-amine and 7.2 mL (41.2 mmol, 3 equiv) of N,N-diisopropylethylamine were provided in 20 mL of DMF at room temperature. A solution of 2.90 g (13.7 mmol) of the compound of example 79A in 20 mL of DMF and 16.0 mL (27.5 mmol, 2 equiv) of a 50% solution of 2,4,6-tripropyl-1,3,5,2,4,6-trioxatriphosphinane 2,4,6-trioxide (T3P) in DMF were added, and the mixture was stirred at room temperature over night. The resulting mixture was concentrated and the residue was purified using MPLC (Biotage Isolera; silica gel; hexane/ethyl acetate gradient). 4.20 g (84% of theory) of the title compound were obtained.

¹H-NMR (400 MHz, DMSO-d₆): δ [ppm]=3.41 (s, 3H), 4.84 (s, 2H), 7.32-7.38 (m, 1H), 7.43-7.50 (m, 2H), 7.65-7.73 (m, 4H), 7.86-7.93 (m, 3H), 8.36 (dd, 1H), 8.66 (d, 1H), 10.62 (s, 1H).

LC-MS (Method 4): Rₜ=1.38 min; MS (ESIpos): m/z=363 [M+H]⁺.

### Example 81A

### 3-amino-N-(biphenyl-4-yl)-4-(methoxymethyl)benzamide

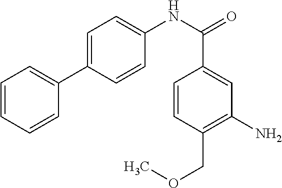 [SMILES: COCc1ccc(C(=O)Nc2ccc(-c3ccccc3)cc2)cc1N]

To a solution of the compound of example 80A (4.20 g, 11.6 mmol) in 130 mL of tetrahydrofuran was added a 15% solution of titanium(III) chloride in 10% hydrogen chloride dropwise (98.5 mL, 116 mmol, 10 equiv) at 0° C. The reaction mixture was allowed to warm up to room temperature and was stirred for three days. The pH of the mixture was adjusted under stirring with solid sodium bicarbonate to 7. The suspension was saturated with solid sodium chloride and stirred with 250 mL of a mixture of tetrahydrofuran/ethyl acetate 1:1 for 2 h. The suspension was filtered and the filtrate was washed with brine, dried over sodium sulfate and concentrated under reduced pressure. The residue (4.15 g) was purified using MPLC (Biotage Isolera; silica gel; dichloromethane/methanol gradient) to yield 3.42 g (80% of theory) of the title compound.

¹H-NMR (400 MHz, DMSO-d₆): δ [ppm]=3.30 (s, 3H), 4.39 (s, 2H), 5.18 (s, 2H), 7.13 (dd, 1H), 7.17-7.24 (m, 2H), 7.30-7.37 (m, 1H), 7.42-7.49 (m, 2H), 7.63-7.69 (m, 4H), 7.84-7.91 (m, 2H), 10.17 (s, 1H).

LC-MS (Method 4): Rₜ=1.22 min; MS (ESIpos): m/z=333 [M+H]⁺.

### Example 82A

### methyl 4-chloro-3-({[1-(morpholin-4-yl)cyclopropyl]carbonyl}amino)benzoate

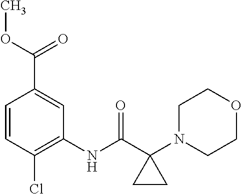 [SMILES: COC(=O)c1ccc(Cl)c(NC(=O)C2(N3CCOCC3)CC2)c1]

To a solution of methyl 3-amino-4-chlorobenzoate (3.00 g, 16.2 mmol) and 1-(morpholin-4-yl)cyclopropanecarboxylic acid hydrochloride (1:1) (example 65A, 6.71 g, 32.3 mmol, 2 equiv) in DMF (50 mL) was added (benzotriazol-1-yloxy)tripyrrolidinophosphonium hexafluorophosphate (PYBOP, 16.8 g, 32.3 mmol, 2 equiv) and diisopropylethylamine (14.1 mL, 80.8 mmol, 5 equiv). The resulting mixture was stirred at room temperature for 3 days. (Benzotriazol-1-yloxy)tripyrrolidinophosphonium hexafluorophosphate (PYBOP, 16.8 g, 32.3 mmol, 2 equiv) and diisopropylethylamine (14.1 mL, 80.8 mmol, 5 equiv) were added and the resulting mixture was stirred at 60° C. over night. The mixture was concentrated under reduced pressure, was then dissolved in dichloromethane, was washed with 1N aqueous hydrogen chloride solution and saturated, aqueous sodium bicarbonate solution, was dried over sodium sulfate and concentrated under reduced pressure. The remaining solids were then triturated with ethanol (40 mL), and the resulting mixture was stirred for 30 minutes. The remaining solids were removed by filtration, washed with ethanol, and were dried at 50° C. under reduced pressure. The remaining solids were then triturated with ethanol (70 mL), and the resulting mixture was stirred under reflux. After cooling to room temperature, the remaining solids were removed by filtration, washed with ethanol, and were dried at 50° C. under reduced pressure to give the title compound (3.60 g).

LC-MS (Method 4): Rₜ=1.23 min; MS (ESIpos): m/z=339 [M+H]⁺.

### Example 83A

### 4-chloro-3-({[1-(morpholin-4-yl)cyclopropyl]carbonyl}amino)benzoic acid

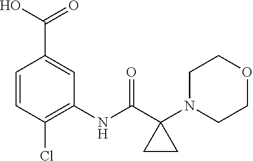 [SMILES: O=C(O)c1ccc(Cl)c(NC(=O)C2(N3CCOCC3)CC2)c1]

3.60 g (10.6 mmol) of the compound of example 82A were provided in 45 mL of dioxane, 509 mg (21.3 mmol) of lithium hydroxide and 19 mL of water were added at room temperature and the mixture was stirred at room temperature for 5 hours. Water and a 2N aqueous hydrogen chloride solution were then added until an acidic pH of 1.5-2 was achieved. After stirring for 15 minutes, the precipitate was filtered off, washed with water and dried. 2.67 g (77% of theory) of the title compound were obtained, which were used without further purification.

¹H-NMR (300 MHz, DMSO-d₆): δ [ppm]=1.10-1.18 (m, 2H), 1.23-1.31 (m, 2H), 2.43-2.49 (m, 4H), 3.68-3.77 (m, 4H), 7.61-7.70 (m, 2H), 8.97 (s, 1H), 10.75 (s, 1H), 13.17 (s, 1H).

LC-MS (Method 1): Rₜ=1.01 min; MS (ESIpos): m/z=325 [M+H]⁺.

### Example 84A

### 4-(biphenyl-4-ylcarbamoyl)-2-[(morpholin-4-ylacetyl)amino]benzoic acid

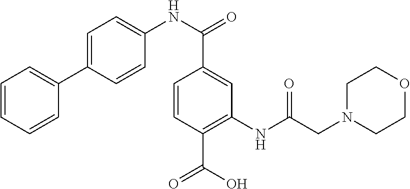 [SMILES: O=C(CN1CCOCC1)Nc1cc(C(=O)Nc2ccc(-c3ccccc3)cc2)ccc1C(=O)O]

1.90 g (4.01 mmol) of the compound of example 12 were provided in mixture of 40 mL of THF and 20 mL of methanol. 8.0 mL (40.1 mmol) of 5N aqueous solution of sodium hydroxide were added at room temperature and the mixture was stirred at room temperature over night. Ethyl acetate and water were added and the mixture was acidified by addition of a 5N aqueous hydrogen chloride solution. The phases were separated and the aqueous phase was extracted with ethyl acetate. The combined organic phases were dried over sodium sulfate and concentrated. 1.80 g (98% of theory) of the title compound were obtained, which were used without further purification.

LC-MS (Method 1): Rₜ=1.04 min; MS (ESIpos): m/z=460 [M+H]⁺.

### Example 85A

### N-(biphenyl-4-yl)-4-(methylsulfanyl)-3-nitrobenzamide

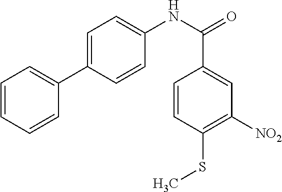 [SMILES: CSc1ccc(C(=O)Nc2ccc(-c3ccccc3)cc2)cc1[N+](=O)[O-]]

To 5 g (23.45 mmol) of 4-(methylsulfanyl)-3-nitrobenzoic acid in 150 mL of anh DMF were added 4.76 g (28.14 mmol) of biphenyl-4-amine, 14.64 g (28.14 mmol) of PYBOP and 4.9 mL (28.14 mmol) of N-ethyl-N-isopropylpropan-2-amine. It was stirred for 3 h at rt. 80 mL of water were added and the solid material was filtered off and washed three times with water. The solid was stirred for 30 min at 50° C. in 100 mL of EtOAc. The solid material was isolated by suction filtration, washed twice with EtOAc and dried under vacuum yielding 7.8 g (75%) of the title compound.

¹H-NMR (300 MHz, DMSO-d₆): δ [ppm]=2.61 (s, 3H), 7.30-7.37 (m, 1H), 7.41-7.49 (m, 2H), 7.64-7.78 (m, 5H), 7.85-7.92 (m, 2H), 8.31 (dd, 1H), 8.87 (d, 1H), 10.61 (s, 1H).

LC-MS (Method 4): Rₜ=1.36 min; MS (ESIpos): m/z=365 [M+H]⁺.

### Example 86A

### 3-amino-N-(biphenyl-4-yl)-4-(methylsulfanyl)benzamide

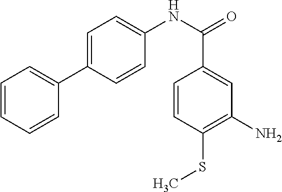 [SMILES: CSc1ccc(C(=O)Nc2ccc(-c3ccccc3)cc2)cc1N]

1.5 g (4.12 mmol) of N-(biphenyl-4-yl)-4-(methylsulfanyl)-3-nitrobenzamide (example 85A) were suspended in 160 mL of a mixture of methanol/THF 1:1. 263 mg of 10% palladium on charcoal (50% water) and two drops of water were added. It was stirred over night at 60° C. under an atmosphere of hydrogen. 80 mL of DMF were added and warm mixture was suction filtered over a Whatmanfilter containing a layer of celite. The filtrate was concentrated and triturated in a mixture of methanol/dichloromethane 1:1. The solid material was filtered off and dried under vacuum affording 1.1 g (79%) of the title compound.

¹H-NMR (300 MHz, DMSO-d₆): δ [ppm]=2.41 (s, 3H), 5.31 (s, 2H), 7.15-7.20 (m, 1H), 7.23-7.28 (m, 2H), 7.29-7.36 (m, 1H), 7.40-7.48 (m, 2H), 7.62-7.69 (m, 4H), 7.86 (d, 2H), 10.17 (s, 1H).

LC-MS (Method 4): Rₜ=1.25 min; MS (ESIpos): m/z=334 [M+H]⁺.

### Example 87A

### N-(biphenyl-4-yl)-4-(cyclopropyloxy)-3-nitrobenzamide

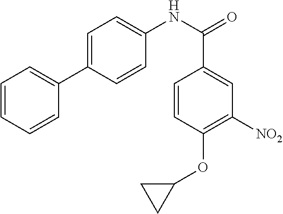 [SMILES: O=C(Nc1ccc(-c2ccccc2)cc1)c1ccc(OC2CC2)c([N+](=O)[O-])c1]

2.275 g (13.44 mmol) of biphenyl-4-amine, 2.5 g (11.20 mmol) of 4-(cyclopropyloxy)-3-nitrobenzoic acid and 7.00 g (13.44 mmol) of PYBOP were dissolved in 72 mL of anh DMF. 2.34 mL (13.44 mmol) of N-ethyl-N-isopropylpropan-2-amine were added. It was stirred for 3 h at rt. The reaction mixture was concentrated to approximately half of the original volume. It was added dropwise into water. The solid material was filtered off, washed three times with water and three times with a small volume of EtOAc. The crude product was crystallized from methanol, suction filtered and washed three times with cold methanol. It was purified on silica gel (gradient: hexane to EtOAc) yielding 3.32 g (58%) of the title compound.

¹H-NMR (400 MHz, DMSO-d₆): δ [ppm]=0.75-0.81 (m, 2H), 0.87-0.94 (m, 2H), 4.17-4.23 (m, 1H), 7.31-7.36 (m, 1H), 7.42-7.48 (m, 2H), 7.64-7.71 (m, 4H), 7.77 (d, 1H), 7.83-7.89 (m, 2H), 8.31 (dd, 1H), 8.53 (d, 1H), 10.43 (s, 1H).

LC-MS (Method 4): Rₜ=1.40 min; MS (ESIpos): m/z=375 [M+H]⁺.

### Example 88A

### 3-Amino-N-(biphenyl-4-yl)-4-(cyclopropyloxy)benzamide

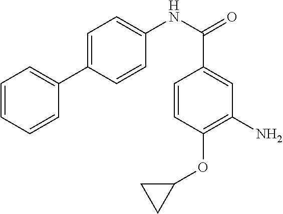 [SMILES: Nc1cc(C(=O)Nc2ccc(-c3ccccc3)cc2)ccc1OC1CC1]

3.29 g (8.79 mmol) of N-(biphenyl-4-yl)-4-(cyclopropyloxy)-3-nitrobenzamide (example 87A) were suspended in 150 mL of a mixture of methanol/THF 1:1. 494 mg of 10% palladium on charcoal (50% water) were added. It was stirred for 2 h at rt under an atmosphere of hydrogen. 165 mg of 10% palladium on charcoal (50% water) were added and it was stirred over night under an atmosphere of hydrogen. The catalyst was filtered through a layer of celite. The filtrate was concentrated to dryness. The residue was stirred at 55° C. in methanol. Then it was cooled down and the remaining solid material was suction filtered yielding 2.17 g (72%) of the title compound.

¹H-NMR (400 MHz, DMSO-d₆): δ [ppm]=0.67-0.72 (m, 2H), 0.77-0.83 (m, 2H), 3.88-3.94 (m, 1H), 4.85 (s, 2H), 7.14-7.18 (m, 1H), 7.20-7.24 (m, 2H), 7.29-7.35 (m, 1H), 7.41-7.47 (m, 2H), 7.61-7.68 (m, 4H), 7.83-7.87 (m, 2H), 10.03 (s, 1H).

LC-MS (Method 4): Rₜ=1.28 min; MS (ESIpos): m/z=345 [M+H]⁺.

### Example 89A

### tert-butyl {[4′-({4-methoxy-3-[(morpholin-4-ylacetyl)amino]phenyl}carbamoyl)biphenyl-4-yl]methyl}-carbamate

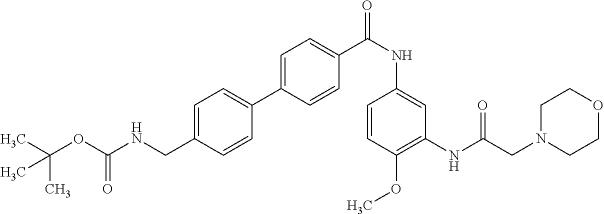 [SMILES: COc1ccc(NC(=O)c2ccc(-c3ccc(CNC(=O)OC(C)(C)C)cc3)cc2)cc1NC(=O)CN1CCOCC1]

The title compound was prepared in a manner analogous to that described in example 118 starting from 150 mg (335 μmol) of intermediate 59A and 126 mg (502 μmol) of (4-{[(tert-butoxycarbonyl)amino]methyl}phenyl)boronic acid. To work up the reaction, the mixture was poured into water. The resulting precipitate was collected by filtration and dried to yield the desired compound 89A (104 mg, 27%), which was used in the next step without further purification.

LC-MS (Method 4): Rₜ=1.11 min; MS (ESIpos): m/z=575 [M+H]⁺.

### Example 90A

### 4-bromo-3-fluoro-N-{4-methoxy-3-[(morpholin-4-ylacetyl)amino]phenyl}benzamide

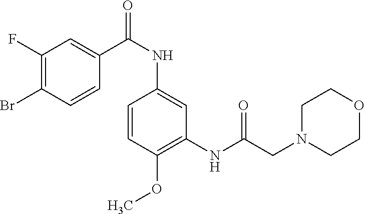 [SMILES: COc1ccc(NC(=O)c2ccc(Br)c(F)c2)cc1NC(=O)CN1CCOCC1]

A solution of the compound of example 56A (250 mg, 942 μmol) and 4-bromo-3-fluorobenzoic acid (227 mg, 1.04 mmol) in DMF (6.0 mL) was treated with N,N-diisopropylethylamine (492 μL, 2.83 mmol) and (benzotriazol-1-yloxy)tripyrrolidinophosphonium hexafluorophosphate (PYBOP, 736 mg, 1.41 mmol). The mixture was stirred over night at 60° C. After cooling to room temperature the mixture was poured into water. The precipitate was collected by filtration, washed with water and dried under reduced pressure at 60° C. to give the desired compound (401 mg, 91%).

¹H-NMR (400 MHz, DMSO-d₆): δ [ppm]=2.53-2.61 (m, 4H), 3.15 (s, 2H), 3.61-3.73 (m, 4H), 3.89 (s, 3H), 7.06 (d, 1H), 7.56-7.60 (m, 1H), 7.73-7.79 (m, 1H), 7.86-7.91 (m, 1H), 7.93-7.95 (m, 1H), 8.55 (d, 1H), 9.76 (s, 1H), 10.32 (s, 1H).

LC-MS (Method 4): Rₜ=0.94 min; MS (ESIpos): m/z=466 [M+H]⁺.

### Example 91A

### methyl 4-(bromomethyl)-3-nitrobenzoate

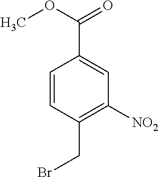

A solution of 4-(bromomethyl)-3-nitrobenzoic acid (2.00 g, 7.59 mmol) in methanol (20.0 mL) was treated with three drops of concentrated sulfuric acid and was refluxed for 2 days. After cooling to room temperature the mixture was concentrated in vacuum. The residue was dissolved in ethyl acetate. The organic phase was subsequently washed two times with water, one time with an aqueous, saturated NaHCO₃-solution, again one time with water and brine. The organic layer was dried over MgSO₄ and concentrated under reduced pressure to yield the desired product 91A (1.70 g, 81%).

¹H-NMR (300 MHz, DMSO-d₆): δ [ppm]=3.92 (s, 3H), 4.98 (s, 2H), 7.93 (d, 1H), 8.28-8.30 (m, 1H), 8.44-8.52 (m, 1H).

### Example 92A

### methyl 4-[(methylsulfonyl)methyl]-3-nitrobenzoate

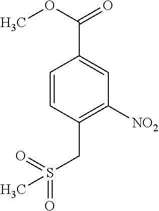 [SMILES: COC(=O)c1ccc(CS(C)(=O)=O)c([N+](=O)[O-])c1]

A solution of the compound of example 91A (1.97 g, 7.18 mmol) in ethanol (19.7 mL) was treated with methanesulfinic acid sodium salt (1.10 g, 10.8 mmol). The reaction mixture was refluxed for 6 h. After cooling to room temperature the mixture was diluted with water. The resulting suspension was stirred for 30 min. The solid was collected by filtration, washed with water and dried under reduced pressure at 60° C. to yield the desired compound 92A 82% pure (680 mg, 28%).

¹H-NMR (400 MHz, DMSO-d₆): δ [ppm]=3.03 (s, 3H), 3.93 (s, 3H), 5.08 (s, 2H), 7.85 (d, 1H), 8.30-8.32 (m, 1H), 8.49 (d, 1H).

LC-MS (Method 4): Rₜ=0.83 min; MS (ESIneg): m/z=272 [M−H]⁻.

### Example 93A

### 4-[(methylsulfonyl)methyl]-3-nitrobenzoic acid

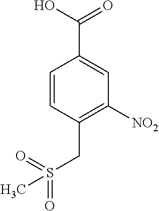 [SMILES: CS(=O)(=O)Cc1ccc(C(=O)O)cc1[N+](=O)[O-]]

A solution of the compound of example 92A (680 mg, 2.49 mmol) in a mixture of THF/water (12.5 mL/12.5 mL) was treated with an aqueous solution of lithium hydroxide (4.98 mL, 1 M, 4.98 mmol). The mixture was stirred for 2 h at room temperature. The organic solvent was removed in vacuum. The pH of the resulting solution was adjusted to 2 by the addition of 3M aqueous hydrochloric acid. The suspension was stirred for 30 min, afterwards the solid was collected by filtration and dried under reduced pressure at 60° C. to give the desired product 93A (530 mg, 82%).

¹H-NMR (300 MHz, DMSO-d₆): δ [ppm]=3.03 (s, 3H), 5.07 (s, 2H), 7.74-7.86 (m, 1H), 8.26-8.32 (m, 1H), 8.45-8.52 (m, 1H), 13.75 (br. s, 1H).

LC-MS (Method 1): Rₜ=0.83 min; MS (ESIneg): m/z=258 [M−H]⁻.

### Example 94A

### N-(biphenyl-4-yl)-4-[(methylsulfonyl)methyl]-3-nitrobenzamide

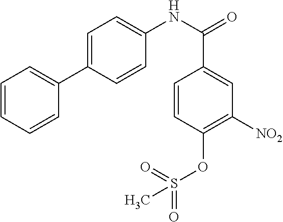 [SMILES: CS(=O)(=O)Oc1ccc(C(=O)Nc2ccc(-c3ccccc3)cc2)cc1[N+](=O)[O-]]

To a solution of the compound of example 93A (530 mg, 2.04 mmol) and biphenyl-4-amine (415 mg, 2.45 mmol) in DMF (13.1 mL) were added 1.43 mL (2.45 mmol) of a 50% solution of 2,4,6-tripropyl-1,3,5,2,4,6-trioxatriphosphinane 2,4,6-trioxide (T3P) in DMF and N,N-diisopropylethylamine (1.07 mL). The mixture was stirred over night at room temperature. The volume of the reaction mixture was reduced under reduced pressure. The residue was poured into water, the precipitate was collected by filtration. The crude product was recrystallized from ethanol to yield the desired compound 94A (410 mg, 50%).

LC-MS (Method 4): Rₜ=1.23 min; MS (ESIpos): m/z=411 [M+H]⁺.

### Example 95A

### 3-amino-N-(biphenyl-4-yl)-4-[(methylsulfonyl)methyl]benzamide

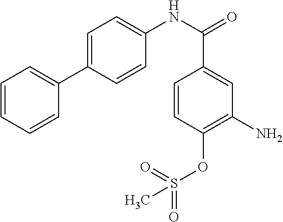 [SMILES: CS(=O)(=O)Oc1ccc(C(=O)Nc2ccc(-c3ccccc3)cc2)cc1N]

A solution of the compound of example 94A (410 mg, 1.00 mmol) in THF (16.9 mL) was treated with palladium on charcoal (10% Pd, 163 mg, 1.53 mmol) and was stirred over night under a hydrogen atmosphere at room temperature. The reaction mixture was filtered over a pad of Celite. The Celite was washed with methanol, the filtrate was concentrated to deliver 180 mg of the desired product 95A. The Celite was stirred in a mixture of DCM/isopropanol (8:2), after filtration, the solvent was removed. The residue yielded additional 120 mg of the desired compound. In total 300 mg of intermediate 95A (79%) were obtained.

LC-MS (Method 4): Rₜ=1.12 min; MS (ESIpos): m/z=381 [M+H]⁺.

### Example 96A

### N-(biphenyl-4-yl)-3-[(chloroacetyl)amino]-4-[(methylsulfonyl)methyl]benzamide

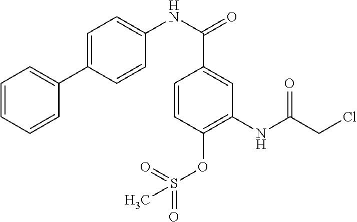 [SMILES: CS(=O)(=O)Oc1ccc(C(=O)Nc2ccc(-c3ccccc3)cc2)cc1NC(=O)CCl]

To a mixture of the compound of example 95A (300 mg, 0.79 mmol) and pyridine (70.1 μL, 867 μmol) in DCM (2.14 mL) was added chloroacetyl chloride (66.0 μL, 828 μmol). The reaction mixture was stirred at room temperature over night. Additionally 1.05 eq of chloroacetyl chloride were added and the mixture was stirred one further night at room temperature. The reaction mixture was diluted with water and was extracted two times with DCM. The combined organic layers were dried by the use of a silicon filter and the solvent was removed under reduced pressure to deliver the desired crude product 96A (330 mg, 71%), which was used in the next step without further purification.

LC-MS (Method 4): Rₜ=1.17 min; MS (ESIpos): m/z=457 [M+H]⁺.

### Example 97A

### tert-butyl (3-{4-(biphenyl-4-ylcarbamoyl)-2-[(morpholin-4-ylacetyl)amino]phenoxy}propyl)carbamate

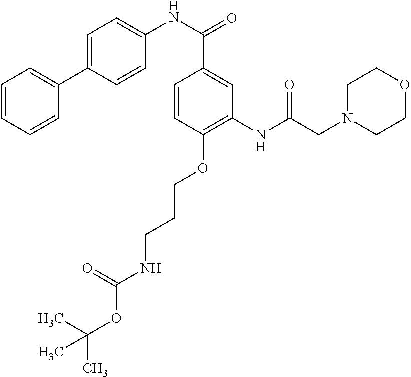 [SMILES: CC(C)(C)OC(=O)NCCCOc1ccc(C(=O)Nc2ccc(-c3ccccc3)cc2)cc1NC(=O)CN1CCOCC1]

A solution of tert-butyl(3-hydroxypropyl)carbamate (86.4 mg, 493 μmol) and triethylamine (137 μL, 986 μmol) in DCM (1.7 mL) was treated with methanesulfonyl chloride (53 μL, 740 μmol). The mixture was stirred 1.5 h at room temperature, afterwards water was added and the mixture was extracted with ethyl acetate. The organic layer was washed with a saturated, aqueous NaHCO₃-solution and brine, and was dried over Na₂SO₄. The solvent was removed in vacuum. The residue was dissolved in DMF (1.0 mL) and poured into a suspension of the compound of example 24A (152 mg, 352 μmol) and cesium carbonate (230 mg, 705 μmol) in DMF (1.0 mL). The resulting mixture was stirred over night at 70° C. After cooling to room temperature the mixture was diluted with water and was extracted with DCM. The organic phase was concentrated to yield the desired crude product 97A as mixture with DMF (235 mg). The crude product was used in the next reaction without further purification.

¹H-NMR (400 MHz, DMSO-d₆): δ [ppm]=1.37 (s, 9H), 1.92-2.03 (m, 2H), 2.52-2.59 (m, 4H), 3.14-3.22 (m, 4H), 3.62-3.69 (m, 4H), 4.18 (s, 2H), 6.93-7.02 (m, 1H), 7.15-7.20 (m, 1H), 7.30-7.37 (m, 1H), 7.41-7.49 (m, 2H), 7.64-7.71 (m, 4H), 7.73-7.78 (m, 1H), 7.87 (d, 2H), 8.82-8.86 (m, 1H), 9.67-9.73 (m, 1H), 10.22 (s, 1H).

LC-MS (Method 4): Rₜ=1.21 min; MS (ESIpos): m/z=589 [M+H]⁺.

### Example 98A

### 4-(3-aminopropoxy)-N-(biphenyl-4-yl)-3-[(morpholin-4-ylacetyl)amino]benzamide dihydrochloride

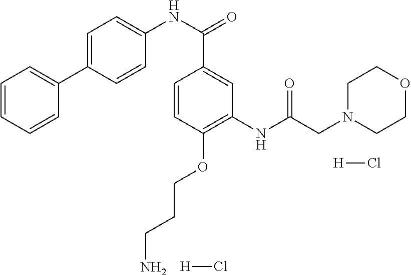

Example 97A (230 mg, 391 μmol) was dissolved in a 4M solution of hydrochloric acid in dioxane (4.88 mL) and stirred over night at room temperature. The resulting precipitate was collected by filtration and washed carefully with ethanol to yield the desired product 98A (30.0 mg, 14%). The filtrate was concentrated in vacuum to obtain additional 250 mg crude product, which was used in the next step without further purification.

¹H-NMR (400 MHz, DMSO-d₆): δ [ppm]=2.05-2.22 (m, 2H), 3.05 (dd, 2H), 3.33 (br. s, 2H), 3.82-4.04 (m, 4H), 4.25 (t, 2H), 4.39-4.51 (m, 2H), 7.22-7.24 (m, 1H), 7.30-7.38 (m, 1H), 7.42-7.46 (m, 2H), 7.65-7.67 (m, 4H), 7.85-7.98 (m, 3H), 8.24 (br. s, 3H), 8.41 (br. s, 1H), 10.14 (br. s, 1H), 10.30 (s, 1H), 10.80 (m, 1H).

LC-MS (Method 4): Rₜ=0.83 min; MS (ESIpos): m/z=489 [M+H]⁺.

### Examples of General Formula (I)

### Example 1

### N-(biphenyl-4-yl)-4-methoxy-3-[(morpholin-4-ylacetyl)amino]benzamide

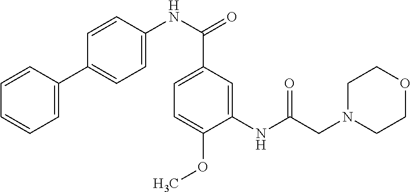 [SMILES: COc1ccc(C(=O)Nc2ccc(-c3ccccc3)cc2)cc1NC(=O)CN1CCOCC1]

To a solution of 4-methoxy-3-[(morpholin-4-ylacetyl)amino]benzoic acid (prepared in a manner analogous to that described in example 62A, 0.10 g, 0.34 mmol) and biphenyl-4-amine (0.058 g, 0.34 mmol, 1.0 equiv) in DMF (2.5 mL) was added propanephosphonic acid cyclic anhydride solution (50% in ethyl acetate, 0.20 mL, 0.34 mmol, 1.0 equiv) followed by diisopropylethylamine (0.18 mL, 1.02 mmol, 3.0 equiv). The resulting mixture was stirred at room temperature for 24 h, was then treated with (benzotriazol-1-yloxy)tripyrrolidinophosphonium hexafluorophosphate (PYBOP, 0.177 g, 0.34 mmol, 1.0 equiv) and diisopropylethylamine (0.18 mL, 1.02 mmol, 3.0 equiv). The resulting mixture was stirred at room temperature for 24 h, was then treated with water (5 mL). The resulting mixture was extracted with ethyl acetate (10 mL). The organic phase was dried (Na₂SO₄ anh), and concentrated under reduced pressure. The residue (0.25 g) was purified using HPLC (method 3) to give N-(biphenyl-4-yl)-4-methoxy-3-[(morpholin-4-ylacetyl)amino]benzamide (0.055 g, 36%).

¹H-NMR (400 MHz, DMSO-d₆): δ [ppm]=2.51-2.55 (m, 4H), 3.15 (s, 2H), 3.62-3.66 (m, 4H), 3.95 (s, 3H), 7.17 (d, J=8.7 Hz, 1H), 7.30 (t, J=7.3 Hz, 1H), 7.42 (t, J=7.7, 2H), 7.61-7.66 (m, 4H), 7.73 (dd, J=2.3, 8.5 Hz, 1H), 7.83 (d, J=8.7 Hz, 2H), 8.74 (d, J=1.9 Hz, 1H), 9.75 (s, 1H), 10.19 (s, 1H).

LC-MS (Method 4): Rₜ=1.31 min; MS (ESIpos): m/z=446 ([M+H]⁺, 100%), 891 ([2M+H]⁺, 20%); MS (ESIneg): m/z=444 ([M−H]⁻, 100%).

### Example 2

### N-(biphenyl-4-yl)-4-methoxy-3-[(1H-pyrazol-1-ylacetyl)amino]benzamide

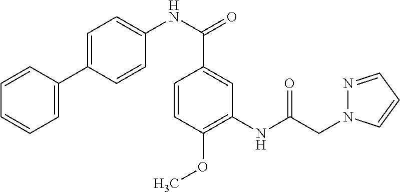 [SMILES: COc1ccc(C(=O)Nc2ccc(-c3ccccc3)cc2)cc1NC(=O)Cn1cccn1]

200 mg (628 μmol) of the compound from example 7A and 328 μL (1.89 mmol) of N,N-diisopropylethylamine were provided in 3 mL of DMF. 95.0 mg (754 μmol) of 1H-pyrazol-1-ylacetic acid and 440 μL (754 μmol) of a 50% solution of 2,4,6-tripropyl-1,3,5,2,4,6-trioxatriphosphinane 2,4,6-trioxide (T3P) in DMF were added, and the mixture was stirred over night at room temperature. After filtration, purification by HPLC (method 2) yielded 88.0 mg (32% of theory) of the title compound.

¹H-NMR (400 MHz, DMSO-d₆): δ [ppm]=3.94 (s, 3H), 5.15 (s, 2H), 6.32 (t, 1H), 7.20 (d, 1H), 7.29-7.38 (m, 1H), 7.40-7.49 (m, 2H), 7.51-7.56 (m, 1H), 7.62-7.71 (m, 4H), 7.76-7.90 (m, 4H), 8.61 (s, 1H), 9.52 (s, 1H), 10.21 (s, 1H).

LC-MS (Method 1): Rₜ=1.22 min; MS (ESIpos): m/z=427 [M+H]⁺.

### Example 3

### N-(biphenyl-4-yl)-3-[(1H-pyrazol-1-ylacetyl)amino]-4-(trifluoromethyl)benzamide

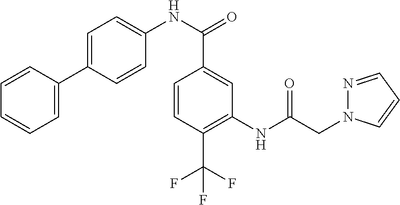 [SMILES: O=C(Cn1cccn1)Nc1cc(C(=O)Nc2ccc(-c3ccccc3)cc2)ccc1C(F)(F)F]

150 mg (421 μmol) of the compound from example 8A and 220 μL (1.26 mmol) of N,N-diisopropylethylamine were dissolved in 2 mL of DMF. 64.0 mg (505 μmol) of 1H-pyrazol-1-ylacetic acid and 295 μL (505 μmol) of a 50% solution of 2,4,6-tripropyl-1,3,5,2,4,6-trioxatriphosphinane 2,4,6-trioxide (T3P) in DMF were added, and the mixture was stirred over night at room temperature.

After filtration, purification by HPLC (method 2) yielded 129 mg (65% of theory) of the title compound.

¹H-NMR (400 MHz, DMSO-d₆): δ [ppm]=5.14 (s, 2H), 6.30 (t, 1H), 7.32-7.38 (m, 1H), 7.43-7.49 (m, 2H), 7.51 (d, 1H), 7.65-7.72 (m, 4H), 7.80 (d, 1H), 7.85-7.89 (m, 2H), 7.94 (d, 1H), 8.04 (d, 1H), 8.16 (s, 1H), 9.95 (s, 1H), 10.59 (s, 1H).

LC-MS (Method 1): Rₜ=1.29 min; MS (ESIpos): m/z=465 [M+H]⁺.

### Example 4

### N-(biphenyl-4-yl)-3-{[2-methyl-2-(1H-pyrazol-1-yl)propanoyl]amino}-4-(trifluoromethyl)benzamide

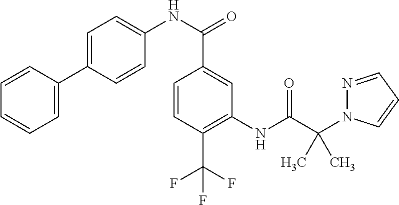 [SMILES: CC(C)(C(=O)Nc1cc(C(=O)Nc2ccc(-c3ccccc3)cc2)ccc1C(F)(F)F)n1cccn1]

150 mg (973 μmol) of 2-methyl-2-(1H-pyrazol-1-yl)propanoic acid were stirred in 1.5 mL of dichloromethane at room temperature. 3.7 μL (49 μmol) of DMF and 0.17 mL (1.95 mmol) of oxalyl chloride were added, and the mixture was stirred for additional 5 h at 50° C. after the gas formation had stopped. After concentration, 136 mg of raw material were obtained, which were used without further purification. 187 mg (525 μmol) of the compound from example 8A were dissolved in 2 mL of DMF, and 110 μL (789 μmol) of triethylamine and 136 mg of the acid chloride were added. The mixture was stirred at room temperature over night. Since the reaction was not complete, another batch of acid chloride was synthesized: 300 mg (1.95 mmol) of 2-methyl-2-(1H-pyrazol-1-yl)propanoic acid were stirred in 3 mL of dichloromethane at room temperature. 7.5 μL (97 μmol) of DMF and 0.34 mL (3.89 mmol) of oxalyl chloride were added, and the mixture was stirred for additional 4 h at 50° C. after the gas formation had stopped. After concentration, 328 mg of raw material were obtained, which were used without further purification. 265 μL (1.90 mmol) of triethylamine and the 328 mg of the acid chloride were added to the reaction mixture, which was then stirred at room temperature over night. After filtration, purification by HPLC (method 2) yielded 24 mg (9% of theory) of the title compound.

¹H-NMR (500 MHz, DMSO-d₆): δ [ppm]=1.87 (s, 6H), 6.40 (s, 1H), 7.32-7.37 (m, 1H), 7.43-7.49 (m, 2H), 7.64-7.73 (m, 5H), 7.84-7.93 (m, 3H), 7.97-8.03 (m, 2H), 8.21 (s, 1H), 9.11 (s, 1H), 10.59 (s, 1H).

LC-MS (Method 1): Rₜ=1.44 min; MS (ESIpos): m/z=493 [M+H]⁺.

### Example 5

### N-(biphenyl-4-yl)-2-chloro-4-methoxy-5-{[2-(morpholin-4-yl)propanoyl]amino}benzamide

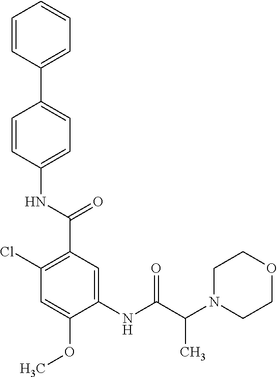 [SMILES: COc1cc(Cl)c(C(=O)Nc2ccc(-c3ccccc3)cc2)cc1NC(=O)C(C)N1CCOCC1]

110 mg of 90% purity (281 μmol) of the compound from example 9A and 147 μL (842 μmol) of N,N-diisopropylethylamine were provided in 1.5 mL of DMF. A solution of 53.6 mg (337 μmol) of 2-(morpholin-4-yl)propanoic acid in 0.5 mL of DMF and 197 μL (337 μmol) of a 50% solution of 2,4,6-tripropyl-1,3,5,2,4,6-trioxatriphosphinane 2,4,6-trioxide (T3P) in DMF were added, and the mixture was stirred over night at room temperature. 53.6 mg (337 μmol) of 2-(morpholin-4-yl)propanoic acid and 197 μL (337 μmol) of a 50% solution of 2,4,6-tripropyl-1,3,5,2,4,6-trioxatriphosphinane 2,4,6-trioxide (T3P) in DMF were added, and the mixture was stirred over night at room temperature. After filtration, purification by HPLC (method 2) yielded 56.3 mg (38% of theory) of the title compound.

¹H-NMR (400 MHz, DMSO-d₆): δ [ppm]=1.20 (d, 3H), 2.48-2.61 (m, 4H), 3.32-3.43 (m, 1H), 3.62-3.74 (m, 4H), 3.99 (s, 3H), 7.28 (s, 1H), 7.31-7.37 (m, 1H), 7.42-7.49 (m, 2H), 7.63-7.70 (m, 4H), 7.78-7.84 (m, 2H), 8.42 (s, 1H), 10.00 (s, 1H), 10.49 (s, 1H).

LC-MS (Method 4): Rₜ=1.16 min; MS (ESIpos): m/z=494 [M+H]⁺.

### Example 6

### N-(biphenyl-4-yl)-2-chloro-4-methoxy-5-[(morpholin-4-ylacetyl)amino]benzamide

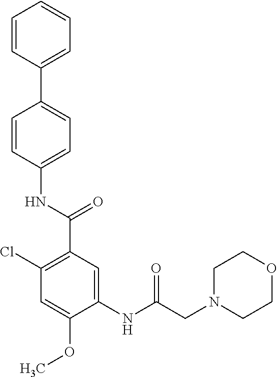 [SMILES: COc1cc(Cl)c(C(=O)Nc2ccc(-c3ccccc3)cc2)cc1NC(=O)CN1CCOCC1]

110 mg of 90% purity (281 μmol) of the compound from example 9A and 147 μL (842 μmol) of N,N-diisopropylethylamine were provided in 1.5 mL of DMF. A solution of 48.9 mg (337 μmol) of morpholin-4-ylacetic acid in 0.5 mL of DMF and 197 μL (337 μmol) of a 50% solution of 2,4,6-tripropyl-1,3,5,2,4,6-trioxatriphosphinane 2,4,6-trioxide (T3P) in DMF were added, and the mixture was stirred over night at room temperature. After filtration, purification by HPLC (method 2) yielded 65.6 mg (49% of theory) of the title compound.

¹H-NMR (400 MHz, DMSO-d₆): δ [ppm]=2.53-2.59 (m, 4H), 3.19 (s, 2H), 3.64-3.70 (m, 4H), 3.99 (s, 3H), 7.29 (s, 1H), 7.31-7.37 (m, 1H), 7.41-7.50 (m, 2H), 7.62-7.70 (m, 4H), 7.77-7.84 (m, 2H), 8.42 (s, 1H), 9.81 (s, 1H), 10.50 (s, 1H).

LC-MS (Method 4): Rₜ=1.12 min; MS (ESIpos): m/z=480 [M+H]⁺.

### Example 7

### N-(biphenyl-4-yl)-4-methoxy-3-{[(1S,4S)-2-oxa-5-azabicyclo[2.2.1]hept-5-ylacetyl]amino}benzamide

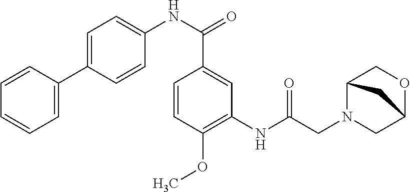 [SMILES: COc1ccc(C(=O)Nc2ccc(-c3ccccc3)cc2)cc1NC(=O)CN1C[C@@H]2C[C@H]1CO2]

200 mg (507 μmol) of the compound from example 12A were provided in 2 mL of DMF. 212 μL (1.52 mmol) of triethylamine, 103 mg (760 μmol) of (1S,4S)-2-oxa-5-azabicyclo[2.2.1]heptane hydrochloride and 13.0 mg (79 μmol) of potassium iodide were added, and the mixture was stirred at room temperature over night. After filtration, purification by HPLC (method 2) yielded 32.0 mg (13% of theory) of the title compound.

¹H-NMR (300 MHz, DMSO-d₆): δ [ppm]=1.61-1.77 (m, 1H), 1.78-1.94 (m, 1H), 2.64-2.80 (m, 1H), 2.81-2.99 (m, 1H), 3.33-3.47 (m, 2H), 3.51-3.71 (m, 2H), 3.80-3.92 (m, 1H), 3.97 (s, 3H), 4.40-4.52 (m, 1H), 7.21 (d, 1H), 7.29-7.38 (m, 1H), 7.40-7.51 (m, 2H), 7.62-7.72 (m, 4H), 7.73-7.82 (m, 1H), 7.83-7.91 (m, 2H), 8.79 (s, 1H), 9.82 (s, 1H), 10.23 (s, 1H).

LC-MS (Method 1): Rₜ=0.94 min; MS (ESIpos): m/z=458 [M+H]⁺.

### Example 8

### N-(biphenyl-4-yl)-4-methoxy-3-[(8-oxa-3-azabicyclo[3.2.1]oct-3-ylacetyl)amino]benzamide

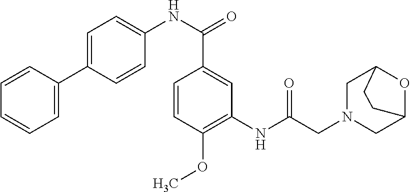 [SMILES: COc1ccc(C(=O)Nc2ccc(-c3ccccc3)cc2)cc1NC(=O)CN1CC2CCC(C1)O2]

The preparation of the title compound took place analogously to the synthesis of the compound from example 7 starting with 200 mg (507 μmol) of the compound from example 12A and 114 mg (760 μmol) of 8-oxa-3-azabicyclo[3.2.1]octane hydrochloride. 131 mg (53% of theory) of the title compound were obtained.

¹H-NMR (300 MHz, DMSO-d₆): δ [ppm]=1.81-1.94 (m, 2H), 2.04-2.13 (m, 2H), 2.42-2.52 (m, 2H), 2.60-2.69 (m, 2H), 3.11 (s, 2H), 3.98 (s, 3H), 4.25-4.32 (m, 2H), 7.23 (d, 1H), 7.30-7.37 (m, 1H), 7.41-7.50 (m, 2H), 7.62-7.71 (m, 4H), 7.77 (dd, 1H), 7.83-7.91 (m, 2H), 8.89 (d, 1H), 9.78 (s, 1H), 10.23 (s, 1H).

LC-MS (Method 1): Rₜ=1.25 min; MS (ESIpos): m/z=472 [M+H]⁺.

### Example 9

### N-(biphenyl-4-yl)-3-[(morpholin-4-ylacetyl)amino]-4-(trifluoromethyl)benzamide

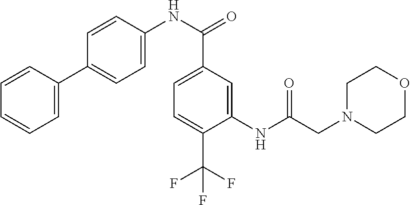 [SMILES: O=C(CN1CCOCC1)Nc1cc(C(=O)Nc2ccc(-c3ccccc3)cc2)ccc1C(F)(F)F]

To a solution of N-(biphenyl-4-yl)-3-[(chloroacetyl)amino]-4-(trifluoromethyl)benzamide (prepared in a manner analogous to that described in example 13A, 0.11 g, 0.25 mmol) in DMF (1 mL) was added morpholine (0.032 mL, 0.37 mmol, 1.5 equiv), triethylamine (0.051 mL, 0.37 mmol, 1.5 equiv) and potassium iodide (0.006 g, 0.038 mmol, 0.16 equiv). The reaction mixture was stirred at room temperature for 16 h. The resulting mixture was diluted with water (2 mL). The resulting solution was extracted with ethyl acetate (3×5 mL). The resulting mixture was washed with a half-saturated NaCl solution, dried (Na₂SO₄ anh) and concentrated under reduced pressure to give N-(biphenyl-4-yl)-3-[(morpholin-4-ylacetyl)amino]-4-(trifluoromethyl)benzamide (0.076 g, 64%).

¹H-NMR (400 MHz, DMSO-d₆): δ [ppm]=2.53-2.57 (m, 4H), 3.20 (s, 2H), 3.60-3.65 (m, 4H), 7.31 (t, J=7.3 Hz, 1H), 7.43 (t, J=7.5 Hz, 2H), 7.61-7.70 (m, 4H), 7.81-7.93 (m, 4H), 8.67 (s, 1H), 9.95 (s, 1H), 10.55 (s, 1H).

LC-MS (Method 3): Rₜ=1.36 min; MS (ESIpos): m/z=484 ([M+H]⁺, 100%), 967 ([2M+H]⁺, 50%); MS (ESIneg): m/z=482 ([M−H]⁻, 100%), 965 ([2M−H]⁻, 10%).

### Example 10

### N-(biphenyl-4-yl)-3-[(8-oxa-3-azabicyclo[3.2.1]oct-3-ylacetyl)amino]-4-(trifluoromethyl)benzamide

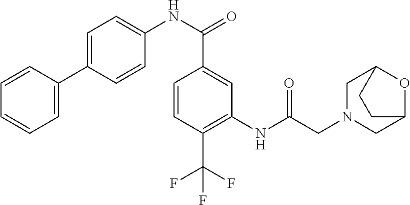 [SMILES: O=C(CN1CC2CCC(C1)O2)Nc1cc(C(=O)Nc2ccc(-c3ccccc3)cc2)ccc1C(F)(F)F]

150 mg (347 μmol) of the compound from example 13A were provided in 2 mL of DMF. 121 μL (866 μmol) of triethylamine, 77.8 mg (520 μmol) of 8-oxa-3-azabicyclo[3.2.1]octane hydrochloride and 8.9 mg (54 μmol) of potassium iodide were added, and the mixture was stirred at room temperature over night. After filtration, purification by HPLC (method 2) yielded 115 mg (65% of theory) of the title compound.

¹H-NMR (500 MHz, DMSO-d₆): δ [ppm]=1.77-1.83 (m, 2H), 1.94-2.00 (m, 2H), 2.44 (dd, 2H), 2.68 (d, 2H), 3.18 (s, 2H), 4.25-4.29 (m, 2H), 7.33-7.37 (m, 1H), 7.44-7.48 (m, 2H), 7.66-7.72 (m, 4H), 7.86-7.90 (m, 2H), 7.93-7.98 (m, 2H), 8.55 (s, 1H), 9.47 (s, 1H), 10.58 (s, 1H).

LC-MS (Method 4): Rₜ=1.37 min; MS (ESIpos): m/z=510 [M+H]⁺.

### Example 11

### N-(biphenyl-4-yl)-3-{[(1S,4S)-2-oxa-5-azabicyclo[2.2.1]hept-5-ylacetyl]amino}-4-(trifluoromethyl)benzamide

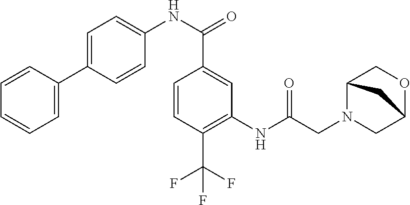 [SMILES: O=C(CN1C[C@@H]2C[C@H]1CO2)Nc1cc(C(=O)Nc2ccc(-c3ccccc3)cc2)ccc1C(F)(F)F]

The preparation of the title compound took place analogously to the synthesis of the compound from example 10 starting with 150 mg (347 μmol) of the compound from example 13A and 70.5 mg (520 μmol) of (1S,4S)-2-oxa-5-azabicyclo[2.2.1]heptane hydrochloride. 107 mg (60% of theory) of the title compound were obtained.

¹H-NMR (500 MHz, DMSO-d₆): δ [ppm]=1.67-1.72 (m, 1H), 1.79-1.84 (m, 1H), 2.67-2.72 (m, 1H), 2.88-2.93 (m, 1H), 3.41-3.51 (m, 2H), 3.60-3.64 (m, 2H), 3.81-3.86 (m, 1H), 4.43-4.47 (m, 1H), 7.32-7.37 (m, 1H), 7.43-7.49 (m, 2H), 7.66-7.72 (m, 4H), 7.85-7.95 (m, 4H), 8.77 (s, 1H), 10.13 (s, 1H), 10.58 (s, 1H).

LC-MS (Method 4): Rₜ=1.04 min; MS (ESIpos): m/z=496 [M+H]⁺.

### Example 12

### methyl 4-(biphenyl-4-ylcarbamoyl)-2-[(morpholin-4-ylacetyl)amino]benzoate

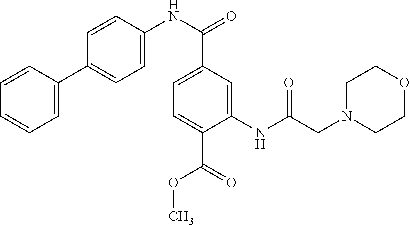 [SMILES: COC(=O)c1ccc(C(=O)Nc2ccc(-c3ccccc3)cc2)cc1NC(=O)CN1CCOCC1]

To a solution of methyl 4-(biphenyl-4-ylcarbamoyl)-2-[(chloroacetyl)amino]benzoate (prepared in a manner analogous to that described in example 14A, 2.95 g, 6.98 mmol) in DMF (30 mL) was added morpholine (0.91 mL, 10.5 mmol, 1.5 equiv), triethylamine (1.46 mL, 10.5 mmol, 1.5 equiv) and potassium iodide (0.18 g, 1.08 mmol, 0.16 equiv). The reaction mixture was stirred at room temperature for 16 h. The resulting mixture was diluted with water (30 mL). The resulting precipitate was washed with water and ethanol, was then dried at 50° C. to give methyl 4-(biphenyl-4-ylcarbamoyl)-2-[(morpholin-4-ylacetyl)amino]benzoate (3.10 g, 90%).

¹H-NMR (300 MHz, DMSO-d₆): δ [ppm]=2.53-2.61 (m, 4H), 3.21 (s, 2H), 3.69-3.79 (m, 4H), 3.95 (s, 3H), 7.30-7.39 (m, 1H), 7.41-7.50 (m, 2H), 7.64-7.75 (m, 5H), 7.84-7.91 (m, 2H), 8.12 (d, 1H), 9.16 (d, 1H), 10.57 (s, 1H), 11.90 (s, 1H).

LC-MS (Method 3): Rₜ=1.36 min; MS (ESIpos): m/z=474 [M+H]⁺.

### Example 13

### N-(biphenyl-4-yl)-4-bromo-3-[(morpholin-4-ylacetyl)amino]benzamide

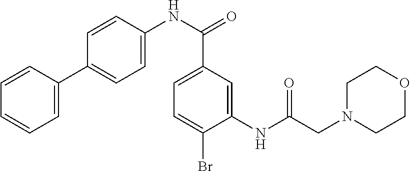 [SMILES: O=C(CN1CCOCC1)Nc1cc(C(=O)Nc2ccc(-c3ccccc3)cc2)ccc1Br]

To a solution of N-(biphenyl-4-yl)-4-bromo-3-[(chloroacetyl)amino]benzamide (prepared in a manner analogous to that described in example 15A, 3.00 g, 6.67 mmol) in DMF (30 mL) was added morpholine (0.88 mL, 10.1 mmol, 1.5 equiv), triethylamine (1.41 mL, 10.1 mmol, 1.5 equiv) and potassium iodide (0.17 g, 1.05 mmol, 0.16 equiv). The reaction mixture was stirred at room temperature for 16 h. The resulting mixture was diluted with water (30 mL). The resulting precipitate was washed with water, was then dried at 50° C. to give N-(biphenyl-4-yl)-4-bromo-3-[(morpholin-4-ylacetyl)amino]benzamide (3.20 g, 94%).

¹H-NMR (400 MHz, DMSO-d₆): δ [ppm]=2.55-2.59 (m, 4H), 3.19 (s, 2H), 3.67-3.70 (m, 4H), 7.30 (t, J=7.4 Hz, 1H), 7.45 (t, J=7.7 Hz, 2H), 7.62-7.66 (m, 5H), 7.82-7.85 (m, 3H), 8.75 (d, J=2.3 Hz, 1H), 10.01 (s, 1H), 10.41 (s, 1H).

LC-MS (Method 3): Rₜ=1.36 min; MS (ESIpos): m/z=494 ([M+H]⁺, 90%), 987 ([2M+H]⁺, 30%); MS (ESIneg): m/z=492 ([M−H]⁻, 100%).

### Example 14

### N-(biphenyl-4-yl)-3-{[2-(morpholin-4-yl)propanoyl]amino}-4-(trifluoromethyl)benzamide

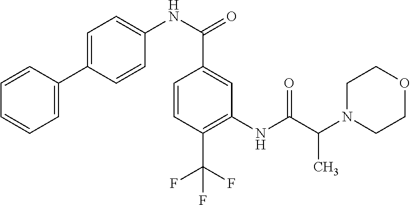 [SMILES: CC(C(=O)Nc1cc(C(=O)Nc2ccc(-c3ccccc3)cc2)ccc1C(F)(F)F)N1CCOCC1]

3.00 g (6.71 mmol) of the compound from example 16A were provided in 35 mL of DMF. 2.8 mL (20.1 mmol) of triethylamine, 1.8 mL (20.1 mmol) of morpholine and 223 mg (1.34 mmol) of potassium iodide were added, and the mixture was stirred at 50° C. over night. 0.6 mL (6.71 mmol) of morpholine were added, and the mixture was stirred at 50° C. for 4 h. After filtration, purification by HPLC (column: chromatorex C18, 10 μm, 195×51 mm, mobile phase: acetonitrile/water gradient with the addition of 0.1% formic acid) yielded 2.30 g (69% of theory) of the title compound.

¹H-NMR (300 MHz, DMSO-d₆): δ [ppm]=1.24 (d, 3H), 2.53-2.63 (m, 4H), 3.38 (q, 1H), 3.61-3.72 (m, 4H), 7.31-7.39 (m, 1H), 7.41-7.50 (m, 2H), 7.65-7.73 (m, 4H), 7.84-7.95 (m, 4H), 8.64 (s, 1H), 10.05 (s, 1H), 10.58 (s, 1H).

LC-MS (Method 1): Rₜ=1.25 min; MS (ESIpos): m/z=498 [M+H]⁺.

### Examples 15 and 16

### N-(biphenyl-4-yl)-3-{[(2S)-2-(morpholin-4-yl)propanoyl]amino}-4-(trifluoromethyl)benzamide

### N-(biphenyl-4-yl)-3-{[(2R)-2-(morpholin-4-yl)propanoyl]amino}-4-(trifluoromethyl)benzamide

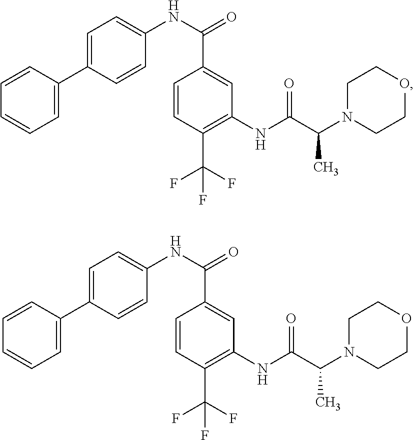 [SMILES: C[C@@H](C(=O)Nc1cc(C(=O)Nc2ccc(-c3ccccc3)cc2)ccc1C(F)(F)F)N1CCOCC1 ; C[C@H](C(=O)Nc1cc(C(=O)Nc2ccc(-c3ccccc3)cc2)ccc1C(F)(F)F)N1CCOCC1]

Chiral chromatography (system: Sepiatec Prep SFC100, column: Chiralpak IC 5 μm 250×20 mm, solvent: CO₂/ethanol 70/30, rate: 60 mL/min, pressure (outlet): 150 bar, temperature: 40° C., detection: UV 254 nm) of 2.30 g of the compound from example 14 provided:

### Example 15

### 995 mg

¹H-NMR (300 MHz, DMSO-d₆): δ [ppm]=1.24 (d, 3H), 2.53-2.63 (m, 4H), 3.38 (q, 1H), 3.61-3.72 (m, 4H), 7.31-7.39 (m, 1H), 7.41-7.50 (m, 2H), 7.65-7.73 (m, 4H), 7.84-7.95 (m, 4H), 8.64 (s, 1H), 10.05 (s, 1H), 10.58 (s, 1H).

LC-MS (Method 1): Rₜ=1.23 min; MS (ESIpos): m/z=498 [M+H]⁺.

LC-MS (system: Waters Alliance 2695, DAD 996, ESA Corona, column: Chiralpak IC 3 μm 100×4.6 mm, solvent: ethanol+0.1% diethylamine, rate: 1.0 mL/min, temperature: 25° C., injection: 5.0 μL, detection: DAD 254 nm): Rₜ=4.84 min, 94% enantiomeric excess.

Optical rotation (Method 6): [α]=+6.4° (c=1.01, CHCl₃).

### Example 16

### 962 mg

¹H-NMR (300 MHz, DMSO-d₆): δ [ppm]=1.24 (d, 3H), 2.53-2.63 (m, 4H), 3.38 (q, 1H), 3.61-3.72 (m, 4H), 7.31-7.39 (m, 1H), 7.41-7.50 (m, 2H), 7.65-7.73 (m, 4H), 7.84-7.95 (m, 4H), 8.64 (s, 1H), 10.05 (s, 1H), 10.58 (s, 1H).

LC-MS (Method 1): Rₜ=1.24 min; MS (ESIpos): m/z=498 [M+H]⁺.

LC-MS (system: Waters Alliance 2695, DAD 996, ESA Corona, column: Chiralpak IC 3 μm 100×4.6 mm, solvent: ethanol+0.1% diethylamine, rate: 1.0 mL/min, temperature: 25° C., injection: 5.0 μL, detection: DAD 254 nm): Rₜ=3.48 min, 95% enantiomeric excess.

Optical rotation (Method 6): [α]=−9.3° (c=1.08, CHCl₃).

### Example 17

### N-(biphenyl-4-yl)-3-{[2-methyl-2-(morpholin-4-yl)propanoyl]amino}-4-(trifluoromethyl)benzamide

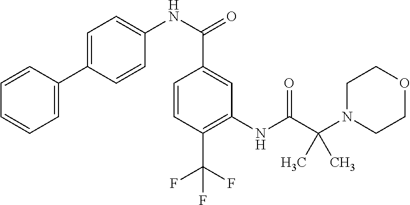 [SMILES: CC(C)(C(=O)Nc1cc(C(=O)Nc2ccc(-c3ccccc3)cc2)ccc1C(F)(F)F)N1CCOCC1]

101 mg (199 μmol) of the compound from example 17A were provided in 2 mL of DMF. 42 μL (298 μmol) of triethylamine and 26 μL (298 μmol) of morpholine were added, and the mixture was stirred at room temperature for 5 h and at 120° C. for 10 h. After filtration, purification by HPLC (1. method 2; 2. system: Waters Autopurificationsystem, column: XBrigde C18 5 μm 100×30 mm, solvent: water/methanol+0.1% formic acid gradient, rate: 50 mL/min, temperature: room temperature) yielded 18.9 mg (18% of theory) of the title compound.

¹H-NMR (500 MHz, DMSO-d₆): δ [ppm]=1.24 (s, 6H), 2.50-2.54 (m, 4H), 3.65-3.70 (m, 4H), 7.32-7.37 (m, 1H), 7.44-7.49 (m, 2H), 7.67-7.72 (m, 4H), 7.85-7.94 (m, 4H), 8.66 (s, 1H), 9.98 (s, 1H), 10.58 (s, 1H).

LC-MS (Method 1): Rₜ=1.42 min; MS (ESIpos): m/z=512 [M+H]⁺.

### Example 18

### N-(biphenyl-4-yl)-4-cyano-3-[(morpholin-4-ylacetyl)amino]benzamide

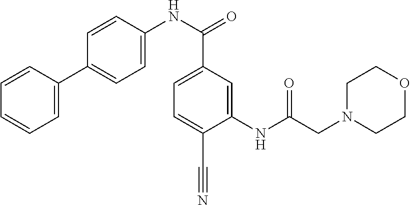 [SMILES: N#Cc1ccc(C(=O)Nc2ccc(-c3ccccc3)cc2)cc1NC(=O)CN1CCOCC1]

To a solution of N-(biphenyl-4-yl)-4-bromo-3-[(morpholin-4-ylacetyl)amino]benzamide (prepared in a manner analogous to that described in example 13, 0.15 g, 0.30 mmol) in DMF (3 mL) under argon was added tetrakis(triphenylphosphine)palladium(0) (35 mg, 0.030 mmol, 10 mol %), and zinc cyanide (37 mg, 0.32 mmol, 1.05 equiv). The resulting mixture was heated at 90° C. for 20 h, was then added to ice water (10 mL). The resulting precipitate was filtered, washed with water followed by ethanol, and dried at 50° C. under reduced pressure. The resulting solids were purified by HPLC to give N-(biphenyl-4-yl)-4-cyano-3-[(morpholin-4-ylacetyl)amino]benzamide (59 mg, 43%).

¹H-NMR (400 MHz, DMSO-d₆): δ [ppm]=2.55-2.59 (m, 4H), 3.20 (s, 2H), 3.64-3.69 (m, 4H), 7.31 (t, J=7.3 Hz, 1H), 7.42 (t, J=7.5 Hz, 2H), 7.64 (d, J=7.2 Hz, 2H), 7.66 (d, J=8.7 Hz, 2H), 7.81-7.86 (m, 3H), 8.01 (d, J=8.1 Hz, 1H), 8.51 (d, J=1.3 Hz, 1H), 10.28 (s, 1H), 10.57 (s, 1H).

LC-MS (Method 3): Rₜ=1.27 min; MS (ESIpos): m/z=441 ([M+H]⁺, 100%), 881 ([2M+H]⁺, 60%); MS (ESIneg): m/z=439 ([M−H]⁻, 100%), 879 ([2M−H]⁻, 10%).

### Example 19

### N-(biphenyl-4-yl)-3-[(morpholin-4-ylacetyl)amino]-4-(2-thienyl)benzamide

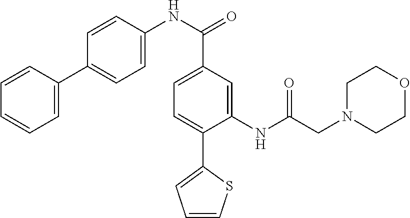 [SMILES: O=C(CN1CCOCC1)Nc1cc(C(=O)Nc2ccc(-c3ccccc3)cc2)ccc1-c1cccs1]

To a microwave vial was added N-(biphenyl-4-yl)-4-bromo-3-[(morpholin-4-ylacetyl)amino]benzamide (prepared in a manner analogous to that described in example 13, 0.12 g, 0.243 mmol), 2-thienylboronic acid (0.062 g, 0.49 mmol, 2.0 equiv), sodium carbonate (0.077 mg, 0.73 mmol, 3.0 equiv), dioxane (2.6 mL) and water (0.4 mL). The resulting suspension was purged with argon, treated with [1,1′-bis(diphenylphosphino)ferrocene]palladium(II) chloride CH₂Cl₂ complex (Pd(dppf)Cl₂.CH₂Cl₂, 0.020 g, 0.024 mmol, 10 mol %) and sealed. The resulting mixture was heated with a microwave apparatus at 105° C. for 1 h, was then cooled to room temperature. The reaction mixture was poured onto water, and extracted with a 4:1 mixture of CH₂Cl₂ and isopropanol. The combined organic phases were dried (Na₂SO₄ anh), and concentrated under reduced pressure. The residue was then purified by HPLC (method 2) to give N-(biphenyl-4-yl)-3-[(morpholin-4-ylacetyl)amino]-4-(2-thienyl)benzamide (68 mg, 56%).

¹H-NMR (300 MHz, DMSO-d₆): δ [ppm]=2.40-2.47 (m, 4H), 3.12 (s, 2H), 3.39-3.50 (m, 4H), 7.30 (dd, 1H), 7.35 (d, 1H), 7.42-7.50 (m, 3H), 7.61 (d, 1H), 7.65-7.73 (m, 4H), 7.77-7.84 (m, 2H), 7.86-7.94 (m, 2H), 8.76 (d, 1H), 9.88 (s, 1H), 10.44 (s, 1H).

LC-MS (Method 3): Rₜ=1.37 min; MS (ESIpos): m/z=498 [M+H]⁺.

### Example 20

### N-(biphenyl-4-yl)-4-(2-furyl)-3-[(morpholin-4-ylacetyl)amino]benzamide

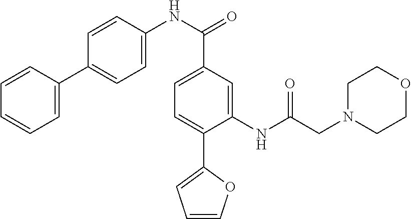 [SMILES: O=C(CN1CCOCC1)Nc1cc(C(=O)Nc2ccc(-c3ccccc3)cc2)ccc1-c1ccco1]

To a microwave vial was added N-(biphenyl-4-yl)-4-bromo-3-[(morpholin-4-ylacetyl)amino]benzamide (prepared in a manner analogous to that described in example 13, 0.12 g, 0.243 mmol), 2-furylboronic acid (0.054 g, 0.49 mmol, 2.0 equiv), sodium carbonate (0.077 mg, 0.73 mmol, 3.0 equiv), dioxane (2.6 mL) and water (0.4 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.020 g, 0.024 mmol, 10 mol %) and sealed. The resulting mixture was heated with a microwave apparatus at 105° C. for 1 h, was then cooled to room temperature. The reaction mixture was poured onto water, and extracted with a 4:1 mixture of CH₂Cl₂ and isopropanol. The combined organic phases were dried (Na₂SO₄ anh), and concentrated under reduced pressure. The residue was then purified by HPLC (method 2) to give N-(biphenyl-4-yl)-4-(2-furyl)-3-[(morpholin-4-ylacetyl)amino]benzamide (33 mg, 28%).

¹H-NMR (400 MHz, DMSO-d₆): δ [ppm]=2.53-2.59 (m, 4H), 3.21 (s, 2H), 3.58-3.67 (m, 4H), 6.78 (dd, 1H), 7.09 (d, 1H), 7.31-7.37 (m, 1H), 7.42-7.49 (m, 2H), 7.65-7.72 (m, 4H), 7.79-7.84 (m, 2H), 7.86-7.92 (m, 2H), 7.98 (d, 1H), 8.78 (s, 1H), 10.20 (s, 1H), 10.41 (s, 1H).

LC-MS (Method 3): Rₜ=1.33 min; MS (ESIpos): m/z=482 [M+H]⁺.

### Example 21

### N⁴-(biphenyl-4-yl)-N¹,N¹-dimethyl-2-[(morpholin-4-ylacetyl)amino]terephthalamide

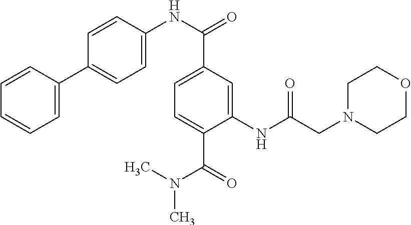 [SMILES: CN(C)C(=O)c1ccc(C(=O)Nc2ccc(-c3ccccc3)cc2)cc1NC(=O)CN1CCOCC1]

A mixture of dilithium N-(biphenyl-4-yl)-4-carboxy-3-{(Z)-[2-(morpholin-4-yl)-1-oxidanidylethylidene]amino}benzenecarboximidate (prepared in a manner analogous to that described in example 18A, 100 mg, 0.21 mmol) and a 2M solution of dimethylamine in THF (1.06 mL, 2.12 mmol, 10 equiv) in DMF (2.5 mL) was treated with (benzotriazol-1-yloxy)tripyrrolidinophosphonium hexafluorophosphate (PYBOP, 166 mg, 0.32 mmol, 1.50 equiv) and diisopropylethylamine (0.19 mL, 1.06 mmol, 5.0 equiv). The resulting mixture was stirred at room temperature for 24 h. The resulting mixture was treated with water and extracted with a dichloromethane/isopropanol mixture (4:1). The combined organic phases were dried (Na₂SO₄ anh), and concentrated under reduced pressure. The residue was recrystallized from methanol to give N⁴-(biphenyl-4-yl)-N¹,N¹-dimethyl-2-[(morpholin-4-ylacetyl)amino]terephthalamide (71.5 mg, 68%).

¹H-NMR (300 MHz, DMSO-d₆): δ [ppm]=2.47-2.56 (m, 4H), 2.92 (s, 3H), 3.06 (s, 3H), 3.14 (s, 2H), 3.63-3.74 (m, 4H), 7.30-7.38 (m, 1H), 7.41-7.55 (m, 3H), 7.63-7.71 (m, 4H), 7.75 (dd, 1H), 7.83-7.92 (m, 2H), 8.70 (d, 1H), 10.08 (s, 1H), 10.43 (s, 1H).

LC-MS (Method 3): Rₜ=1.16 min; MS (ESIpos): m/z=487 [M+H]⁺.

### Example 22

### N⁴-(biphenyl-4-yl)-N¹-methyl-2-[(morpholin-4-ylacetyl)amino]terephthalamide

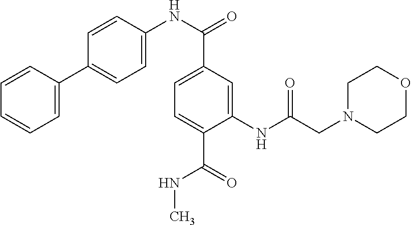 [SMILES: CNC(=O)c1ccc(C(=O)Nc2ccc(-c3ccccc3)cc2)cc1NC(=O)CN1CCOCC1]

A mixture of dilithium N-(biphenyl-4-yl)-4-carboxy-3-{(Z)-[2-(morpholin-4-yl)-1-oxidanidylethylidene]amino}benzenecarboximidate (prepared in a manner analogous to that described in example 18A, 100 mg, 0.21 mmol) and a 2M solution of methylamine in THF (1.06 mL, 2.12 mmol, 10 equiv) in DMF (2.5 mL) was treated with (benzotriazol-1-yloxy)tripyrrolidinophosphonium hexafluorophosphate (PYBOP, 166 mg, 0.32 mmol, 1.50 equiv) and diisopropylethylamine (0.11 mL, 0.64 mmol, 3.0 equiv). The resulting mixture was stirred at room temperature for 24 h. The resulting mixture was treated with water and extracted with a dichloromethane/isopropanol mixture (4:1). The combined organic phases were dried (Na₂SO₄ anh), and concentrated under reduced pressure. The residue was recrystallized from methanol to give N⁴-(biphenyl-4-yl)-N¹-methyl-2-[(morpholin-4-ylacetyl)amino]terephthalamide (64 mg, 61%).

¹H-NMR (300 MHz, DMSO-d₆): δ [ppm]=2.47-2.56 (m, 4H), 2.84 (d, 3H), 3.16 (s, 2H), 3.70-3.79 (m, 4H), 7.31-7.38 (m, 1H), 7.42-7.50 (m, 2H), 7.64-7.74 (m, 5H), 7.75-7.82 (m, 1H), 7.84-7.92 (m, 2H), 8.70-8.79 (m, 1H), 9.00-9.06 (m, 1H), 10.45 (s, 1H), 11.87 (s, 1H).

LC-MS (Method 3): Rₜ=1.16 min; MS (ESIpos): m/z=473 [M+H]⁺.

### Example 23

### N-(biphenyl-4-yl)-3-[(morpholin-4-ylacetyl)amino]-4-(trifluoromethoxy)benzamide

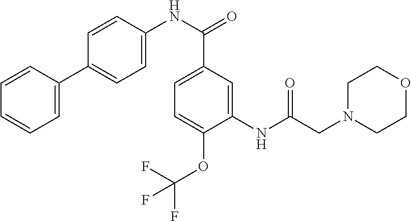 [SMILES: O=C(CN1CCOCC1)Nc1cc(C(=O)Nc2ccc(-c3ccccc3)cc2)ccc1OC(F)(F)F]

To a solution of 3-[(morpholin-4-ylacetyl)amino]-4-(trifluoromethoxy)benzoic acid (prepared in a manner analogous to that described in example 20A, 0.20 g, 0.57 mmol) and biphenyl-4-amine (0.097 g, 0.57 mmol, 1.0 equiv) in DMF (4 mL) was added (benzotriazol-1-yloxy)tripyrrolidinophosphonium hexafluorophosphate (PYBOP, 0.30 g, 0.57 mmol, 1.0 equiv) followed by diisopropylethylamine (0.30 mL, 1.72 mmol, 3.0 equiv). The resulting mixture was stirred at room temperature for 24 h, was then treated with water (5 mL). The resulting mixture was extracted with ethyl acetate (10 mL). The organic phase was dried (Na₂SO₄ anh), and concentrated under reduced pressure. The residue (0.25 g) was purified using HPLC (method 3) to give N-(biphenyl-4-yl)-3-[(morpholin-4-ylacetyl)amino]-4-(trifluoromethoxy)benzamide (0.080 g, 28%).

¹H-NMR (400 MHz, DMSO-d₆): δ [ppm]=2.53-2.57 (m, 4H), 3.20 (s, 2H), 3.60-3.64 (m, 4H), 7.37 (tm, J=7.2 Hz, 1H), 7.42 (t, J=7.7 Hz, 2H), 7.60 (dd, J=1.5, 8.6 Hz, 1H), 7.63-7.67 (m, 3H) 7.79 (dd, J=2.3, 8.6 Hz, 1H), 7.83 (d, J=8.8 Hz, 2H), 8.72 (d, J=2.0 Hz, 1H), 8.96 (d, J=2.5 Hz, 1H), 9.88 (s, 1H), 10.44 (s, 1H).

LC-MS (Method 3): Rₜ=1.38 min; MS (ESIpos): m/z=500 ([M+H]⁺, 30%), 999 ([2M+H]⁺, 50%); MS (ESIneg): m/z=498 ([M−H]⁻, 100%).

### Example 24

### 4-(benzyloxy)-N-(biphenyl-4-yl)-3-[(morpholin-4-ylacetyl)amino]benzamide

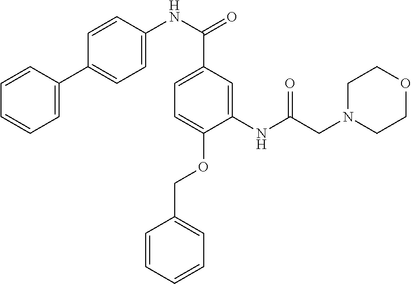 [SMILES: O=C(CN1CCOCC1)Nc1cc(C(=O)Nc2ccc(-c3ccccc3)cc2)ccc1OCc1ccccc1]

A mixture of lithium 4-(benzyloxy)-3-[(morpholin-4-ylacetyl)amino]benzoate (2.10 g, 5.58 mmol) (prepared in a manner analogous to that described in example 23A, 2.15 g, 5.59 mmol) and biphenyl-4-amine (1.32 g, 7.81 mmol, 1.4 equiv) in DMF (39 mL) was treated with propanephosphonic anhydride (50%, 4.6 mL, 7.81 mmol, 1.4 equiv), followed by diisopropylethylamine (2.9 mL, 16.7 mmol, 3.0 equiv). The resulting mixture was stirred at room temperature for 24 h. The resulting mixture was then treated with (benzotriazol-1-yloxy)tripyrrolidinophosphonium hexafluorophosphate (PYBOP, 4.36 g, 8.37 mmol, 1.50 mmol) and diisopropylethylamine (2.9 mL, 16.7 mmol, 3.0 equiv). The resulting mixture was stirred at room temperature for 12 h. The resulting mixture was concentrated under reduced pressure and treated with an ethanol/ethyl acetate mixture (1:1, 40 mL). The resulting solids were removed by filtration and washed with ethyl acetate to give 4-(benzyloxy)-N-(biphenyl-4-yl)-3-[(morpholin-4-ylacetyl)amino]benzamide (1.36 g, 47%).

¹H-NMR (300 MHz, DMSO-d₆): δ [ppm]=2.37-2.42 (m, 4H), 3.08 (s, 2H), 3.23-3.28 (m, 4H), 5.24 (s, 2H), 7.27-7.34 (m, 2H), 7.38-7.45 (m, 5H), 7.52-7.56 (m, 2H), 7.61-7.66 (m, 4H), 7.74 (dd, J=2.1, 8.7 Hz, 1H), 7.82-7.86 (m, 2H), 8.84 (d, J=2.1 Hz, 1H), 9.73 (s, 1H), 10.23 (s, 1H).

LC-MS (Method 1): Rₜ=1.42 min; MS (ESIpos): m/z=522 ([M+H]⁺, 100%); MS (ESIneg): m/z=520 ([M−H]⁻, 100%).

### Example 25

### N-(biphenyl-4-yl)-4-isopropoxy-3-[(morpholin-4-ylacetyl)amino]benzamide

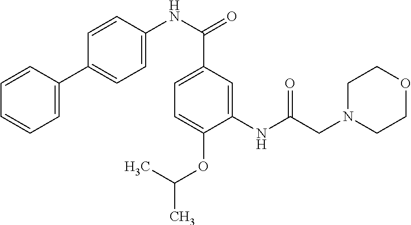 [SMILES: CC(C)Oc1ccc(C(=O)Nc2ccc(-c3ccccc3)cc2)cc1NC(=O)CN1CCOCC1]

A mixture of N-(biphenyl-4-yl)-4-hydroxy-3-[(morpholin-4-ylacetyl)amino]benzamide (prepared in a manner analogous to that described in example 24A, 0.11 g, 0.26 mmol), 2-iodopropane (0.076 mL, 0.77 mmol, 3.0 equiv), and Cs₂CO₃ (0.33 g, 1.02 mmol, 4.0 equiv) in DMF (2.6 mL) was heated at 60° C. for 6 h, was then cooled to room temperature and treated with water (5 mL). The resulting mixture was extracted with a CH₂Cl₂/isopropanol mixture (4:1, 3×10 mL). The combined organic phases were dried (Na₂CO₃ anh) and concentrated under reduced pressure. The residue (0.12 g) was recrystallized from ethanol to give N-(biphenyl-4-yl)-4-isopropoxy-3-[(morpholin-4-ylacetyl)amino]benzamide (0.053 g, 43%).

¹H-NMR (300 MHz, DMSO-d₆): δ [ppm]=1.36 (d, J=6.1 Hz, 6H), 2.52-2.56 (m, 4H), 3.15 (s, 2H), 3.65-3.68 (m, 4H), 4.84 (sept, J=6.1 Hz, 1H), 7.20 (d, J=8.8 Hz, 1H), 7.30 (t, J=7.3 Hz, 1H), 7.42 (t, J=7.7 Hz, 2H), 7.61-7.65 (m, 4H), 7.69 (dd, J=2.3, 8.6 Hz, 1H), 7.83 (d, J=8.6 Hz, 2H), 8.84 (d, J=2.3 Hz, 1H), 9.77 (s, 1H), 10.19 (s, 1H).

LC-MS (Method 3): Rₜ=1.37 min; MS (ESIpos): m/z=474 ([M+H]⁺, 100%); MS (ESIneg): m/z=472 ([M−H]⁻, 100%).

### Example 26

### N-(biphenyl-4-yl)-4-ethoxy-3-[(morpholin-4-ylacetyl)amino]benzamide

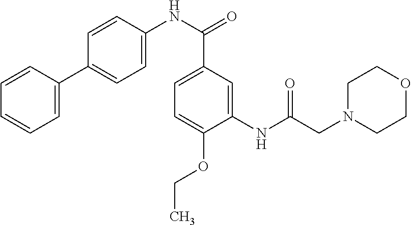 [SMILES: CCOc1ccc(C(=O)Nc2ccc(-c3ccccc3)cc2)cc1NC(=O)CN1CCOCC1]

A mixture of N-(biphenyl-4-yl)-4-hydroxy-3-[(morpholin-4-ylacetyl)amino]benzamide (prepared in a manner analogous to that described in example 24A, 0.10 g, 0.23 mmol), iodoethane (0.023 mL, 0.290 mmol, 1.25 equiv), and Cs₂CO₃ (0.15 g, 0.46 mmol, 2.0 equiv) in DMF (2.4 mL) was stirred at room temperature for 24 h, was then treated with water (5 mL). The resulting mixture was extracted with a CH₂Cl₂/isopropanol mixture (4:1, 3×5 mL). The combined organic phases were dried (Na₂CO₃ anh) and concentrated under reduced pressure. The residue (0.12 g) was recrystallized from ethanol to give N-(biphenyl-4-yl)-4-ethoxy-3-[(morpholin-4-ylacetyl)amino]benzamide (0.059 g, 54%).

¹H-NMR (300 MHz, DMSO-d₆): δ [ppm]=1.45 (t, J=7.1 Hz, 3H), 2.52-2.56 (m, 4H), 3.14 (s, 2H), 3.64-3.67 (m, 4H), 4.20 (q, J=7.1 Hz, 2H), 7.16 (d, J=8.8 Hz, 1H), 7.30 (t, J=7.4 Hz, 1H), 7.42 (t, J=7.7 Hz, 2H), 7.61-7.65 (m, 4H), 7.71 (dd, J=2.3, 8.6 Hz, 1H), 7.83 (d, J=8.6 Hz, 2H), 8.81 (d, J=2.0 Hz, 1H), 9.81 (s, 1H), 10.19 (s, 1H).

LC-MS (Method 3): Rₜ=1.29 min; MS (ESIpos): m/z=460 ([M+H]⁺, 100%), 919 ([2M+H]⁺, 60%); MS (ESIneg): m/z=458 ([M−H]⁻, 100%), 917 ([2M−H]⁻, 10%).

### Example 27

### N-{4-methoxy-3-[(1H-pyrazol-1-ylacetyl)amino]phenyl}biphenyl-4-carboxamide

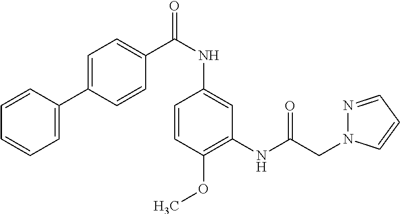 [SMILES: COc1ccc(NC(=O)c2ccc(-c3ccccc3)cc2)cc1NC(=O)Cn1cccn1]

120 mg (377 μmol) of the compound from example 28A and 197 μL (1.13 mmol) of N,N-diisopropylethylamine were provided in 2 mL of DMF. 57.0 mg (452 μmol) of 1H-pyrazol-1-ylacetic acid and 264 μL (452 μmol) of a 50% solution of 2,4,6-tripropyl-1,3,5,2,4,6-trioxatriphosphinane 2,4,6-trioxide (T3P) in DMF were added, and the mixture was stirred over night at room temperature. 64.0 mg (507 μmol) of 1H-pyrazol-1-ylacetic acid and 264 μL (452 μmol) of a 50% solution of 2,4,6-tripropyl-1,3,5,2,4,6-trioxatriphosphinane 2,4,6-trioxide (T3P) in DMF were added, and the mixture was stirred for 24 h at room temperature. After filtration, purification by HPLC (method 2) yielded 99.0 mg (62% of theory) of the title compound.

¹H-NMR (300 MHz, DMSO-d₆): δ [ppm]=3.83 (s, 3H), 5.13 (s, 2H), 6.32 (t, 1H), 7.04 (d, 1H), 7.37-7.64 (m, 5H), 7.72-7.86 (m, 5H), 8.01-8.09 (m, 2H), 8.44 (d, 1H), 9.33 (s, 1H), 10.19 (s, 1H).

LC-MS (Method 4): Rₜ=1.20 min; MS (ESIpos): m/z=427 [M+H]⁺.

### Example 28

### N-(4-methoxy-3-{[2-methyl-2-(morpholin-4-yl)propanoyl]amino}phenyl)biphenyl-4-carboxamide

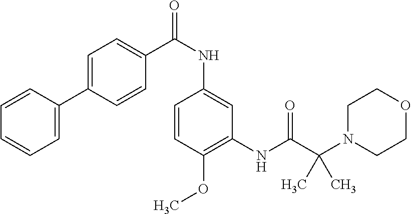 [SMILES: COc1ccc(NC(=O)c2ccc(-c3ccccc3)cc2)cc1NC(=O)C(C)(C)N1CCOCC1]

120 mg (377 μmol) of the compound from example 28A and 197 μL (1.13 mmol) of N,N-diisopropylethylamine were provided in 2 mL of DMF. 78.0 mg (452 μmol) of 2-methyl-2-(morpholin-4-yl)propanoic acid and 264 μL (452 μmol) of a 50% solution of 2,4,6-tripropyl-1,3,5,2,4,6-trioxatriphosphinane 2,4,6-trioxide (T3P) in DMF were added, and the mixture was stirred over night at room temperature. 78.0 mg (452 μmol) of 2-methyl-2-(morpholin-4-yl)propanoic acid and 264 μL (452 μmol) of a 50% solution of 2,4,6-tripropyl-1,3,5,2,4,6-trioxatriphosphinane 2,4,6-trioxide (T3P) in DMF were added, and the mixture was stirred for 24 h at room temperature and for 8 h at 50° C. After filtration, purification by HPLC (column: chromatorex C18, 10 μm, 195×51 mm, mobile phase: acetonitrile/water gradient with the addition of 0.1% formic acid) yielded 38.0 mg (19% of theory) of the title compound.

¹H-NMR (400 MHz, DMSO-d₆): δ [ppm]=1.21 (s, 6H), 2.51-2.58 (m, 4H), 3.67-3.73 (m, 4H), 3.90 (s, 3H), 7.04 (d, 1H), 7.39-7.46 (m, 1H), 7.47-7.57 (m, 3H), 7.72-7.85 (m, 4H), 8.04-8.10 (m, 2H), 8.59 (d, 1H), 9.94 (s, 1H), 10.20 (s, 1H).

LC-MS (Method 4): Rₜ=1.25 min; MS (ESIpos): m/z=474 [M+H]⁺.

### Example 29

### N-{4-fluoro-3-[(morpholin-4-ylacetyl)amino]phenyl}biphenyl-4-carboxamide

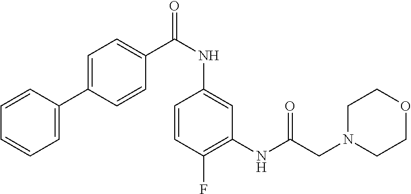 [SMILES: O=C(CN1CCOCC1)Nc1cc(NC(=O)c2ccc(-c3ccccc3)cc2)ccc1F]

100 mg (326 μmol) of the compound from example 29A and 171 μL (979 μmol) of N,N-diisopropylethylamine were provided in 2 mL of DMF at room temperature. 57.0 mg (392 μmol) of morpholin-4-ylacetic acid and 229 μL (392 μmol) of a 50% solution of 2,4,6-tripropyl-1,3,5,2,4,6-trioxatriphosphinane 2,4,6-trioxide (T3P) in DMF were added, and the mixture was stirred over night at room temperature. After filtration, purification by HPLC (method 2) yielded 68 mg of 91% purity (44% of theory) of the title compound.

¹H-NMR (300 MHz, DMSO-d₆): δ [ppm]=2.56-2.68 (m, 4H), 3.15-3.35 (m, 2H), 3.61-3.72 (m, 4H), 7.22-7.32 (m, 1H), 7.38-7.46 (m, 1H), 7.47-7.56 (m, 2H), 7.59-7.68 (m, 1H), 7.73-7.79 (m, 2H), 7.80-7.87 (m, 2H), 8.02-8.11 (m, 2H), 8.44 (d, 1H), 9.68 (s, 1H), 10.38 (s, 1H).

LC-MS (Method 4): Rₜ=1.00 min; MS (ESIpos): m/z=434 [M+H]⁺.

### Examples 30 and 31

### N-(4-fluoro-3-{[(2S)-2-(morpholin-4-yl)propanoyl]amino}phenyl)biphenyl-4-carboxamide

### N-(4-fluoro-3-{[(2R)-2-(morpholin-4-yl)propanoyl]amino}phenyl)biphenyl-4-carboxamide

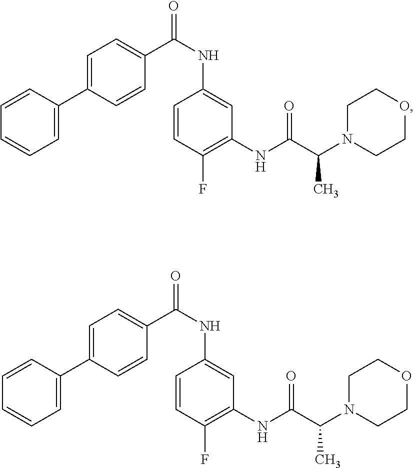 [SMILES: C[C@@H](C(=O)Nc1cc(NC(=O)c2ccc(-c3ccccc3)cc2)ccc1F)N1CCOCC1 ; C[C@H](C(=O)Nc1cc(NC(=O)c2ccc(-c3ccccc3)cc2)ccc1F)N1CCOCC1]

300 mg (979 μmol) of the compound from example 29A and 512 μL (2.94 mmol) of N,N-diisopropylethylamine were provided in 5 mL of DMF at room temperature. 230 mg (1.18 mmol) of 2-(morpholin-4-yl)propanoic acid and 686 μL (1.18 mmol) of a 50% solution of 2,4,6-tripropyl-1,3,5,2,4,6-trioxatriphosphinane 2,4,6-trioxide (T3P) in DMF were added, and the mixture was stirred over night at room temperature. 230 mg (1.18 mmol) of 2-(morpholin-4-yl)propanoic acid and 686 μL (1.18 mmol) of a 50% solution of 2,4,6-tripropyl-1,3,5,2,4,6-trioxatriphosphinane 2,4,6-trioxide (T3P) in DMF were added, and the mixture was stirred for 24 h at room temperature. After filtration, purification by HPLC (column: chromatorex C18, 10 μm, 195×51 mm, mobile phase: acetonitrile/water gradient with the addition of 0.1% formic acid) yielded 296 mg (61% of theory) of the racemate of the title compound. Chiral chromatography (system: Agilent Prep 1200, column: Chiralpak IC 5 μm 250×20 mm, solvent: hexane/ethanol 7/3+0.1% diethylamine, rate: 30 mL/min, temperature: room temperature, detection: UV 280 nm) of 260 mg of the racemate provided:

### Example 30

### 88.0 mg

¹H-NMR (300 MHz, DMSO-d₆): δ [ppm]=1.21 (d, 3H), 2.51-2.63 (m, 4H), 3.37 (q, 1H), 3.60-3.69 (m, 4H), 7.27 (dd, 1H), 7.39-7.47 (m, 1H), 7.47-7.55 (m, 2H), 7.64 (ddd, 1H), 7.73-7.80 (m, 2H), 7.80-7.87 (m, 2H), 8.04-8.11 (m, 2H), 8.43 (dd, 1H), 9.76 (s, 1H), 10.38 (s, 1H).

LC-MS (Method 1): Rₜ=1.06 min; MS (ESIpos): m/z=448 [M+H]⁺.

LC-MS (system: Waters Alliance 2695, DAD 996, ESA Corona, column: Chiralpak IC 3 μm 100×4.6 mm, solvent: hexane/ethanol 7/3+0.1% diethylamine, rate: 1.0 mL/min, temperature: 25° C., injection: 5.0 μL, detection: DAD 280 nm): Rₜ=7.2 min, 100% enantiomeric excess.

### Example 31

### 84.0 mg

¹H-NMR (300 MHz, DMSO-d₆): δ [ppm]=1.21 (d, 3H), 2.51-2.63 (m, 4H), 3.37 (q, 1H), 3.60-3.69 (m, 4H), 7.27 (dd, 1H), 7.39-7.47 (m, 1H), 7.47-7.55 (m, 2H), 7.64 (ddd, 1H), 7.73-7.80 (m, 2H), 7.80-7.87 (m, 2H), 8.04-8.11 (m, 2H), 8.43 (dd, 1H), 9.76 (s, 1H), 10.38 (s, 1H).

LC-MS (Method 1): Rₜ=1.06 min; MS (ESIpos): m/z=448 [M+H]⁺.

LC-MS (system: Waters Alliance 2695, DAD 996, ESA Corona, column: Chiralpak IC 3 μm 100×4.6 mm, solvent: hexane/ethanol 7/3+0.1% diethylamine, rate: 1.0 mL/min, temperature: 25° C., injection: 5.0 μL, detection: DAD 280 nm): Rₜ=9.5 min, 100% enantiomeric excess.

### Example 32

### N-{4-methoxy-3-[(8-oxa-3-azabicyclo[3.2.1]oct-3-ylacetyl)amino]phenyl}biphenyl-4-carboxamide

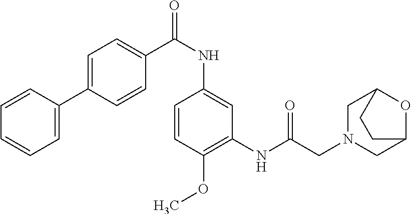 [SMILES: COc1ccc(NC(=O)c2ccc(-c3ccccc3)cc2)cc1NC(=O)CN1CC2CCC(C1)O2]

100 mg (253 μmol) of the compound from example 31A were provided in 2 mL of DMF. 88 μL (633 μmol) of triethylamine, 56.8 mg (380 μmol) of 8-oxa-3-azabicyclo[3.2.1]octane hydrochloride and 6.5 mg (39 μmol) of potassium iodide were added, and the mixture was stirred at room temperature over night. After filtration, purification by HPLC (method 2) yielded 80.7 mg (68% of theory) of the title compound.

¹H-NMR (300 MHz, DMSO-d₆): δ [ppm]=1.81-1.94 (m, 2H), 2.02-2.14 (m, 2H), 2.42-2.49 (m, 2H), 2.59-2.68 (m, 2H), 3.08 (s, 2H), 3.89 (s, 3H), 4.23-4.33 (m, 2H), 7.07 (d, 1H), 7.38-7.46 (m, 1H), 7.47-7.56 (m, 2H), 7.61 (dd, 1H), 7.72-7.79 (m, 2H), 7.79-7.86 (m, 2H), 8.03-8.11 (m, 2H), 8.68 (d, 1H), 9.73 (s, 1H), 10.26 (s, 1H).

LC-MS (Method 1): Rₜ=1.25 min; MS (ESIpos): m/z=472 [M+H]⁺.

### Example 33

### N-(4-methoxy-3-{[(1S,4S)-2-oxa-5-azabicyclo[2.2.1]hept-5-ylacetyl]amino}phenyl)biphenyl-4-carboxamide

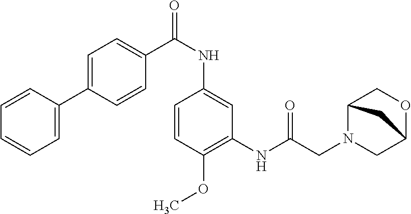 [SMILES: COc1ccc(NC(=O)c2ccc(-c3ccccc3)cc2)cc1NC(=O)CN1C[C@@H]2C[C@H]1CO2]

The preparation of the title compound took place analogously to the synthesis of the compound from example 32 starting with 100 mg (253 μmol) of the compound from example 31A and 51.5 mg (380 μmol) of (1S,4S)-2-oxa-5-azabicyclo[2.2.1]heptane hydrochloride. 62.5 mg (54% of theory) of the title compound were obtained.

¹H-NMR (300 MHz, DMSO-d₆): δ [ppm]=1.63-1.78 (m, 1H), 1.78-1.94 (m, 1H), 2.62-2.82 (m, 1H), 2.84-3.01 (m, 1H), 3.33-3.49 (m, 2H), 3.57-3.70 (m, 2H), 3.81-3.93 (m, 4H), 4.41-4.49 (m, 1H), 7.06 (d, 1H), 7.38-7.46 (m, 1H), 7.47-7.55 (m, 2H), 7.58 (dd, 1H), 7.72-7.79 (m, 2H), 7.79-7.86 (m, 2H), 8.03-8.10 (m, 2H), 8.61 (d, 1H), 9.77 (s, 1H), 10.24 (s, 1H).

LC-MS (Method 4): Rₜ=0.92 min; MS (ESIpos): m/z=458 [M+H]⁺.

### Example 34

### N-[3-({[(2R)-2-(hydroxymethyl)morpholin-4-yl]acetyl}amino)-4-methoxyphenyl]biphenyl-4-carboxamide

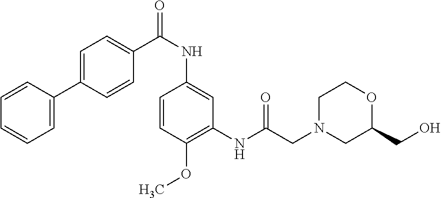 [SMILES: COc1ccc(NC(=O)c2ccc(-c3ccccc3)cc2)cc1NC(=O)CN1CCO[C@@H](CO)C1]

The preparation of the title compound took place analogously to the synthesis of the compound from example 32 starting with 100 mg (253 μmol) of the compound from example 31A and 44.5 mg (380 μmol) of (2R)-morpholin-2-ylmethanol with the exception that 53 μL (380 μmol) of triethylamine were used. 78.3 mg (65% of theory) of the title compound were obtained.

¹H-NMR (400 MHz, DMSO-d₆): δ [ppm]=2.04-2.14 (m, 1H), 2.27-2.38 (m, 1H), 2.73 (d, 1H), 2.88 (d, 1H), 3.11-3.21 (m, 2H), 3.33-3.39 (m, 1H), 3.42-3.63 (m, 3H), 3.83-3.92 (m, 4H), 4.71 (t, 1H), 7.06 (d, 1H), 7.39-7.46 (m, 1H), 7.48-7.54 (m, 2H), 7.60 (dd, 1H), 7.73-7.79 (m, 2H), 7.79-7.85 (m, 2H), 8.04-8.10 (m, 2H), 8.59 (d, 1H), 9.73 (s, 1H), 10.24 (s, 1H).

LC-MS (Method 4): Rₜ=0.96 min; MS (ESIpos): m/z=476 [M+H]⁺.

### Example 35

### N-(3-{[(4-cyclopropylpiperazin-1-yl)acetyl]amino}-4-methoxyphenyl)biphenyl-4-carboxamide

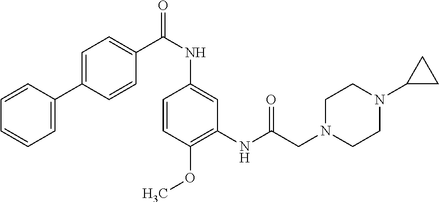 [SMILES: COc1ccc(NC(=O)c2ccc(-c3ccccc3)cc2)cc1NC(=O)CN1CCN(C2CC2)CC1]

The preparation of the title compound took place analogously to the synthesis of the compound from example 32 starting with 100 mg (253 μmol) of the compound from example 31A and 76.0 mg (380 μmol) of 1-cyclopropylpiperazine dihydrochloride with the exception that 159 μL (1.14 mmol) of triethylamine were used. 111 mg (90% of theory) of the title compound were obtained.

¹H-NMR (300 MHz, DMSO-d₆): δ [ppm]=3.89 (s, 3H), 7.07 (d, 1H), 7.38-7.46 (m, 1H), 7.47-7.63 (m, 3H), 7.72-7.79 (m, 2H), 7.79-7.86 (m, 2H), 8.02-8.11 (m, 2H), 9.83 (br. s, 1H), 10.25 (s, 1H).

LC-MS (Method 4): Rₜ=1.01 min; MS (ESIpos): m/z=485 [M+H]⁺.

### Example 36

### N-{4-methoxy-3-[(1,4-oxazepan-4-ylacetyl)amino]phenyl}biphenyl-4-carboxamide

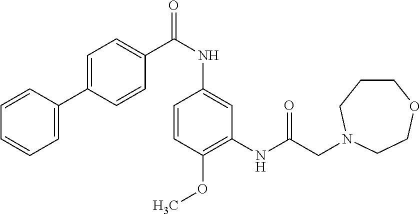 [SMILES: COc1ccc(NC(=O)c2ccc(-c3ccccc3)cc2)cc1NC(=O)CN1CCCOCC1]

The preparation of the title compound took place analogously to the synthesis of the compound from example 32 starting with 100 mg (253 μmol) of the compound from example 31A and 52.3 mg (380 μmol) of 1,4-oxazepane hydrochloride. 64.4 mg (52% of theory) of the title compound were obtained.

¹H-NMR (400 MHz, DMSO-d₆): δ [ppm]=1.85-1.94 (m, 2H), 2.73-2.84 (m, 4H), 3.27-3.33 (m, 2H), 3.66-3.73 (m, 2H), 3.79 (t, 2H), 3.89 (s, 3H), 7.06 (d, 1H), 7.39-7.46 (m, 1H), 7.48-7.54 (m, 2H), 7.60 (dd, 1H), 7.73-7.79 (m, 2H), 7.79-7.85 (m, 2H), 8.04-8.10 (m, 2H), 8.62 (d, 1H), 9.82 (s, 1H), 10.24 (s, 1H).

LC-MS (Method 4): Rₜ=0.94 min; MS (ESIpos): m/z=460 [M+H]⁺.

### Example 37

### N-{4-methoxy-3-[(thiomorpholin-4-ylacetyl)amino]phenyl}biphenyl-4-carboxamide

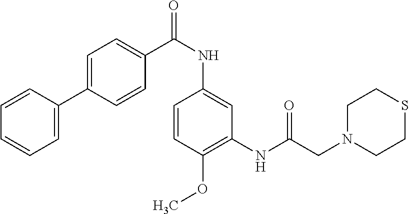 [SMILES: COc1ccc(NC(=O)c2ccc(-c3ccccc3)cc2)cc1NC(=O)CN1CCSCC1]

The preparation of the title compound took place analogously to the synthesis of the compound from example 32 starting with 100 mg (253 μmol) of the compound from example 31A and 39.0 mg (380 μmol) of thiomorpholine with the exception that 53 μL (380 μmol) of triethylamine were used. 86.0 mg (74% of theory) of the title compound were obtained.

¹H-NMR (400 MHz, DMSO-d₆): δ [ppm]=2.68-2.75 (m, 4H), 2.80 (d, 4H), 3.16 (s, 2H), 3.90 (s, 3H), 7.06 (d, 1H), 7.38-7.46 (m, 1H), 7.48-7.54 (m, 2H), 7.60 (dd, 1H), 7.73-7.78 (m, 2H), 7.79-7.85 (m, 2H), 8.04-8.10 (m, 2H), 8.58 (d, 1H), 9.67 (s, 1H), 10.24 (s, 1H).

LC-MS (Method 4): Rₜ=1.11 min; MS (ESIpos): m/z=462 [M+H]⁺.

### Example 38

### N-(4-methoxy-3-{[(3-methoxypiperidin-1-yl)acetyl]amino}phenyl)biphenyl-4-carboxamide

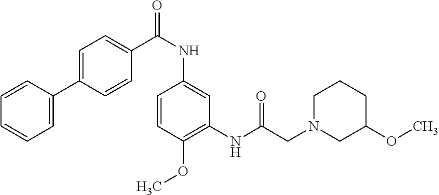 [SMILES: COc1ccc(NC(=O)c2ccc(-c3ccccc3)cc2)cc1NC(=O)CN1CCCC(OC)C1]

The preparation of the title compound took place analogously to the synthesis of the compound from example 32 starting with 100 mg (253 μmol) of the compound from example 31A and 43.8 mg (380 μmol) of 3-methoxypiperidine with the exception that 53 μL (380 μmol) of triethylamine were used. 57.8 mg (48% of theory) of the title compound were obtained.

¹H-NMR (300 MHz, DMSO-d₆): δ [ppm]=1.10-1.33 (m, 1H), 1.41-1.64 (m, 1H), 1.68-1.84 (m, 1H), 1.86-2.02 (m, 1H), 2.10-2.31 (m, 2H), 2.61-2.76 (m, 1H), 2.90-3.04 (m, 1H), 3.15 (s, 2H), 3.24-3.38 (m, 4H), 3.88 (s, 3H), 7.06 (d, 1H), 7.38-7.46 (m, 1H), 7.47-7.55 (m, 2H), 7.59 (dd, 1H), 7.72-7.78 (m, 2H), 7.79-7.86 (m, 2H), 8.02-8.11 (m, 2H), 8.59 (s, 1H), 9.73 (s, 1H), 10.24 (s, 1H).

LC-MS (Method 4): Rₜ=0.98 min; MS (ESIpos): m/z=474 [M+H]⁺.

### Example 39

### N-(4-methoxy-3-{[(4-methoxypiperidin-1-yl)acetyl]amino}phenyl)biphenyl-4-carboxamide

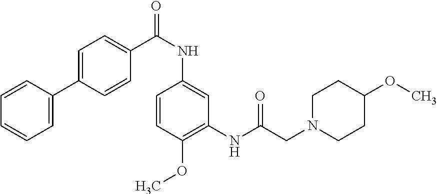 [SMILES: COc1ccc(NC(=O)c2ccc(-c3ccccc3)cc2)cc1NC(=O)CN1CCC(OC)CC1]

The preparation of the title compound took place analogously to the synthesis of the compound from example 32 starting with 100 mg (253 μmol) of the compound from example 31A and 44.0 mg (380 μmol) of 4-methoxypiperidine with the exception that 53 μL (380 μmol) of triethylamine were used. 84.0 mg (69% of theory) of the title compound were obtained.

¹H-NMR (400 MHz, DMSO-d₆): δ [ppm]=1.44-1.65 (m, 2H), 1.84-1.99 (m, 2H), 2.27-2.42 (m, 2H), 2.69-2.83 (m, 2H), 3.12 (s, 2H), 3.24-3.28 (m, 4H), 3.87 (s, 3H), 7.06 (d, 1H), 7.39-7.46 (m, 1H), 7.47-7.62 (m, 3H), 7.72-7.78 (m, 2H), 7.79-7.85 (m, 2H), 8.03-8.10 (m, 2H), 8.58 (s, 1H), 9.79 (s, 1H), 10.23 (s, 1H).

LC-MS (Method 4): Rₜ=0.99 min; MS (ESIpos): m/z=474 [M+H]⁺.

### Example 40

### N-[3-({[(3S)-3-hydroxypiperidin-1-yl]acetyl}amino)-4-methoxyphenyl]biphenyl-4-carboxamide

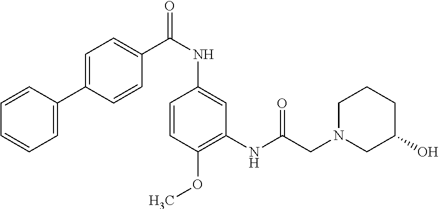 [SMILES: COc1ccc(NC(=O)c2ccc(-c3ccccc3)cc2)cc1NC(=O)CN1CCC[C@H](O)C1]

The preparation of the title compound took place analogously to the synthesis of the compound from example 32 starting with 100 mg (253 μmol) of the compound from example 31A and 52.3 mg (380 μmol) of (3S)-piperidin-3-ol hydrochloride. 88.4 mg (76% of theory) of the title compound were obtained.

¹H-NMR (400 MHz, DMSO-d₆): δ [ppm]=1.05-1.25 (m, 1H), 1.45-1.64 (m, 1H), 1.65-1.94 (m, 2H), 1.95-2.25 (m, 2H), 2.62-2.79 (m, 1H), 2.80-2.99 (m, 1H), 3.12 (s, 2H), 3.50-3.68 (m, 1H), 3.87 (s, 3H), 4.75 (s, 1H), 7.06 (d, 1H), 7.39-7.46 (m, 1H), 7.48-7.54 (m, 2H), 7.58 (d, 1H), 7.73-7.79 (m, 2H), 7.79-7.85 (m, 2H), 8.03-8.10 (m, 2H), 8.58 (s, 1H), 9.76 (s, 1H), 10.24 (s, 1H).

LC-MS (Method 4): Rₜ=0.91 min; MS (ESIpos): m/z=460 [M+H]⁺.

### Example 41

### N-(3-{[(2,2-dimethylmorpholin-4-yl)acetyl]amino}-4-methoxyphenyl)biphenyl-4-carboxamide

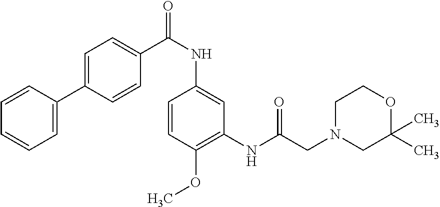 [SMILES: COc1ccc(NC(=O)c2ccc(-c3ccccc3)cc2)cc1NC(=O)CN1CCOC(C)(C)C1]

The preparation of the title compound took place analogously to the synthesis of the compound from example 32 starting with 100 mg (253 μmol) of the compound from example 31A and 43.8 mg (380 μmol) of 2,2-dimethylmorpholine with the exception that 53 μL (380 μmol) of triethylamine were used. 76.8 mg (64% of theory) of the title compound were obtained.

¹H-NMR (400 MHz, DMSO-d₆): δ [ppm]=1.26 (s, 6H), 2.33-2.40 (m, 2H), 2.43-2.48 (m, 2H), 3.10 (s, 2H), 3.67-3.74 (m, 2H), 3.86 (s, 3H), 7.06 (d, 1H), 7.39-7.45 (m, 1H), 7.48-7.54 (m, 2H), 7.59 (dd, 1H), 7.73-7.78 (m, 2H), 7.79-7.84 (m, 2H), 8.04-8.10 (m, 2H), 8.66 (d, 1H), 9.73 (s, 1H), 10.24 (s, 1H).

LC-MS (Method 4): Rₜ=1.25 min; MS (ESIpos): m/z=474 [M+H]⁺.

### Example 42

### N-(4-methoxy-3-{[N-(2-methoxyethyl)glycyl]amino}phenyl)biphenyl-4-carboxamide

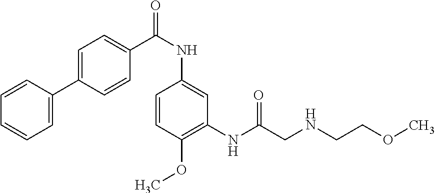 [SMILES: COCCNCC(=O)Nc1cc(NC(=O)c2ccc(-c3ccccc3)cc2)ccc1OC]

The preparation of the title compound took place analogously to the synthesis of the compound from example 32 starting with 88.0 mg (223 μmol) of the compound from example 31A and 29 μL (334 μmol) of 2-methoxyethanamine with the exception that 47 μL (334 μmol) of triethylamine were used. 23.9 mg (24% of theory) of the title compound were obtained.

¹H-NMR (400 MHz, DMSO-d₆): δ [ppm]=2.74 (t, 2H), 3.26 (s, 3H), 3.31 (s, 2H), 3.44 (t, 2H), 3.87 (s, 3H), 7.04 (d, 1H), 7.39-7.45 (m, 1H), 7.48-7.54 (m, 2H), 7.58 (dd, 1H), 7.74-7.78 (m, 2H), 7.79-7.84 (m, 2H), 8.04-8.10 (m, 2H), 8.61 (d, 1H), 9.88 (s, 1H), 10.23 (s, 1H).

LC-MS (Method 4): Rₜ=0.91 min; MS (ESIpos): m/z=434 [M+H]⁺.

### Example 43

### N-[3-({[(3R)-3-hydroxypyrrolidin-1-yl]acetyl}amino)-4-methoxyphenyl]biphenyl-4-carboxamide

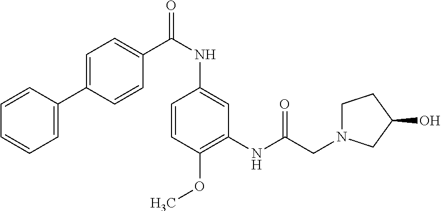 [SMILES: COc1ccc(NC(=O)c2ccc(-c3ccccc3)cc2)cc1NC(=O)CN1CC[C@@H](O)C1]

The preparation of the title compound took place analogously to the synthesis of the compound from example 32 starting with 100 mg (253 μmol) of the compound from example 31A and 47.0 mg (380 μmol) of (3R)-pyrrolidin-3-ol hydrochloride. 76.1 mg (61% of theory) of the title compound were obtained.

¹H-NMR (300 MHz, DMSO-d₆): δ [ppm]=1.60-1.73 (m, 1H), 1.99-2.14 (m, 1H), 2.57 (dd, 1H), 2.62-2.75 (m, 1H), 2.77-2.95 (m, 2H), 3.21-3.43 (m, 2H), 3.85 (s, 3H), 4.23-4.34 (m, 1H), 4.75-4.90 (m, 1H), 7.05 (d, 1H), 7.38-7.46 (m, 1H), 7.47-7.55 (m, 2H), 7.59 (dd, 1H), 7.72-7.79 (m, 2H), 7.79-7.86 (m, 2H), 8.03-8.10 (m, 2H), 8.54 (d, 1H), 9.56 (s, 1H), 10.23 (s, 1H).

LC-MS (Method 4): Rₜ=0.90 min; MS (ESIpos): m/z=446 [M+H]⁺.

### Example 44

### N-[3-({[(3R)-3-(2-hydroxyethyl)morpholin-4-yl]acetyl}amino)-4-methoxyphenyl]biphenyl-4-carboxamide

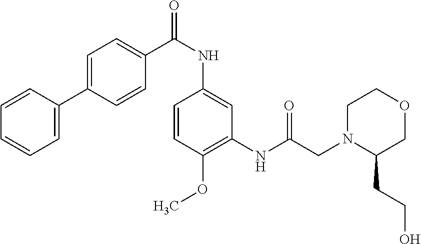 [SMILES: COc1ccc(NC(=O)c2ccc(-c3ccccc3)cc2)cc1NC(=O)CN1CCOC[C@H]1CCO]

The preparation of the title compound took place analogously to the synthesis of the compound from example 32 starting with 100 mg (253 μmol) of the compound from example 31A and 49.8 mg (380 μmol) of 2-[(3R)-morpholin-3-yl]ethanol with the exception that 53 μL (380 μmol) of triethylamine were used. 48.0 mg (39% of theory) of the title compound were obtained.

¹H-NMR (300 MHz, DMSO-d₆): δ [ppm]=1.43-1.71 (m, 2H), 2.45-2.55 (m, 1H), 2.57-2.68 (m, 1H), 2.76-2.86 (m, 1H), 3.04-3.15 (m, 1H), 3.34-3.52 (m, 4H), 3.55-3.66 (m, 1H), 3.67-3.76 (m, 1H), 3.76-3.84 (m, 1H), 3.90 (s, 3H), 4.50 (t, 1H), 7.06 (d, 1H), 7.38-7.46 (m, 1H), 7.47-7.55 (m, 2H), 7.59 (dd, 1H), 7.72-7.79 (m, 2H), 7.79-7.86 (m, 2H), 8.03-8.11 (m, 2H), 8.61 (d, 1H), 9.95 (s, 1H), 10.26 (s, 1H).

LC-MS (Method 1): Rₜ=1.05 min; MS (ESIpos): m/z=490 [M+H]⁺.

### Example 45

### N-(3-{[(4-hydroxypiperidin-1-yl)acetyl]amino}-4-methoxyphenyl)biphenyl-4-carboxamide

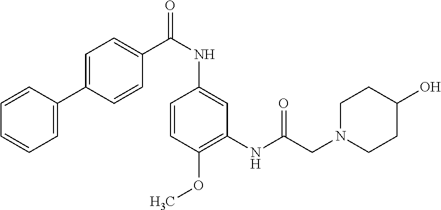 [SMILES: COc1ccc(NC(=O)c2ccc(-c3ccccc3)cc2)cc1NC(=O)CN1CCC(O)CC1]

The preparation of the title compound took place analogously to the synthesis of the compound from example 32 starting with 100 mg (253 μmol) of the compound from example 31A and 38.0 mg (380 μmol) of piperidin-4-ol with the exception that 53 μL (380 μmol) of triethylamine were used. 105 mg (90% of theory) of the title compound were obtained.

¹H-NMR (400 MHz, DMSO-d₆): δ [ppm]=1.39-1.66 (m, 2H), 1.73-1.93 (m, 2H), 2.19-2.43 (m, 2H), 2.65-2.90 (m, 2H), 2.97-3.23 (m, 2H), 3.45-3.65 (m, 1H), 3.88 (s, 3H), 4.65 (s, 1H), 7.06 (d, 1H), 7.38-7.46 (m, 1H), 7.47-7.54 (m, 2H), 7.58 (d, 1H), 7.72-7.86 (m, 4H), 8.03-8.10 (m, 2H), 8.58 (s, 1H), 9.83 (s, 1H), 10.24 (s, 1H).

LC-MS (Method 4): Rₜ=0.93 min; MS (ESIpos): m/z=460 [M+H]⁺.

### Example 46

### N-{4-methoxy-3-[(1-oxa-6-azaspiro[3.4]oct-6-ylacetyl)amino]phenyl}biphenyl-4-carboxamide

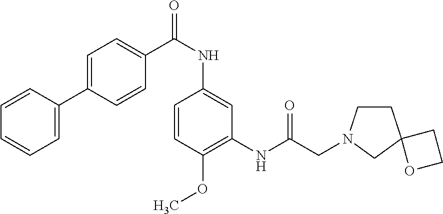 [SMILES: COc1ccc(NC(=O)c2ccc(-c3ccccc3)cc2)cc1NC(=O)CN1CCC2(CCO2)C1]

The preparation of the title compound took place analogously to the synthesis of the compound from example 32 starting with 100 mg (253 μmol) of the compound from example 31A and 43.0 mg (380 μmol) of 1-oxa-6-azaspiro[3.4]octane with the exception that 53 μL (380 μmol) of triethylamine were used. After filtration, purification by HPLC (Waters Autopurificationsystem SQD; column: Waters XBrigde C18 5 μm 100×30 mm, mobile phase: acetonitrile/water gradient with the addition of 0.1% trifluoroacetic acid) yielded 19.0 mg (16% of theory) of the title compound.

LC-MS (Method 4): Rₜ=0.95 min; MS (ESIpos): m/z=472 [M+H]⁺.

### Example 47

### N-(4-methoxy-3-{[(4-methylpiperazin-1-yl)acetyl]amino}phenyl)biphenyl-4-carboxamide

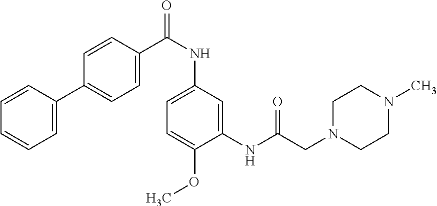 [SMILES: COc1ccc(NC(=O)c2ccc(-c3ccccc3)cc2)cc1NC(=O)CN1CCN(C)CC1]

The preparation of the title compound took place analogously to the synthesis of the compound from example 32 starting with 250 mg (633 μmol) of the compound from example 31A and 95.0 mg (950 μmol) of 1-methylpiperazine with the exception that 132 μL (950 μmol) of triethylamine were used. After filtration, purification by HPLC (column: chromatorex C18, 10 μm, 195×51 mm, mobile phase: acetonitrile/water gradient) yielded 274 mg (94% of theory) of the title compound.

¹H-NMR (300 MHz, DMSO-d₆): δ [ppm]=2.21 (s, 3H), 2.34-2.46 (m, 4H), 2.54-2.62 (m, 4H), 3.13 (s, 2H), 3.89 (s, 3H), 7.05 (d, 1H), 7.39-7.46 (m, 1H), 7.46-7.55 (m, 2H), 7.59 (dd, 1H), 7.73-7.85 (m, 4H), 8.03-8.12 (m, 2H), 8.59 (d, 1H), 9.77 (s, 1H), 10.24 (s, 1H).

LC-MS (Method 4): Rₜ=0.95 min; MS (ESIpos): m/z=459 [M+H]⁺.

### Example 48

### N-[4-methoxy-3-({[(3S)-3-methylmorpholin-4-yl]acetyl}amino)phenyl]biphenyl-4-carboxamide

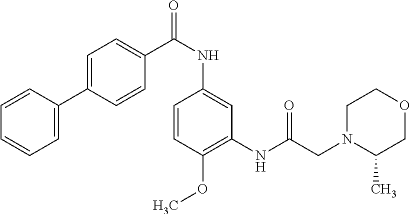 [SMILES: COc1ccc(NC(=O)c2ccc(-c3ccccc3)cc2)cc1NC(=O)CN1CCOC[C@@H]1C]

The preparation of the title compound took place analogously to the synthesis of the compound from example 32 starting with 100 mg (253 μmol) of the compound from example 31A and 38.4 mg (380 μmol) of (3S)-3-methylmorpholine with the exception that 53 μL (380 μmol) of triethylamine were used. After filtration, purification by HPLC (column: Chiralpak IC 5 μm 250×20 mm, solvent: methanol, rate: 20 mL/min, temperature: room temperature, detection: UV 280 nm) yielded 25.9 mg (22% of theory) of the title compound.

¹H-NMR (300 MHz, DMSO-d₆): δ [ppm]=0.94 (d, 3H), 2.53-2.64 (m, 2H), 2.69-2.84 (m, 1H), 3.04 (d, 1H), 3.19 (dd, 1H), 3.37 (d, 1H), 3.52-3.64 (m, 1H), 3.68-3.82 (m, 2H), 3.90 (s, 3H), 7.06 (d, 1H), 7.38-7.46 (m, 1H), 7.47-7.55 (m, 2H), 7.59 (dd, 1H), 7.72-7.79 (m, 2H), 7.79-7.86 (m, 2H), 8.03-8.11 (m, 2H), 8.58 (d, 1H), 9.91 (s, 1H), 10.25 (s, 1H).

LC-MS (Method 1): Rₜ=1.11 min; MS (ESIpos): m/z=460 [M+H]⁺.

### Example 49

### N-(4-methoxy-3-{[N-(2-methoxyethyl)-N-methylglycyl]amino}phenyl)biphenyl-4-carboxamide

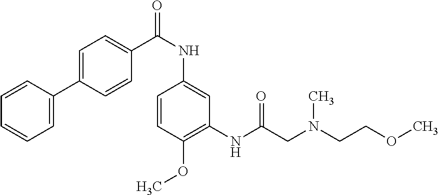 [SMILES: COCCN(C)CC(=O)Nc1cc(NC(=O)c2ccc(-c3ccccc3)cc2)ccc1OC]

The preparation of the title compound took place analogously to the synthesis of the compound from example 32 starting with 88.0 mg (223 μmol) of the compound from example 31A and 29.8 mg (334 μmol) of 2-methoxy-N-methylethanamine with the exception that 47 μL (334 μmol) of triethylamine were used. 40.0 mg (39% of theory) of the title compound were obtained.

¹H-NMR (400 MHz, DMSO-d₆): δ [ppm]=2.43 (s, 3H), 2.63-2.84 (m, 2H), 3.14-3.40 (m, 5H), 3.51 (t, 2H), 3.87 (s, 3H), 7.05 (d, 1H), 7.39-7.46 (m, 1H), 7.48-7.54 (m, 2H), 7.58 (dd, 1H), 7.73-7.79 (m, 2H), 7.79-7.85 (m, 2H), 8.04-8.10 (m, 2H), 8.56-8.62 (m, 1H), 9.66 (s, 1H), 10.23 (s, 1H).

LC-MS (Method 4): Rₜ=0.94 min; MS (ESIpos): m/z=448 [M+H]⁺.

### Example 50

### N-(3-{[(4-ethylpiperazin-1-yl)acetyl]amino}-4-methoxyphenyl)biphenyl-4-carboxamide

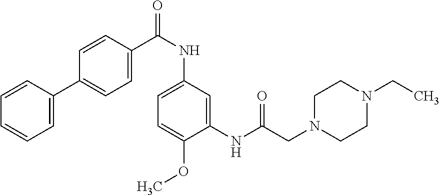 [SMILES: CCN1CCN(CC(=O)Nc2cc(NC(=O)c3ccc(-c4ccccc4)cc3)ccc2OC)CC1]

The preparation of the title compound took place analogously to the synthesis of the compound from example 32 starting with 100 mg (253 μmol) of the compound from example 31A and 43.0 mg (380 μmol) of 1-ethylpiperazine with the exception that 53 μL (380 μmol) of triethylamine were used. 110 mg (91% of theory) of the title compound were obtained.

¹H-NMR (300 MHz, DMSO-d₆): δ [ppm]=1.12 (t, 3H), 2.60-2.95 (m, 10H), 3.22 (s, 2H), 3.89 (s, 3H), 7.06 (d, 1H), 7.38-7.47 (m, 1H), 7.47-7.55 (m, 2H), 7.58 (dd, 1H), 7.72-7.86 (m, 4H), 8.02-8.10 (m, 2H), 8.56 (d, 1H), 9.62 (s, 1H), 10.23 (s, 1H).

LC-MS (Method 4): Rₜ=0.93 min; MS (ESIpos): m/z=473 [M+H]⁺.

### Example 51

### N-[4-methoxy-3-({[4-(methylsulfonyl)piperazin-1-yl]acetyl}amino)phenyl]biphenyl-4-carboxamide

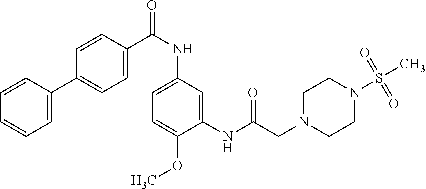 [SMILES: COc1ccc(NC(=O)c2ccc(-c3ccccc3)cc2)cc1NC(=O)CN1CCN(S(C)(=O)=O)CC1]

The preparation of the title compound took place analogously to the synthesis of the compound from example 32 starting with 100 mg (253 μmol) of the compound from example 31A and 62.0 mg (380 μmol) of 1-(methylsulfonyl)piperazine with the exception that 53 μL (380 μmol) of triethylamine were used. 38.0 mg (29% of theory) of the title compound were obtained.

¹H-NMR (300 MHz, DMSO-d₆): δ [ppm]=2.63-2.71 (m, 4H), 2.96 (s, 3H), 3.16-3.26 (m, 6H), 3.89 (s, 3H), 7.06 (d, 1H), 7.38-7.46 (m, 1H), 7.47-7.55 (m, 2H), 7.61 (dd, 1H), 7.72-7.85 (m, 4H), 8.03-8.11 (m, 2H), 8.55 (d, 1H), 9.62 (s, 1H), 10.24 (s, 1H).

LC-MS (Method 1): Rₜ=1.16 min; MS (ESIpos): m/z=523 [M+H]⁺.

### Example 52

### N-{4-methoxy-3-[(morpholin-4-ylacetyl)amino]phenyl}biphenyl-4-carboxamide

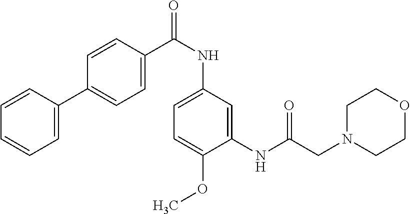 [SMILES: COc1ccc(NC(=O)c2ccc(-c3ccccc3)cc2)cc1NC(=O)CN1CCOCC1]

To a solution of N-{3-[(chloroacetyl)amino]-4-methoxyphenyl}biphenyl-4-carboxamide (prepared in a manner analogous to that described in example 31A, 2.96 g, 7.50 mmol) in DMF (35 mL) was added morpholine (0.99 mL, 11.2 mmol, 1.5 equiv), triethylamine (1.57 mL, 11.2 mmol, 1.5 equiv) and potassium iodide (0.19 g, 1.16 mmol, 0.16 equiv). The resulting mixture was stirred at room temperature for 16 h, was then poured onto water (50 mL). The resulting mixture was extracted with ethyl acetate (3×50 mL). The combined organic phases were washed with a half-saturated NaCl solution, dried (Na₂SO₄ anh) and concentrated under reduced pressure. The residue was triturated with ethanol to give N-{4-methoxy-3-[(morpholin-4-ylacetyl)amino]phenyl}biphenyl-4-carboxamide (3.29 g, 99%).

¹H-NMR (300 MHz, DMSO-d₆): δ [ppm]=2.50-2.54 (m, 4H), 3.12 (s, 2H), 3.61-3.66 (m, 4H), 3.86 (s, 3H), 7.02 (d, J=9.0 Hz, 1H), 7.38 (t, J=7.3 Hz, 1H), 7.47 (t, J=7.3 Hz, 2H), 7.56 (dd, J=2.5, 9.0 Hz, 1H), 7.72 (d, J=7.2 Hz, 2H), 7.78 (d, J=8.5 Hz, 2H), 8.03 (d, J=8.3 Hz, 2H), 8.55 (d, J=2.6 Hz, 1H), 9.71 (s, 1H), 10.22 (s, 1H).

LC-MS (Method 3): Rₜ=1.29 min; MS (ESIpos): m/z=446 ([M+H]⁺, 100%), 919 ([2M+H]⁺, 60%); MS (ESIneg): m/z=444 ([M−H]⁻, 100%), 917 ([2M−H]⁻, 10%).

### Example 53

### N-(4-methoxy-3-{[2-(morpholin-4-yl)propanoyl]amino}phenyl)biphenyl-4-carboxamide

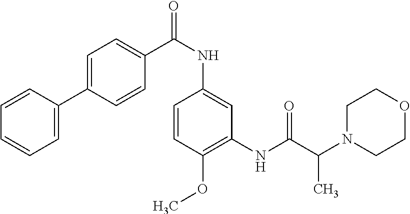 [SMILES: COc1ccc(NC(=O)c2ccc(-c3ccccc3)cc2)cc1NC(=O)C(C)N1CCOCC1]

435 mg (1.06 mmol) of the compound from example 32A were provided in 5 mL of DMF. 0.22 mL (1.60 mmol) of triethylamine, 0.14 mL (1.60 mmol) of morpholine and 27.4 mg (0.17 mmol) of potassium iodide were added, and the mixture was stirred at room temperature over night. 0.45 mL (3.19 mmol) of triethylamine and 0.28 mL (3.19 mmol) of morpholine were added, and the mixture was stirred at 50° C. over night. After filtration, purification by HPLC (column: chromatorex C18, 10 μm, 195×51 mm, mobile phase: acetonitrile/water gradient with the addition of 0.1% formic acid) yielded 308 mg (63% of theory) of the title compound.

¹H-NMR (300 MHz, DMSO-d₆): δ [ppm]=1.22 (d, 3H), 2.54-2.66 (m, 4H), 3.63-3.76 (m, 4H), 3.89 (s, 3H), 7.05 (d, 1H), 7.38-7.46 (m, 1H), 7.47-7.61 (m, 3H), 7.72-7.87 (m, 4H), 8.03-8.11 (m, 2H), 8.58 (d, 1H), 9.90 (s, 1H), 10.22 (s, 1H).

LC-MS (Method 4): Rₜ=1.04 min; MS (ESIpos): m/z=460 [M+H]⁺.

### Examples 54 and 55

### N-(4-methoxy-3-{[(2S)-2-(morpholin-4-yl)propanoyl]amino}phenyl)biphenyl-4-carboxamide

### N-(4-methoxy-3-{[(2R)-2-(morpholin-4-yl)propanoyl]amino}phenyl)biphenyl-4-carboxamide

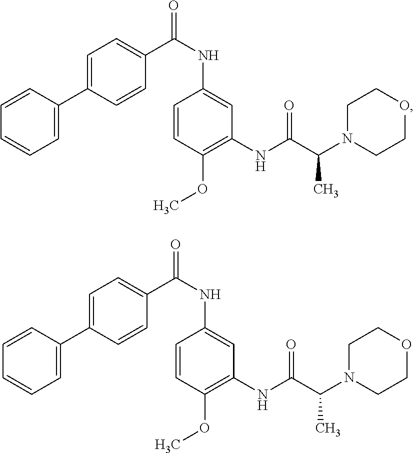

Chiral chromatography (system: Agilent Prep 1200, column: Chiralpak IC 5 μm 250×30 mm, solvent: hexane/ethanol 7/3+0.1% formic acid, rate: 60 mL/min, temperature: room temperature, detection: UV 254 nm) of 300 mg of the compound from example 53 provided:

### Example 54

### 107 mg

¹H-NMR (500 MHz, DMSO-d₆): δ [ppm]=1.20 (d, 3H), 2.54-2.59 (m, 4H), 3.65-3.73 (m, 4H), 3.90 (s, 3H), 7.05 (d, 1H), 7.40-7.45 (m, 1H), 7.48-7.54 (m, 2H), 7.57 (dd, 1H), 7.73-7.78 (m, 2H), 7.79-7.84 (m, 2H), 8.04-8.09 (m, 2H), 8.59 (d, 1H), 9.90 (s, 1H), 10.22 (s, 1H).

LC-MS (Method 4): Rₜ=1.04 min; MS (ESIpos): m/z=460 [M+H]⁺.

LC-MS (system: Waters Alliance 2695, DAD 996, ESA Corona, column: Chiralpak IC 3 μm 100×4.6 mm, solvent: ethanol+0.1% formic acid, rate: 1.0 mL/min, temperature: 25° C., injection: 5.0 μL, detection: DAD 254 nm): Rₜ=14.98 min, 90% enantiomeric excess.

### Example 55

### 88.0 mg

¹H-NMR (500 MHz, DMSO-d₆): δ [ppm]=1.20 (d, 3H), 2.54-2.59 (m, 4H), 3.65-3.73 (m, 4H), 3.90 (s, 3H), 7.05 (d, 1H), 7.40-7.45 (m, 1H), 7.48-7.54 (m, 2H), 7.57 (dd, 1H), 7.73-7.78 (m, 2H), 7.79-7.84 (m, 2H), 8.04-8.09 (m, 2H), 8.59 (d, 1H), 9.90 (s, 1H), 10.22 (s, 1H).

LC-MS (Method 4): Rₜ=1.03 min; MS (ESIpos): m/z=460 [M+H]⁺.

LC-MS (system: Waters Alliance 2695, DAD 996, ESA Corona, column: Chiralpak IC 3 μm 100×4.6 mm, solvent: ethanol+0.1% formic acid, rate: 1.0 mL/min, temperature: 25° C., injection: 5.0 μL, detection: DAD 254 nm): Rₜ=17.19 min, 97% enantiomeric excess.

### Examples 56 and 57

### N-(4-methoxy-3-{[(2S)-2-(8-oxa-3-azabicyclo[3.2.1]oct-3-yl)propanoyl]amino}phenyl)biphenyl-4-carboxamide

### N-(4-methoxy-3-{[(2R)-2-(8-oxa-3-azabicyclo[3.2.1]oct-3-yl)propanoyl]amino}phenyl)biphenyl-4-carboxamide

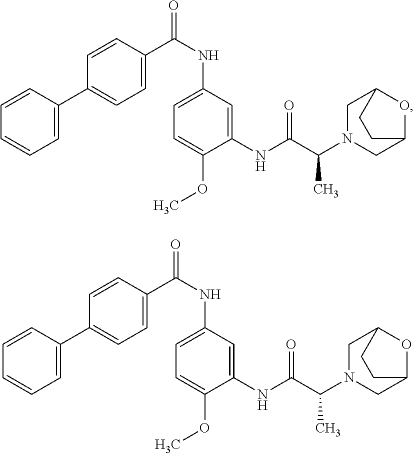

435 mg (1.06 mmol) of the compound from example 32A were provided in 5 mL of DMF. 0.37 mL (2.66 mmol) of triethylamine, 239 mg (1.60 mmol) of 8-oxa-3-azabicyclo[3.2.1]octane hydrochloride and 27.4 mg (0.17 mmol) of potassium iodide were added, and the mixture was stirred at room temperature over night. 0.52 mL (3.72 mmol) of triethylamine and 478 mg (3.19 mmol) of 8-oxa-3-azabicyclo[3.2.1]octane hydrochloride were added, and the mixture was stirred at 50° C. over night. After filtration, purification by HPLC (column: chromatorex C18, 10 μm, 195×51 mm, mobile phase: acetonitrile/water gradient with the addition of 0.1% formic acid) yielded 390 mg (75% of theory) of the racemate of the title compound. Chiral chromatography (system: Agilent Prep 1200, column: Chiralpak IB 5 μm 250×20 mm, solvent: hexane/ethanol 7/3+0.1% diethylamine, rate: 20 mL/min, temperature: room temperature, detection: UV 254 nm) of 385 mg of the racemate provided:

### Example 56

### 95.0 mg

¹H-NMR (300 MHz, DMSO-d₆): δ [ppm]=1.17 (d, 3H), 1.79-1.93 (m, 2H), 2.03-2.16 (m, 2H), 2.34-2.47 (m, 2H), 2.55-2.62 (m, 2H), 3.23 (q, 1H), 3.88 (s, 3H), 4.25-4.32 (m, 2H), 7.06 (d, 1H), 7.37-7.47 (m, 1H), 7.47-7.61 (m, 3H), 7.73-7.79 (m, 2H), 7.79-7.86 (m, 2H), 8.02-8.14 (m, 2H), 8.69 (d, 1H), 9.77 (s, 1H), 10.24 (s, 1H).

LC-MS (Method 4): Rₜ=1.28 min; MS (ESIpos): m/z=486 [M+H]⁺.

LC-MS (system: Waters Alliance 2695, DAD 996, ESA Corona, column: Chiralpak IB 3 μm 100×4.6 mm, solvent: hexane/ethanol 7/3+0.1% diethylamine, rate: 1.0 mL/min, temperature: 25° C., injection: 5.0 μL, detection: DAD 254 nm): Rₜ=4.25 min, 100% enantiomeric excess.

Optical rotation (Method 6): [α]=−9.8° (c=0.77, CHCl₃).

### Example 57

### 110 mg

¹H-NMR (300 MHz, DMSO-d₆): δ [ppm]=1.17 (d, 3H), 1.79-1.92 (m, 2H), 2.02-2.15 (m, 2H), 2.35-2.47 (m, 2H), 2.55-2.62 (m, 2H), 3.23 (q, 1H), 3.88 (s, 3H), 4.24-4.33 (m, 2H), 7.06 (d, 1H), 7.37-7.47 (m, 1H), 7.47-7.61 (m, 3H), 7.73-7.79 (m, 2H), 7.79-7.86 (m, 2H), 8.03-8.12 (m, 2H), 8.69 (d, 1H), 9.77 (s, 1H), 10.24 (s, 1H).

LC-MS (Method 4): Rₜ=1.28 min; MS (ESIpos): m/z=486 [M+H]⁺.

LC-MS (system: Waters Alliance 2695, DAD 996, ESA Corona, column: Chiralpak IB 3 μm 100×4.6 mm, solvent: hexane/ethanol 7/3+0.1% diethylamine, rate: 1.0 mL/min, temperature: 25° C., injection: 5.0 μL, detection: DAD 254 nm): Rₜ=4.95 min, 96% enantiomeric excess.

Optical rotation (Method 6): [α]=+7.7° (c=0.80, CHCl₃).

### Example 58

### N-[3-{[2-(8-oxa-3-azabicyclo[3.2.1]oct-3-yl)propanoyl]amino}-4-(trifluoromethoxy)phenyl]biphenyl-4-carboxamide

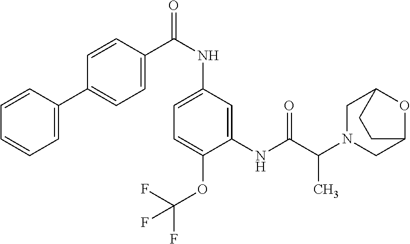 [SMILES: CC(C(=O)Nc1cc(NC(=O)c2ccc(-c3ccccc3)cc2)ccc1OC(F)(F)F)N1CC2CCC(C1)O2]

595 mg (1.29 mmol) of the compound from example 33A were provided in 4 mL of DMF. 0.72 mL (5.14 mmol) of triethylamine, 577 mg (3.86 mmol) of 8-oxa-3-azabicyclo[3.2.1]octane hydrochloride and 42.7 mg (0.26 mmol) of potassium iodide were added, and the mixture was stirred at 50° C. over night. After filtration, purification by HPLC (column: Xbrigde C18 5 μm 150×50 mm, mobile phase: acetonitrile/water gradient with the addition of 0.1% formic acid) yielded 373 mg (53% of theory) of the title compound.

¹H-NMR (300 MHz, DMSO-d₆): δ [ppm]=1.18 (d, 3H), 1.75-1.88 (m, 2H), 1.88-2.02 (m, 2H), 2.43-2.48 (m, 2H), 2.55-2.61 (m, 2H), 3.28 (q, 1H), 4.22-4.30 (m, 2H), 7.38-7.47 (m, 2H), 7.47-7.56 (m, 2H), 7.71-7.80 (m, 3H), 7.81-7.88 (m, 2H), 8.04-8.12 (m, 2H), 8.68 (d, 1H), 9.52 (s, 1H), 10.50 (s, 1H).

LC-MS (Method 1): Rₜ=1.42 min; MS (ESIpos): m/z=540 [M+H]⁺.

### Examples 59 and 60

### N-[3-{[(2S)-2-(8-oxa-3-azabicyclo[3.2.1]oct-3-yl)propanoyl]amino}-4-(trifluoromethoxy)phenyl]biphenyl-4-carboxamide

### N-[3-{[(2R)-2-(8-oxa-3-azabicyclo[3.2.1]oct-3-yl)propanoyl]amino}-4-(trifluoromethoxy)phenyl]biphenyl-4-carboxamide

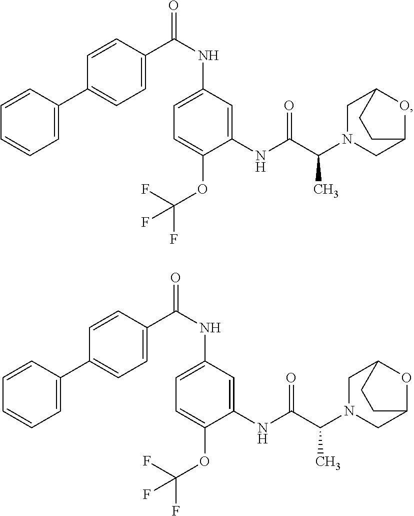

Chiral chromatography (system: Agilent Prep 1200, column: Chiralpak IA 5 μm 250×20 mm, solvent: hexane/dichloromethane/ethanol 8/1/1, rate: 40 mL/min, temperature: room temperature, detection: UV 254 nm) of 307 mg of the compound from example 58 provided:

### Example 59

### 130 mg

¹H-NMR (300 MHz, DMSO-d₆): δ [ppm]=1.18 (d, 3H), 1.72-1.85 (m, 2H), 1.88-2.02 (m, 2H), 2.42-2.48 (m, 2H), 2.55-2.61 (m, 2H), 3.28 (q, 1H), 4.22-4.31 (m, 2H), 7.39-7.47 (m, 2H), 7.47-7.56 (m, 2H), 7.71-7.80 (m, 3H), 7.81-7.89 (m, 2H), 8.04-8.12 (m, 2H), 8.68 (d, 1H), 9.54 (s, 1H), 10.52 (s, 1H).

LC-MS (Method 1): Rₜ=1.42 min; MS (ESIpos): m/z=540 [M+H]⁺.

LC-MS (system: Agilent: 1260 AS, MWD, Aurora SFC-Modul, column: Chiralpak IB 5 μm 100×4.6 mm, solvent: CO₂/ethanol 85/15, rate: 4 mL/min, pressure (outlet): 150 bar, temperature: 40° C., detection: UV 254 nm): Rₜ=3.91 min, 100% enantiomeric excess.

Optical rotation (Method 6): [α]=−3.1° (c=1.15, CHCl₃).

### Example 60

### 130 mg

¹H-NMR (300 MHz, DMSO-d₆): δ [ppm]=1.17 (d, 3H), 1.73-1.83 (m, 2H), 1.88-2.01 (m, 2H), 2.42-2.47 (m, 2H), 2.55-2.61 (m, 2H), 3.27 (q, 1H), 4.22-4.30 (m, 2H), 7.38-7.47 (m, 2H), 7.47-7.55 (m, 2H), 7.71-7.80 (m, 3H), 7.80-7.88 (m, 2H), 8.03-8.12 (m, 2H), 8.68 (d, 1H), 9.53 (s, 1H), 10.52 (s, 1H).

LC-MS (Method 1): Rₜ=1.42 min; MS (ESIpos): m/z=540 [M+H]⁺.

LC-MS (system: Agilent: 1260 AS, MWD, Aurora SFC-Modul, column: Chiralpak IB 5 μm 100×4.6 mm, solvent: CO₂/ethanol 85/15, rate: 4 mL/min, pressure (outlet): 150 bar, temperature: 40° C., detection: UV 254 nm): Rₜ=4.54 min, 95% enantiomeric excess.

Optical rotation (Method 6): [α]=+2.6° (c=1.05, CHCl₃).

### Example 61

### N-[3-{[2-(morpholin-4-yl)propanoyl]amino}-4-(trifluoromethoxy)phenyl]biphenyl-4-carboxamide

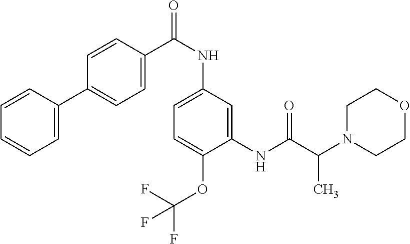 [SMILES: CC(C(=O)Nc1cc(NC(=O)c2ccc(-c3ccccc3)cc2)ccc1OC(F)(F)F)N1CCOCC1]

595 mg (1.29 mmol) of the compound from example 33A were provided in 4 mL of DMF. 0.54 mL (3.86 mmol) of triethylamine, 0.34 mL (3.86 mmol) of morpholine and 42.7 mg (0.26 mmol) of potassium iodide were added, and the mixture was stirred at 50° C. over night. After filtration, purification by HPLC (column: Xbrigde C18 5 μm 150×50 mm, mobile phase: acetonitrile/water gradient with the addition of 0.1% formic acid) yielded 444 mg (66% of theory) of the title compound.

¹H-NMR (300 MHz, DMSO-d₆): δ [ppm]=1.22 (d, 3H), 2.50-2.62 (m, 4H), 3.38 (q, 1H), 3.60-3.71 (m, 4H), 7.39-7.47 (m, 2H), 7.47-7.56 (m, 2H), 7.70-7.80 (m, 3H), 7.81-7.88 (m, 2H), 8.04-8.12 (m, 2H), 8.68 (d, 1H), 9.89 (s, 1H), 10.51 (s, 1H).

LC-MS (Method 1): Rₜ=1.24 min; MS (ESIpos): m/z=514 [M+H]⁺.

### Examples 62 and 63

### N-[3-{[(2S)-2-(morpholin-4-yl)propanoyl]amino}-4-(trifluoromethoxy)phenyl]biphenyl-4-carboxamide

### N-[3-{[(2R)-2-(morpholin-4-yl)propanoyl]amino}-4-(trifluoromethoxy)phenyl]biphenyl-4-carboxamide

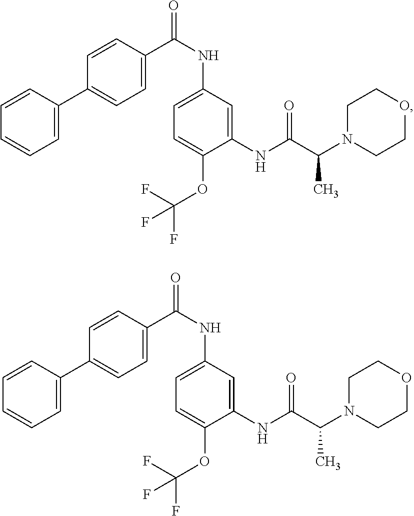

Chiral chromatography (system: Agilent Prep 1200, column: Chiralpak IA 5 μm 250×20 mm, solvent: hexane/dichloromethane/ethanol 8/1/1, rate: 40 mL/min, temperature: room temperature, detection: UV 254 nm) of 380 mg of the compound from example 61 provided:

### Example 62

### 124 mg

¹H-NMR (300 MHz, DMSO-d₆): δ [ppm]=1.22 (d, 3H), 2.50-2.62 (m, 4H), 3.38 (q, 1H), 3.61-3.70 (m, 4H), 7.39-7.47 (m, 2H), 7.48-7.57 (m, 2H), 7.70-7.81 (m, 3H), 7.81-7.89 (m, 2H), 8.04-8.13 (m, 2H), 8.68 (d, 1H), 9.91 (s, 1H), 10.53 (s, 1H).

LC-MS (Method 4): Rₜ=1.27 min; MS (ESIpos): m/z=514 [M+H]⁺.

LC-MS (system: Agilent: 1260 AS, MWD, Aurora SFC-Modul, column: Chiralpak IB 5 μm 100×4.6 mm, solvent: CO₂/ethanol 8/2, rate: 4 mL/min, pressure (outlet): 150 bar, temperature: 40° C., detection: UV 254 nm): Rₜ=2.00 min, 99% enantiomeric excess.

Optical rotation (Method 6): [α]=−3.1° (c=0.98, CHCl₃).

### Example 63

### 120 mg

¹H-NMR (300 MHz, DMSO-d₆): δ [ppm]=1.22 (d, 3H), 2.50-2.62 (m, 4H), 3.38 (q, 1H), 3.60-3.70 (m, 4H), 7.39-7.47 (m, 2H), 7.47-7.57 (m, 2H), 7.70-7.81 (m, 3H), 7.81-7.90 (m, 2H), 8.04-8.13 (m, 2H), 8.68 (d, 1H), 9.91 (s, 1H), 10.53 (s, 1H).

LC-MS (Method 1): Rₜ=1.23 min; MS (ESIpos): m/z=514 [M+H]⁺.

LC-MS (system: Agilent: 1260 AS, MWD, Aurora SFC-Modul, column: Chiralpak IB 5 μm 100×4.6 mm, solvent: CO₂/ethanol 8/2, rate: 4 mL/min, pressure (outlet): 150 bar, temperature: 40° C., detection: UV 254 nm): Rₜ=2.39 min, 97% enantiomeric excess.

Optical rotation (Method 6): [α]=+3.2° (c=0.88, CHCl₃).

### Example 64

### N-{3-[benzyl(morpholin-4-ylacetyl)amino]-4-methoxyphenyl}biphenyl-4-carboxamide

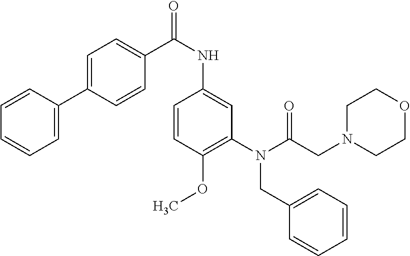 [SMILES: COc1ccc(NC(=O)c2ccc(-c3ccccc3)cc2)cc1N(Cc1ccccc1)C(=O)CN1CCOCC1]

100 mg (245 μmol) of the compound from example 34A and 128 μL (734 μmol) of N,N-diisopropylethylamine were provided in 1.5 mL of DMF at room temperature. A solution of 42.6 mg (294 μmol) of morpholin-4-ylacetic acid in 0.5 mL of DMF and 86 μL (294 μmol) of a 50% solution of 2,4,6-tripropyl-1,3,5,2,4,6-trioxatriphosphinane 2,4,6-trioxide (T3P) in DMF were added, and the mixture was stirred over night at room temperature. 42.6 mg (294 μmol) of morpholin-4-ylacetic acid and 86 μL (294 μmol) of a 50% solution of 2,4,6-tripropyl-1,3,5,2,4,6-trioxatriphosphinane 2,4,6-trioxide (T3P) in DMF were added, and the mixture was stirred for 5 h at room temperature and over night at 50° C. 107 mg (734 μmol) of morpholin-4-ylacetic acid and 214 μL (734 μmol) of a 50% solution of 2,4,6-tripropyl-1,3,5,2,4,6-trioxatriphosphinane 2,4,6-trioxide (T3P) in DMF were added, and the mixture was stirred for 14 d at room temperature. After filtration, purification by HPLC (method 2) yielded 49.2 mg, which were taken up in dichloromethane and were washed with a saturated aqueous sodium bicarbonate solution, dried over sodium sulfate, filtered and concentrated. 35.5 mg (27% of theory) of the title compound were obtained.

¹H-NMR (400 MHz, DMSO-d₆): δ [ppm]=2.27-2.40 (m, 4H), 2.87 (d, 1H), 2.95 (d, 1H), 3.49 (t, 4H), 3.75 (s, 3H), 4.38 (d, 1H), 5.07 (d, 1H), 7.11 (d, 1H), 7.18-7.24 (m, 3H), 7.25-7.32 (m, 2H), 7.39-7.45 (m, 1H), 7.48-7.54 (m, 3H), 7.72-7.78 (m, 3H), 7.79-7.85 (m, 2H), 7.99-8.04 (m, 2H), 10.18 (s, 1H).

LC-MS (Method 4): Rₜ=1.10 min; MS (ESIpos): m/z=536 [M+H]⁺.

### Example 65

### N-{4-methoxy-3-[methyl(morpholin-4-ylacetyl)amino]phenyl}biphenyl-4-carboxamide

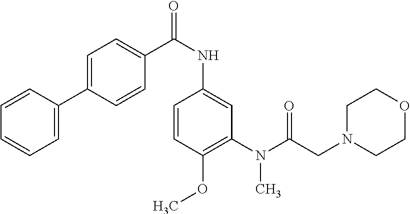 [SMILES: COc1ccc(NC(=O)c2ccc(-c3ccccc3)cc2)cc1N(C)C(=O)CN1CCOCC1]

100 mg (301 μmol) of the compound from example 35A and 157 μL (903 μmol) of N,N-diisopropylethylamine were provided in 2 mL of DMF at room temperature. 52.0 mg (361 μmol) of morpholin-4-ylacetic acid and 211 μL (361 μmol) of a 50% solution of 2,4,6-tripropyl-1,3,5,2,4,6-trioxatriphosphinane 2,4,6-trioxide (T3P) in DMF were added, and the mixture was stirred over night at room temperature. 52.0 mg (361 μmol) of morpholin-4-ylacetic acid and 211 μL (361 μmol) of a 50% solution of 2,4,6-tripropyl-1,3,5,2,4,6-trioxatriphosphinane 2,4,6-trioxide (T3P) in DMF were added, and the mixture was stirred for 24 h at room temperature. After filtration, purification by HPLC (method 2) yielded 126 mg, which were taken up in dichloromethane and were washed with a saturated aqueous sodium bicarbonate solution, dried over sodium sulfate, filtered and concentrated. 76.0 mg (54% of theory) of the title compound were obtained.

¹H-NMR (300 MHz, DMSO-d₆): δ [ppm]=2.21-2.37 (m, 4H), 2.75-2.93 (m, 2H), 3.05 (s, 3H), 3.43-3.50 (m, 4H), 3.83 (s, 3H), 7.11-7.20 (m, 1H), 7.37-7.46 (m, 1H), 7.47-7.56 (m, 2H), 7.70-7.80 (m, 4H), 7.81-7.89 (m, 2H), 8.01-8.10 (m, 2H), 10.28 (s, 1H).

LC-MS (Method 4): Rₜ=0.99 min; MS (ESIpos): m/z=460 [M+H]⁺.

### Example 66

### N-{3-[(morpholin-4-ylacetyl)amino]-4-(trifluoromethoxy)phenyl}biphenyl-4-carboxamide

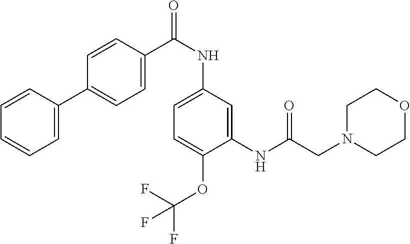 [SMILES: O=C(CN1CCOCC1)Nc1cc(NC(=O)c2ccc(-c3ccccc3)cc2)ccc1OC(F)(F)F]

To a solution of N-[5-amino-2-(trifluoromethoxy)phenyl]-2-(morpholin-4-yl)acetamide (prepared in a manner analogous to that described in example 51A, 2.46 g, 7.71 mmol) and biphenyl-4-carboxylic acid (2.29 g, 11.6 mmol, 1.5 equiv) in DMF (80 mL) was added (benzotriazol-1-yloxy)tripyrrolidinophosphonium hexafluorophosphate (PYBOP, 6.01 g, 11.6 mmol, 1.5 equiv) followed by diisopropylethylamine (5.3 mL, 30.8 mmol, 4.0 equiv). The resulting mixture was stirred at room temperature for 24 h. To the resulting mixture was added additional biphenyl-4-carboxylic acid (1.14 g, 5.78 mmol, 0.75 equiv), (benzotriazol-1-yloxy)tripyrrolidinophosphonium hexafluorophosphate (PYBOP, 3.01 g, 5.78 mmol, 0.75 equiv) and diisopropylethylamine (2.7 mL, 15.4 mmol, 2.0 equiv). The resulting mixture was stirred at room temperature for 12 h, was then concentrated under reduced pressure. The residue was treated with water (100 mL). The resulting mixture was extracted with ethyl acetate (100 mL). The organic phase was dried (Na₂SO₄ anh), and concentrated under reduced pressure. The residue (0.25 g) was purified using MPLC (Biotage Isolera; 10 g SNAP cartridge: 100% hexane 2.0 min., gradient to 50% hexane/50% EtOAc 5.5 min., 50% hexane/50% EtOAc 5.0 min., gradient to 100% EtOAc 7.0 min., 100% EtOAc 4.8 min.) to give N-{3-[(morpholin-4-ylacetyl)amino]-4-(trifluoromethoxy)phenyl}biphenyl-4-carboxamide (01.33 g, 34%).

¹H-NMR (400 MHz, DMSO-d₆): δ [ppm]=2.51-2.56 (m, 4H), 3.17 (s, 2H), 3.59-3.64 (m, 4H), 7.36-7.43 (m, 2H), 7.48 (t, J=7.3 Hz, 2H), 7.70-7.75 (m, 3H), 7.81 (d, 8.3 Hz, 2H), 8.04 (d, J=8.5 Hz, 2H), 8.69 (d, J=2.5 Hz, 1H), 9.76 (s, 1H), 10.51 (s, 1H).

LC-MS (Method 3): Rₜ=1.40 min; MS (ESIpos): m/z=500 ([M+H]⁺, 100%), 999 ([2M+H]⁺, 70%); MS (ESIneg): m/z=498 ([M−H]⁻, 100%).

### Example 67

### N-{4-tert-butyl-3-[(morpholin-4-ylacetyl)amino]phenyl}biphenyl-4-carboxamide

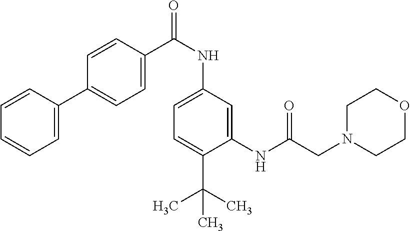 [SMILES: CC(C)(C)c1ccc(NC(=O)c2ccc(-c3ccccc3)cc2)cc1NC(=O)CN1CCOCC1]

To a solution of N-(5-amino-2-tert-butylphenyl)-2-(morpholin-4-yl)acetamide (prepared in a manner analogous to that described in example 52A, 0.090 g, 0.31 mmol) and biphenyl-4-carboxylic acid (0.077 g, 0.39 mmol, 1.25 equiv) in DMF (2.4 mL) was added propanephosphonic acid cyclic anhydride solution (50% in ethyl acetate, 0.23 mL, 0.39 mmol, 1.25 equiv) followed by diisopropylethylamine (0.16 mL, 0.93 mmol, 3.0 equiv). The resulting mixture was stirred at room temperature for 24 h, was then concentrated under reduced pressure. The residue was then treated with water (50 mL). The resulting mixture was extracted with ethyl acetate (50 mL). The organic phase was dried (Na₂SO₄ anh), and concentrated under reduced pressure. The residue was purified by MPLC (Biotage Isolera; 10 g SNAP cartridge: 100% hexane 2.0 min., gradient to 50% hexane/50% EtOAc 2.5 min., 50% hexane/50% EtOAc 3.5 min., gradient to 100% EtOAc 7.5 min., 100% EtOAc 3.8 min.) to give N-{4-tert-butyl-3-[(morpholin-4-ylacetyl)amino]phenyl}biphenyl-4-carboxamide (28 mg, 19%).

¹H-NMR (400 MHz, DMSO-d₆): δ [ppm]=1.36 (s, 9H), 2.55-2.60 (m, 4H), 3.14 (s, 2H), 3.61-3.65 (m, 4H), 7.32 (d, J=8.8 Hz, 1H), 7.39 (t, J=7.3 Hz, 1H), 7.48 (t, J=7.5 Hz, 2H), 7.65 (dd, J=2.5, 8.6 Hz, 1H), 7.73 (d, J=7.1 Hz, 2H), 7.79 (d, J=8.6 Hz, 2H), 8.03 (d, J=8.6 Hz, 2H), 8.09 (d, J=2.3 Hz, 1H), 9.37 (s, 1H), 10.27 (s, 1H).

LC-MS (Method 3): Rₜ=1.38 min; MS (ESIpos): m/z=472 ([M+H]⁺, 100%), 943 ([2M+H]⁺, 30%); MS (ESIneg): m/z=470 ([M−H]⁻, 100%), 941 ([2M−H]⁻, 10%).

### Example 68

### N-{4-bromo-3-[(morpholin-4-ylacetyl)amino]phenyl}biphenyl-4-carboxamide

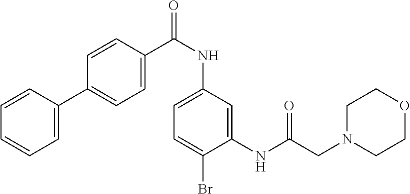 [SMILES: O=C(CN1CCOCC1)Nc1cc(NC(=O)c2ccc(-c3ccccc3)cc2)ccc1Br]

To a solution of N-(5-amino-2-bromophenyl)-2-(morpholin-4-yl)acetamide (prepared in a manner analogous to that described in example 53A, 1.10 g, 3.50 mmol) and biphenyl-4-carboxylic acid (1.04 g, 5.53 mmol, 1.5 equiv) in DMF (37 mL) was added (benzotriazol-1-yloxy)tripyrrolidinophosphonium hexafluorophosphate (PYBOP, 2.73 g, 5.25 mmol, 1.5 equiv) followed by diisopropylethylamine (2.4 mL, 14.0 mmol, 4.0 equiv). The resulting mixture was stirred at room temperature for 24 h, was then concentrated under reduced pressure. The residue was treated with water (25 mL). The resulting mixture was extracted with ethyl acetate (25 mL). The organic phase was dried (Na₂SO₄ anh), and concentrated under reduced pressure. The residue (3.5 g) was crystallized from ethanol to give N-{4-bromo-3-[(morpholin-4-ylacetyl)amino]phenyl}biphenyl-4-carboxamide (0.91 g, 52%).

¹H-NMR (400 MHz, DMSO-d₆): δ [ppm]=2.55-2.59 (m, 4H), 3.17 (s, 2H), 3.66-3.69 (m, 4H), 7.39 (t, J=7.3 Hz, 1H), 7.48 (t, J=7.6 Hz, 2H), 7.58-7.64 (m, 2H), 7.73 (d, J=7.3 Hz, 2H), 7.80 (d, J=8.3 Hz, 2H), 8.04 (d, J=8.6 Hz, 2H), 8.71 (d, J=2.0 Hz, 1H), 9.88 (s, 1H), 10.46 (s, 1H).

LC-MS (Method 3): Rₜ=1.38 min; MS (ESIpos): m/z=494 ([M+H]⁺, 100%); (ESIneg): m/z=492 ([M−H]⁻, 100%).

### Example 69

### N-{4-chloro-3-[(morpholin-4-ylacetyl)amino]phenyl}biphenyl-4-carboxamide

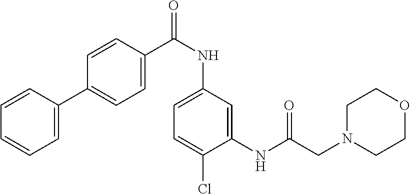 [SMILES: O=C(CN1CCOCC1)Nc1cc(NC(=O)c2ccc(-c3ccccc3)cc2)ccc1Cl]

To a solution of N-(5-amino-2-chlorophenyl)-2-(morpholin-4-yl)acetamide (prepared in a manner analogous to that described in example 54A, 0.16 g, 0.59 mmol) and biphenyl-4-carboxylic acid (0.17 g, 0.88 mmol, 1.5 equiv) in DMF (5 mL) was added (benzotriazol-1-yloxy)tripyrrolidinophosphonium hexafluorophosphate (PYBOP, 0.46 g, 0.88 mmol, 1.5 equiv) followed by diisopropylethylamine (0.41 mL, 2.34 mmol, 4.0 equiv). The resulting mixture was stirred at room temperature for 24 h, was then concentrated under reduced pressure. The residue was treated with water (10 mL). The resulting mixture was extracted with ethyl acetate (10 mL). The organic phase was dried (Na₂SO₄ anh), and concentrated under reduced pressure. The residue (3.5 g) was purified by HPLC (method 3) to give N-{4-chloro-3-[(morpholin-4-ylacetyl)amino]phenyl}biphenyl-4-carboxamide (29 mg, 11%).

¹H-NMR (400 MHz, DMSO-d₆): δ [ppm]=2.53-2.58 (m, 4H), 3.17 (s, 2H), 3.63-3.67 (m, 4H), 7.39 (t, J=7.3 Hz, 1H), 7.44-7.51 (m, 3H), 7.68 (dd, J=2.5, 8.9 Hz, 1H), 7.73 (d, J=7.2 Hz, 2H), 7.80 (d, J=8.5 Hz, 2H), 8.04 (d, J=8.5 Hz, 2H), 8.69 (d, J=2.5 Hz, 1H), 9.88 (s, 1H), 10.45 (s, 1H).

### Example 70

### N-{4-methyl-3-[(morpholin-4-ylacetyl)amino]phenyl}biphenyl-4-carboxamide

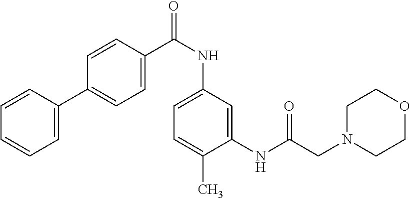 [SMILES: Cc1ccc(NC(=O)c2ccc(-c3ccccc3)cc2)cc1NC(=O)CN1CCOCC1]

100 mg (401 μmol) of the compound from example 55A and 103 mg (521 μmol) of biphenyl-4-carboxylic acid were provided in 4 mL of DMF at room temperature. 304 μL (521 μmol) of a 50% solution of 2,4,6-tripropyl-1,3,5,2,4,6-trioxatriphosphinane 2,4,6-trioxide (T3P) in DMF and 279 μL (1.60 mmol) of N,N-diisopropylethylamine were added, and the mixture was stirred for 16 h at room temperature. Water and ethyl acetate were added, and the phases were separated. The organic phase was dried over sodium sulfate, filtered and concentrated. Purification of the remaining material by HPLC (method 2) yielded 44.2 mg (25% of theory) of the title compound.

¹H-NMR (400 MHz, DMSO-d₆): δ [ppm]=2.22 (s, 3H), 2.55-2.61 (m, 4H), 3.16 (s, 2H), 3.64-3.70 (m, 4H), 7.20 (d, 1H), 7.40-7.45 (m, 1H), 7.48-7.54 (m, 2H), 7.57 (dd, 1H), 7.73-7.79 (m, 2H), 7.81-7.85 (m, 2H), 8.04-8.09 (m, 2H), 8.19 (d, 1H), 9.41 (s, 1H), 10.28 (s, 1H).

LC-MS (Method 3): Rₜ=1.22 min; MS (ESIpos): m/z=430 [M+H]⁺.

### Example 71

### N-{4-methoxy-3-[(morpholin-4-ylacetyl)amino]phenyl}-3′-methylbiphenyl-4-carboxamide

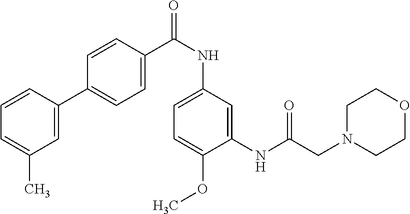 [SMILES: COc1ccc(NC(=O)c2ccc(-c3cccc(C)c3)cc2)cc1NC(=O)CN1CCOCC1]

To a solution of N-(5-amino-2-methoxyphenyl)-2-(morpholin-4-yl)acetamide (prepared in a manner analogous to that described in example 56A, 0.085 g, 0.32 mmol) and 3′-methylbiphenyl-4-carboxylic acid (0.082 g, 0.38 mmol, 1.20 equiv) in DMF (2.5 mL) was added propanephosphonic acid cyclic anhydride solution (50% in ethyl acetate, 0.22 mL, 0.38 mmol, 1.20 equiv) followed by diisopropylethylamine (0.17 mL, 0.96 mmol, 3.0 equiv). The resulting mixture was stirred at room temperature for 24 h, was then concentrated under reduced pressure. The residue was then treated with water (10 mL). The resulting mixture was extracted with ethyl acetate (10 mL). The organic phase was dried (Na₂SO₄ anh), and concentrated under reduced pressure. The residue was purified by HPLC (method 3) to give N-{4-methoxy-3-[(morpholin-4-ylacetyl)amino]phenyl}-3′-methylbiphenyl-4-carboxamide (32 mg, 20%).

¹H-NMR (400 MHz, DMSO-d₆): δ [ppm]=2.36 (s, 3H), 2.50-2.55 (m, 4H), 3.12 (s, 2H), 3.62-3.66 (m, 4H), 3.86 (s, 3H), 7.02 (d, J=9.0 Hz, 1H), 7.20 (d, J=7.4 Hz, 1H), 7.35 (t, J=7.5 Hz, 1H), 7.48-7.57 (m, 3H), 7.76 (d, J=8.5 Hz, 2H), 8.02 (d, J=8.5 Hz, 2H), 8.54 (d, J=2.6 Hz, 1H), 9.70 (s, 1H), 10.19 (s, 1H).

LC-MS (Method 3): Rₜ=1.31 min; MS (ESIpos): m/z=460 ([M+H]⁺, 50%), 919 ([2M+H]⁺, 50%); MS (ESIneg): m/z=458 ([M−H]⁻, 100%).

### Example 72

### 3′-cyano-N-{4-methoxy-3-[(morpholin-4-ylacetyl)amino]phenyl}biphenyl-4-carboxamide

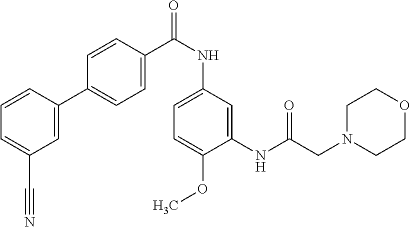 [SMILES: COc1ccc(NC(=O)c2ccc(-c3cccc(C#N)c3)cc2)cc1NC(=O)CN1CCOCC1]

To a solution of N-(5-amino-2-methoxyphenyl)-2-(morpholin-4-yl)acetamide (prepared in a manner analogous to that described in example 56A, 0.085 g, 0.32 mmol) and 3′-cyanobiphenyl-4-carboxylic acid (0.086 g, 0.38 mmol, 1.20 equiv) in DMF (2.5 mL) was added propanephosphonic acid cyclic anhydride solution (50% in ethyl acetate, 0.22 mL, 0.38 mmol, 1.20 equiv) followed by diisopropylethylamine (0.17 mL, 0.96 mmol, 3.0 equiv). The resulting mixture was stirred at room temperature for 24 h, was then concentrated under reduced pressure. The residue was then treated with water (10 mL). The resulting mixture was extracted with ethyl acetate (10 mL). The organic phase was dried (Na₂SO₄ anh), and concentrated under reduced pressure. The residue was purified by HPLC (method 3) to give 3′-cyano-N-{4-methoxy-3-[(morpholin-4-ylacetyl)amino]phenyl}biphenyl-4-carboxamide (37 mg, 25%).

¹H-NMR (400 MHz, DMSO-d₆): δ [ppm]=2.51-2.54 (m, 4H), 3.12 (s, 2H), 3.62-3.66 (m, 4H), 3.86 (s, 3H), 7.02 (d, J=9.1 Hz, 1H), 7.57 (dd, J=2.5, 8.7 Hz, 1H), 7.68 (t, J=7.8 Hz, 1H), 7.85 (dt, J=1.3, 7.8 Hz, 1H), 7.88 (d, J=8.6 Hz, 2H), 8.06 (d, J=8.6 Hz, 2H), 8.08-8.11 (m, 1H), 8.24 (t, J=1.5 Hz, 1H), 8.55 (d, J=2.5 Hz, 1H), 9.71 (s, 1H), 10.24 (s, 1H).

LC-MS (Method 3): Rₜ=1.17 min; MS (ESIpos): m/z=471 ([M+H]⁺, 100%), 941 ([2M+H]⁺, 70%); MS (ESIneg): m/z=469 ([M−H]⁻, 100%), 939 ([2M−H]⁻, 10%).

### Example 73

### 3′-chloro-N-{4-methoxy-3-[(morpholin-4-ylacetyl)amino]phenyl}biphenyl-4-carboxamide

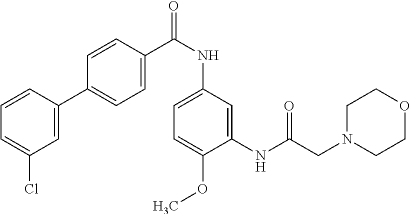 [SMILES: COc1ccc(NC(=O)c2ccc(-c3cccc(Cl)c3)cc2)cc1NC(=O)CN1CCOCC1]

To a solution of N-(5-amino-2-methoxyphenyl)-2-(morpholin-4-yl)acetamide (prepared in a manner analogous to that described in example 56A, 0.085 g, 0.32 mmol) and 3′-chlorobiphenyl-4-carboxylic acid (0.089 g, 0.38 mmol, 1.20 equiv) in DMF (2.5 mL) was added propanephosphonic acid cyclic anhydride solution (50% in ethyl acetate, 0.22 mL, 0.38 mmol, 1.20 equiv) followed by diisopropylethylamine (0.17 mL, 0.96 mmol, 3.0 equiv). The resulting mixture was stirred at room temperature for 24 h, was then concentrated under reduced pressure. The residue was then treated with water (10 mL). The resulting mixture was extracted with ethyl acetate (10 mL). The organic phase was dried (Na₂SO₄ anh), and concentrated under reduced pressure. The residue was purified by HPLC (method 3) to give 3′-chloro-N-{4-methoxy-3-[(morpholin-4-ylacetyl)amino]phenyl}biphenyl-4-carboxamide (12 mg, 8%).

¹H-NMR (400 MHz, DMSO-d₆): δ [ppm]=2.50-2.55 (m, 4H), 3.12 (s, 2H), 3.62-3.66 (m, 4H), 3.86 (s, 3H), 7.02 (d, J=9.0 Hz, 1H), 7.45 (dt, J=1.8, 7.9 Hz, 1H), 7.50 (t, J=7.7 Hz, 1H), 7.56 (dd, J=2.6, 8.9 Hz, 1H), 7.71 (dt, J=1.6, 7.4 Hz, 1H), 7.79 (t, J=1.7 Hz, 1H), 7.82 (d, J=8.5 Hz, 2H), 8.04 (d, J=8.7 Hz, 2H), 8.55 (d, J=2.6 Hz, 1H), 9.70 (s, 1H), 10.22 (s, 1H).

LC-MS (Method 3): Rₜ=1.32 min; MS (ESIpos): m/z=480 ([M+H]⁺, 80%); MS (ESIneg): m/z=478 ([M−H]⁻, 60%).

### Example 74

### 3′-fluoro-N-{3-[(morpholin-4-ylacetyl)amino]-4-(trifluoromethoxy)phenyl}biphenyl-4-carboxamide

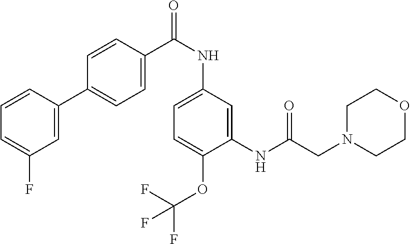 [SMILES: O=C(CN1CCOCC1)Nc1cc(NC(=O)c2ccc(-c3cccc(F)c3)cc2)ccc1OC(F)(F)F]

To a microwave vial was added 4-bromo-N-{3-[(morpholin-4-ylacetyl)amino]-4-(trifluoromethoxy)phenyl}benzamide (prepared in a manner analogous to that described in example 58A, 0.10 g, 0.20 mmol), (3-fluorophenyl)boronic acid (0.056 g, 0.40 mmol, 2.0 equiv), a 2 N sodium carbonate solution (0.30 mL, 0.60 mmol, 3.0 equiv) and dioxane (2.1 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.016 g, 0.019 mmol, 10 mol %) and sealed. The resulting mixture was heated with a microwave apparatus at 105° C. for 1 h, was then cooled to room temperature. The reaction mixture was poured onto ice water (10 mL), and extracted with ethyl acetate (3×10 mL). The combined organic phases were dried (Na₂SO₄ anh), and concentrated under reduced pressure. The resulting material was purified by HPLC (method 3) to give 3′-fluoro-N-{3-[(morpholin-4-ylacetyl)amino]-4-(trifluoromethoxy)phenyl}biphenyl-4-carboxamide (71 mg, 65%).

¹H-NMR (400 MHz, DMSO-d₆): δ [ppm]=2.52-2.56 (m, 4H), 3.17 (s, 2H), 3.59-3.63 (m, 4H), 7.23 (tm, J=8.7, 1H), 7.41 (dd, J=1.3, 9.1 Hz, 1H), 7.48-7.51 (m, 1H), 7.58-7.63 (m, 2H), 7.72 (dd, J=2.5, 9.1 Hz, 1H), 7.86 (d, J=8.3 Hz, 2H), 8.05 (d, J=8.3 Hz, 2H), 8.69 (d, J=2.5 Hz, 1H), 9.76 (s, 1H), 10.53 (s, 1H).

LC-MS (Method 3): Rₜ=1.39 min; MS (ESIpos): m/z=518 ([M+H]⁺, 100%); MS (ESIneg): m/z=516 ([M−H]⁻, 100%).

### Example 75

### 4′-fluoro-N-{3-[(morpholin-4-ylacetyl)amino]-4-(trifluoromethoxy)phenyl}biphenyl-4-carboxamide

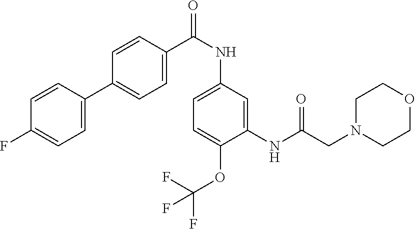 [SMILES: O=C(CN1CCOCC1)Nc1cc(NC(=O)c2ccc(-c3ccc(F)cc3)cc2)ccc1OC(F)(F)F]

To a microwave vial was added 4-bromo-N-{3-[(morpholin-4-ylacetyl)amino]-4-(trifluoromethoxy)phenyl}benzamide (prepared in a manner analogous to that described in example 58A, 0.10 g, 0.20 mmol), (4-fluorophenyl)boronic acid (0.056 g, 0.40 mmol, 2.0 equiv), a 2 N sodium carbonate solution (0.30 mL, 0.60 mmol, 3.0 equiv) and dioxane (2.1 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.016 g, 0.019 mmol, 10 mol %) and sealed. The resulting mixture was heated with a microwave apparatus at 105° C. for 1 h, was then cooled to room temperature. The reaction mixture was poured onto ice water (10 mL), and extracted with ethyl acetate (3×10 mL). The combined organic phases were dried (Na₂SO₄ anh), and concentrated under reduced pressure. The resulting material was purified by HPLC (method 3) to give 4′-fluoro-N-{3-[(morpholin-4-ylacetyl)amino]-4-(trifluoromethoxy)phenyl}biphenyl-4-carboxamide (70 mg, 67%).

¹H-NMR (400 MHz, DMSO-d₆): δ [ppm]=2.52-2.55 (m, 4H), 3.17 (s, 2H), 3.59-3.63 (m, 4H), 7.31 (t, J=8.5 Hz, 2H), 7.41 (dd, J=1.3, 9.1 Hz, 1H), 7.72 (dd, J=2.5, 9.1 Hz, 1H), 7.76-7.81 (m, 4H), 8.05 (d, J=8.3 Hz, 2H), 8.69 (d, J=2.5 Hz, 1H), 9.76 (s, 1H), 10.51 (s, 1H).

LC-MS (Method 3): Rₜ=1.39 min; MS (ESIpos): m/z=518 ([M+H]⁺, 100%); MS (ESIneg): m/z=516 ([M−H]⁻, 100%).

### Example 76

### 4′-amino-N-{3-[(morpholin-4-ylacetyl)amino]-4-(trifluoromethoxy)phenyl}biphenyl-4-carboxamide

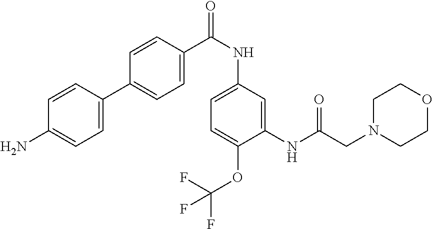 [SMILES: Nc1ccc(-c2ccc(C(=O)Nc3ccc(OC(F)(F)F)c(NC(=O)CN4CCOCC4)c3)cc2)cc1]

To a microwave vial was added 4-bromo-N-{3-[(morpholin-4-ylacetyl)amino]-4-(trifluoromethoxy)phenyl}benzamide (prepared in a manner analogous to that described in example 58A, 0.10 g, 0.20 mmol), (4-aminophenyl)boronic acid HCl salt (0.069 g, 0.40 mmol, 2.0 equiv), a 2N sodium carbonate solution (0.40 mL, 0.80 mmol, 4.0 equiv) and dioxane (2.1 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.016 g, 0.019 mmol, 10 mol %) and sealed. The resulting mixture was heated with a microwave apparatus at 105° C. for 1 h, was then cooled to room temperature. The reaction mixture was poured onto ice water (10 mL), and extracted with ethyl acetate (3×10 mL). The combined organic phases were dried (Na₂SO₄ anh), and concentrated under reduced pressure. The resulting material was purified by HPLC (method 3) to give 4′-amino-N-{3-[(morpholin-4-ylacetyl)amino]-4-(trifluoromethoxy)phenyl}biphenyl-4-carboxamide (60 mg, 59%).

¹H-NMR (400 MHz, DMSO-d₆): δ [ppm]=2.51-2.56 (m, 4H), 3.17 (s, 2H), 3.59-3.63 (m, 4H), 5.35 (s, 2H), 6.63 (d, J=8.5 Hz, 2H), 7.40 (dd, J=1.1, 9.2 Hz, 1H), 7.44 (d, J=8.7 Hz, 2H), 7.66 (d, J=8.5 Hz, 2H), 7.71 (dd, J=2.5, 8.9 Hz, 1H), 7.95 (d, J=8.5 Hz, 2H), 8.68 (d, J=2.5 Hz, 1H), 9.75 (s, 1H), 10.41 (s, 1H).

LC-MS (Method 3): Rₜ=1.22 min; MS (ESIpos): m/z=515 ([M+H]⁺, 100%); MS (ESIneg): m/z=513 ([M−H]⁻, 100%).

### Example 77

### methyl 4′-({4-methoxy-3-[(morpholin-4-ylacetyl)amino]phenyl}carbamoyl)biphenyl-3-carboxylate

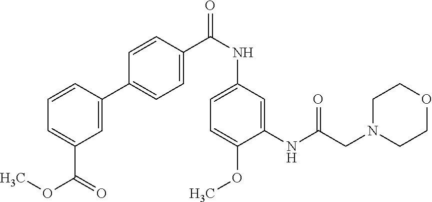 [SMILES: COC(=O)c1cccc(-c2ccc(C(=O)Nc3ccc(OC)c(NC(=O)CN4CCOCC4)c3)cc2)c1]

To a microwave vial was added 4-bromo-N-{4-methoxy-3-[(morpholin-4-ylacetyl)amino]phenyl}benzamide (prepared in a manner analogous to that described in example 59A, 0.075 g, 0.167 mmol), [3-(methoxycarbonyl)phenyl]boronic acid (0.060 g, 0.33 mmol, 2.0 equiv), a 2N sodium carbonate solution (0.25 mL, 0.50 mmol, 3.0 equiv) and dioxane (1.8 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.013 g, 0.016 mmol, 10 mol %) and sealed. The resulting mixture was heated with a microwave apparatus at 105° C. for 1 h, was then cooled to room temperature. The reaction mixture was poured onto water (10 mL), and extracted with ethyl acetate (3×10 mL). The combined organic phases were dried (Na₂SO₄ anh), and concentrated under reduced pressure. The resulting material was purified by HPLC (method 3) to give methyl 4′-({4-methoxy-3-[(morpholin-4-ylacetyl)amino]phenyl}carbamoyl)biphenyl-3-carboxylate (39 mg, 46%).

¹H-NMR (400 MHz, DMSO-d₆): δ [ppm]=2.50-2.54 (m, 4H), 3.12 (s, 2H), 3.62-3.66 (m, 4H), 3.86 (s, 3H), 3.87 (s, 3H), 7.02 (d, J=9.0 Hz, 1H), 7.56 (dd, J=2.6, 8.7 Hz, 1H), 7.64 (t, H=7.7, 1H), 7.83 (d, 8.5 Hz, 2H), 7.95-8.04 (m, 2H), 8.06 (d, J=8.3 Hz, 2H), 8.22-8.24 (m, 1H), 8.55 (d, J=2.5 Hz, 1H), 9.71 (s, 1H), 10.23 (s, 1H).

LC-MS (Method 3): Rₜ=1.14 min; MS (ESIpos): m/z=504 ([M+H]⁺, 100%); MS (ESIneg): m/z=502 ([M−H]⁻, 100%).

### Example 78

### N-{4-methoxy-3-[(morpholin-4-ylacetyl)amino]phenyl}-3′-(trifluoromethyl)biphenyl-4-carboxamide

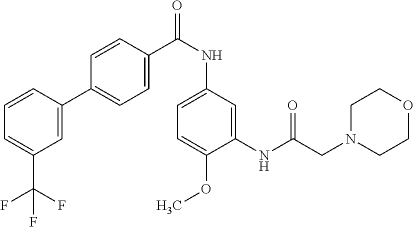 [SMILES: COc1ccc(NC(=O)c2ccc(-c3cccc(C(F)(F)F)c3)cc2)cc1NC(=O)CN1CCOCC1]

To a microwave vial was added 4-bromo-N-{4-methoxy-3-[(morpholin-4-ylacetyl)amino]phenyl}benzamide (prepared in a manner analogous to that described in example 59A, 0.075 g, 0.167 mmol), [3-(trifluoromethyl)phenyl]boronic acid (0.063 g, 0.33 mmol, 2.0 equiv), a 2N sodium carbonate solution (0.25 mL, 0.50 mmol, 3.0 equiv) and dioxane (1.8 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.013 g, 0.016 mmol, 10 mol %) and sealed. The resulting mixture was heated with a microwave apparatus at 105° C. for 1 h, was then cooled to room temperature. The reaction mixture was poured onto water (10 mL), and extracted with ethyl acetate (3×10 mL). The combined organic phases were dried (Na₂SO₄ anh), and concentrated under reduced pressure. The resulting material was purified by HPLC (method 3) to give N-{4-methoxy-3-[(morpholin-4-ylacetyl)amino]phenyl}-3′-(trifluoromethyl)biphenyl-4-carboxamide (33 mg, 39%).

¹H-NMR (400 MHz, DMSO-d₆): δ [ppm]=2.50-2.55 (m, 4H), 3.12 (s, 2H), 3.62-3.66 (m, 4H), 3.86 (s, 3H), 7.02 (d, J=9.0 Hz, 1H), 7.56 (dd, J=2.5, 8.9 Hz, 1H), 7.68-7.78 (m, 2H), 7.88 (d, 8.5 Hz, 2H), 8.02-8.09 (m, 4H), 8.55 (d, J=2.5 Hz, 1H), 9.71 (s, 1H), 10.25 (s, 1H).

LC-MS (Method 3): Rₜ=1.27 min; MS (ESIpos): m/z=514 ([M+H]⁺, 100%); MS (ESIneg): m/z=512 ([M−H]⁻, 100%).

### Example 79

### methyl 4′-({4-methoxy-3-[(morpholin-4-ylacetyl)amino]phenyl}carbamoyl)biphenyl-4-carboxylate

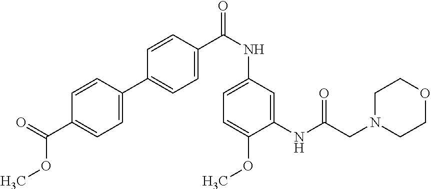 [SMILES: COC(=O)c1ccc(-c2ccc(C(=O)Nc3ccc(OC)c(NC(=O)CN4CCOCC4)c3)cc2)cc1]

To a microwave vial was added 4-bromo-N-{4-methoxy-3-[(morpholin-4-ylacetyl)amino]phenyl}benzamide (prepared in a manner analogous to that described in example 59A, 0.075 g, 0.167 mmol), [4-(methoxycarbonyl)phenyl]boronic acid (0.060 g, 0.33 mmol, 2.0 equiv), a 2N sodium carbonate solution (0.25 mL, 0.50 mmol, 3.0 equiv) and dioxane (1.8 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.013 g, 0.016 mmol, 10 mol %) and sealed. The resulting mixture was heated with a microwave apparatus at 105° C. for 1 h, was then cooled to room temperature. The reaction mixture was poured onto water (10 mL), and extracted with ethyl acetate (3×10 mL). The combined organic phases were dried (Na₂SO₄ anh), and concentrated under reduced pressure. The residue was triturated with ethanol, was then purified by HPLC (method 3) to give methyl 4′-({4-methoxy-3-[(morpholin-4-ylacetyl)amino]phenyl}carbamoyl)biphenyl-4-carboxylate (30 mg, 35%).

¹H-NMR (400 MHz, DMSO-d₆): δ [ppm]=2.50-2.55 (m, 4H), 3.12 (s, 2H), 3.61-3.66 (m, 4H), 3.86 (s, 6H), 7.02 (d, J=9.0 Hz, 1H), 7.56 (dd, J=2.5, 8.9 Hz, 1H), 7.85-7.91 (m, 4H), 8.02-8.09 (m, 4H), 8.55 (d, J=2.5 Hz, 1H), 9.71 (s, 1H), 10.24 (s, 1H).

LC-MS (Method 3): Rₜ=1.18 min; MS (ESIpos): m/z=504 ([M+H]⁺, 100%); MS (ESIneg): m/z=502 ([M−H]⁻, 100%).

### Example 80

### 3′-methoxy-N-{4-methoxy-3-[(morpholin-4-ylacetyl)amino]phenyl}biphenyl-4-carboxamide

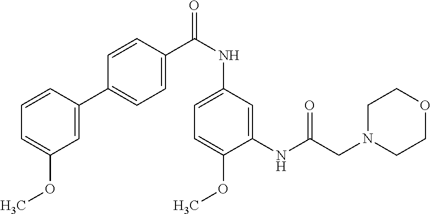 [SMILES: COc1cccc(-c2ccc(C(=O)Nc3ccc(OC)c(NC(=O)CN4CCOCC4)c3)cc2)c1]

To a microwave vial was added 4-bromo-N-{4-methoxy-3-[(morpholin-4-ylacetyl)amino]phenyl}benzamide (prepared in a manner analogous to that described in example 59A, 0.075 g, 0.167 mmol), (3-methoxyphenyl)boronic acid (0.051 g, 0.33 mmol, 2.0 equiv), a 2N sodium carbonate solution (0.25 mL, 0.50 mmol, 3.0 equiv) and dioxane (1.8 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.013 g, 0.016 mmol, 10 mol %) and sealed. The resulting mixture was heated with a microwave apparatus at 105° C. for 1 h, was then cooled to room temperature. The reaction mixture was poured onto water (10 mL), and extracted with ethyl acetate (3×10 mL). The combined organic phases were dried (Na₂SO₄ anh), and concentrated under reduced pressure. The residue was then purified by HPLC (method 3) to give 3′-methoxy-N-{4-methoxy-3-[(morpholin-4-ylacetyl)amino]phenyl}biphenyl-4-carboxamide (46 mg, 57%).

¹H-NMR (400 MHz, DMSO-d₆): δ [ppm]=2.51-2.54 (m, 4H), 3.12 (s, 2H), 3.62-3.66 (m, 4H), 3.82 (s, 3H), 3.86 (s, 3H), 6.96 (dd, J=1.8, 6.3 Hz, 1H), 7.02 (d, J=8.8 Hz, 1H), 7.23-7.25 (m, 1H), 7.28 (d, J=7.8 Hz, 1H), 7.38 (t, J=8.0 Hz, 1H), 7.56 (dd, J=2.5, 9.1 Hz, 1H), 7.78 (d, J=8.6 Hz, 2H), 8.02 (d, J=8.6 Hz, 2H), 8.55 (d, J=2.5 Hz, 1H), 9.71 (s, 1H), 10.20 (s, 1H).

LC-MS (Method 3): Rₜ=1.16 min; MS (ESIpos): m/z=476 ([M+H]⁺, 100%), 951 ([2M+H]⁺, 70%); MS (ESIneg): m/z=474 ([M−H]⁻, 100%).

### Example 81

### 3′-fluoro-N-{4-methoxy-3-[(morpholin-4-ylacetyl)amino]phenyl}biphenyl-4-carboxamide

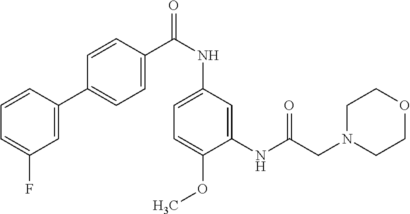 [SMILES: COc1ccc(NC(=O)c2ccc(-c3cccc(F)c3)cc2)cc1NC(=O)CN1CCOCC1]

To a microwave vial was added 4-bromo-N-{4-methoxy-3-[(morpholin-4-ylacetyl)amino]phenyl}benzamide (prepared in a manner analogous to that described in example 59A, 0.075 g, 0.167 mmol), (3-fluorophenyl)boronic acid (0.047 g, 0.33 mmol, 2.0 equiv), a 2N sodium carbonate solution (0.25 mL, 0.50 mmol, 3.0 equiv) and dioxane (1.8 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.013 g, 0.016 mmol, 10 mol %) and sealed. The resulting mixture was heated with a microwave apparatus at 105° C. for 1 h, was then cooled to room temperature. The reaction mixture was poured onto water (10 mL), and extracted with ethyl acetate (3×10 mL). The combined organic phases were dried (Na₂SO₄ anh), and concentrated under reduced pressure. The residue was then purified by HPLC (method 3) to give 3′-fluoro-N-{4-methoxy-3-[(morpholin-4-ylacetyl)amino]phenyl}biphenyl-4-carboxamide (43 mg, 56%).

¹H-NMR (400 MHz, DMSO-d₆): δ [ppm]=2.50-2.54 (m, 4H), 3.12 (s, 2H), 3.61-3.66 (m, 4H), 3.86 (s, 3H), 7.02 (d, J=9.0 Hz, 1H), 7.18-7.26 (m, 1H), 7.47-7.62 (m, 4H), 7.83 (d, J=8.3 Hz, 2H), 8.04 (d, J=8.3 Hz, 2H), 8.55 (d, J=2.5 Hz, 1H), 9.71 (s, 1H), 10.24 (s, 1H).

LC-MS (Method 3): Rₜ=1.19 min; MS (ESIpos): m/z=464 ([M+H]⁺, 100%), 927 ([2M+H]⁺, 40%); MS (ESIneg): m/z=462 ([M−H]⁻, 100%).

### Example 82

### 2′-fluoro-N-{4-methoxy-3-[(morpholin-4-ylacetyl)amino]phenyl}biphenyl-4-carboxamide

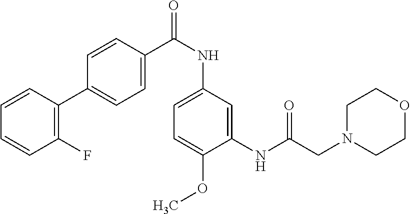 [SMILES: COc1ccc(NC(=O)c2ccc(-c3ccccc3F)cc2)cc1NC(=O)CN1CCOCC1]

To a microwave vial was added 4-bromo-N-{4-methoxy-3-[(morpholin-4-ylacetyl)amino]phenyl}benzamide (prepared in a manner analogous to that described in example 59A, 0.075 g, 0.167 mmol), (2-fluorophenyl)boronic acid (0.047 g, 0.33 mmol, 2.0 equiv), a 2N sodium carbonate solution (0.25 mL, 0.50 mmol, 3.0 equiv) and dioxane (1.8 mL). The resulting suspension was purged with argon, treated with [1,1′-bis(diphenylphosphino)ferrocene]palladium(II) chloride CH₂Cl₂ complex (Pd(dppf)Cl₂CH₂Cl₂, 0.013 g, 0.016 mmol, 10 mol %) and sealed. The resulting mixture was heated with a microwave apparatus at 105° C. for 1 h, was then cooled to room temperature. The reaction mixture was poured onto water (10 mL), and extracted with ethyl acetate (3×10 mL). The combined organic phases were dried (Na₂SO₄ anh), and concentrated under reduced pressure. The residue was then purified by HPLC (method 3) to give 2′-fluoro-N-{4-methoxy-3-[(morpholin-4-ylacetyl)amino]phenyl}biphenyl-4-carboxamide (39 mg, 50%).

¹H-NMR (400 MHz, DMSO-d₆): δ [ppm]=2.50-2.54 (m, 4H), 3.12 (s, 2H), 3.61-3.66 (m, 4H), 3.86 (s, 3H), 7.02 (d, J=9.0 Hz, 1H), 7.28-7.36 (m, 2H), 7.40-7.48 (m, 1H), 7.54-7.60 (m, 2H), 7.66 (d, J=8.1 Hz, 2H), 8.03 (d, J=8.3 Hz, 2H), 8.55 (d, J=2.5 Hz, 1H), 9.72 (s, 1H), 10.25 (s, 1H).

LC-MS (Method 3): Rₜ=1.18 min; MS (ESIpos): m/z=464 ([M+H]⁺, 100%), 927 ([2M+H]⁺, 40%); MS (ESIneg): m/z=462 [M−H]⁻, 100%), 925 ([2M−H]⁻, 20%).

### Example 83

### 4′-amino-N-{4-methoxy-3-[(morpholin-4-ylacetyl)amino]phenyl}biphenyl-4-carboxamide

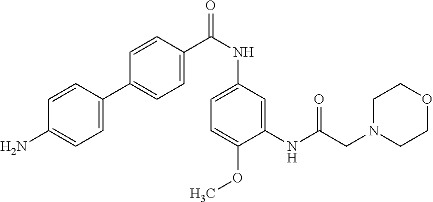 [SMILES: COc1ccc(NC(=O)c2ccc(-c3ccc(N)cc3)cc2)cc1NC(=O)CN1CCOCC1]

A solution of tert-butyl[4′-({4-methoxy-3-[(morpholin-4-ylacetyl)amino]phenyl}carbamoyl)biphenyl-4-yl]carbamate (prepared in a manner analogous to that described in example 60A, 0.097 g, 0.173 mmol) in dioxane (3 mL) was treated with HCl (4M in dioxane, 0.43 mL, 1.73 mmol, 10 equiv), and the resulting solution was stirred at room temperature for 24 h. Additional HCl (4M in dioxane, 0.43 mL, 1.73 mmol, 10 equiv) was added, and the resulting mixture was stirred at room temperature for 24 h. The resulting solids were removed by filtration, washed with ethyl acetate, and dried at 50° C. under reduced pressure. Purification by HPLC (method 3) afforded 4′-amino-N-{4-methoxy-3-[(morpholin-4-ylacetyl)amino]phenyl}biphenyl-4-carboxamide (15 mg, 19%).

¹H-NMR (400 MHz, DMSO-d₆): δ [ppm]=2.56-2.59 (m, 4H), 3.16 (s, 2H), 3.68-3.70 (m, 4H), 3.90 (s, 3H), 5.36 (s, 2H), 6.68 (d, J=8.7 Hz, 2H), 7.06 (d, J=9.0 Hz, 1H), 7.48 (d, J=8.7 Hz, 2H), 7.59 (dd, J=2.6, 9.0 Hz, 1H), 7.69 (d, J=8.7 Hz, 2H), 7.99 (d, J=8.3 Hz, 2H), 8.58 (d, J=2.6 Hz, 1H), 9.74 (s, 1H), 10.24 (s, 1H).

LC-MS (Method 3): Rₜ=1.19 min; MS (ESIpos): m/z=461 ([M+H]⁺, 100%).

### Example 84

### N-{4-hydroxy-3-[(morpholin-4-ylacetyl)amino]phenyl}biphenyl-4-carboxamide

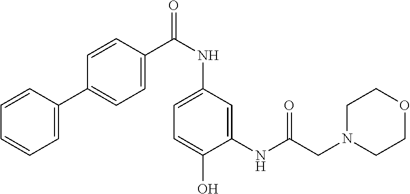 [SMILES: O=C(CN1CCOCC1)Nc1cc(NC(=O)c2ccc(-c3ccccc3)cc2)ccc1O]

To a solution of N-(5-amino-2-{[tert-butyl(dimethyl)silyl]oxy}phenyl)-2-(morpholin-4-yl)acetamide (prepared in a manner analogous to that described in example 57A, 1.15 g, 3.15 mmol) and biphenyl-4-carboxylic acid (0.81 g, 4.09 mmol, 1.3 equiv) in DMF (25 mL) was added propanephosphonic acid cyclic anhydride solution (50% in ethyl acetate, 2.39 mL, 4.09 mmol, 1.3 equiv) followed by diisopropylethylamine (1.92 mL, 11.0 mmol, 3.5 equiv). The resulting mixture was stirred at room temperature for 24 h, was then treated with water (25 mL). The resulting mixture was extracted with ethyl acetate (3×25 mL). The combined organic phases were dried (Na₂SO₄ anh), and concentrated under reduced pressure (1.20 g).

LC-MS (Method 1): Rₜ=0.85 min; MS (ESIpos): m/z=432 ([M+H]⁺, 100%), 863 ([2M+H]⁺, 10%); MS (ESIneg): m/z=430 ([M−H]⁻, 100%), 861 ([2M−H]⁻, 20%).

A solution of the resulting residue (1.20 g) in THF (20 mL) at room temperature was treated with a tetrabutylammonium fluoride solution (1.0M in THF, 6.6 mL, 6.60 mmol, 3.0 equiv). The resulting solution was stirred at room temperature for 12 h. The resulting THF solution was diluted with water (50 mL). The resulting mixture was extracted with ethyl acetate (3×25 mL). The combined organic phases were dried (Na₂SO₄ anh) and concentrated under reduced pressure to give N-{4-hydroxy-3-[(morpholin-4-ylacetyl)amino]phenyl}biphenyl-4-carboxamide (0.55 g, 40% overall).

¹H-NMR (400 MHz, DMSO-d₆): δ [ppm]=2.49-2.54 (m, 4H), 3.12 (s, 2H), 3.60-3.65 (m, 4H), 6.80 (d, J=8.7 Hz, 1H), 7.36-7.41 (m, 2H), 7.47 (t, J=7.5 Hz, 2H), 7.72 (d, J=7.4 Hz, 2H), 7.77 (d, J=8.3 Hz, 2H), 8.02 (d, J=8.5 Hz, 2H), 8.45 (d, J=2.5 Hz, 1H), 9.61 (s, 1H), 9.93 (br s, 1H), 10.13 (s, 1H).

LC-MS (Method 1): MS (ESIpos): m/z=432 ([M+H]⁺, 100%), 863 ([2M+H]⁺, 20%); MS (ESIneg): m/z=430 ([M−H]⁻, 100%), 861 ([2M−H]⁻, 40%).

### Example 85

### N-{4-ethoxy-3-[(morpholin-4-ylacetyl)amino]phenyl}biphenyl-4-carboxamide

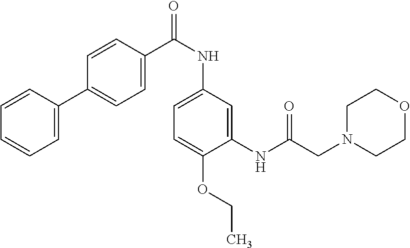 [SMILES: CCOc1ccc(NC(=O)c2ccc(-c3ccccc3)cc2)cc1NC(=O)CN1CCOCC1]

A mixture of N-{4-hydroxy-3-[(morpholin-4-ylacetyl)amino]phenyl}biphenyl-4-carboxamide (prepared in a manner analogous to that described in example 84, 0.11 g, 0.255 mmol), iodoethane (0.025 mL, 0.319 mmol, 1.25 equiv), and Cs₂CO₃ (0.166 g, 0.510 mmol, 2.0 equiv) in DMF (2.6 mL) was stirred at 60° C. for 6 h, was then treated with water (5 mL). The resulting mixture was extracted with a CH₂Cl₂/isopropanol mixture (4:1, 3×5 mL). The combined organic phases were dried (Na₂CO₃ anh) and concentrated under reduced pressure. The residue (0.12 g) was recrystallized from ethanol to give N-{4-ethoxy-3-[(morpholin-4-ylacetyl)amino]phenyl}biphenyl-4-carboxamide (0.080 g, 68%).

¹H-NMR (300 MHz, DMSO-d₆): δ [ppm]=1.41 (t, J=6.9 Hz, 3H), 2.51-2.56 (m, 4H), 3.12 (s, 2H), 3.62-3.67 (m, 4H), 4.08 (q, J=7.0 Hz, 2H), 7.00 (d, J=8.9 Hz, 1H), 7.38 (t, J=7.3 Hz, 1H), 7.47 (t, J=7.4 Hz, 2H), 7.54 (dd, J=2.5, 8.9 Hz, 1H), 7.70-7.80 (m, 4H), 8.03 (d, J=8.3 Hz, 2H), 8.62 (d, J=2.4 Hz, 1H), 9.77 (s, 1H), 10.21 (s, 1H).

LC-MS (Method 3): Rₜ=1.32 min; MS (ESIpos): m/z=460 ([M+H]⁺, 50%), 919 ([2M+H]⁺, 80%); MS (ESIneg): m/z=458 ([M−H]⁻, 100%).

### Example 86

### N-(biphenyl-4-yl)-4-[(2-methoxyethoxy)methyl]-3-[(morpholin-4-ylacetyl)amino]benzamide

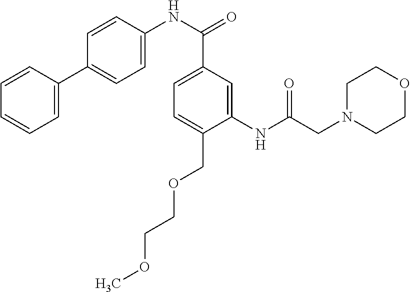 [SMILES: COCCOCc1ccc(C(=O)Nc2ccc(-c3ccccc3)cc2)cc1NC(=O)CN1CCOCC1]

70.0 mg (160 μmol) of the compound of example 69A were provided in 1.5 mL of DMF. 32 μL (0.23 mmol) of triethylamine, 20 μL (0.23 mmol) of morpholine and 4.0 mg (0.02 mmol) of potassium iodide were added, and the mixture was stirred at room temperature over night. After filtration, purification by HPLC (method 2) yielded 17.0 mg (22% of theory) of the title compound.

¹H-NMR (300 MHz, DMSO-d₆): δ [ppm]=2.54-2.62 (m, 4H), 3.17 (s, 2H), 3.24 (s, 3H), 3.47-3.60 (m, 4H), 3.65-3.73 (m, 4H), 4.64 (s, 2H), 7.30-7.38 (m, 1H), 7.41-7.54 (m, 3H), 7.64-7.75 (m, 5H), 7.84-7.92 (m, 2H), 8.56 (d, 1H), 9.96 (s, 1H), 10.37 (s, 1H).

LC-MS (Method 4): Rₜ=1.06 min; MS (ESIpos): m/z=504 [M+H]⁺.

### Example 87

### N-(biphenyl-4-yl)-4-[(3-methoxypropoxy)methyl]-3-[(morpholin-4-ylacetyl)amino]benzamide

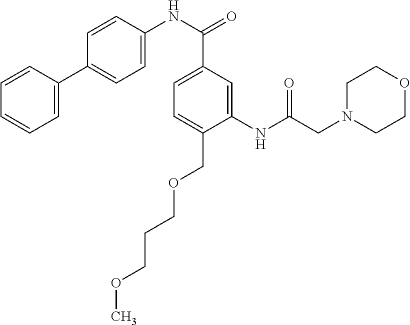 [SMILES: COCCCOCc1ccc(C(=O)Nc2ccc(-c3ccccc3)cc2)cc1NC(=O)CN1CCOCC1]

115 mg (250 μmol) of the compound of example 73A were provided in 1.5 mL of DMF. 51 μL (0.37 mmol) of triethylamine, 32 μL (0.37 mmol) of morpholine and 6.0 mg (0.04 mmol) of potassium iodide were added, and the mixture was stirred at room temperature for 3 h. After filtration, purification by HPLC (method 2) yielded 64.0 mg (49% of theory) of the title compound.

¹H-NMR (300 MHz, DMSO-d₆): δ [ppm]=1.81 (quin, 2H), 2.54-2.63 (m, 4H), 3.18 (s, 2H), 3.19 (s, 3H), 3.39 (t, 2H), 3.48 (t, 2H), 3.63-3.74 (m, 4H), 4.61 (s, 2H), 7.30-7.39 (m, 1H), 7.42-7.54 (m, 3H), 7.64-7.75 (m, 5H), 7.84-7.92 (m, 2H), 8.57 (d, 1H), 9.96 (s, 1H), 10.37 (s, 1H).

LC-MS (Method 4): Rₜ=1.14 min; MS (ESIpos): m/z=518 [M+H]⁺.

### Example 88

### 4-(benzyloxy)-N-(biphenyl-4-yl)-3-({[1-(morpholin-4-yl)cyclopropyl]carbonyl}amino)benzamide

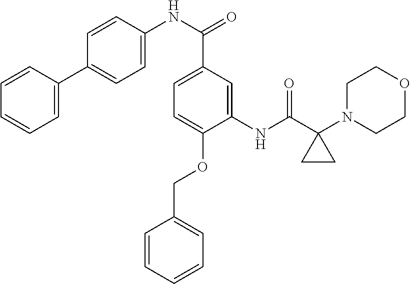 [SMILES: O=C(Nc1ccc(-c2ccccc2)cc1)c1ccc(OCc2ccccc2)c(NC(=O)C2(N3CCOCC3)CC2)c1]

To a solution of biphenyl-4-amine (768 mg, 4.54 mmol) and the compound of example 75A (1.50 g, 3.78 mmol) in DMF (14 mL) was added (benzotriazol-1-yloxy)tripyrrolidinophosphonium hexafluorophosphate (PYBOP, 3.94 g, 7.57 mmol) and diisopropylethylamine (2.64 mL, 15.1 mmol). The resulting mixture was stirred at room temperature over night, was concentrated under reduced pressure, was then dissolved in dichloromethane, was washed with 1N aqueous hydrogen chloride solution and saturated, aqueous sodium bicarbonate solution, was dried over sodium sulfate and concentrated under reduced pressure. The remaining solids were then triturated with ethanol (20 mL), and the resulting mixture was stirred for 30 minutes. The remaining solids were removed by filtration, washed with ethanol, and were dried under reduced pressure. The remaining solids were then triturated with ethanol (50 mL), and the resulting mixture was stirred under reflux. The remaining solids were removed by filtration while the mixture was still warm, were washed with ethanol, and were dried under reduced pressure to give the title compound (1.46 g, 70% of theory).

¹H-NMR (300 MHz, DMSO-d₆): δ [ppm]=1.05-1.20 (m, 4H), 2.23-2.32 (m, 4H), 3.15-3.29 (m, 4H), 5.29 (s, 2H), 7.30-7.40 (m, 2H), 7.40-7.50 (m, 5H), 7.55-7.61 (m, 2H), 7.63-7.70 (m, 4H), 7.74 (dd, 1H), 7.87 (s, 2H), 8.92 (d, 1H), 10.24 (s, 1H), 10.44 (s, 1H).

LC-MS (Method 4): Rₜ=1.49 min; MS (ESIpos): m/z=548 [M+H]⁺.

### Example 89

### 4-(3-aminopropoxy)-N-(biphenyl-4-yl)-3-({[1-(morpholin-4-yl)cyclopropyl]carbonyl}amino)benzamide hydrochloride (1:1)

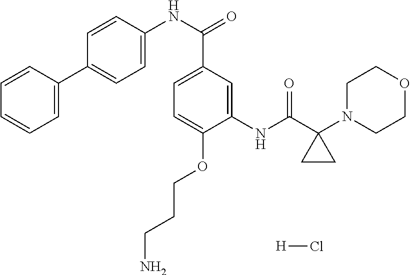 [SMILES: NCCCOc1ccc(C(=O)Nc2ccc(-c3ccccc3)cc2)cc1NC(=O)C1(N2CCOCC2)CC1 ; Cl]

560 mg (0.91 mmol) of the compound of example 77A were treated with HCl (4M in dioxane, 11.4 mL, 45.6 mmol, 50 equiv), and the resulting mixture was stirred at room temperature over night. After concentration, the remaining solids were triturated with ethanol and stirred for 30 minutes. The precipitate was removed by filtration, washed with ethanol, and dried under reduced pressure affording 183 mg (36% of theory) of the title compound.

¹H-NMR (400 MHz, DMSO-d₆): δ [ppm]=1.09-1.35 (m, 4H), 2.13-2.23 (m, 2H), 2.36-2.50 (m, 4H), 2.96-3.08 (m, 2H), 2.97-3.07 (m, 2H), 4.33 (t, 2H), 7.23 (d, 1H), 7.30-7.37 (m, 1H), 7.42-7.49 (m, 2H), 7.63-7.70 (m, 4H), 7.79 (d, 1H), 7.84-7.92 (m, 2H), 8.08 (s, 3H), 8.86 (s, 1H), 10.25 (s, 1H), 10.33 (s, 1H).

LC-MS (Method 4): Rₜ=1.11 min; MS (ESIpos): m/z=515 [M+H−HCl]⁺.

### Example 90

### 4-(3-acetamidopropoxy)-N-(biphenyl-4-yl)-3-({[1-(morpholin-4-yl)cyclopropyl]carbonyl}amino)benzamide

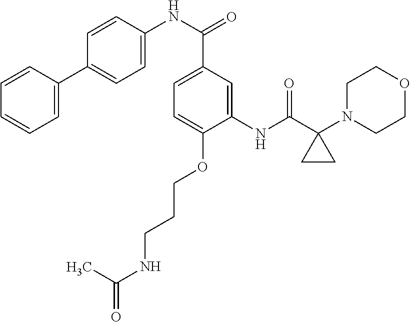 [SMILES: CC(=O)NCCCOc1ccc(C(=O)Nc2ccc(-c3ccccc3)cc2)cc1NC(=O)C1(N2CCOCC2)CC1]

218 mg (0.32 mmol) of the compound of example 89 were provided in 3 mL of dichloromethane and treated with 0.26 mL (3.24 mmol) of pyridine, 0.57 mL (3.24 mmol) of N,N-diisopropylethylamine and 0.06 mL (0.65 mmol) of acetic anhydride, and the resulting mixture was stirred at room temperature over night. After concentration, the remaining solids were triturated with water and ethanol and stirred for 30 minutes. The precipitate was removed by filtration, washed with ethanol, and dried under reduced pressure affording 59.0 mg (32% of theory) of the title compound.

¹H-NMR (300 MHz, DMSO-d₆): δ [ppm]=1.09-1.17 (m, 2H), 1.18-1.27 (m, 2H), 1.82 (s, 3H), 1.95-2.08 (m, 2H), 2.39-2.47 (m, 4H), 3.21-3.30 (m, 2H), 3.64-3.76 (m, 4H), 4.23 (t, 2H), 7.20 (d, 1H), 7.29-7.37 (m, 1H), 7.45 (s, 2H), 7.62-7.70 (m, 4H), 7.73 (dd, 1H), 7.82-7.90 (m, 2H), 7.94-8.03 (m, 1H), 8.89 (d, 1H), 10.23 (s, 1H), 10.40 (s, 1H).

LC-MS (Method 4): Rₜ=1.24 min; MS (ESIpos): m/z=557 [M+H]⁺.

### Example 91

### N-(biphenyl-4-yl)-4-(3-methoxypropoxy)-3-[(morpholin-4-ylacetyl)amino]benzamide

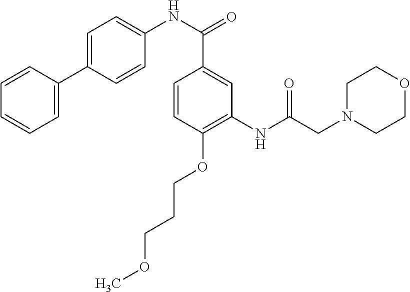 [SMILES: COCCCOc1ccc(C(=O)Nc2ccc(-c3ccccc3)cc2)cc1NC(=O)CN1CCOCC1]

65.0 mg (150 μmol) of the compound of example 24A and 18.0 mg (170 μmol) of 1-chloro-3-methoxypropane were provided in 2 mL of DMF. 62.5 mg (0.45 mmol) of potassium carbonate were added, and the mixture was stirred at 100° C. for 3 days. After filtration, purification by HPLC (column: chromatorex C18, 10 μm, 125×30 mm, mobile phase: acetonitrile/water+0.1% formic acid gradient) yielded 35.5 mg (47% of theory) of the title compound.

¹H-NMR (400 MHz, DMSO-d₆): δ [ppm]=2.05-2.14 (m, 2H), 2.54-2.62 (m, 4H), 3.19 (s, 2H), 3.28 (s, 3H), 3.58 (t, 2H), 3.64-3.71 (m, 4H), 4.23 (t, 2H), 7.21 (d, 1H), 7.30-7.37 (m, 1H), 7.41-7.49 (m, 2H), 7.63-7.70 (m, 4H), 7.75 (dd, 1H), 7.83-7.90 (m, 2H), 8.85 (d, 1H), 9.74 (s, 1H), 10.23 (s, 1H).

LC-MS (Method 1): Rₜ=1.12 min; MS (ESIpos): m/z=504 [M+H]⁺.

### Example 92

### N-(biphenyl-4-yl)-4-(2-methoxyethoxy)-3-[(morpholin-4-ylacetyl)amino]benzamide

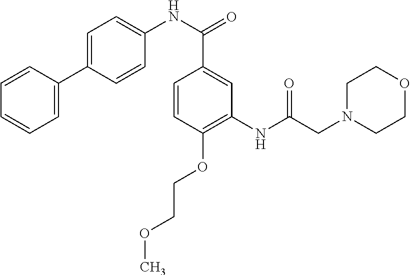 [SMILES: COCCOc1ccc(C(=O)Nc2ccc(-c3ccccc3)cc2)cc1NC(=O)CN1CCOCC1]

65.0 mg (150 μmol) of the compound of example 24A and 15.7 mg (170 μmol) of 1-chloro-2-methoxyethane were provided in 2 mL of DMF. 62.5 mg (0.45 mmol) of potassium carbonate were added, and the mixture was stirred at 100° C. for 3 days. After filtration, purification by HPLC (column: chromatorex C18, 10 μm, 125×30 mm, mobile phase: acetonitrile/water+0.1% formic acid gradient) yielded 36.5 mg (49% of theory) of the title compound.

¹H-NMR (400 MHz, DMSO-d₆): δ [ppm]=2.53-2.60 (m, 4H), 3.18 (s, 2H), 3.36 (s, 3H), 3.68-3.74 (m, 4H), 3.76-3.81 (m, 2H), 4.28-4.34 (m, 2H), 7.22 (d, 1H), 7.30-7.36 (m, 1H), 7.42-7.48 (m, 2H), 7.63-7.70 (m, 4H), 7.74 (dd, 1H), 7.83-7.90 (m, 2H), 8.88 (d, 1H), 9.85 (s, 1H), 10.23 (s, 1H).

LC-MS (Method 1): Rₜ=1.03 min; MS (ESIpos): m/z=490 [M+H]⁺.

### Example 93

### N-(biphenyl-4-yl)-4-(2-hydroxyethoxy)-3-[(morpholin-4-ylacetyl)amino]benzamide

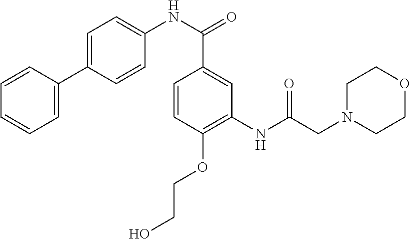 [SMILES: O=C(CN1CCOCC1)Nc1cc(C(=O)Nc2ccc(-c3ccccc3)cc2)ccc1OCCO]

75.0 mg (170 μmol) of the compound of example 24A and 15.4 mg (190 μmol) of 2-chloroethanol were provided in 2 mL of DMF. 72.1 mg (0.52 mmol) of potassium carbonate were added, and the mixture was stirred at 100° C. for 3 days. After filtration, purification by HPLC (column: chromatorex C18, 10 μm, 125×30 mm, mobile phase: acetonitrile/water+0.1% formic acid gradient) yielded 53.0 mg (58% of theory) of the title compound.

¹H-NMR (400 MHz, DMSO-d₆): δ [ppm]=2.54-2.59 (m, 4H), 3.17 (s, 2H), 3.68-3.73 (m, 4H), 3.82-3.89 (m, 2H), 4.21 (t, 2H), 4.94 (t, 1H), 7.21 (d, 1H), 7.30-7.37 (m, 1H), 7.42-7.49 (m, 2H), 7.63-7.70 (m, 4H), 7.74 (dd, 1H), 7.84-7.90 (m, 2H), 8.85 (d, 1H), 9.87 (s, 1H), 10.23 (s, 1H).

LC-MS (Method 4): Rₜ=0.94 min; MS (ESIpos): m/z=476 [M+H]⁺.

### Example 94

### N-(biphenyl-4-yl)-3-({[1-(morpholin-4-yl)cyclopropyl]carbonyl}amino)-4-(trifluoromethoxy)benzamide

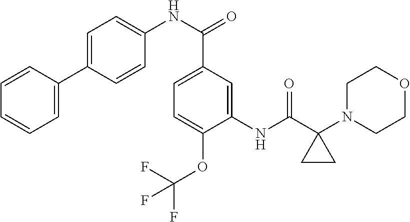 [SMILES: O=C(Nc1ccc(-c2ccccc2)cc1)c1ccc(OC(F)(F)F)c(NC(=O)C2(N3CCOCC3)CC2)c1]

415 mg (2.00 mmol) of 1-(morpholin-4-yl)cyclopropanecarboxylic acid hydrochloride (1:1) (example 65A) were stirred in 10 mL of dichloromethane at room temperature. 15.4 μL (0.20 mmol) of DMF and 0.35 mL (4.00 mmol) of oxalyl chloride were added, and the mixture was stirred for additional 2 h at 50° C. after the gas formation had stopped. After concentration, 440 mg of a raw material were obtained, of which 137 mg (0.60 mmol) were added to a solution of 150 mg (0.40 mmol) of the compound of example 78A and 0.28 mL (2.01 mmol) of triethylamine in a mixture of 2 mL of dichloromethane and 2 mL of THF. The resulting mixture was stirred at room temperature over night. After concentration, the remaining solids were then triturated with water and the mixture was extracted with ethyl acetate. The combined organic phases were washed with 1N aqueous hydrogen chloride solution and saturated, aqueous sodium bicarbonate solution, was dried over sodium sulfate and concentrated under reduced pressure. Purification by HPLC (method 2) yielded 86.2 mg (41% of theory) of the title compound.

¹H-NMR (300 MHz, DMSO-d₆): δ [ppm]=1.12-1.22 (m, 2H), 1.22-1.32 (m, 2H), 2.42-2.49 (m, 4H), 3.64-3.76 (m, 4H), 7.30-7.38 (m, 1H), 7.41-7.50 (m, 2H), 7.61-7.73 (m, 5H), 7.80 (dd, 1H), 7.83-7.89 (m, 2H), 8.90 (d, 1H), 10.47 (s, 1H), 10.54 (s, 1H).

LC-MS (Method 4): Rₜ=1.47 min; MS (ESIpos): m/z=526 [M+H]⁺.

### Example 95

### N-(biphenyl-4-yl)-4-(methoxymethyl)-3-({[1-(morpholin-4-yl)cyclopropyl]carbonyl}amino)benzamide

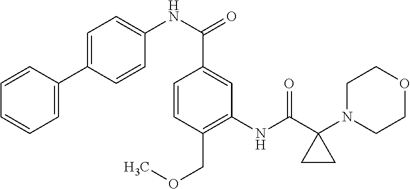 [SMILES: COCc1ccc(C(=O)Nc2ccc(-c3ccccc3)cc2)cc1NC(=O)C1(N2CCOCC2)CC1]

To a solution of the compound of example 81A (100 mg, 0.30 mmol) and the compound of example 65A (125 mg, 0.60 mmol) in DMF (1.5 mL) was added (benzotriazol-1-yloxy)tripyrrolidinophosphonium hexafluorophosphate (PYBOP, 313 mg, 0.60 mmol) and diisopropylethylamine (0.26 mL, 1.50 mmol). The resulting mixture was stirred at room temperature over night. After filtration, purification by HPLC (method 2) yielded 64.0 mg (44% of theory) of the title compound.

¹H-NMR (300 MHz, DMSO-d₆): δ [ppm]=1.09-1.17 (m, 2H), 1.18-1.26 (m, 2H), 2.41-2.47 (m, 4H), 3.31 (s, 3H), 3.67-3.77 (m, 4H), 4.64 (s, 2H), 7.29-7.38 (m, 1H), 7.42-7.54 (m, 3H), 7.64-7.71 (m, 5H), 7.85-7.90 (m, 2H), 8.68 (d, 1H), 10.36 (s, 1H), 10.64 (s, 1H).

LC-MS (Method 4): Rₜ=1.38 min; MS (ESIpos): m/z=486 [M+H]⁺.

### Example 96

### N-(biphenyl-4-yl)-4-(methoxymethyl)-3-({[1-(4-methylpiperazin-1-yl)cyclopropyl]carbonyl}amino)benzamide hydrochloride (1:1)

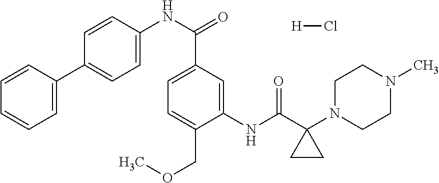 [SMILES: COCc1ccc(C(=O)Nc2ccc(-c3ccccc3)cc2)cc1NC(=O)C1(N2CCN(C)CC2)CC1 ; Cl]

To a solution of the compound of example 81A (145 mg, 0.39 mmol) and the compound of example 63A (72.0 mg, 0.33 mmol) in DMF (1.25 mL) was added (benzotriazol-1-yloxy)tripyrrolidinophosphonium hexafluorophosphate (PYBOP, 341 mg, 0.66 mmol) and diisopropylethylamine (0.23 mL, 1.31 mmol). The resulting mixture was stirred at room temperature over night. The compound of example 63A (72.0 mg, 0.33 mmol), (benzotriazol-1-yloxy)tripyrrolidinophosphonium hexafluorophosphate (PYBOP, 341 mg, 0.66 mmol) and diisopropylethylamine (0.23 mL, 1.31 mmol) were added and the resulting mixture was stirred at room temperature over night. After filtration, purification by HPLC (method 2) yielded 78.0 mg (45% of theory) of the title compound.

¹H-NMR (400 MHz, DMSO-d₆): δ [ppm]=1.21 (s, 4H), 2.79 (s, 3H), 4.65 (s, 2H), 7.31-7.38 (m, 1H), 7.42-7.49 (m, 2H), 7.53 (d, 1H), 7.64-7.71 (m, 4H), 7.76 (dd, 1H), 7.84-7.91 (m, 2H), 8.51 (s, 1H), 9.39 (s, 1H), 10.26 (s, 1H), 10.35 (s, 1H).

LC-MS (Method 1): Rₜ=1.04 min; MS (ESIpos): m/z=499 [M+H−HCl]⁺.

### Example 97

### N-(biphenyl-4-yl)-4-chloro-3-({[1-(morpholin-4-yl)cyclopropyl]carbonyl}amino)benzamide

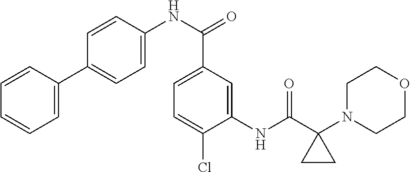 [SMILES: O=C(Nc1ccc(-c2ccccc2)cc1)c1ccc(Cl)c(NC(=O)C2(N3CCOCC3)CC2)c1]

To a solution of biphenyl-4-amine (94.0 mg, 0.55 mmol) and the compound of example 83A (150 mg, 0.46 mmol) in DMF (1.8 mL) was added (benzotriazol-1-yloxy)tripyrrolidinophosphonium hexafluorophosphate (PYBOP, 481 mg, 0.92 mmol) and diisopropylethylamine (0.32 mL, 1.85 mmol). The resulting mixture was stirred at room temperature over night. After filtration, the filtrate was concentrated. The remaining material was then triturated with ethanol (15 mL), and the resulting mixture was stirred for 30 minutes. The remaining solids were removed by filtration, washed with ethanol, and were dried under reduced pressure to give the title compound (168 mg, 75% of theory).

¹H-NMR (300 MHz, DMSO-d₆): δ [ppm]=1.13-1.20 (m, 2H), 1.25-1.32 (m, 2H), 2.45-2.50 (m, 4H), 3.71-3.78 (m, 4H), 7.29-7.39 (m, 1H), 7.41-7.51 (m, 2H), 7.65-7.71 (m, 4H), 7.72-7.76 (m, 2H), 7.82-7.91 (m, 2H), 8.91 (s, 1H), 10.44 (s, 1H), 10.77 (s, 1H).

LC-MS (Method 4): Rₜ=1.43 min; MS (ESIpos): m/z=476 [M+H]⁺.

### Example 98

### N-(biphenyl-4-yl)-3-({[1-(morpholin-4-yl)cyclopropyl]carbonyl}amino)-4-(trifluoromethyl)benzamide

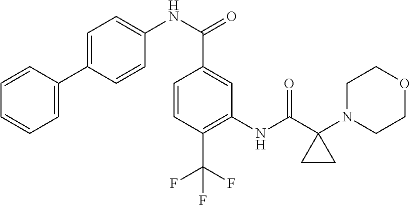 [SMILES: O=C(Nc1ccc(-c2ccccc2)cc1)c1ccc(C(F)(F)F)c(NC(=O)C2(N3CCOCC3)CC2)c1]

200 mg (0.96 mmol, 2 equiv) of the compound of example 65A were stirred in 4 mL of dichloromethane at room temperature. 0.09 mL (1.20 mmol, 2.5 equiv) of DMF and 0.08 mL (0.96 mmol, 2 equiv) of oxalyl chloride were added and the mixture was stirred for additional 0.5 h at room temperature. 0.27 mL (2.41 mmol, 5 equiv) of 4-methylmorpholine and 172 mg (0.48 mmol) of the compound of example 8A were added and the mixture was stirred at room temperature over night and another 24 h at 40° C. The reaction mixture was poured into water and extracted with ethyl acetate. The combined organic phases were dried (Na₂SO₄ anh), and concentrated under reduced pressure. Purification by HPLC (method 2) yielded 12 mg (5% of theory) of the title compound.

¹H-NMR (300 MHz, DMSO-d₆): δ [ppm]=1.14-1.21 (m, 2H), 1.26-1.34 (m, 2H), 2.42-2.49 (m, 4H), 3.65-3.75 (m, 4H), 7.30-7.39 (m, 1H), 7.41-7.51 (m, 2H), 7.64-7.74 (m, 4H), 7.81-7.97 (m, 4H), 8.81 (s, 1H), 10.56 (s, 1H), 10.64 (s, 1H).

LC-MS (Method 4): Rₜ=1.46 min; MS (ESIpos): m/z=510 [M+H]⁺.

### Example 99

### N-(biphenyl-4-yl)-4-methoxy-3-{[2-(morpholin-4-yl)butanoyl]amino}benzamide

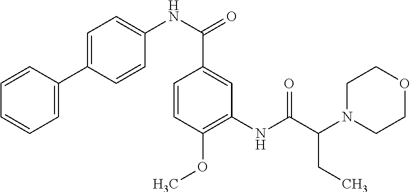 [SMILES: CCC(C(=O)Nc1cc(C(=O)Nc2ccc(-c3ccccc3)cc2)ccc1OC)N1CCOCC1]

To a solution of the compound of example 7A (150 mg, 0.47 mmol) and 2-(morpholin-4-yl)butanoic acid hydrochloride (1:1) (148 mg, 0.71 mmol) in DMF (2 mL) was added (benzotriazol-1-yloxy)tripyrrolidinophosphonium hexafluorophosphate (PYBOP, 490 mg, 0.94 mmol) and diisopropylethylamine (0.41 mL, 2.36 mmol). The resulting mixture was stirred at room temperature over night. (Benzotriazol-1-yloxy)tripyrrolidinophosphonium hexafluorophosphate (PYBOP, 245 mg, 0.47 mmol) and diisopropylethylamine (0.05 mL, 0.47 mmol) were added and the resulting mixture was stirred at room temperature for 3 days. After filtration, purification by HPLC (method 2) yielded 137 mg (60% of theory) of the title compound.

¹H-NMR (300 MHz, DMSO-d₆): δ [ppm]=0.96 (t, 3H), 1.62-1.82 (m, 2H), 2.54-2.67 (m, 4H), 3.14 (t, 1H), 3.60-3.71 (m, 4H), 3.97 (s, 3H), 7.20 (d, 1H), 7.30-7.37 (m, 1H), 7.41-7.50 (m, 2H), 7.63-7.70 (m, 4H), 7.78 (dd, 1H), 7.84-7.90 (m, 2H), 8.67 (d, 1H), 9.69 (s, 1H), 10.25 (s, 1H).

LC-MS (Method 1): Rₜ=1.10 min; MS (ESIpos): m/z=474 [M+H]⁺.

### Example 100

### N-(biphenyl-4-yl)-4-methoxy-3-({[1-(4-methylpiperazin-1-yl)cyclopropyl]carbonyl}amino)benzamide hydrochloride (1:1)

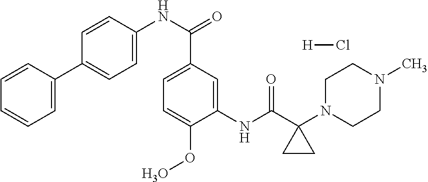

To a solution of the compound of example 7A (125 mg, 0.39 mmol) and the compound of example 63A (72.0 mg, 0.33 mmol) in DMF (1.25 mL) was added (benzotriazol-1-yloxy)tripyrrolidinophosphonium hexafluorophosphate (PYBOP, 341 mg, 0.66 mmol) and diisopropylethylamine (0.23 mL, 1.31 mmol). The resulting mixture was stirred at room temperature over night. The compound of example 63A (72.0 mg, 0.33 mmol), (benzotriazol-1-yloxy)tripyrrolidinophosphonium hexafluorophosphate (PYBOP, 341 mg, 0.66 mmol) and diisopropylethylamine (0.23 mL, 1.31 mmol) were added and the resulting mixture was stirred at room temperature over night. After filtration, purification by HPLC (method 2) yielded 95.0 mg (55% of theory) of the title compound.

¹H-NMR (400 MHz, DMSO-d₆): δ [ppm]=1.14-1.24 (m, 4H), 2.45-2.65 (m, 2H), 2.75-3.17 (m, 4H), 2.86 (s, 3H), 3.35-3.63 (m, 2H), 4.03 (s, 3H), 7.23 (d, 1H), 7.31-7.37 (m, 1H), 7.42-7.49 (m, 2H), 7.63-7.70 (m, 4H), 7.80 (dd, 1H), 7.84-7.89 (m, 2H), 8.70 (d, 1H), 9.43 (s, 1H), 10.08 (s, 1H), 10.22 (s, 1H).

LC-MS (Method 1): Rₜ=1.03 min; MS (ESIpos): m/z=485 [M+H−HCl]⁺.

### Example 101

### N-(biphenyl-4-yl)-4-methoxy-3-({[1-(morpholin-4-yl)cyclopropyl]carbonyl}amino)benzamide

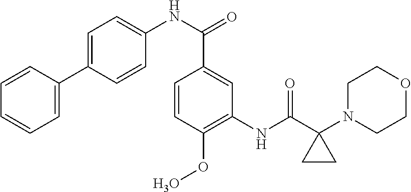 [SMILES: O=C(Nc1ccc(-c2ccccc2)cc1)c1ccc(OO)c(NC(=O)C2(N3CCOCC3)CC2)c1]

To a solution of the compound of example 7A (57.5 mg, 0.18 mmol) and the compound of example 65A (45 mg, 0.22 mmol) in DMF (1 mL) was added (benzotriazol-1-yloxy)tripyrrolidinophosphonium hexafluorophosphate (PYBOP, 188 mg, 0.36 mmol) and diisopropylethylamine (0.16 mL, 0.90 mmol). The resulting mixture was stirred at room temperature over night. After filtration, purification by HPLC (column: chromatorex C18, 10 μm, 125×30 mm, mobile phase: acetonitrile/water gradient with the addition of 0.1% formic acid) yielded 40.6 mg (44% of theory) of the title compound.

¹H-NMR (400 MHz, DMSO-d₆): δ [ppm]=1.10-1.15 (m, 2H), 1.19-1.24 (m, 2H), 2.43-2.48 (m, 4H), 3.69-3.76 (m, 4H), 4.03 (s, 3H), 7.22 (d, 1H), 7.30-7.36 (m, 1H), 7.42-7.49 (m, 2H), 7.63-7.70 (m, 4H), 7.74 (dd, 1H), 7.83-7.89 (m, 2H), 8.85 (d, 1H), 10.22 (s, 1H), 10.63 (s, 1H).

LC-MS (Method 1): Rₜ=1.35 min; MS (ESIpos): m/z=472 [M+H]⁺.

### Example 102

N⁴-(biphenyl-4-yl)-N¹-ethyl-2-[(morpholin-4-ylacetyl)amino]terephthalamide

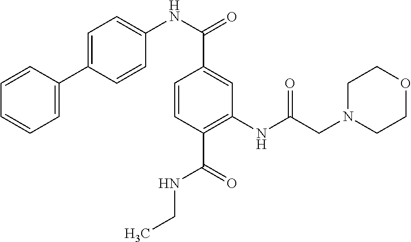 [SMILES: CCNC(=O)c1ccc(C(=O)Nc2ccc(-c3ccccc3)cc2)cc1NC(=O)CN1CCOCC1]

To a solution of the compound of example 84A (100 mg, 0.22 mmol) and ethanamine hydrochloride (1:1) (35.5 mg, 0.44 mmol) in DMF (2 mL) was added (benzotriazol-1-yloxy)tripyrrolidinophosphonium hexafluorophosphate (PYBOP, 227 mg, 0.44 mmol) and diisopropylethylamine (0.19 mL, 1.09 mmol). The resulting mixture was stirred at room temperature over night. After filtration, purification by HPLC (method 2) yielded 45.8 mg (43% of theory) of the title compound.

¹H-NMR (400 MHz, DMSO-d₆): δ [ppm]=1.17 (t, 3H), 2.46-2.58 (m, 4H), 3.16 (s, 2H), 3.32-3.39 (m, 2H), 3.69-3.81 (m, 4H), 7.31-7.38 (m, 1H), 7.42-7.50 (m, 2H), 7.61-7.75 (m, 5H), 7.75-7.82 (m, 1H), 7.84-7.93 (m, 2H), 8.75-8.85 (m, 1H), 9.02 (s, 1H), 10.45 (s, 1H), 11.83 (s, 1H).

LC-MS (Method 1): Rₜ=1.04 min; MS (ESIpos): m/z=487 [M+H]⁺.

### Example 103

N⁴-(biphenyl-4-yl)-2-[(morpholin-4-ylacetyl)amino]-N-[3-(pyrrolidin-1-yl)propyl]terephthalamide

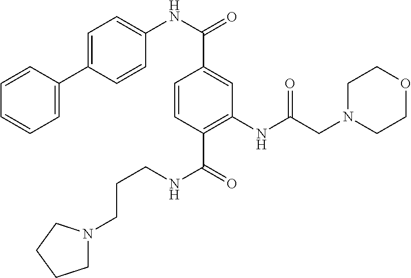 [SMILES: O=C(CN1CCOCC1)Nc1cc(C(=O)Nc2ccc(-c3ccccc3)cc2)ccc1C(=O)NCCCN1CCCC1]

To a solution of the compound of example 84A (100 mg, 0.22 mmol) and 3-(pyrrolidin-1-yl)propan-1-amine (55.8 mg, 0.44 mmol) in DMF (2 mL) was added (benzotriazol-1-yloxy)tripyrrolidinophosphonium hexafluorophosphate (PYBOP, 227 mg, 0.44 mmol) and diisopropylethylamine (0.19 mL, 1.09 mmol). The resulting mixture was stirred at room temperature over night. After filtration, purification by HPLC (Instrument: Waters Autopurificationsystem SQD; column: Waters XBrigde C18 5μ 100×30 mm; water+0.2% vol. ammonia/acetonitrile gradient) yielded 26.3 mg (21% of theory) of the title compound.

¹H-NMR (300 MHz, DMSO-d₆): δ [ppm]=1.63-1.80 (m, 6H), 2.39-2.48 (m, 6H), 2.49-2.60 (m, 4H), 3.15 (s, 2H), 3.36-3.42 (m, 2H), 3.69-3.79 (m, 4H), 7.30-7.38 (m, 1H), 7.42-7.50 (m, 2H), 7.65-7.74 (m, 5H), 7.74-7.80 (m, 1H), 7.84-7.92 (m, 2H), 8.89 (t, 1H), 9.03 (d, 1H), 10.46 (s, 1H), 11.88 (s, 1H).

LC-MS (Method 3): Rₜ=1.41 min; MS (ESIpos): m/z=570 [M+H]⁺.

### Example 104

### N⁴-(biphenyl-4-yl)-N¹-[3-(dimethylamino)propyl]-2-[(morpholin-4-ylacetyl)amino]terephthalamide

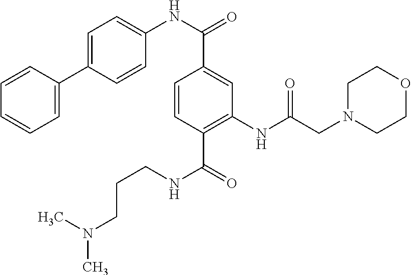 [SMILES: CN(C)CCCNC(=O)c1ccc(C(=O)Nc2ccc(-c3ccccc3)cc2)cc1NC(=O)CN1CCOCC1]

To a solution of the compound of example 84A (100 mg, 0.22 mmol) and N,N-dimethylpropane-1,3-diamine (44.5 mg, 0.44 mmol) in DMF (2 mL) was added (benzotriazol-1-yloxy)tripyrrolidinophosphonium hexafluorophosphate (PYBOP, 227 mg, 0.44 mmol) and diisopropylethylamine (0.19 mL, 1.09 mmol). The resulting mixture was stirred at room temperature over night. After filtration, purification by HPLC (Instrument: Waters Autopurificationsystem SQD; column: Waters XBrigde C18 5μ 100×30 mm; water+0.2% vol. ammonia/methanol gradient) yielded 50.1 mg (42% of theory) of the title compound.

¹H-NMR (300 MHz, DMSO-d₆): δ [ppm]=1.70 (quin, 2H), 2.15 (s, 6H), 2.29 (t, 2H), 2.50-2.56 (m, 4H), 3.15 (s, 2H), 3.33-3.39 (m, 2H), 3.70-3.79 (m, 4H), 7.29-7.39 (m, 1H), 7.41-7.52 (m, 2H), 7.63-7.74 (m, 5H), 7.75-7.81 (m, 1H), 7.84-7.94 (m, 2H), 8.83 (t, 1H), 9.03 (d, 1H), 10.45 (s, 1H), 11.85 (s, 1H).

LC-MS (Method 3): Rₜ=1.27 min; MS (ESIpos): m/z=544 [M+H]⁺.

### Example 105

### formic acid —N⁴-(biphenyl-4-yl)-N¹-[2-(dimethylamino)ethyl]-2-[(morpholin-4-ylacetyl)amino]terephthalamide (1:1)

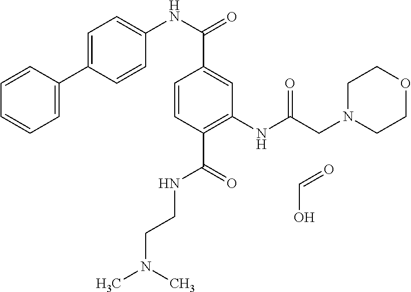

To a solution of the compound of example 84A (100 mg, 0.22 mmol) and N,N-dimethylethane-1,2-diamine (38.4 mg, 0.44 mmol) in DMF (2 mL) was added (benzotriazol-1-yloxy)tripyrrolidinophosphonium hexafluorophosphate (PYBOP, 227 mg, 0.44 mmol) and diisopropylethylamine (0.19 mL, 1.09 mmol). The resulting mixture was stirred at room temperature over night. After filtration, purification by HPLC (method 2) yielded 47.1 mg (34% of theory) of the title compound.

¹H-NMR (300 MHz, DMSO-d₆): δ [ppm]=2.25 (s, 6H), 2.50-2.56 (m, 6H), 3.16 (s, 2H), 3.42 (q, 2H), 3.70-3.80 (m, 4H), 7.30-7.39 (m, 1H), 7.41-7.50 (m, 2H), 7.64-7.74 (m, 5H), 7.75-7.82 (m, 1H), 7.84-7.91 (m, 2H), 8.18 (s, 1H), 8.79 (t, 1H), 9.04 (d, 1H), 10.47 (s, 1H), 11.86 (s, 1H).

LC-MS (Method 4): Rₜ=0.87 min; MS (ESIpos): m/z=530 [M+H−HCO₂H]⁺.

### Example 106

### N⁴-(biphenyl-4-yl)-N¹-(2-methoxyethyl)-2-[(morpholin-4-ylacetyl)amino]terephthalamide

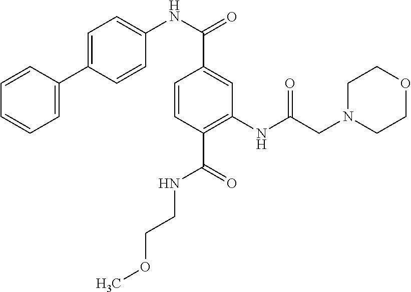 [SMILES: COCCNC(=O)c1ccc(C(=O)Nc2ccc(-c3ccccc3)cc2)cc1NC(=O)CN1CCOCC1]

To a solution of the compound of example 84A (100 mg, 0.22 mmol) and 2-methoxyethanamine (32.7 mg, 0.44 mmol) in DMF (2 mL) was added (benzotriazol-1-yloxy)tripyrrolidinophosphonium hexafluorophosphate (PYBOP, 227 mg, 0.44 mmol) and diisopropylethylamine (0.19 mL, 1.09 mmol). The resulting mixture was stirred at room temperature over night. After filtration, purification by HPLC (method 2) yielded 48.7 mg (42% of theory) of the title compound.

¹H-NMR (300 MHz, DMSO-d₆): δ [ppm]=2.40-2.60 (m, 4H), 3.16 (s, 2H), 3.30 (s, 3H), 3.45-3.55 (m, 4H), 3.70-3.85 (m, 4H), 7.30-7.39 (m, 1H), 7.41-7.51 (m, 2H), 7.65-7.90 (m, 8H), 8.86 (s, 1H), 9.01 (s, 1H), 10.47 (s, 1H), 11.81 (s, 1H).

LC-MS (Method 1): Rₜ=1.02 min; MS (ESIpos): m/z=517 [M+H]⁺.

### Example 107

### N⁴-(biphenyl-4-yl)-N¹-cyclopropyl-2-[(morpholin-4-ylacetyl)amino]terephthalamide

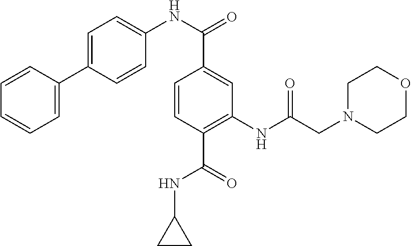 [SMILES: O=C(CN1CCOCC1)Nc1cc(C(=O)Nc2ccc(-c3ccccc3)cc2)ccc1C(=O)NC1CC1]

To a solution of the compound of example 84A (100 mg, 0.22 mmol) and cyclopropanamine (24.9 mg, 0.44 mmol) in DMF (2 mL) was added (benzotriazol-1-yloxy)tripyrrolidinophosphonium hexafluorophosphate (PYBOP, 227 mg, 0.44 mmol) and diisopropylethylamine (0.19 mL, 1.09 mmol). The resulting mixture was stirred at room temperature over night. After filtration, purification by HPLC (1. method 2; 2. Waters Autopurificationsystem, column: XBrigde C18 5 μm 100×30 mm, solvent: water/acetonitrile+0.2% ammonia (32%) gradient, rate: 70 mL/min, temperature: room temperature) yielded 27.0 mg (25% of theory) of the title compound.

¹H-NMR (300 MHz, DMSO-d₆): δ [ppm]=0.58-0.67 (m, 2H), 0.71-0.80 (m, 2H), 2.50-2.58 (m, 4H), 2.86-2.98 (m, 1H), 3.16 (s, 2H), 3.72-3.81 (m, 4H), 7.30-7.38 (m, 1H), 7.41-7.50 (m, 2H), 7.64-7.77 (m, 6H), 7.83-7.91 (m, 2H), 8.76 (d, 1H), 8.99 (d, 1H), 10.44 (s, 1H), 11.74 (s, 1H).

LC-MS (Method 1): Rₜ=1.07 min; MS (ESIpos): m/z=499 [M+H]⁺.

### Example 108

N⁴-(biphenyl-4-yl)-N¹-(3-methoxypropyl)-2-[(morpholin-4-ylacetyl)amino]terephthalamide

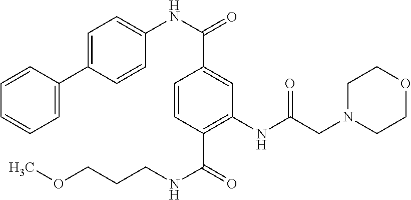 [SMILES: COCCCNC(=O)c1ccc(C(=O)Nc2ccc(-c3ccccc3)cc2)cc1NC(=O)CN1CCOCC1]

To a solution of the compound of example 84A (100 mg, 0.22 mmol) and 3-methoxypropan-1-amine (38.8 mg, 0.44 mmol) in DMF (2 mL) was added (benzotriazol-1-yloxy)tripyrrolidinophosphonium hexafluorophosphate (PYBOP, 227 mg, 0.44 mmol) and diisopropylethylamine (0.19 mL, 1.09 mmol). The resulting mixture was stirred at room temperature over night. After filtration, purification by HPLC (method 2) yielded 47.9 mg (37% of theory) of the title compound.

¹H-NMR (300 MHz, DMSO-d₆): δ [ppm]=1.81 (quin, 2H), 2.50-2.60 (m, 4H), 3.16 (s, 2H), 3.26 (s, 3H), 3.34-3.45 (m, 4H), 3.65-3.84 (m, 4H), 7.30-7.39 (m, 1H), 7.41-7.52 (m, 2H), 7.64-7.84 (m, 6H), 7.84-7.94 (m, 2H), 8.80 (s, 1H), 9.01 (s, 1H), 10.46 (s, 1H), 11.79 (s, 1H).

LC-MS (Method 4): Rₜ=1.07 min; MS (ESIpos): m/z=531 [M+H]⁺.

### Example 109

### N-(biphenyl-4-yl)-4-(methylsulfanyl)-3-[(morpholin-4-ylacetyl)amino]benzamide

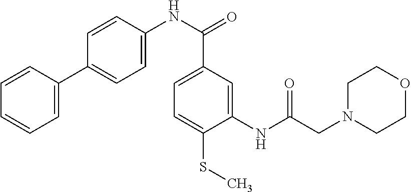 [SMILES: CSc1ccc(C(=O)Nc2ccc(-c3ccccc3)cc2)cc1NC(=O)CN1CCOCC1]

To 1 g (2.99 mmol) of 3-amino-N-(biphenyl-4-yl)-4-(methylsulfanyl)benzamide (example 86A) dissolved in 30 mL of anh DMF were added 521 mg (3.59 mmol) of morpholin-4-ylacetic acid, 1.87 g (3.59 mmol) of PYBOP and 625 μL (3.59 mmol) of N-ethyl-N-isopropylpropan-2-amine. It was stirred over night at 50° C. 217 mg (1.50 mmol) of morpholin-4-ylacetic acid and 778 mg (1.50 mmol) of PYBOP were added. It was stirred for 6 h at 50° C. 217 mg (1.50 mmol) of morpholin-4-ylacetic acid, 778 mg (1.50 mmol) of PYBOP and 0.6 mL (3.44 mmol) of N-ethyl-N-isopropylpropan-2-amine were added and it was stirred at 60° C. over night. The reaction was allowed to reach rt and water was added. It was stirred for 30 min. The solid was removed by suction filtration and washed three times with water. An agglomerate was separated from the fine solid. They were dried under vacuum. To the fine solid was added methanol and it was stirred for 3 h under reflux. It was allowed to reach rt and filtered off yielding 500 mg (36%) of the title compound. The agglomerate was stirred in methanol until a fine solid was obtained. It was stirred at 50° C. The compound was filtered off and dried to afford further 610 mg (43%) of the title compound. 100 mg of the second batch was purified by HPLC (method 2) to yield 34 mg of the title product.

¹H-NMR (300 MHz, DMSO-d₆): δ [ppm]=2.55 (s, 3H), 2.57-2.62 (m, 4H), 3.19 (s, 2H), 3.66-3.74 (m, 4H), 7.29-7.36 (m, 1H), 7.41-7.49 (m, 2H), 7.54 (d, 1H), 7.63-7.71 (m, 4H), 7.78 (dd, 1H), 7.84-7.90 (m, 2H), 8.53 (d, 1H), 9.88 (s, 1H), 10.35 (s, 1H).

LC-MS (method 4): Rₜ=1.12 min; MS (ESIpos): m/z=462 [M+H]⁺.

### Example 110

### N-(biphenyl-4-yl)-4-(methylsulfinyl)-3-[(morpholin-4-ylacetyl)amino]benzamide

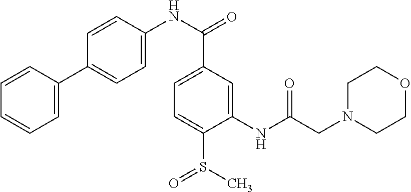 [SMILES: CS(=O)c1ccc(C(=O)Nc2ccc(-c3ccccc3)cc2)cc1NC(=O)CN1CCOCC1]

200 mg (0.43 mmol) of N-(biphenyl-4-yl)-4-(methylsulfanyl)-3-[(morpholin-4-ylacetyl)amino]benzamide (example 109) were dissolved in 1 mL of acetone, 550 μL of methanol and 200 μL of water. 100 mg (0.47 mmol) of sodium periodate were added and it was stirred for 5 days at 45° C. 14 mg (0.065 mmol) of sodium periodate were added and stirred for 3 h at 45° C. The reaction mixture was cooled down and the solid was suction filtered and washed with acetone. The solid was stirred in water. The solid was suction filtered, washed twice with water and dried for 2 days at 45° C. 43.4 mg (21%) of the title compound was isolated.

¹H-NMR (300 MHz, DMSO-d₆): δ [ppm]=2.52-2.59 (m, 4H), 2.88 (s, 3H), 3.12-3.25 (m, 2H), 3.62-3.73 (m, 4H), 7.29-7.38 (m, 1H), 7.40-7.50 (m, 2H), 7.64-7.72 (m, 4H), 7.81-7.95 (m, 4H), 8.47-8.51 (m, 1H), 10.51 (s, 1H), 10.70 (s, 1H).

LC-MS (method 4): Rₜ=0.95 min; MS (ESIpos): m/z=478 [M+H]⁺.

### Example 111

### N-(biphenyl-4-yl)-4-(methylsulfonyl)-3-[(morpholin-4-ylacetyl)amino]benzamide

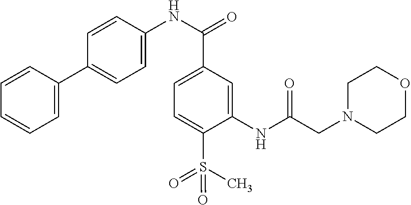 [SMILES: CS(=O)(=O)c1ccc(C(=O)Nc2ccc(-c3ccccc3)cc2)cc1NC(=O)CN1CCOCC1]

80 mg (0.17 mmol) of N-(biphenyl-4-yl)-4-(methylsulfinyl)-3-[(morpholin-4-ylacetyl)amino]benzamide (example 110) were suspended in 7 mL of methanol. 51 mg (0.17 mmol) of Oxone in 2 mL of water were added. It was stirred for 2.5 h at rt. 15 mL of dichloromethane and of aqueous sodium hydrogen sulfite solution (39%) were added and stirred for 10 min. The solid was suction filtered and stirred in 5 mL of water. The residue was filtered off, washed with water and dried under vacuum affording 27 mg (33%) of the title compound.

¹H-NMR (500 MHz, DMSO-d₆): δ [ppm]=2.57-2.63 (m, 4H), 3.23 (s, 2H), 3.35 (s, 3H), 3.69-3.73 (m, 4H), 7.33-7.37 (m, 1H), 7.44-7.48 (m, 2H), 7.66-7.71 (m, 4H), 7.85-7.90 (m, 3H), 8.04 (d, 1H), 9.05-9.07 (m, 1H), 10.59 (s, 1H), 11.03 (s, 1H).

LC-MS (method 4): Rₜ=1.17 min; MS (ESIpos): m/z=494 [M+H]⁺.

### Example 112

### N-(biphenyl-4-yl)-4-(cyclopropyloxy)-3-[(morpholin-4-ylacetyl)amino]benzamide

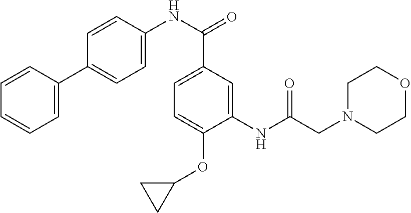 [SMILES: O=C(CN1CCOCC1)Nc1cc(C(=O)Nc2ccc(-c3ccccc3)cc2)ccc1OC1CC1]

To 50 mg (0.15 mmol) of 3-amino-N-(biphenyl-4-yl)-4-(cyclopropyloxy)benzamide (example 88A) in 3.6 mL of anh DMF were added 25.3 mg (0.17 mmol) of morpholin-4-ylacetic acid, 90.7 mg (0.17 mmol) of PYBOP and 76 μL (0.44 mmol) of N-ethyl-N-isopropylpropan-2-amine. It was stirred for 6 h at rt. The reaction mixture was poured into water. It was extracted three times with dichlormethane. The combined organic phases were partly concentrated and the solid was filtered off. The solid was purified by HPLC (Waters Autopurificationsystem SQD; column: XBridge C18 5μ 100×30 mm; eluent A: water+0.2% vol. ammonia (32%), eluent B: acetonitrile; gradient: 0-8.0 min 47-65% B, 70 mL/min; temperature: room temperature; injection: 500-1000 μL; DAD scan: 210-400 nm) to yield 29.3 mg 52%) of the title compound.

¹H-NMR (400 MHz, DMSO-d₆): δ [ppm]=0.73-0.80 (m, 2H), 0.88-0.96 (m, 2H), 2.52-2.58 (m, 4H), 3.15 (s, 2H), 3.63-3.70 (m, 4H), 4.07-4.14 (m, 1H), 7.30-7.36 (m, 1H), 7.41-7.48 (m, 3H), 7.63-7.69 (m, 4H), 7.76 (dd, 1H), 7.83-7.89 (m, 2H), 8.77 (d, 1H), 9.68 (s, 1H), 10.23 (s, 1H).

LC-MS (method 4): Rₜ=1.33 min; MS (ESIpos): m/z=472 [M+H]⁺.

### Example 113

### N-(biphenyl-4-yl)-4-(cyclopropyloxy)-3-({[1-(morpholin-4-yl)cyclopropyl]carbonyl}amino)benzamide

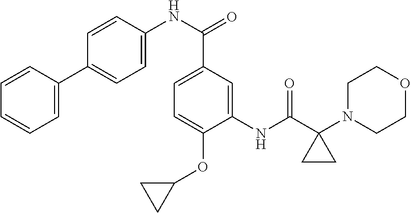 [SMILES: O=C(Nc1ccc(-c2ccccc2)cc1)c1ccc(OC2CC2)c(NC(=O)C2(N3CCOCC3)CC2)c1]

To 50 mg (0.15 mmol) of 3-amino-N-(biphenyl-4-yl)-4-(cyclopropyloxy)benzamide (example 88A) in 3.6 mL of anh DMF were added 29.8 mg (0.17 mmol) of 1-(morpholin-4-yl)cyclopropanecarboxylic acid (example 65A), 90.7 mg (0.17 mmol) of PYBOP and 76 μL (0.44 mmol) of N-ethyl-N-isopropylpropan-2-amine. It was stirred for 3 h at rt and over night at 45° C. The tip of a spatula with PYBOP was added and it was stirred for 4 h at 45° C. The reaction mixture was poured into water. It was extracted three times with dichlormethane. The combined organic layers and the aqueous phase were concentrated and purified by HPLC (method 5) obtaining 32 mg (44%) of the title compound.

¹H-NMR (400 MHz, DMSO-d₆): δ [ppm]=0.78-0.84 (m, 2H), 0.91-0.98 (m, 2H), 1.08-1.15 (m, 2H), 1.16-1.23 (m, 2H), 2.39-2.46 (m, 4H), 3.68-3.75 (m, 4H), 4.09-4.15 (m, 1H), 7.30-7.35 (m, 1H), 7.41-7.48 (m, 3H), 7.63-7.69 (m, 4H), 7.71-7.75 (m, 1H), 7.82-7.88 (m, 2H), 8.84-8.86 (m, 1H), 10.22 (s, 1H), 10.45 (s, 1H).

LC-MS (method 4): Rₜ=1.42 min; MS (ESIpos): m/z=498 [M+H]⁺.

### Example 114

### N-(biphenyl-4-yl)-4-(cyclopropyloxy)-3-({[1-(4-methylpiperazin-1-yl)cyclopropyl]carbonyl}amino)benzamide

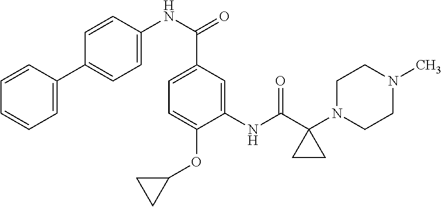 [SMILES: CN1CCN(C2(C(=O)Nc3cc(C(=O)Nc4ccc(-c5ccccc5)cc4)ccc3OC3CC3)CC2)CC1]

To 50 mg (0.15 mmol) of 3-amino-N-(biphenyl-4-yl)-4-(cyclopropyloxy)benzamide (example 88A) in 3.6 mL of anh DMF were added 32.1 mg (0.17 mmol) of 1-(4-methylpiperazin-1-yl)cyclopropanecarboxylic acid (example 63A), 90.7 mg (0.17 mmol) of PYBOP and 76 μL (0.44 mmol) of N-ethyl-N-isopropylpropan-2-amine. It was stirred for 3 h at rt and over night at 45° C. The reaction mixture was poured into water. It was extracted three times with dichlormethane. The combined organic layers were concentrated and purified by HPLC (Waters Autopurificationsystem SQD; column: XBridge C18 5μ 100×30 mm; eluent A: water+0.2% vol. ammonia (32%), eluent B: acetonitrile; gradient: 0-0.5 min 50% B, 25 mL/min to 70 mL/min, 0.5-5.5 min 50-60% B, 70 mL/min; temperature: room temperature; injection: 900 μL; DAD scan: 210-400 nm) obtaining 15.1 mg (19%) of the title compound.

¹H-NMR (300 MHz, DMSO-d₆): δ [ppm]=0.83-0.90 (m, 2H), 0.91-0.98 (m, 2H), 1.08-1.13 (m, 2H), 1.14-1.19 (m, 2H), 2.24 (s, 3H), 2.38-2.46 (m, 4H), 4.08-4.16 (m, 1H), 7.29-7.36 (m, 1H), 7.40-7.49 (m, 3H), 7.61-7.69 (m, 4H), 7.72 (dd, 1H), 7.82-7.88 (m, 2H), 8.88 (d, 1H), 10.22 (s, 1H), 10.45 (s, 1H).

LC-MS (method 4): Rₜ=1.44 min; MS (ESIpos): m/z=511 [M+H]⁺.

### Example 115

### N-(biphenyl-4-yl)-4-(cyclopropyloxy)-3-({[1-(4-cyclopropylpiperazin-1-yl)cyclopropyl]carbonyl}amino)benzamide

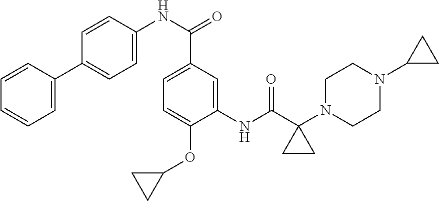 [SMILES: O=C(Nc1ccc(-c2ccccc2)cc1)c1ccc(OC2CC2)c(NC(=O)C2(N3CCN(C4CC4)CC3)CC2)c1]

To 50 mg (0.15 mmol) of 3-amino-N-(biphenyl-4-yl)-4-(cyclopropyloxy)benzamide (example 88A) in 3.6 mL of anh DMF were added 36.6 mg (0.17 mmol) of 1-(4-cyclopropylpiperazin-1-yl)cyclopropanecarboxylic acid (example Example 64A), 90.7 mg (0.17 mmol) of PYBOP and 76 μL (0.44 mmol) of N-ethyl-N-isopropylpropan-2-amine. It was stirred for 3 h at rt and over night at 45° C. The tip of a spatula with PYBOP was added and it was stirred for 4 h at 45° C. The reaction mixture was poured into water. It was extracted three times with dichlormethane. The combined organic layers and the aqueous phase were concentrated and purified by HPLC (method 5) affording 28 mg (36%) of the title compound.

¹H-NMR (400 MHz, DMSO-d₆): δ [ppm]=0.26-0.33 (m, 2H), 0.42-0.49 (m, 2H), 0.84-0.91 (m, 2H), 0.92-0.99 (m, 2H), 1.06-1.12 (m, 2H), 1.12-1.18 (m, 2H), 1.63-1.70 (m, 1H), 2.30-2.44 (m, 4H), 2.58-2.76 (m, 4H), 4.10-4.16 (m, 1H), 7.29-7.35 (m, 1H), 7.41-7.48 (m, 3H), 7.62-7.68 (m, 4H), 7.72 (dd, 1H), 7.82-7.88 (m, 2H), 8.88 (d, 1H), 10.22 (s, 1H), 10.49 (s, 1H).

LC-MS (method 3): Rₜ=1.55 min; MS (ESIpos): m/z=537 [M+H]⁺.

### Example 116

### N⁴-(Biphenyl-4-yl)-2-[(morpholin-4-ylacetyl)amino]terephthalamide

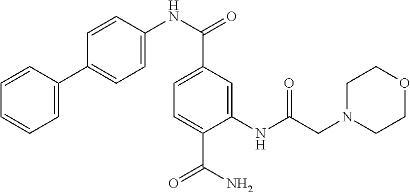 [SMILES: NC(=O)c1ccc(C(=O)Nc2ccc(-c3ccccc3)cc2)cc1NC(=O)CN1CCOCC1]

500 mg (1.06 mmol) of the compound of example 12 were dissolved in 1.5 mL of methanol and 6 mL of THF. 1.37 mL (1.37 mmol) of an aqueous lithium hydroxide solution (1.0M) was added and it was stirred for 4 h at rt. The volatiles were removed and the residue was triturated with dichloromethane. The solvent was removed yielding 510 mg (104%) of a lithium salt, which was used without further purification. 100 mg of the crude material and 4.2 mL (2.10 mmol) of ammonia (5.0 M in THF) were dissolved in 2.5 mL of anh DMF. 165.6 mg (0.32 mmol) of PYBOP and 111 μL (0.64 mmol) of N-ethyl-N-isopropylpropan-2-amine were added. It was stirred for 24 h at rt. The reaction mixture was poured into water. It was extracted three times with a mixture of dichloromethane/isopropanol 4:1. The combined organic phases were dried over sodium sulfate, concentrated and crystallized from methanol to give 70 mg of solid material, which was purified by HPLC (Waters Autopurificationsystem SQD; column: XBridge C18 5μ 100×30 mm; eluent A: water+0.1% vol. formic acid (99%), eluent B: acetonitrile; gradient: 0.0-8.0 min 15-50% B, 50 mL/min; temperature: room temperature; injection: 500 μL; DAD scan: 210-400 nm) yielding 10.2 mg (11%) of the title compound.

¹H-NMR (500 MHz, DMSO-d₆): δ [ppm]=2.52-2.56 (m, 4H, and DMSO signal), 3.17 (s, 2H), 3.72-3.76 (m, 4H), 7.33-7.38 (m, 1H), 7.45-7.49 (m, 2H), 7.67-7.72 (m, 5H), 7.85-7.91 (m, 4H), 8.31 (s, 1H), 9.11 (d, 1H), 10.46 (s, 1H), 12.19 (s, 1H).

LC-MS (method 3): Rₜ=1.12 min; MS (ESIpos): m/z=459 [M+H]⁺.

### Example 117

### N-(biphenyl-4-yl)-4-(2-hydroxypropan-2-yl)-3-[(morpholin-4-ylacetyl)amino]benzamide

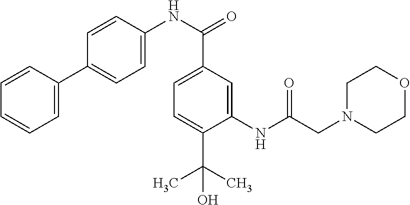 [SMILES: CC(C)(O)c1ccc(C(=O)Nc2ccc(-c3ccccc3)cc2)cc1NC(=O)CN1CCOCC1]

75 mg (0.16 mmol) of the compound of example 12 were dissolved in 5 mL of anh THF. 566 μL (0.79 mmol) of methyl magnesium bromide (1.4M in THF/toluene 1:3) were added according to the following procedure. First two equivalents were added and it was stirred for 30 min at rt. Then three equivalents were added and it was stirred for 8 h at rt. The reaction mixture was poured into saturated aqueous ammonium chloride solution. It was extracted three times with a mixture of dichloromethane/isopropanol 4:1. The combined organic phases were dried over sodium sulfate and concentrated. It was purified by HPLC (Waters Autopurificationsystem SQD; column: XBridge C18 5μ100×30 mm; eluent A: water+0.2% vol. ammonia (32%), eluent B: acetonitrile; gradient: 0.0-8.0 min 40-80% B, 50 mL/min; temperature: room temperature; injection: 600 μL; DAD scan: 210-400 nm) affording 32 mg (43%) of the title compound.

¹H-NMR (300 MHz, DMSO-d₆): δ [ppm]=1.54 (s, 6H), 2.49-2.55 (m, 4H, and DMSO signal), 3.11 (s, 2H), 3.63-3.70 (m, 4H), 5.91 (s, 1H), 7.26-7.34 (m, 1H), 7.37-7.46 (m, 3H), 7.57 (dd, 1H), 7.60-7.67 (m, 4H), 7.80-7.87 (m, 2H), 8.87 (d, 1H), 10.25 (s, 1H), 11.47 (s, 1H).

LC-MS (method 3): Rₜ=1.23 min; MS (ESIpos): m/z=474 [M+H]⁺.

### Example 118

### 4′-acetamido-N-{4-methoxy-3-[(morpholin-4-ylacetyl)amino]phenyl}biphenyl-4-carboxamide

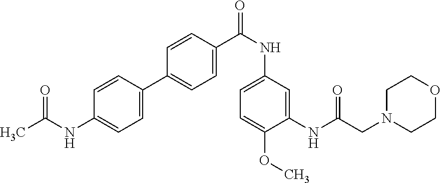 [SMILES: COc1ccc(NC(=O)c2ccc(-c3ccc(NC(C)=O)cc3)cc2)cc1NC(=O)CN1CCOCC1]

A solution of the compound of example 59A (152 mg, 339 μmol) and (4-acetamidophenyl)boronic acid (91.0 mg, 509 μmol) in a mixture of DMF/water (1.96 mL/190 μL) was treated with sodium carbonate (108 mg, 1.02 mmol). Argon was bubbled through this suspension for 5 min, afterwards [1,1′-bis(diphenylphosphino)ferrocene]palladium(II) chloride (Pd(dppf)Cl₂, 24.8 mg, 24 μmol) was added and the tube was sealed. The reaction mixture was stirred for 3 days at 90° C. After cooling to room temperature the mixture was filtered over a pad of Celite. The filtrate was purified by preparative HPLC (method 5) to yield the desired product 118 (22 mg, 13%).

¹H-NMR (400 MHz, DMSO-d₆): δ [ppm]=2.08 (s, 3H), 2.55-2.57 (m, 4H), 3.16 (s, 2H), 3.67-3.69 (m, 4H), 3.89 (s, 3H), 7.02-7.09 (m, 1H), 7.54-7.63 (m, 1H), 7.67-7.73 (m, 4H), 7.75-7.80 (m, 2H), 8.00-8.06 (m, 2H), 8.54-8.62 (m, 1H), 9.67-9.77 (m, 1H), 10.06 (s, 1H), 10.21 (s, 1H).

LC-MS (Method 4): Rₜ=1.02 min; MS (ESIpos): m/z=503 [M+H]⁺.

### Example 119

### N-{4-methoxy-3-[(morpholin-4-ylacetyl)amino]phenyl}-4′-(methylamino)biphenyl-4-carboxamide

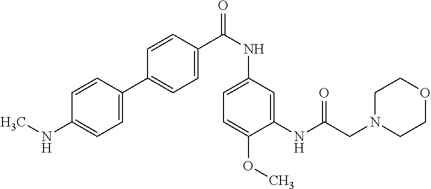 [SMILES: CNc1ccc(-c2ccc(C(=O)Nc3ccc(OC)c(NC(=O)CN4CCOCC4)c3)cc2)cc1]

To a solution of the compound of example 83 (761 mg, 1.65 mmol) and paraformaldehyde (49.6 mg, 1.65 mmol) in a mixture of THF/methanol 1:1 (4.41 mL/4.41 mL) was added sodium cyanoborohydride (437 mg, 6.61 mmol). The reaction mixture was stirred at 40° C. over night. The mixture was diluted with ethyl acetate and brine. The resulting precipitate was removed by filtration and the layers were separated. The organic layer was dried by the use of a silicon filter and concentrated. The remaining residue was purified by preparative HPLC (method 5) to obtain the desired material (84.9 mg, 10%).

¹H-NMR (400 MHz, DMSO-d₆): δ [ppm]=2.54-2.58 (m, 4H), 2.73 (d, 3H), 3.15 (s, 2H), 3.66-3.69 (m, 4H), 3.89 (s, 3H), 5.90-5.95 (m, 1H), 6.60-6.68 (m, 2H), 7.08-7.00 (m, 1H), 7.53-7.60 (m, 3H), 7.65-7.74 (m, 2H), 7.95-8.03 (m, 2H), 8.58-8.60 (m, 1H), 10.13 (s, 1H).

LC-MS (Method 4): Rₜ=0.89 min; MS (ESIpos): m/z=475 [M+H]⁺.

### Example 120

### 4′-(aminomethyl)-N-{4-methoxy-3-[(morpholin-4-ylacetyl)amino]phenyl}biphenyl-4-carboxamide

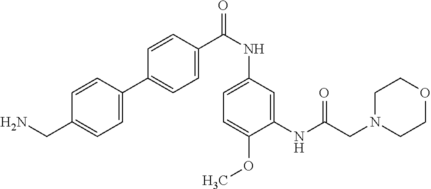 [SMILES: COc1ccc(NC(=O)c2ccc(-c3ccc(CN)cc3)cc2)cc1NC(=O)CN1CCOCC1]

A solution of the compound of example 89A (104 mg, 89 μmol) in DCM (1.42 mL) was treated with trifluoroacetic acid (137 μL, 1.77 mmol) and was stirred over night at room temperature. The mixture was diluted with aqueous, half-saturated NaHCO₃-solution and stirred over night. The precipitate was collected by filtration and purified by flash-chromatography (eluent: hexane/DCM, DCM/methanol, gradient) to obtain the desired compound 120 (14.5 mg, 30 μmol, 34%).

¹H-NMR (400 MHz, DMSO-d₆): δ [ppm]=1.83-2.03 (br. s, 2H), 2.54-2.61 (m, 4H), 3.18 (s, 2H), 3.65-3.71 (m, 4H), 3.74 (s, 2H), 3.99 (s, 3H), 7.18-7.24 (m, 1H), 7.41 (s, 2H), 7.57-7.68 (m, 4H), 7.74-7.80 (m, 1H), 7.85 (d, 2H), 8.75-8.80 (m, 1H), 9.75-9.81 (m, 1H), 10.18-10.24 (m, 1H).

LC-MS (Method 1): Rₜ=0.67 min; MS (ESIpos): m/z=475 [M+H]⁺.

### Example 121

### N-(biphenyl-4-yl)-4-(3-hydroxypropoxy)-3-[(morpholin-4-ylacetyl)amino]benzamide

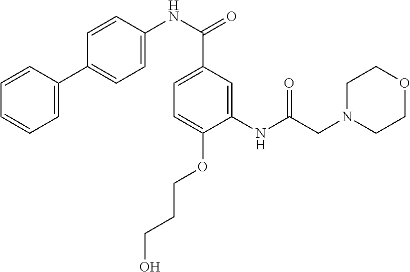 [SMILES: O=C(CN1CCOCC1)Nc1cc(C(=O)Nc2ccc(-c3ccccc3)cc2)ccc1OCCCO]

Example 24A (471 mg, 1.04 mmol) was dissolved in DMF (10.8 mL) and potassium iodide (429.9 mg, 3.11 mmol) and 3-chloro-1-propanol (95 μL, 1.14 mmol) were added. The reaction mixture was stirred in a sealed tube at 100° C. over night. After cooling to room temperature the mixture was filtered. The filtrate was concentrated in vacuum, the residue was purified by preparative HPLC (eluent: acetonitrile/water+NH₃, gradient) to yield the desired product 121 (110 mg, 21.5%).

¹H-NMR (400 MHz, DMSO-d₆): δ [ppm]=2.00 (t, 2H), 2.53-2.60 (m, 4H), 3.18 (s, 2H), 3.58-3.76 (m, 6H), 4.25 (t, 2H), 4.65 (t, 1H), 7.20-7.23 (m, 1H), 7.30-7.38 (m, 1H), 7.42-7.49 (m, 2H), 7.65-7.68 (m, 4H), 7.73-7.75 (m, 1H), 7.85-7.88 (m, 2H), 8.86 (d, 1H), 9.74 (s, 1H), 10.22 (s, 1H).

LC-MS (Method 4): Rₜ=1.01 min; MS (ESIpos): m/z=490 [M+H]⁺.

### Example 122

### 4-(2-amino-2-oxoethoxy)-N-(biphenyl-4-yl)-3-[(morpholin-4-ylacetyl)amino]benzamide

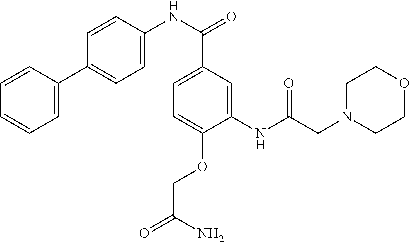 [SMILES: NC(=O)COc1ccc(C(=O)Nc2ccc(-c3ccccc3)cc2)cc1NC(=O)CN1CCOCC1]

A solution of the compound of example 24A (150 mg, 348 μmol) in DMF (5.0 mL) was treated with 2-bromoacetamide (76.6 mg, 556 μmol), cesium carbonate (197 mg, 6.50 mmol) and tetrabutylammonium iodide (5.01 mg, 13.6 μmol). The reaction mixture was stirred at 90° C. in a sealed tube under argon. After cooling to room temperature the mixture was poured into water and the solvent was removed under reduced pressure. The residue was purified by preparative HPLC (method 5) to obtain the desired compound 122 (7.0 mg, 4%).

¹H-NMR (400 MHz, DMSO-d₆): δ [ppm]=2.53-2.59 (m, 4H), 3.18 (s, 2H), 3.62-3.73 (m, 4H), 4.67 (s, 2H), 7.12-7.14 (m, 1H), 7.33-7.36 (m, 1H), 7.43-7.48 (m, 3H), 7.64-7.77 (m, 6H), 7.84-7.88 (m, 2H), 8.71 (d, 1H), 9.86 (s, 1H), 10.24 (s, 1H).

LC-MS (Method 4): Rₜ=0.94 min; MS (ESIpos): m/z=489 [M+H]⁺.

### Example 123

### 4-methoxy-3-[(morpholin-4-ylacetyl)amino]-N-(2,3′,5′-trifluorobiphenyl-4-yl)benzamide

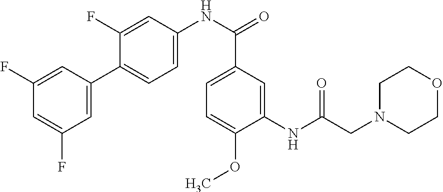 [SMILES: COc1ccc(C(=O)Nc2ccc(-c3cc(F)cc(F)c3)c(F)c2)cc1NC(=O)CN1CCOCC1]

The title compound was prepared in a manner analogous to that described in example 118 starting from 87.5 mg (188 μmol) of example 90A and 59.3 mg (375 μmol) of (3,5-difluorophenyl)boronic acid. To work up the reaction, the mixture was filtered over a pad of Celite. The filtrate was concentrated in vacuum and the residue was purified by preparative HPLC (method 2) to yield 7.1 mg (7%) of the desired compound 123.

¹H-NMR (300 MHz, DMSO-d₆): δ [ppm]=2.54-2.60 (m, 4H), 3.16 (s, 2H), 3.62-3.73 (m, 4H), 3.90 (s, 3H), 7.05-7.08 (m, 1H), 7.30-7.44 (m, 2H), 7.59 (d, 1H), 7.67-7.83 (m, 2H), 7.90-7.98 (m, 2H), 8.58 (d, 1H), 9.75 (s, 1H), 10.33 (s, 1H).

LC-MS (Method 4): Rₜ=1.01 min; MS (ESIpos): m/z=498 [M+H]⁺.

### Example 124

### N-(biphenyl-4-yl)-4-[(methylsulfonyl)methyl]-3-[(morpholin-4-ylacetyl)amino]benzamide

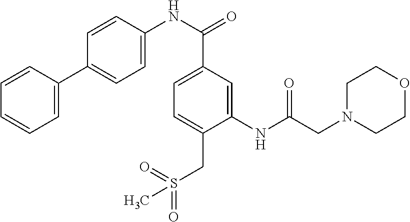 [SMILES: CS(=O)(=O)Cc1ccc(C(=O)Nc2ccc(-c3ccccc3)cc2)cc1NC(=O)CN1CCOCC1]

A solution of the compound of example 96A (100 mg, 219 μmol) in DMF (0.94 mL) was treated with morpholine (29 μL, 328 μmol), triethylamine (46 μL, 328 μmol) and potassium iodide (5.6 mg, 34 lμmol). The mixture was stirred over night at room temperature. After addition of water, the mixture was extracted three times with DCM. The combined organic layers were dried by the use of a silicon filter, the solvent was removed under reduced pressure. The residue was purified by preparative HPLC (method 5) and by trituration with ethanol to yield the desired compound 124 (26.7 mg, 24%).

¹H-NMR (400 MHz, DMSO-d₆): δ [ppm]=2.57-2.63 (m, 4H), 3.05 (s, 3H), 3.17 (s, 2H), 3.63-3.72 (m, 4H), 4.67 (s, 2H), 7.30-7.36 (m, 1H), 7.44-7.48 (m, 2H), 7.58-7.71 (m, 5H), 7.79-7.89 (m, 3H), 8.29 (d, 1H), 9.96 (s, 1H), 10.40 (s, 1H).

LC-MS (Method 4): Rₜ=1.00 min; MS (ESIpos): m/z=508 [M+H]⁺.

### Example 125

### N-(biphenyl-4-yl)-3-{[(4-methylpiperazin-1-yl)acetyl]amino}-4-[(methylsulfonyl)methyl]benzamide

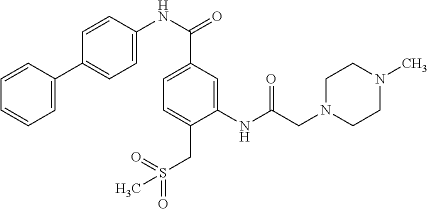 [SMILES: CN1CCN(CC(=O)Nc2cc(C(=O)Nc3ccc(-c4ccccc4)cc3)ccc2CS(C)(=O)=O)CC1]

A solution of the compound of example 96A (100 mg, 219 μmol) in DMF (0.94 mL) was treated with 1-methylpiperazine (33.2 mg, 328 μmol), triethylamine (46 μL, 328 μmol) and potassium iodide (5.6 mg, 34 μmol). The mixture was stirred over night at room temperature. After addition of water, the mixture was extracted three times with DCM. The combined organic layers were dried by the use of a silicon filter, the solvent was removed under reduced pressure. The residue was purified by preparative HPLC (method 5) to yield the desired compound 125 (3.6 mg, 3%).

¹H-NMR (500 MHz, DMSO-d₆): δ [ppm]=2.18 (s, 3H), 2.40-2.45 (m, 4H), 2.55-2.61 (m, 4H), 3.04 (s, 3H), 3.13-3.18 (m, 2H), 4.60-4.67 (m, 2H), 7.31-7.38 (m, 1H), 7.43-7.49 (m, 2H), 7.61-7.65 (m, 1H), 7.66-7.71 (m, 4H), 7.79-7.83 (m, 1H), 7.84-7.90 (m, 2H), 8.27 (s, 1H), 9.856 (s, 1H), 10.40 (s, 1H).

LC-MS (Method 4): Rₜ=1.00 min; MS (ESIpos): m/z=521 [M+H]⁺.

### Example 126

### 4-(3-acetamidopropoxy)-N-(biphenyl-4-yl)-3-[(morpholin-4-ylacetyl)amino]benzamide

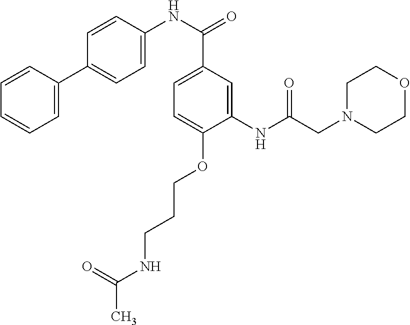 [SMILES: CC(=O)NCCCOc1ccc(C(=O)Nc2ccc(-c3ccccc3)cc2)cc1NC(=O)CN1CCOCC1]

A solution of crude material of example 98A (250 mg), pyridine (385 μL, 4.76 mmol) and N,N-diisopropylethylamine (829 μL, 4.76 mmol) in DCM (2.0 mL) was treated with acetanhydride (89 μL, 952 μmol) and was stirred over night at room temperature. The solvent was removed under reduced pressure and the residue was purified by preparative HPLC (eluent: acetonitrile/water+0.1% HCOOH, gradient) to obtain the desired product 126 (25 mg).

¹H-NMR (500 MHz, DMSO-d₆): δ [ppm]=1.81 (s, 3H), 1.98 (quin, 2H), 2.53-2.59 (m, 4H), 3.19 (s, 2H), 3.63-3.69 (m, 4H), 4.15-4.24 (m, 2H), 7.15-7.22 (m, 1H), 7.29-7.36 (m, 1H), 7.45 (s, 2H), 7.62-7.70 (m, 4H), 7.73-7.79 (m, 1H), 7.85-7.89 (m, 2H), 7.92-8.00 (m, 1H), 8.81-8.87 (m, 1H), 9.69-9.73 (m, 1H), 10.23 (br. s, 1H).

LC-MS (Method 4): Rₜ=0.98 min; MS (ESIpos): m/z=531 [M+H]⁺.

### Example 127

### N-(biphenyl-4-yl)-3-[(morpholin-4-ylacetyl)amino]-4-(2,2,2-trifluoroethoxy)benzamide

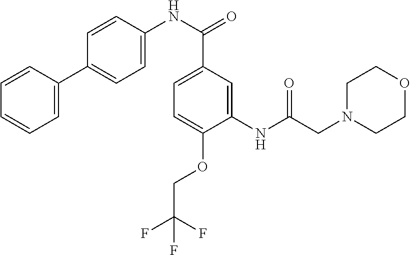 [SMILES: O=C(CN1CCOCC1)Nc1cc(C(=O)Nc2ccc(-c3ccccc3)cc2)ccc1OCC(F)(F)F]

A solution of the compound of example 24A (100 mg, 232 μmol) in DMF (3.33 mL) was treated with potassium carbonate (131 mg, 950 μmol), tetra-n-butylammonium iodide (3.34 mg, 9 μmol) and 2-bromo-1,1,1-trifluoroethane (60.4 mg, 371 μmol). The resulting suspension was stirred in a sealed tube over night at 90° C. After cooling to room temperature the reaction mixture was filtered, the filtrate was concentrated and the residue was purified by preparative HPLC (eluent: acetonitrile/water+0.1% HCOOH, gradient) to yield the desired product 127 (73.6 mg, 59%).

¹H-NMR (400 MHz, DMSO-d₆): δ [ppm]=2.53-2.59 (m, 4H), 3.20 (s, 2H), 3.62-3.68 (m, 4H), 5.02 (q, 2H), 7.29-7.38 (m, 2H), 7.44-7.47 (m, 2H), 7.62-7.70 (m, 4H), 7.78 (dd, 1H), 7.84-7.90 (m, 2H), 8.90 (d, 1H), 9.78 (s, 1H), 10.29 (s, 1H).

LC-MS (Method 1): Rₜ=1.18 min; MS (ESIpos): m/z=514 [M+H]⁺.

The following examples were prepared in analogy to the described methods, supra.

TABLE 1

| | | | Rₜ |
|---|---|---|---|
| Example | | | [min] |
| No | Structure | IUPAC Name | method |
| 128 | 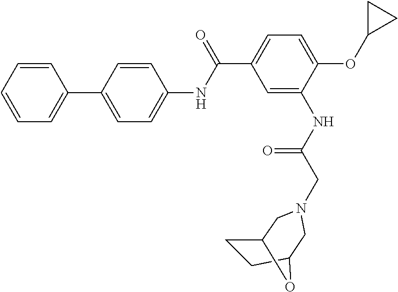 [SMILES: O=C(CN1CC2CCC(C1)O2)Nc1cc(C(=O)Nc2ccc(-c3ccccc3)cc2)ccc1OC1CC1] | N-(biphenyl-4-yl)-4- (cyclopropyloxy)-3-[(8-oxa- 3-azabicyclo[3.2.1]oct-3- ylacetyl)amino]benzamide | 1.277 |
| 129 | 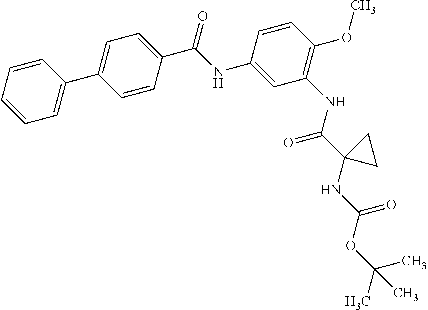 [SMILES: COc1ccc(NC(=O)c2ccc(-c3ccccc3)cc2)cc1NC(=O)C1(NC(=O)OC(C)(C)C)CC1] | tert-butyl[1-({5-[(biphenyl- 4-ylcarbonyl)amino]-2- methoxyphenyl}carbamoyl) cyclopropyl]carbamate | 1.37 |
| 130 | 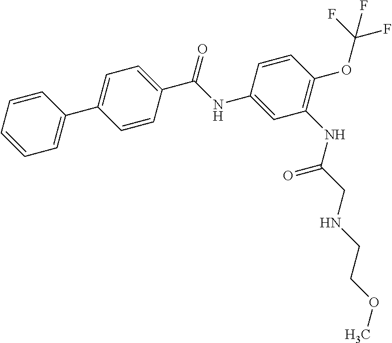 [SMILES: COCCNCC(=O)Nc1cc(NC(=O)c2ccc(-c3ccccc3)cc2)ccc1OC(F)(F)F] | N-[3-{[N-(2- methoxyethyl)glycyl]amino} -4- (trifluoromethoxy)phenyl] biphenyl-4-carboxamide | 0.897 |
| 131 | 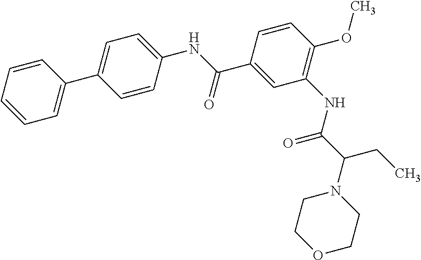 [SMILES: CCC(C(=O)Nc1cc(C(=O)Nc2ccc(-c3ccccc3)cc2)ccc1OC)N1CCOCC1] | N-(biphenyl-4-yl)-4- methoxy-3-{[(2R*)-2- (morpholin-4- yl)butanoyl)amino} benzamide | 0.927 |
| 132 | 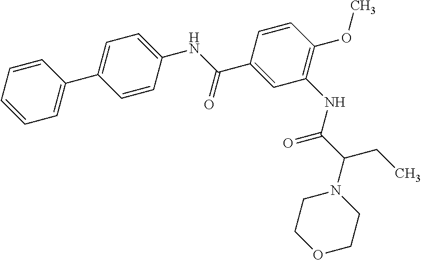 [SMILES: CCC(C(=O)Nc1cc(C(=O)Nc2ccc(-c3ccccc3)cc2)ccc1OC)N1CCOCC1] | N-(biphenyl-4-yl)-4- methoxy-3-{[(2S*)-2- (morpholin-4- yl)butanoyl]amino} benzamide | 0.927 |
| 133 | 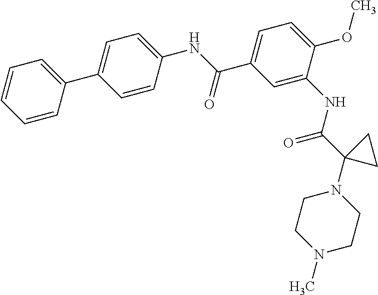 [SMILES: COc1ccc(C(=O)Nc2ccc(-c3ccccc3)cc2)cc1NC(=O)C1(N2CCN(C)CC2)CC1] | N-(biphenyl-4-yl)-4- methoxy-3-({[1-(4- methylpiperazin-1- yl)cyclopropyl]carbonyl} amino)benzamide | 0.877 |
| 134 | 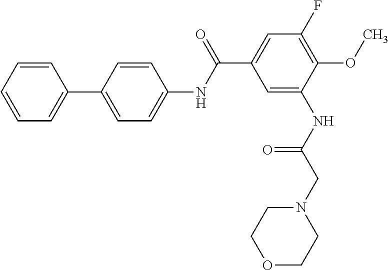 [SMILES: COc1c(F)cc(C(=O)Nc2ccc(-c3ccccc3)cc2)cc1NC(=O)CN1CCOCC1] | N-(biphenyl-4-yl)-3-fluoro-4- methoxy-5-[(morpholin-4- ylacetyl)amino]benzamide | 0.987 |
| 135 | 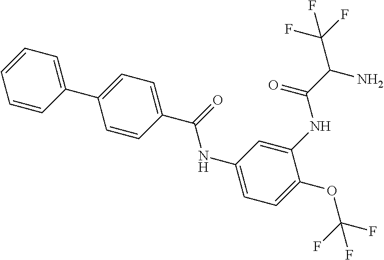 [SMILES: NC(C(=O)Nc1cc(NC(=O)c2ccc(-c3ccccc3)cc2)ccc1OC(F)(F)F)C(F)(F)F] | N-{3-[(333- trifluoroalanyl)amino]-4- [trifluoromethoxy)phenyl) biphenyl-4-carboxamide | |
| 136 | 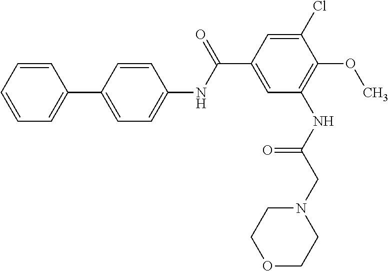 [SMILES: COc1c(Cl)cc(C(=O)Nc2ccc(-c3ccccc3)cc2)cc1NC(=O)CN1CCOCC1] | N-(biphenyl-4-yl)-3-chloro- 4-methoxy-5-[(morpholin-4- ylacetyl]amino]benzamide | 1.057 |
| 137 | 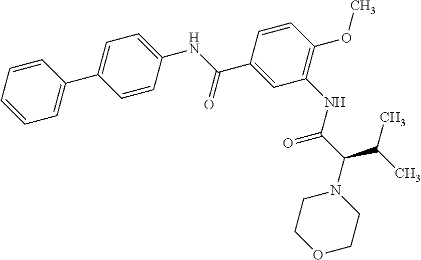 [SMILES: COc1ccc(C(=O)Nc2ccc(-c3ccccc3)cc2)cc1NC(=O)[C@@H](C(C)C)N1CCOCC1] | N-(biphenyl-4-yl)-4- methoxy-3-{[(2R)-3-methyl- 2-(morpholin-4- yl)butanoyl]amino} benzamide | 1.027 |
| 138 | 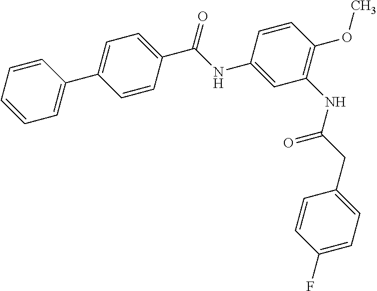 [SMILES: COc1ccc(NC(=O)c2ccc(-c3ccccc3)cc2)cc1NC(=O)Cc1ccc(F)cc1] | N-(3-{[(4- fluorophenyl)acetyl]amino}- 4-methoxyphenyl)biphenyl- 4-carboxamide | 1.317 |
| 139 | 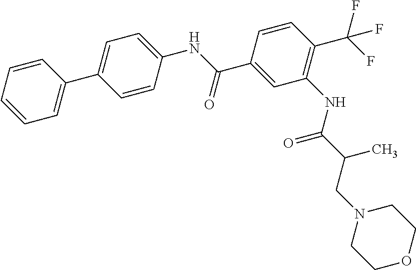 [SMILES: CC(CN1CCOCC1)C(=O)Nc1cc(C(=O)Nc2ccc(-c3ccccc3)cc2)ccc1C(F)(F)F] | N-(biphenyl-4-yl)-3-{[2- methyl-3-(morpholin-4- yl)propanoyl]amino}-4- (trifluoromethyl)benzamide | 0.897 |
| 140 | 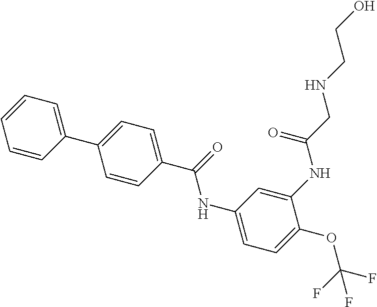 [SMILES: O=C(CNCCO)Nc1cc(NC(=O)c2ccc(-c3ccccc3)cc2)ccc1OC(F)(F)F] | N-[3-{[N-(2- hydroxyethyl)glycyl]amino}- 4- (trifluoromethoxy)phenyl] biphenyl-4-carboxamide | 0.847 |
| 141 | 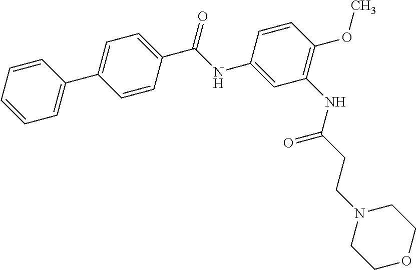 [SMILES: COc1ccc(NC(=O)c2ccc(-c3ccccc3)cc2)cc1NC(=O)CCN1CCOCC1] | N-(4-methoxy-3-{[3- (morpholin-4- yl)propanoyl]amino}phenyl) biphenyl-4-carboxamide | 0.797 |
| 142 | 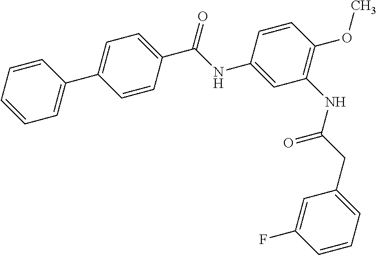 [SMILES: COc1ccc(NC(=O)c2ccc(-c3ccccc3)cc2)cc1NC(=O)Cc1cccc(F)c1] | N-(3-{[(3- fluorophenyl)acetyl]amino}- 4-methoxyphenyl)biphenyl- 4-carboxamide | 1.327 |
| 143 | 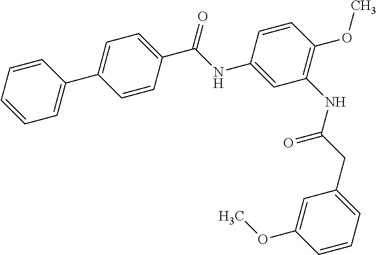 [SMILES: COc1cccc(CC(=O)Nc2cc(NC(=O)c3ccc(-c4ccccc4)cc3)ccc2OC)c1] | N-(4-methoxy-3-{[(3- methoxyphenyl}acetyl] amino}phenyl)biphenyl-4- carboxamide | 1.37 |
| 144 | 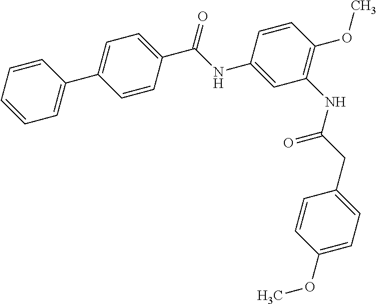 [SMILES: COc1ccc(CC(=O)Nc2cc(NC(=O)c3ccc(-c4ccccc4)cc3)ccc2OC)cc1] | N-(4-methoxy-3-{[(4- methoxyphenyl)acetyl] amino}phenyl)biphenyl-4- carboxamide | 1.37 |
| 145 | 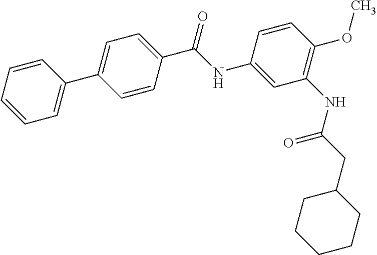 [SMILES: COc1ccc(NC(=O)c2ccc(-c3ccccc3)cc2)cc1NC(=O)CC1CCCCC1] | N-{3- ((cyclohexylacetyl)amino]-4- methoxyphenyl}biphenyl-4- carboxamide | 1.437 |
| 146 | 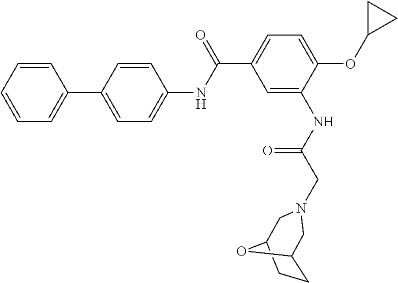 [SMILES: O=C(CN1CC2CCC(C1)O2)Nc1cc(C(=O)Nc2ccc(-c3ccccc3)cc2)ccc1OC1CC1] | N-(biphenyl-4-yl)-4- (methoxymethyl)-3-{[(2R*)- 2-(8-oxa-3- azabicyclo[3.2.1]oct-3- yl)propanoyl)amino} benzamide | 1.127 |
| 147 | 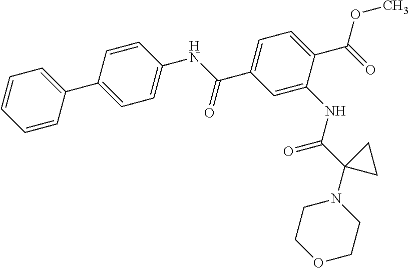 [SMILES: COC(=O)c1ccc(C(=O)Nc2ccc(-c3ccccc3)cc2)cc1NC(=O)C1(N2CCOCC2)CC1] | methyl 4-(biphenyl-4- ylcarbamoyl)-2-({[1- (morpholin-4- yl)cyclopropyl]carbonyl} amino)benzoate | 1.47 |
| 148 | 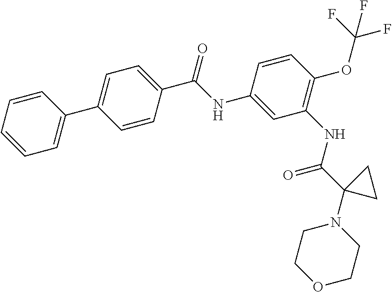 [SMILES: O=C(Nc1ccc(OC(F)(F)F)c(NC(=O)C2(N3CCOCC3)CC2)c1)c1ccc(-c2ccccc2)cc1] | N-[3-({[1-(morpholin-4- yl)cyclopropyl]carbonyl} amino)-4- (trifluoromethoxy)phenyl] biphenyl-4-carboxamide | 1.457 |
| 149 | 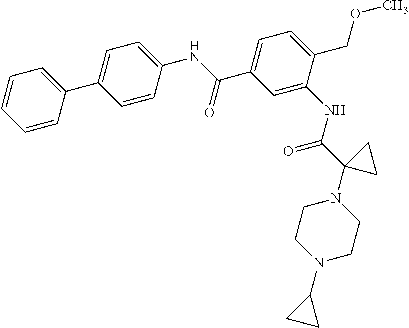 [SMILES: COCc1ccc(C(=O)Nc2ccc(-c3ccccc3)cc2)cc1NC(=O)C1(N2CCN(C3CC3)CC2)CC1] | N-(biphenyl-4-yl)-3-({[1-(4- cyclopropylpiperazin-1- yl)cyclopropyl]carbonyl} amino)-4- (methoxymethyl)benzamide | 0.927 |
| 150 | 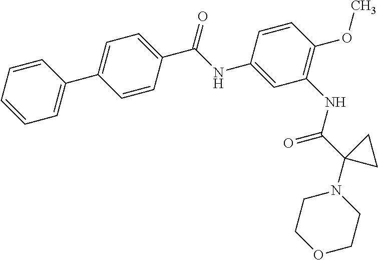 [SMILES: COc1ccc(NC(=O)c2ccc(-c3ccccc3)cc2)cc1NC(=O)C1(N2CCOCC2)CC1] | N-[4-methoxy-3-({[1- (morpholin-4- yl)cyclopropyl]carbonyl} amino)phenyl]biphenyl-4- carboxamide | 1.297 |
| 151 | 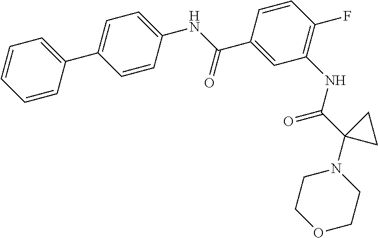 [SMILES: O=C(Nc1ccc(-c2ccccc2)cc1)c1ccc(F)c(NC(=O)C2(N3CCOCC3)CC2)c1] | N-(biphenyl-4-yl)-4-fluoro-3- ({[1-(morpholin-4- yl)cyclopropyl]carbonyl} amino)benzamide | 1.317 |
| 152 | 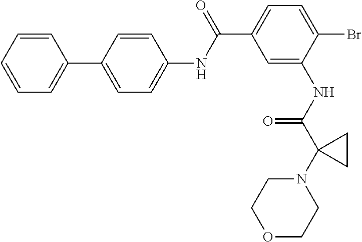 [SMILES: O=C(Nc1ccc(-c2ccccc2)cc1)c1ccc(Br)c(NC(=O)C2(N3CCOCC3)CC2)c1] | N-(biphenyl-4-yl)-4-bromo- 3-({[1-(morpholin-4- yl)cyclopropyl]carbonyl} amino)benzamide | 1.47 |
| 153 | 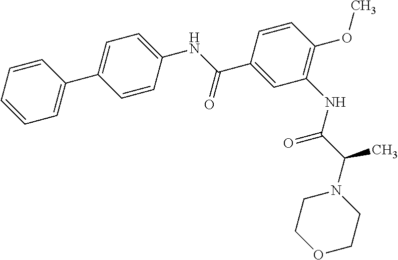 [SMILES: COc1ccc(C(=O)Nc2ccc(-c3ccccc3)cc2)cc1NC(=O)[C@@H](C)N1CCOCC1] | N-(biphenyl-4-yl)-4- methoxy-3-{[(2R)-2- (morpholin-4- yl)propanoyl]amino} benzamide | 0.917 |
| 154 | 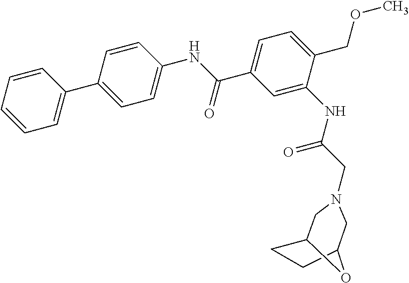 [SMILES: COCc1ccc(C(=O)Nc2ccc(-c3ccccc3)cc2)cc1NC(=O)CN1CC2CCC(C1)O2] | N-(biphenyl-4-yl)-4- (methoxymethyl)-3-[(8-oxa- 3-azabicyclo[3.2.1]oct-3- ylacetyl)amino]benzamide | 1.097 |
| 155 | 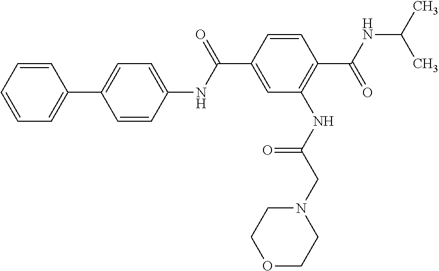 [SMILES: CC(C)NC(=O)c1ccc(C(=O)Nc2ccc(-c3ccccc3)cc2)cc1NC(=O)CN1CCOCC1] | N⁴-(biphenyl-4-yl)-2- [(morpholin-4- ylacetyl)amino]-N¹-(propan- 2-yl)benzene-1,4- dicarboxamide | 0.977 |
| 156 | 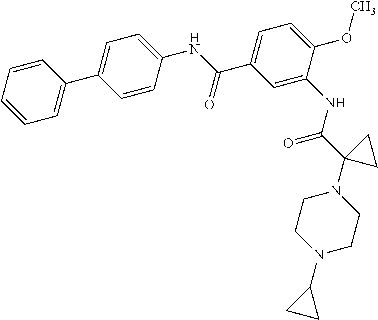 [SMILES: COc1ccc(C(=O)Nc2ccc(-c3ccccc3)cc2)cc1NC(=O)C1(N2CCN(C3CC3)CC2)CC1] | N-(biphenyl-4-yl)-3-({[1-(4- cyclopropylpiperazin-1- yl)cyclopropyl)carbonyl} amino)-4-methoxybenzamide | 0.917 |
| 157 | 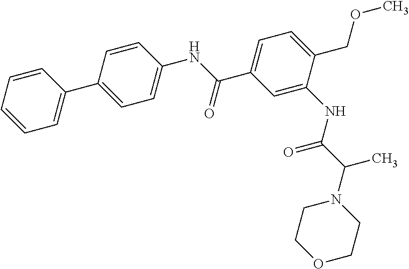 [SMILES: COCc1ccc(C(=O)Nc2ccc(-c3ccccc3)cc2)cc1NC(=O)C(C)N1CCOCC1] | N-(biphenyl-4-yl)-4- (methoxymethyl)-3-{[2- (morpholin-4- yl)propanoyl)amino} benzamide | 0.937 |
| 158 | 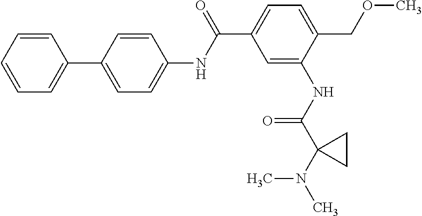 [SMILES: COCc1ccc(C(=O)Nc2ccc(-c3ccccc3)cc2)cc1NC(=O)C1(N(C)C)CC1] | N-(biphenyl-4-yl)-3-({[1- (dimethylamino)cyclopropyl] carbonyl(amino)-4- (methoxymethyl)benzamide | 1.177 |
| 159 | 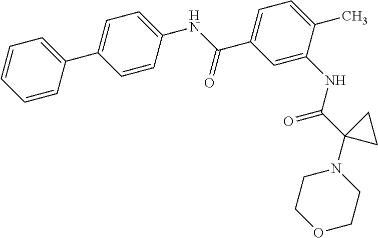 [SMILES: Cc1ccc(C(=O)Nc2ccc(-c3ccccc3)cc2)cc1NC(=O)C1(N2CCOCC2)CC1] | N-(biphenyl-4-yl)-4-methyl- 3-({[1-(morpholin-4- yl)cyclopropyl)carbonyl} amino)benzamide | 1.297 |
| 160 | 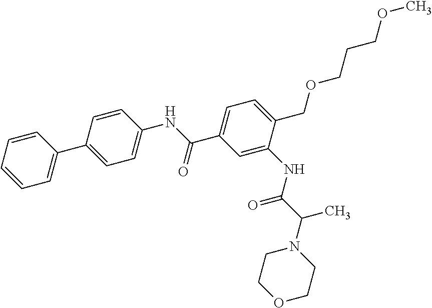 [SMILES: COCCCOCc1ccc(C(=O)Nc2ccc(-c3ccccc3)cc2)cc1NC(=O)C(C)N1CCOCC1] | N-(biphenyl-4-yl)-4-[(3- methoxypropoxy)methyl]-3- ([2-(morpholin-4- yl)propanoyl]amino} benzamide | 0.997 |
| 161 | 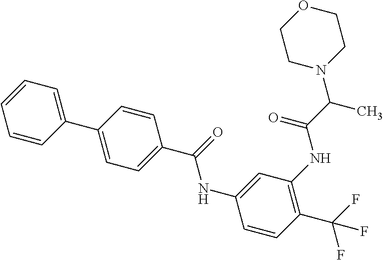 [SMILES: CC(C(=O)Nc1cc(NC(=O)c2ccc(-c3ccccc3)cc2)ccc1C(F)(F)F)N1CCOCC1] | N-[3-{[2-(morpholin-4- yl)propanoyl]amino}-4- (trifluoromethyl)phenyl] biphenyl-4-carboxamide | 1.17 |
| 162 | 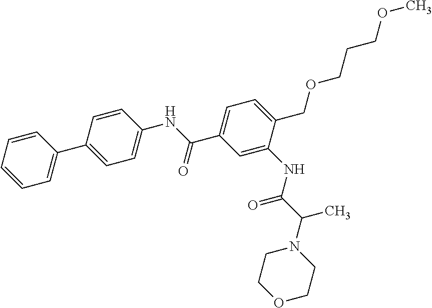 [SMILES: COCCCOCc1ccc(C(=O)Nc2ccc(-c3ccccc3)cc2)cc1NC(=O)C(C)N1CCOCC1] | N-(biphenyl-4-yl)-4-[(2- methoxyethoxy)methyl]-3- {[(2R*)-2-(morpholin-4- yl)propanoyl)amino} benzamide | 0.937 |
| 163 | 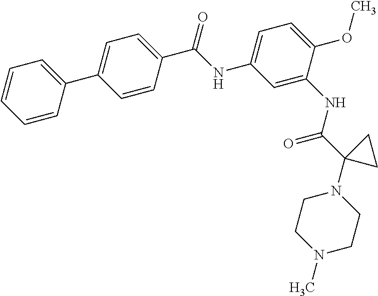 [SMILES: COc1ccc(NC(=O)c2ccc(-c3ccccc3)cc2)cc1NC(=O)C1(N2CCN(C)CC2)CC1] | N-[4-methoxy-3-({[1-(4- methylpiperazin-1- yl)cyclopropyl]carbonyl} amino)phenyl]biphenyl-4- carboxamide hydrochloride (1:1) | 0.857 |
| 164 | 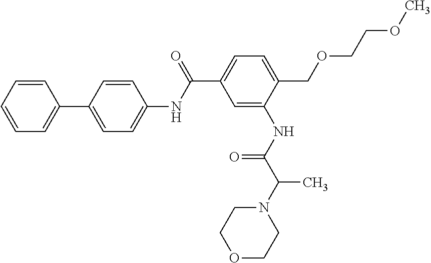 [SMILES: COCCOCc1ccc(C(=O)Nc2ccc(-c3ccccc3)cc2)cc1NC(=O)C(C)N1CCOCC1] | N-(biphenyl-4-yl)-4-[(2- methoxyethoxy)methyl]-3- {[2-(morpholin-4- yl)propanoyl]amino} benzamide | 0.927 |
| 165 | 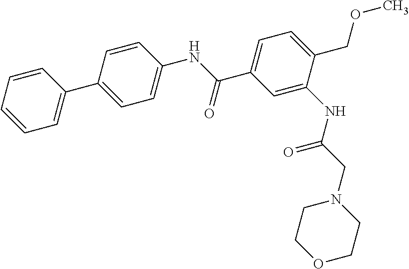 [SMILES: COCc1ccc(C(=O)Nc2ccc(-c3ccccc3)cc2)cc1NC(=O)CN1CCOCC1] | N-(biphenyl-4-yl)-4- (methoxymethyl)-3- [(morpholin-4- ylacetyl)amino]benzamide | 0.927 |
| 166 | 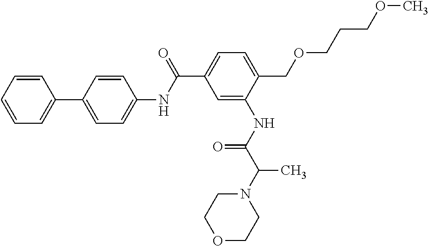 [SMILES: COCCCOCc1ccc(C(=O)Nc2ccc(-c3ccccc3)cc2)cc1NC(=O)C(C)N1CCOCC1] | N-(biphenyl-4-yl)-4-[(3- methoxypropoxy)methyl]-3- {[(2R*)-2-(morpholin-4- yl)propanoyl]amino} benzamide | 0.997 |
| 167 | 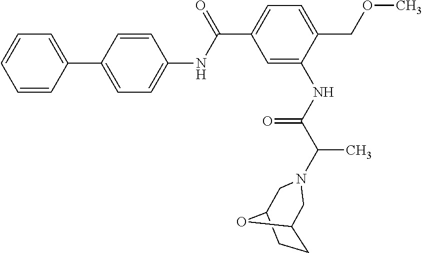 [SMILES: COCc1ccc(C(=O)Nc2ccc(-c3ccccc3)cc2)cc1NC(=O)C(C)N1CC2CCC(C1)O2] | N-(biphenyl-4-yl)-4- (methoxymethyl)-3-{[(2S*)- 2-(8-oxa-3- azabicyclo[3.2.1]oct-3- yl)propanoyl]amino} benzamide | 1.127 |
| 168 | 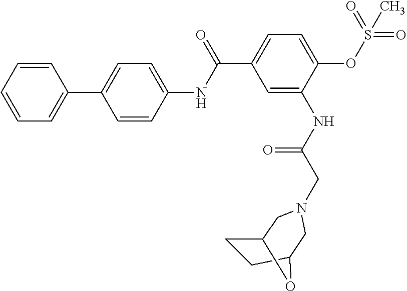 [SMILES: CS(=O)(=O)Oc1ccc(C(=O)Nc2ccc(-c3ccccc3)cc2)cc1NC(=O)CN1CC2CCC(C1)O2] | N-(biphenyl-4-yl)-4- [(methylsulfonyl)methyl]-3- [(8-oxa-3- azabicyclo[3.2.1]oct-3- ylacetyl)amino]benzamide | 0.947 |
| 169 | 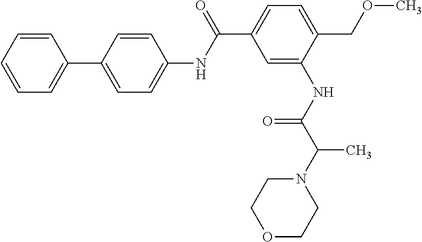 [SMILES: COCc1ccc(C(=O)Nc2ccc(-c3ccccc3)cc2)cc1NC(=O)C(C)N1CCOCC1] | N-(biphenyl-4-yl)-4- (methoxymethyl)-3-{[(2R*)- 2-(morpholin-4- yl)propanoyl]amino} benzamide | 0.937 |
| 170 | 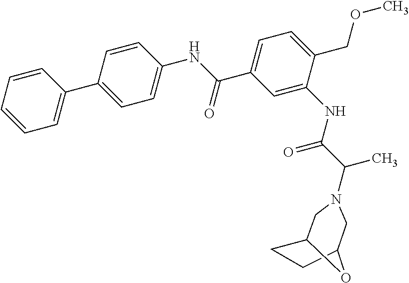 [SMILES: COCc1ccc(C(=O)Nc2ccc(-c3ccccc3)cc2)cc1NC(=O)C(C)N1CC2CCC(C1)O2] | N-(biphenyl-4-yl)-4- (methoxymethyl)-3-{(2-(8- oxa-3-azabicyclo[3.2.1]oct- 3- yl)propanoyl)amino} benzamide | 1.117 |
| 171 | 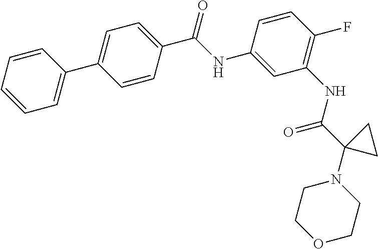 [SMILES: O=C(Nc1ccc(F)c(NC(=O)C2(N3CCOCC3)CC2)c1)c1ccc(-c2ccccc2)cc1] | N-[4-fluoro-3-({[1- (morpholin-4- yl)cyclopropyl]carbonyl} amino)phenyl]biphenyl-4- carboxamide | 1.317 |
| 172 | 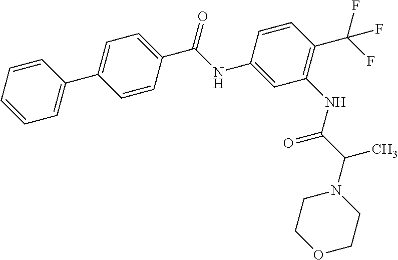 [SMILES: CC(C(=O)Nc1cc(NC(=O)c2ccc(-c3ccccc3)cc2)ccc1C(F)(F)F)N1CCOCC1] | N-[3-{[(2R*)-2-(morpholin-4- yl)propanoyl]amino}-4- (trifluoromethyl)phenyl] biphenyl-4-carboxamide | 1.17 |
| 173 | 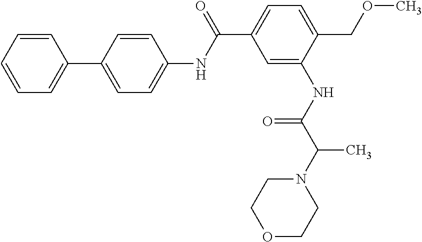 [SMILES: COCc1ccc(C(=O)Nc2ccc(-c3ccccc3)cc2)cc1NC(=O)C(C)N1CCOCC1] | N-(biphenyl-4-yl)-4- (methoxymethyl)-3-{[(2S*)- 2-(morpholin-4- yl)propanoyl]amino} benzamide | 0.937 |
| 174 | 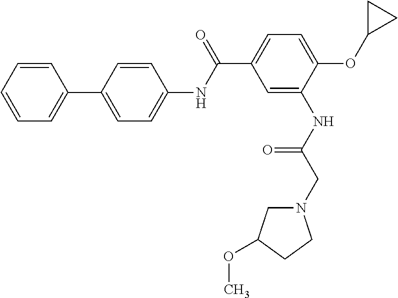 [SMILES: COC1CCN(CC(=O)Nc2cc(C(=O)Nc3ccc(-c4ccccc4)cc3)ccc2OC2CC2)C1] | N-(biphenyl-4-yl)-4- (cyclopropyloxy)-3-{[(3- methoxypyrrolidin-1- yl)acetyl)amino}benzamide | |
| 175 | 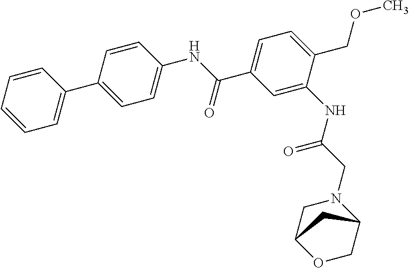 [SMILES: COCc1ccc(C(=O)Nc2ccc(-c3ccccc3)cc2)cc1NC(=O)CN1C[C@@H]2C[C@H]1CO2] | N-(biphenyl-4-yl)-4- (methoxymethyl)-3- {[(1S,4S)-2-oxa-5- azabicyclo[2.2.1]hept-5- ylacetyl)amino}benzamide | 0.817 |
| 176 | 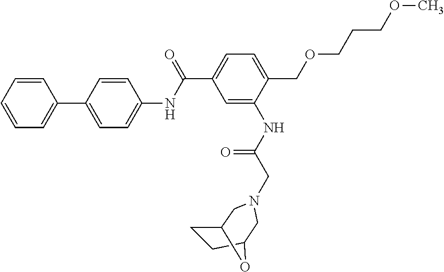 [SMILES: COCCCOCc1ccc(C(=O)Nc2ccc(-c3ccccc3)cc2)cc1NC(=O)CN1CC2CCC(C1)O2] | N-(biphenyl-4-yl)-4-[(3- methoxypropoxy)methyl]-3- [(8-oxa-3- azabicyclo[3.2.1]oct-3- ylacetyl)amino]benzamide | 1.137 |
| 177 | 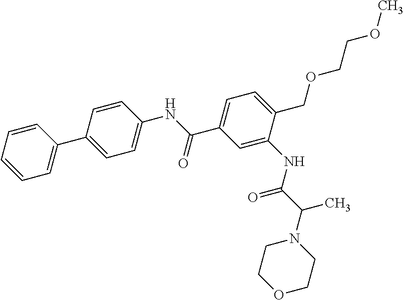 [SMILES: COCCOCc1ccc(C(=O)Nc2ccc(-c3ccccc3)cc2)cc1NC(=O)C(C)N1CCOCC1] | N-(biphenyl-4-yl)-4-[(2- methoxyethoxy)methyl)-3- {[(2S*)-2-(morpholin-4- yl)propanoyl)amino} benzamide | 0.927 |
| 178 | 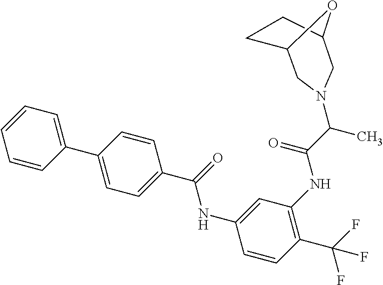 [SMILES: CC(C(=O)Nc1cc(NC(=O)c2ccc(-c3ccccc3)cc2)ccc1C(F)(F)F)N1CC2CCC(C1)O2] | N-[3-{[2-(8-oxa-3- azabicyclo[3.2.1]oct-3- yl)propanoyl]amino}-4- (trifluoromethyl)phenyl) biphenyl-4-carboxamide | 1.297 |
| 179 | 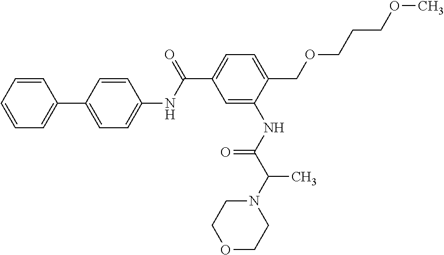 [SMILES: COCCCOCc1ccc(C(=O)Nc2ccc(-c3ccccc3)cc2)cc1NC(=O)C(C)N1CCOCC1] | N-(biphenyl-4-yl)-4-[(3- methoxypropoxy)methyl]-3- {[(2S*)-2-(morpholin-4- yl)propanoyl]amino} benzamide | 0.997 |
| 180 | 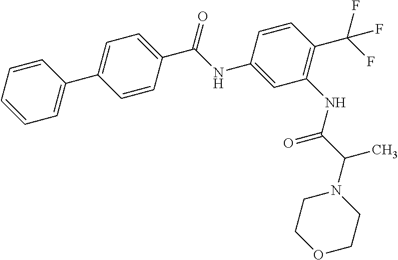 [SMILES: CC(C(=O)Nc1cc(NC(=O)c2ccc(-c3ccccc3)cc2)ccc1C(F)(F)F)N1CCOCC1] | N-(3-{[(2S*)-2-(morpholin-4- yl)propanoyl]amino}-4- (trifluoromethyl)phenyl] biphenyl-4-carboxamide | 1.17 |
| 181 | 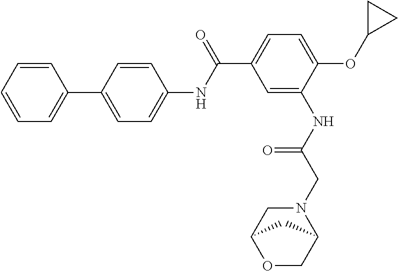 [SMILES: O=C(CN1C[C@H]2C[C@@H]1CO2)Nc1cc(C(=O)Nc2ccc(-c3ccccc3)cc2)ccc1OC1CC1] | N-(biphenyl-4-yl)-4- (cyclopropyloxy)-3- {[(1R,4R)-2-oxa-5- azabicyclo[2.2.1]hept-5- ylacetyl]amino}benzamide | 0.877 |
| 182 | 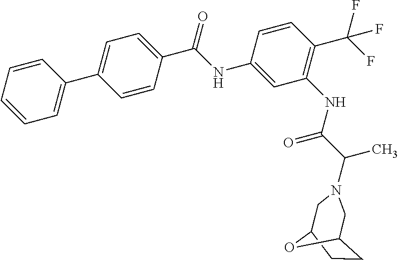 [SMILES: CC(C(=O)Nc1cc(NC(=O)c2ccc(-c3ccccc3)cc2)ccc1C(F)(F)F)N1CC2CCC(C1)O2] | N-[3-{[(2R*)-2-(8-oxa-3- azabicyclo[3.2.1]oct-3- yl)propanoyl]amino}-4- (trifluoromethyl)phenyl] biphenyl-4-carboxamide | 1.297 |
| 183 | 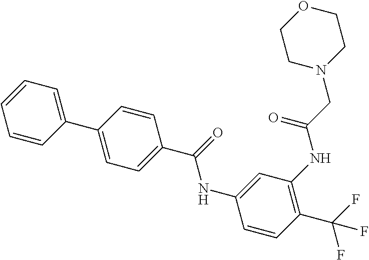 [SMILES: O=C(CN1CCOCC1)Nc1cc(NC(=O)c2ccc(-c3ccccc3)cc2)ccc1C(F)(F)F] | N-{3-[(morpholin-4- ylacetyl)amino]-4- (trifluoromethyl)phenyl} biphenyl-4-carboxamide | 1.17 |
| 184 | 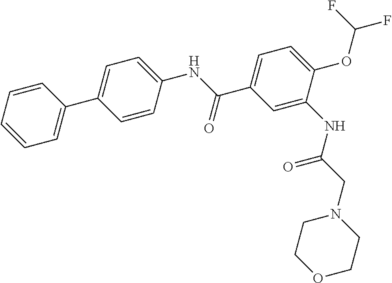 [SMILES: O=C(CN1CCOCC1)Nc1cc(C(=O)Nc2ccc(-c3ccccc3)cc2)ccc1OC(F)F] | N-(biphenyl-4-yl)-4- (difluoromethoxy)-3- [(morpholin-4- ylacetyl)amino]benzamide | 0.997 |
| 185 | 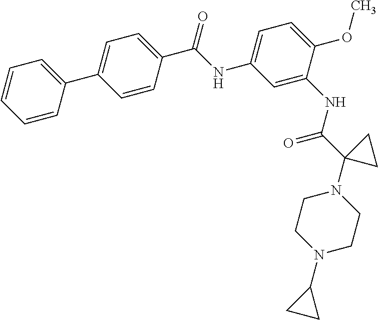 [SMILES: COc1ccc(NC(=O)c2ccc(-c3ccccc3)cc2)cc1NC(=O)C1(N2CCN(C3CC3)CC2)CC1] | N-[3-({[l-(4- cyclopropylpiperazin-1- yl)cyclopropyl]carbonyl}ami no}-4- melhoxyphenyl)biphenyl-4- carboxamide | 0.97 |
| 186 | 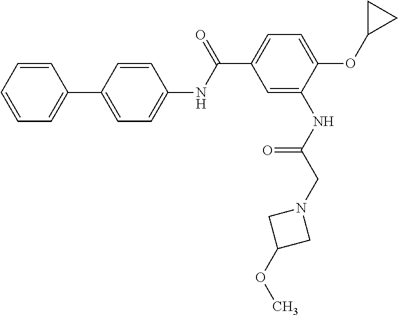 [SMILES: COC1CN(CC(=O)Nc2cc(C(=O)Nc3ccc(-c4ccccc4)cc3)ccc2OC2CC2)C1] | N-(biphenyl-4-yl)-4- (cyclopropyloxy)-3-{[(3- methoxyazetidin-1- yl)acetyl]amino}benzamide | 0.887 |
| 187 | 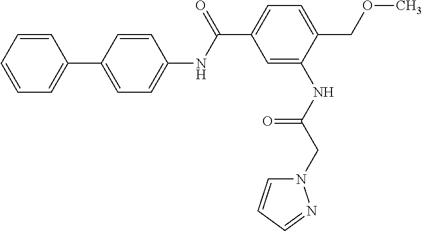 [SMILES: COCc1ccc(C(=O)Nc2ccc(-c3ccccc3)cc2)cc1NC(=O)Cn1cccn1] | N-(biphenyl-4-yl)-4- (methoxymethyl)-3-[(1H- pyrazol-1- ylacetyl)amino]benzamide | 1.147 |
| 188 | 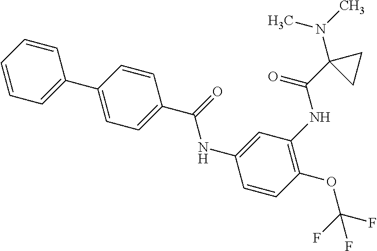 [SMILES: CN(C)C1(C(=O)Nc2cc(NC(=O)c3ccc(-c4ccccc4)cc3)ccc2OC(F)(F)F)CC1] | N-[3-({[1- (dimethylamino)cyclopropyl] carbonyl}amino)-4- (trifluoromethoxy)phenyl] biphenyl-4-carboxamide | 1.427 |
| 189 | 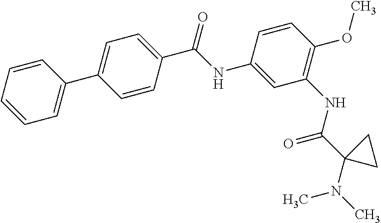 [SMILES: COc1ccc(NC(=O)c2ccc(-c3ccccc3)cc2)cc1NC(=O)C1(N(C)C)CC1] | N-[3-({[1- (dimethylamino)cyclopropyl] carbonyl}amino)-4- methoxyphenyl]biphenyl-4- carboxamide | 1.097 |
| 190 | 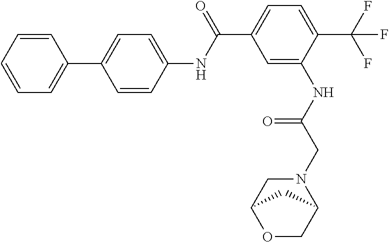 [SMILES: O=C(CN1C[C@H]2C[C@@H]1CO2)Nc1cc(C(=O)Nc2ccc(-c3ccccc3)cc2)ccc1C(F)(F)F] | N-(biphenyl-4-yl)-3- {[(1R,4R)-2-oxa-5- azabicyclo[2.2.1]hept-5- ylacetyl]amino}-4- (trifluoromethyl)benzamide | 0.887 |
| 191 | 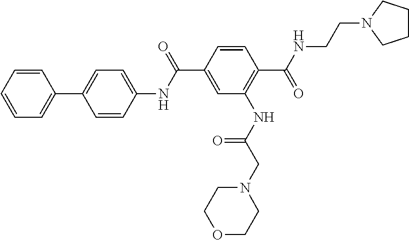 [SMILES: O=C(CN1CCOCC1)Nc1cc(C(=O)Nc2ccc(-c3ccccc3)cc2)ccc1C(=O)NCCN1CCCC1] | N4-(biphenyl-4-yl)-2- [(morpholin-4- ylacetyl)amino]-N1-[2- (pyrrolidin-1- yl)ethyl]terephthalamide | 0.727 |
| 192 | 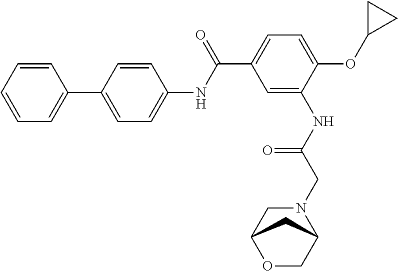 [SMILES: O=C(CN1C[C@@H]2C[C@H]1CO2)Nc1cc(C(=O)Nc2ccc(-c3ccccc3)cc2)ccc1OC1CC1] | N-(biphenyl-4-yl)-4- (cyclopropyloxy)-3-{[(1S,4S)- 2-oxa-S- azabicyclo[2.2.1]hept-5- ylacetyl]amino}benzamide | |
| 193 | 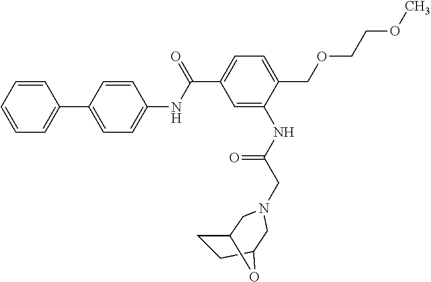 [SMILES: COCCOCc1ccc(C(=O)Nc2ccc(-c3ccccc3)cc2)cc1NC(=O)CN1CC2CCC(C1)O2] | N-(biphenyl-4-yl)-4-[(2- methoxyethoxy)methyl]-3- [(8-oxa-3- azabicyclo[3.2.1]oct-3- ylacetyl)amino]benzamide | 1.057 |
| 194 | 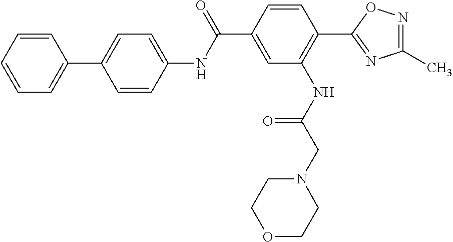 [SMILES: Cc1noc(-c2ccc(C(=O)Nc3ccc(-c4ccccc4)cc3)cc2NC(=O)CN2CCOCC2)n1] | N-(biphenyl-4-yl)-4-(3- methyl-1,2,4-oxadiazol-5- yl)-3-[(morpholin-4- ylacetyl)amino]benzamide | 1.117 |
| 195 | 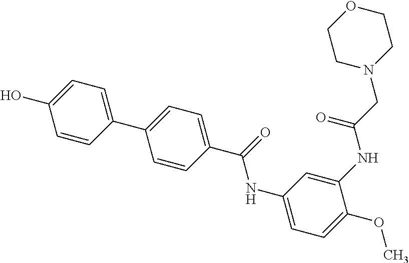 [SMILES: COc1ccc(NC(=O)c2ccc(-c3ccc(O)cc3)cc2)cc1NC(=O)CN1CCOCC1] | 4′-hydroxy-N-{4-methoxy-3- [(morpholin-4- ylacetyl)amino)phenyl} biphenyl-4-carboxamide | 0.77 |
| 196 | 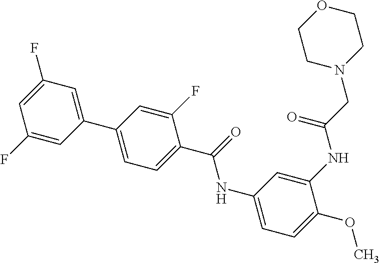 [SMILES: COc1ccc(NC(=O)c2ccc(-c3cc(F)cc(F)c3)cc2F)cc1NC(=O)CN1CCOCC1] | 3,3′,5′-trifluoro-N-{4- methoxy-3-[(morpholin-4- ylacetyl)amino]phenyl} biphenyl-4-carboxamide | |
| 197 | 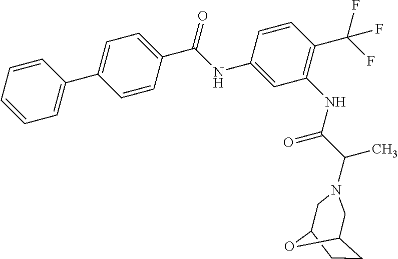 [SMILES: CC(C(=O)Nc1cc(NC(=O)c2ccc(-c3ccccc3)cc2)ccc1C(F)(F)F)N1CC2CCC(C1)O2] | N-[3-{[(2S*)-2-(8-oxa-3- azabicydo[3.2.1]oct-3- yl)propanoyl]amino}-4- (trifluoromethyl)phenyl) biphenyl-4-carboxamide | 1.297 |
| 198 | 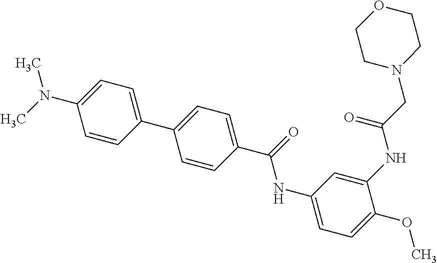 [SMILES: COc1ccc(NC(=O)c2ccc(-c3ccc(N(C)C)cc3)cc2)cc1NC(=O)CN1CCOCC1] | 4′-(dimethylamino)-N-{4- methoxy-3-[(morpholin-4- ylacetyl)amino]phenyl} biphenyl-4-carboxamide | 0.87 |
| 199 | 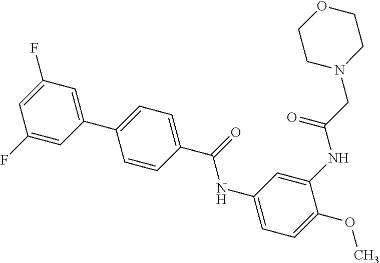 [SMILES: COc1ccc(NC(=O)c2ccc(-c3cc(F)cc(F)c3)cc2)cc1NC(=O)CN1CCOCC1] | 3′,5′-difluoro-N-{4-methoxy- 3-[(morpholin-4- ylacetyl)amino]phenyl} biphenyl-4-carboxamide | 0.927 |
| 200 | 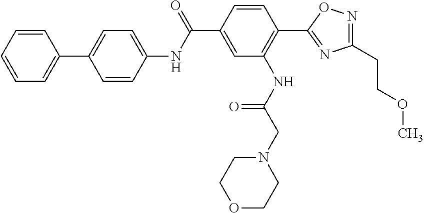 [SMILES: COCCc1noc(-c2ccc(C(=O)Nc3ccc(-c4ccccc4)cc3)cc2NC(=O)CN2CCOCC2)n1] | N-(biphenyl-4-yl)-4-[3-(2- methoxyethyl)-1,2,4- oxadiazol-5-yl]-3- [(morpholin-4- ylacetyl)amino]benzamide | 1.087 |
| 201 | 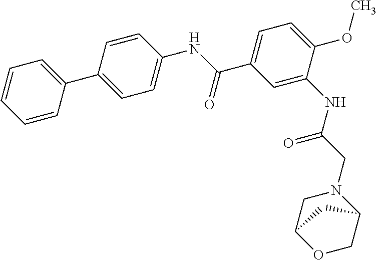 [SMILES: COc1ccc(C(=O)Nc2ccc(-c3ccccc3)cc2)cc1NC(=O)CN1C[C@H]2C[C@@H]1CO2] | N-(biphenyl-4-yl)-4- methoxy-3-{[(1R,4R)-2-oxa- 5-azabicyclo[2.2.1]hept-5- ylacetyl)amino}benzamide | 0.87 |
| 202 | 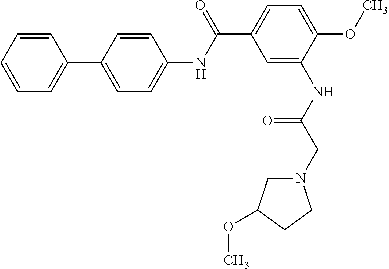 [SMILES: COc1ccc(C(=O)Nc2ccc(-c3ccccc3)cc2)cc1NC(=O)CN1CCC(OC)C1] | N-(biphenyl-4-yl)-4- methoxy-3-{[(3- methoxypyrrolidin-1- yl)acetyl]amino}benzamide | 0.837 |
| 203 | 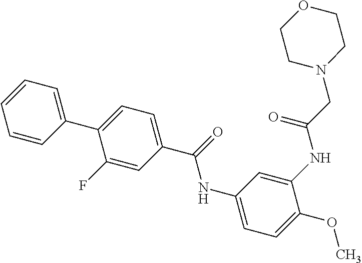 [SMILES: COc1ccc(NC(=O)c2ccc(-c3ccccc3)c(F)c2)cc1NC(=O)CN1CCOCC1] | 2-fluoro-N-{4-methoxy-3- [(morpholin-4- ylacetyl)amino]phenyl} biphenyl-4-carboxamide | |
| 204 | 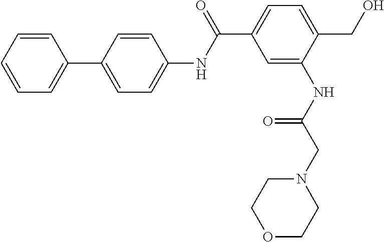 [SMILES: O=C(CN1CCOCC1)Nc1cc(C(=O)Nc2ccc(-c3ccccc3)cc2)ccc1CO] | N-(biphenyl-4-yl)-4- (hydroxymethyl)-3- [(morpholin-4- ylacetyl)amino]benzamide | 0.797 |
| 205 | 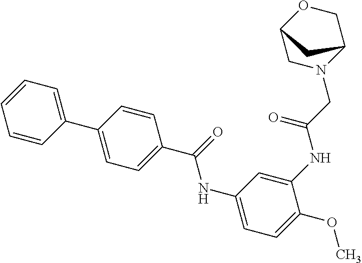 [SMILES: COc1ccc(NC(=O)c2ccc(-c3ccccc3)cc2)cc1NC(=O)CN1C[C@H]2C[C@@H]1CO2] | N-(4-methoxy-3-{[(1R,4R)-2- oxa-5-azabicyclo[2.2.1]hept- 5- ylacetyl]amino}phenyl) biphenyl-4-carboxamide | 0.797 |
| 206 | 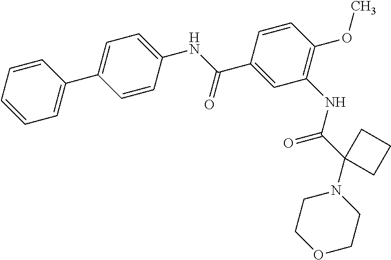 [SMILES: COc1ccc(C(=O)Nc2ccc(-c3ccccc3)cc2)cc1NC(=O)C1(N2CCOCC2)CCC1] | N-(biphenyl-4-yl)-4- methoxy-3-({[1-(morpholin- 4- yl)cyclobutyl)carbonyl} amino)benzamide | 1.297 |
| 207 | 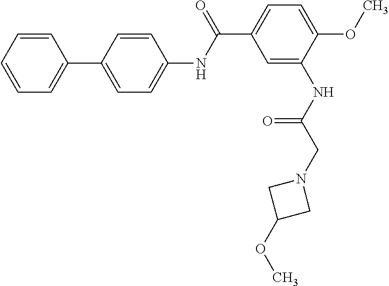 [SMILES: COc1ccc(C(=O)Nc2ccc(-c3ccccc3)cc2)cc1NC(=O)CN1CC(OC)C1] | N-(biphenyl-4-yl)-4- methoxy-3-{[(3- methoxyazetidin-1- yl)acetyl]amino}benzamide | 0.827 |
| 208 | 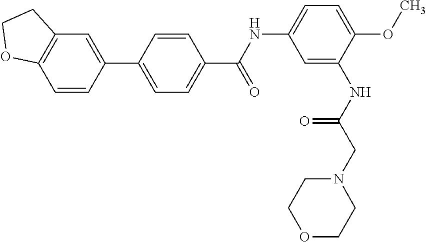 [SMILES: COc1ccc(NC(=O)c2ccc(-c3ccc4c(c3)CCO4)cc2)cc1NC(=O)CN1CCOCC1] | 4-(2,3-dihydro-1- benzofuran-5-yl)-N-{4- methoxy-3-[(morpholin-4- ylacetyl)amino]phenyl} benzamide | 0.867 |
| 209 | 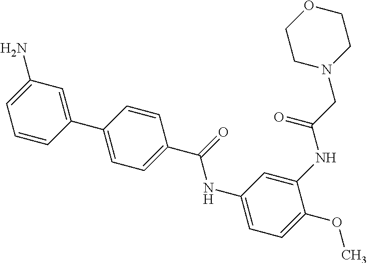 [SMILES: COc1ccc(NC(=O)c2ccc(-c3cccc(N)c3)cc2)cc1NC(=O)CN1CCOCC1] | 3′-amino-N-{4-methoxy-3- [(morpholin-4- ylacetyl)amino]phenyl} biphenyl-4-carboxamide | 0.617 |
| 210 | 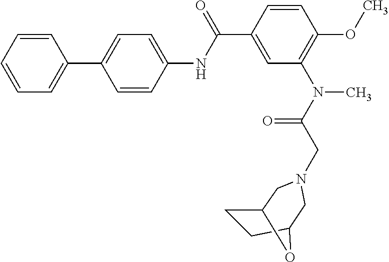 [SMILES: COc1ccc(C(=O)Nc2ccc(-c3ccccc3)cc2)cc1N(C)C(=O)CN1CC2CCC(C1)O2] | N-(biphenyl-4-yl)-4- methoxy-3-[methyl(8-oxa-3- azabicyclo[3.2.1]oct-3- ylacetyl)amino]benzamide | 0.867 |
| 211 | 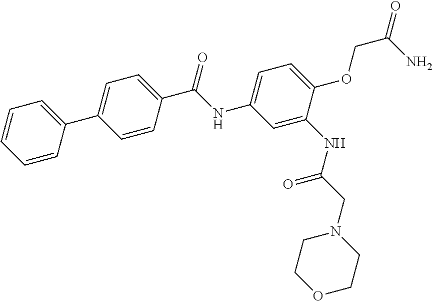 [SMILES: NC(=O)COc1ccc(NC(=O)c2ccc(-c3ccccc3)cc2)cc1NC(=O)CN1CCOCC1] | N-{4-(2-amino-2- oxoethoxy)-3-[(morpholin- 4- ylacetyl)amino]phenyl} biphenyl-4-carboxamide | 0.757 |
| 212 | 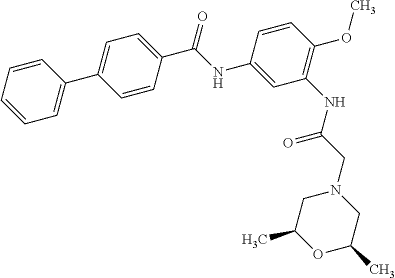 [SMILES: COc1ccc(NC(=O)c2ccc(-c3ccccc3)cc2)cc1NC(=O)CN1C[C@@H](C)O[C@@H](C)C1] | N-[3-({[(2R,6S)-2,6- dimethylmorpholin-4- yl]acetyl}amino)-4- methoxyphenyl]biphenyl-4- carboxamide | 0.977 |
| 213 | 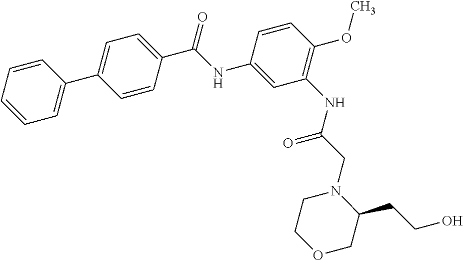 [SMILES: COc1ccc(NC(=O)c2ccc(-c3ccccc3)cc2)cc1NC(=O)CN1CCOC[C@@H]1CCO] | N-[3-({[(3S)-3-(2- hydroxyethyl)morpholin-4- yl]acetyl}amino)-4- methoxyphenyl]biphenyl-4- carboxamide | 0.887 |
| 214 | 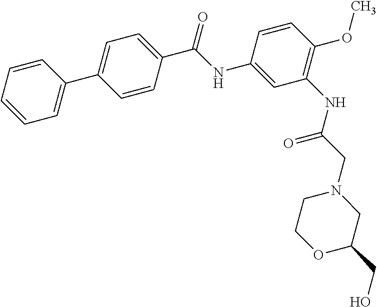 [SMILES: COc1ccc(NC(=O)c2ccc(-c3ccccc3)cc2)cc1NC(=O)CN1CCO[C@H](CO)C1] | N-[3-({[(2S)-2- (hydroxymethyl)morpholin- 4-yl]acetyl}amino)-4- methoxyphenyl]biphenyl-4- carboxamide | 0.837 |
| 215 | 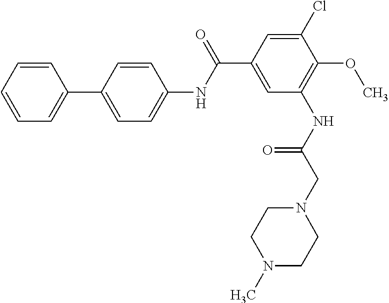 [SMILES: COc1c(Cl)cc(C(=O)Nc2ccc(-c3ccccc3)cc2)cc1NC(=O)CN1CCN(C)CC1] | N-(biphenyl-4-yl)-3-chloro- 4-methoxy-5-{[(4- methylpiperazin-1- yl)acetyl]amino}benzamide | 0.97 |
| 216 | 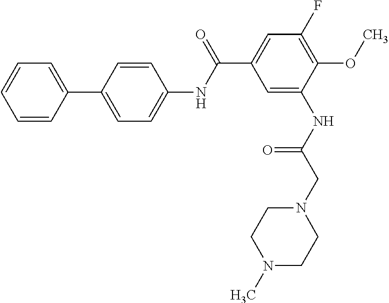 [SMILES: COc1c(F)cc(C(=O)Nc2ccc(-c3ccccc3)cc2)cc1NC(=O)CN1CCN(C)CC1] | N-(biphenyl-4-yl)-3-fluoro-4- methoxy-5-{[(4- methylpiperazin-1- yl)acetyl]amino}benzamide | 0.877 |
| 217 | 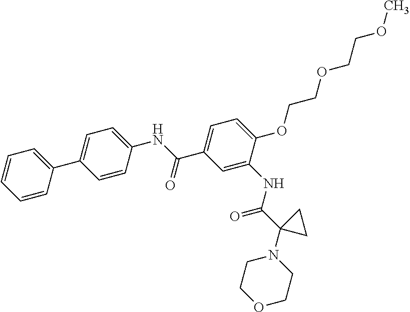 [SMILES: COCCOCCOc1ccc(C(=O)Nc2ccc(-c3ccccc3)cc2)cc1NC(=O)C1(N2CCOCC2)CC1] | N-(biphenyl-4-yl)-4-[2-(2- methoxyethoxy}ethoxy)-3- ({[1-(morpholin-4- yl)cyclopropyl]carbonyl} amino)benzamide | 1.37 |
| 218 | 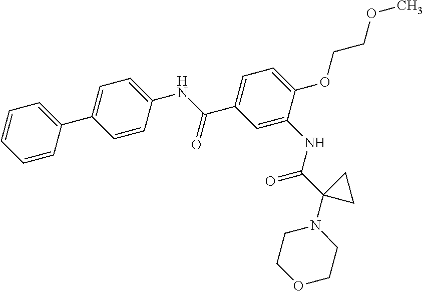 [SMILES: COCCOc1ccc(C(=O)Nc2ccc(-c3ccccc3)cc2)cc1NC(=O)C1(N2CCOCC2)CC1] | N-(biphenyl-4-yl)-4-(2- methoxyethoxy)-3-({[1- (morpholin-4- yl)cyclopropyl]carbonyl) amino)benzamide | 1.37 |
| 219 | 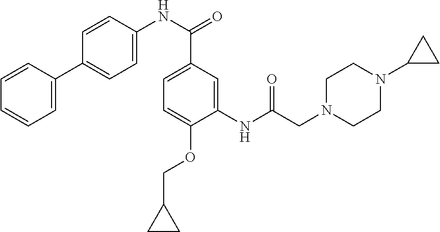 [SMILES: O=C(CN1CCN(C2CC2)CC1)Nc1cc(C(=O)Nc2ccc(-c3ccccc3)cc2)ccc1OCC1CC1] | N-(biphenyl-4-yl)-4- (cyclopropylmethoxy)-3- {[(4-cyclopropylpiperazin-1- yl)acetyl]amino}benzamide | 1.044 |
| 220 | 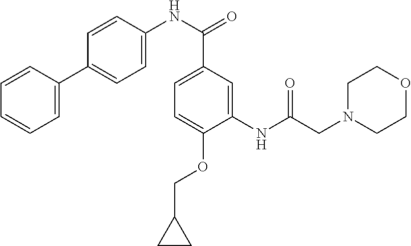 [SMILES: O=C(CN1CCOCC1)Nc1cc(C(=O)Nc2ccc(-c3ccccc3)cc2)ccc1OCC1CC1] | N-(biphenyl-4-yl)-4- (cyclopropylmethoxy)-3- [(morpholin-4- ylacetyl)amino)benzamide | 1.353 |
| 221 | 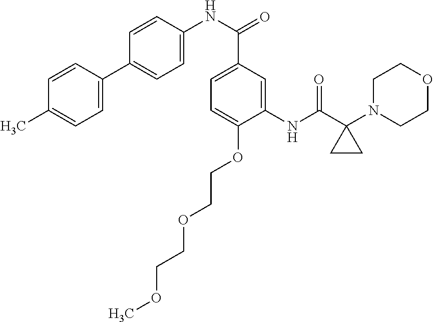 [SMILES: COCCOCCOc1ccc(C(=O)Nc2ccc(-c3ccc(C)cc3)cc2)cc1NC(=O)C1(N2CCOCC2)CC1] | 4-[2-(2- methoxyethoxy)ethoxy]-N- (4′-methylbiphenyl-4-yl)-3- ({[1-(morpholin-4- yl)cyclopropyl]carbonyl} amino)benzamide | 1.424 |
| 222 | 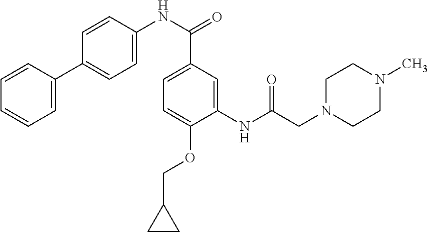 [SMILES: CN1CCN(CC(=O)Nc2cc(C(=O)Nc3ccc(-c4ccccc4)cc3)ccc2OCC2CC2)CC1] | N-(biphenyl-4-yl)-4- (cyclopropylmethoxy)-3- {[(4-methylpiperazin-1- yl)acetyl]amino}benzamide | 1.333 |

Further, the compounds of formula (I) of the present invention can be converted to any salt as described herein, by any method which is known to the person skilled in the art. Similarly, any salt of a compound of formula (I) of the present invention can be converted into the free compound, by any method which is known to the person skilled in the art.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known chemotherapeutic agents or anti-cancer agents, e.g. anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

The term “(chemotherapeutic) anti-cancer agents”, includes but is not limited to 131l-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

### Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

### Measurement of the Inhibitory Activity of Selected Compounds on the Wnt Signaling Cascade

In order to discover and characterize small molecules which inhibit the constitutive active colorectal cancer cell (CRC) Wnt pathway, a cellular reporter assay was employed. The corresponding assay cell was generated by transfection of the colorectal cancer cell line HCT116 (ATCC, #CCL-247) with the Super TopFlash vector (Morin, Science 275, 1997, 1787-1790; Molenaar et al., Cell 86 (3), 1996, 391-399). The HCT116 cell line is cultivated at 37° C. and 5% CO₂ in DMEM/F-12 (Life Technologies, #11320-074), supplemented with 2 mM glutamine, 20 mM HEPES, 1.4 mM pyruvate, 0.15% Na-bicarbonate and 10% foetal bovine serum (GIBCO, #10270), this cancer cell line is pathophysiological relevant since it carries a deletion of position S45 in the β-catenin gene, leading to constitutive active Wnt signaling. Stable transfectants were generated by cotransfection with pcDNA3 and selection of stable transfected cells with 1 mg/ml G418.

In a parallel approach, HCT116 cells were cotransfected with the FOP control vector and pcDNA3. The FOP vector is identical to the TOP construct, but it contains instead of functional TCF elements a randomized, non-functional sequence. For this transfection a stable transfected cell line was generated as well.

In preparation of the assay, the two cell lines were plated 24 hrs before at 10000 cells per well of a 384 micro titre plate (MTP) in 30 μL growth medium. Selective inhibitory activity for small molecules on the mutated Wnt pathway was determined after parallel incubation of both (TOP and FOP) HCT116 reporter cell lines with a compound dilution series from 50 μM to 15 nM in steps of 3.16-fold dilutions in CAFTY buffer (130 mM NaCl, 5 mM KCl, 20 mM HEPES, 1 mM MgCl₂, 5 mM NaHCO₃, pH 7.4) containing 2 mM Ca²⁺ and 0.01% BSA. The compounds were thereby serially prediluted in 100% DMSO and thereafter in addition 50 fold into the CAFTY compound dilution buffer (described above). From this dilution 10 μL were added to the cells in 30 μL growth medium and incubated for 36 hours at 37° C. and 5% CO₂. Thereafter luciferase assay buffer (1:1 mixture of luciferase substrate buffer (20 mM Tricine, 2.67 mM MgSO4, 0.1 mM EDTA, 4 mM DTT, 270 μM Coenzyme A, 470 μM Luciferin, 530 μM ATP, ph adjusted to pH 7.8 with a sufficient volume of 5M NaOH) and Triton buffer (30 mL Triton X-100, 115 mL glycerol, 308 mg Dithiothreitol, 4.45 g Na₂HPO₄.2 H₂O, 3.03 g Tris.HCL, ad 1 l H₂0, pH 7.8) was added as equal volume to the compound solution on the cells to determine luciferase expression as a measure of Wnt signaling activity in a luminometer.

In order to determine the inhibitory activity of compounds for the WT Wnt signaling pathway, the Super TopFlash vector respectively FOP vector were cotransfected with pcDNA3 into HEK293 and stable transfected HEK293 cells were isolated by antibiotic selection. In preparation of compound testing, a dose response curve for the Wnt dependent luciferase expression was recorded by stimulating the assay cells with human recombinant Wnt-3a (R&D, #5036-WN-010) at different concentrations for 16 hrs at 37° C. and 5% CO₂ followed by subsequent luciferase measurement as described above to determine the Wnt-3a EC50 for the HEK293 TOP cell line on the day of testing. The recombinant human Wnt-3a was thereby used between 2500 and 5 ng/ml in two-fold dilution steps. To determine the inhibitory activity of compounds on the WT Wnt pathway they were prepared and diluted as described above for the constitutive active Wnt pathway and coincubated with the EC50 concentration of Wnt-3a for 16 hrs at 37° C. and 5% CO₂ on the HEK293 TOP respectively control HEK293 FOP cells. Measurement of luciferase expression was done as described for the constitutive active Wnt assay.

TABLE 2

| Example | HCT116 TOPFlash | HCT116 FOPFlash |
|---|---|---|
| No | IC₅₀ [mol/L] | IC₅₀ [mol/L] |
| 1 | 1.57E−7 | ≧5.00E−5 |
| 2 | 3.20E−6 | ≧5.00E−5 |
| 3 | 4.30E−7 | ≧5.00E−5 |
| 4 | 1.12E−7 | ≧5.00E−5 |
| 5 | 5.07E−7 | ≧5.00E−5 |
| 6 | 3.92E−6 | ≧5.00E−5 |
| 7 | 9.63E−7 | ≧5.00E−5 |
| 8 | 7.00E−8 | ≧5.00E−5 |
| 9 | 4.00E−8 | ≧5.00E−5 |
| 10 | 1.70E−7 | ≧5.00E−5 |
| 11 | 5.33E−7 | ≧5.00E−5 |
| 12 | 4.00E−8 | ≧5.00E−5 |
| 13 | 9.10E−8 | ≧5.00E−5 |
| 14 | 2.70E−8 | ≧5.00E−5 |
| 15 | 2.96E−8 | 3.95E−5 |
| 16 | 1.79E−8 | ≧5.00E−5 |
| 17 | 2.40E−7 | ≧5.00E−5 |
| 18 | 5.87E−8 | ≧5.00E−5 |
| 19 | 1.36E−7 | ≧5.00E−5 |
| 20 | 6.25E−8 | ≧5.00E−5 |
| 21 | 3.34E−7 | 1.65E−5 |
| 22 | 5.52E−8 | ≧5.00E−5 |
| 23 | 3.20E−8 | ≧5.00E−5 |
| 24 | 3.60E−7 | ≧5.00E−5 |
| 25 | 4.37E−8 | ≧5.00E−5 |
| 26 | 6.37E−8 | ≧5.00E−5 |
| 27 | 1.20E−6 | 2.30E−5 |
| 28 | 1.79E−6 | ≧5.00E−5 |
| 29 | 2.15E−6 | ≧5.00E−5 |
| 30 | 1.64E−6 | ≧5.00E−5 |
| 31 | 3.83E−6 | ≧5.00E−5 |
| 32 | 1.70E−7 | ≧5.00E−5 |
| 33 | 4.35E−7 | ≧5.00E−5 |
| 34 | 1.06E−6 | ≧5.00E−5 |
| 35 | 1.18E−6 | ≧5.00E−5 |
| 36 | 1.55E−6 | ≧5.00E−5 |
| 37 | 1.25E−6 | ≧5.00E−5 |
| 38 | 2.00E−6 | ≧5.00E−5 |
| 39 | 2.20E−6 | ≧5.00E−5 |
| 40 | 2.30E−6 | ≧5.00E−5 |
| 41 | 2.40E−6 | ≧5.00E−5 |
| 42 | 1.11E−6 | ≧5.00E−5 |
| 43 | 2.65E−6 | ≧5.00E−5 |
| 44 | 2.90E−6 | ≧5.00E−5 |
| 45 | 3.85E−6 | ≧5.00E−5 |
| 46 | 3.45E−6 | ≧5.00E−5 |
| 47 | 7.18E−7 | 3.35E−5 |
| 48 | 8.56E−7 | 1.20E−5 |
| 49 | 2.65E−6 | ≧5.00E−5 |
| 50 | 3.64E−6 | 2.30E−5 |
| 51 | 3.18E−6 | ≧5.00E−5 |
| 52 | 4.70E−7 | ≧5.00E−5 |
| 53 | 4.80E−7 | ≧5.00E−5 |
| 54 | 3.50E−7 | ≧5.00E−5 |
| 55 | 8.34E−7 | ≧5.00E−5 |
| 56 | 2.22E−7 | ≧5.00E−5 |
| 57 | 4.90E−7 | ≧5.00E−5 |
| 58 | 5.40E−8 | ≧5.00E−5 |
| 59 | 1.11E−7 | 4.00E−5 |
| 60 | 5.82E−7 | ≧5.00E−5 |
| 61 | 1.30E−7 | ≧5.00E−5 |
| 62 | 6.10E−8 | ≧5.00E−5 |
| 63 | 2.65E−7 | ≧5.00E−5 |
| 64 | 3.50E−6 | 4.80E−5 |
| 65 | 2.20E−6 | 2.80E−5 |
| 66 | 3.92E−8 | ≧5.00E−5 |
| 67 | 2.10E−7 | 9.80E−6 |
| 68 | 5.13E−7 | ≧5.00E−5 |
| 69 | 8.10E−7 | 9.30E−6 |
| 70 | 1.35E−6 | ≧5.00E−5 |
| 71 | 2.25E−6 | ≧5.00E−5 |
| 72 | 3.75E−6 | ≧5.00E−5 |
| 73 | 1.45E−6 | ≧5.00E−5 |
| 74 | 4.70E−8 | ≧5.00E−5 |
| 75 | 1.45E−7 | ≧5.00E−5 |
| 76 | 4.87E−7 | ≧5.00E−5 |
| 77 | 3.95E−6 | ≧5.00E−5 |
| 78 | 2.90E−6 | ≧5.00E−5 |
| 79 | 1.48E−6 | ≧5.00E−5 |
| 80 | 3.30E−6 | ≧5.00E−5 |
| 81 | 2.90E−7 | ≧5.00E−5 |
| 82 | 3.25E−7 | ≧5.00E−5 |
| 83 | 3.19E−7 | ≧5.00E−5 |
| 84 | 2.03E−6 | ≧5.00E−5 |
| 85 | 9.97E−8 | ≧5.00E−5 |
| 86 | 8.70E−8 | ≧5.00E−5 |
| 87 | 1.96E−7 | 3.40E−5 |
| 88 | 4.48E−8 | ≧5.00E−5 |
| 89 | 6.76E−7 | 2.02E−5 |
| 90 | 5.00E−8 | 2.74E−5 |
| 91 | 2.62E−7 | ≧5.00E−5 |
| 92 | 4.48E−7 | ≧5.00E−5 |
| 93 | 1.94E−7 | ≧5.00E−5 |
| 94 | 1.09E−8 | ≧5.00E−5 |
| 95 | 4.58E−9 | ≧5.00E−5 |
| 96 | 3.45E−8 | 8.25E−6 |
| 97 | 2.42E−8 | ≧5.00E−5 |
| 98 | 7.24E−9 | ≧5.00E−5 |
| 99 | 3.80E−7 | ≧5.00E−5 |
| 100 | 2.48E−8 | 2.08E−5 |
| 101 | 5.21E−9 | ≧5.00E−5 |
| 102 | 4.59E−8 | 2.89E−5 |
| 103 | 3.02E−7 | 7.40E−6 |
| 104 | 5.34E−7 | 7.40E−6 |
| 105 | 5.52E−7 | 7.80E−6 |
| 106 | 1.72E−8 | ≧5.00E−5 |
| 107 | 3.85E−8 | 2.10E−5 |
| 108 | 4.45E−8 | 4.35E−5 |
| 109 | 4.88E−8 | ≧5.00E−5 |
| 110 | 3.66E−7 | 6.50E−6 |
| 111 | 1.62E−7 | ≧5.00E−5 |
| 112 | 8.32E−8 | ≧5.00E−5 |
| 113 | 1.59E−7 | ≧5.00E−5 |
| 114 | 1.58E−8 | 2.10E−5 |
| 115 | 3.62E−8 | ≧5.00E−5 |
| 116 | 1.32E−7 | ≧5.00E−5 |
| 117 | 1.53E−7 | ≧5.00E−5 |
| 118 | 9.60E−7 | ≧5.00E−5 |
| 119 | 2.60E−7 | ≧5.00E−5 |
| 120 | 7.55E−7 | ≧5.00E−5 |
| 121 | 2.37E−7 | ≧5.00E−5 |
| 122 | 3.38E−7 | 1.55E−5 |
| 123 | 4.00E−7 | ≧5.00E−5 |
| 124 | 3.40E−8 | ≧5.00E−5 |
| 125 | 1.05E−7 | 8.90E−6 |
| 126 | 2.22E−7 | ≧5.00E−5 |
| 127 | 4.45E−8 | ≧5.00E−5 |
| 128 | 7.40E−8 | ≧5.00E−5 |
| 129 | 1.95E−7 | 1.10E−5 |
| 130 | 2.48E−7 | 2.80E−5 |
| 131 | 1.74E−7 | ≧5.00E−5 |
| 132 | 3.10E−7 | ≧5.00E−5 |
| 133 | 3.62E−7 | 3.00E−5 |
| 134 | 3.98E−7 | ≧5.00E−5 |
| 135 | 5.92E−7 | ≧5.00E−5 |
| 136 | 7.50E−7 | 3.85E−5 |
| 137 | 9.25E−7 | 3.30E−5 |
| 138 | 1.20E−6 | ≧5.00E−5 |
| 139 | 1.43E−6 | ≧5.00E−5 |
| 140 | 1.70E−6 | 2.50E−5 |
| 141 | 2.50E−6 | ≧5.00E−5 |
| 142 | 2.85E−6 | ≧5.00E−5 |
| 143 | 3.30E−6 | ≧5.00E−5 |
| 144 | 3.85E−6 | ≧5.00E−5 |
| 145 | 4.80E−6 | ≧5.00E−5 |
| 146 | 9.98E−9 | 2.70E−5 |
| 147 | 1.02E−8 | ≧5.00E−5 |
| 148 | 1.22E−8 | ≧5.00E−5 |
| 149 | 1.33E−8 | 1.50E−5 |
| 150 | 2.62E−8 | ≧5.00E−5 |
| 151 | 2.82E−8 | ≧5.00E−5 |
| 152 | 3.38E−8 | 2.90E−5 |
| 153 | 4.40E−8 | ≧5.00E−5 |
| 154 | 4.76E−8 | 4.25E−5 |
| 155 | 4.98E−8 | ≧5.00E−5 |
| 156 | 5.27E−8 | ≧5.00E−5 |
| 157 | 6.05E−8 | 4.35E−5 |
| 158 | 6.30E−8 | ≧5.00E−5 |
| 159 | 7.05E−8 | ≧5.00E−5 |
| 160 | 7.95E−8 | 3.00E−5 |
| 161 | 9.80E−8 | ≧5.00E−5 |
| 162 | 1.03E−7 | 1.04E−5 |
| 163 | 1.27E−7 | 1.26E−5 |
| 164 | 1.34E−7 | ≧5.00E−5 |
| 165 | 1.35E−7 | ≧5.00E−5 |
| 166 | 1.35E−7 | 5.40E−6 |
| 167 | 1.39E−7 | 4.12E−5 |
| 168 | 1.45E−7 | ≧5.00E−5 |
| 169 | 1.54E−7 | 1.00E−5 |
| 170 | 1.78E−7 | 7.40E−6 |
| 171 | 2.00E−7 | ≧5.00E−5 |
| 172 | 2.11E−7 | ≧5.00E−5 |
| 173 | 2.14E−7 | 7.50E−6 |
| 174 | 2.15E−7 | ≧5.00E−5 |
| 175 | 2.18E−7 | 7.30E−6 |
| 176 | 2.60E−7 | 3.42E−6 |
| 177 | 2.65E−7 | 1.50E−5 |
| 178 | 2.85E−7 | 4.75E−5 |
| 179 | 3.00E−7 | 7.60E−6 |
| 180 | 3.01E−7 | ≧5.00E−5 |
| 181 | 3.02E−7 | ≧5.00E−5 |
| 182 | 3.11E−7 | ≧5.00E−5 |
| 183 | 3.44E−7 | ≧5.00E−5 |
| 184 | 3.46E−7 | ≧5.00E−5 |
| 185 | 3.74E−7 | ≧5.00E−5 |
| 186 | 3.85E−7 | ≧5.00E−5 |
| 187 | 4.23E−7 | ≧5.00E−5 |
| 188 | 4.56E−7 | 3.30E−5 |
| 189 | 4.79E−7 | ≧5.00E−5 |
| 190 | 5.68E−7 | ≧5.00E−5 |
| 191 | 7.15E−7 | 1.20E−5 |
| 192 | 7.85E−7 | ≧5.00E−5 |
| 193 | 8.10E−7 | 1.70E−5 |
| 194 | 8.64E−7 | ≧5.00E−5 |
| 195 | 9.75E−7 | 3.90E−5 |
| 196 | 1.17E−6 | 1.30E−5 |
| 197 | 1.48E−6 | ≧5.00E−5 |
| 198 | 1.50E−6 | ≧5.00E−5 |
| 199 | 1.75E−6 | ≧5.00E−5 |
| 200 | 1.75E−6 | ≧5.00E−5 |
| 201 | 1.90E−6 | ≧5.00E−5 |
| 202 | 1.90E−6 | ≧5.00E−5 |
| 203 | 2.02E−6 | ≧5.00E−5 |
| 204 | 2.25E−6 | ≧5.00E−5 |
| 205 | 2.45E−6 | ≧5.00E−5 |
| 206 | 2.48E−6 | ≧5.00E−5 |
| 207 | 2.60E−6 | ≧5.00E−5 |
| 208 | 3.00E−6 | ≧5.00E−5 |
| 209 | 3.55E−6 | ≧5.00E−5 |
| 210 | 3.60E−6 | ≧5.00E−5 |
| 211 | 3.90E−6 | ≧5.00E−5 |
| 212 | 4.35E−6 | ≧5.00E−5 |
| 213 | 4.60E−6 | ≧5.00E−5 |
| 214 | 4.90E−6 | ≧5.00E−5 |
| 215 | 7.72E−7 | 1.89E−5 |
| 216 | 5.24E−7 | 6.95E−6 |
| 217 | 2.70E−8 | 1.63E−6 |
| 218 | 1.10E−8 | ≧5.00E−5 |
| 219 | 6.55E−8 | ≧5.00E−5 |
| 220 | 1.60E−7 | ≧5.00E−5 |
| 221 | 1.10E−6 | ≧5.00E−5 |
| 222 | 1.05E−7 | 9.00E−6 |
| Ref. | 1.38E−6 | 3.10E−6 |

“Ref.” in Table 1 means the compound niclosamide disclosed in prior art (compound 1-8 on page 36 of WO2011/035321A1) which is less selective than the compounds of the present invention.

### Measurement of the Inhibitory Activity of Selected Compounds on the Wildtype Wnt Signaling Cascade

In order to discover and characterize small molecules which inhibit the wildtype Wnt pathway, a cellular reporter assay was employed. The corresponding assay cell was generated by transfection of the mammalian cell line HEK293 (ATCC, #CRL-1573) with the Super TopFlash vector (Morin, Science 275, 1997, 1787-1790; Molenaar et al., Cell 86 (3), 1996, 391-399). The HEK293 cell line is cultivated at 37° C. and 5% CO₂ in DMEM (Life Technologies, #41965-039), supplemented with 2 mM glutamine, 20 mM HEPES, 1.4 mM pyruvate, 0.15% Na-bicarbonate and 10% foetal bovine serum (GIBCO, #10270). Stable transfectants were generated by selection with 300 μg/ml Hygromycin.

In a parallel approach, HEK293 cells were cotransfected with the FOP control vector and pcDNA3. The FOP vector is identical to the TOP construct, but it contains instead of functional TCF elements a randomized, non-functional sequence. For this transfection a stable transfected cell line was generated as well, based on selection with Geneticin (1 mg/ml).

In preparation of the assay, the two cell lines were plated 24 hours before beginning the test at 10000 cells per well in a 384 micro titre plate (MTP) in 30 it growth medium. Before compound testing a dose response curve for the Wnt dependent luciferase expression was recorded by stimulating the assay cell line with human recombinant Wnt-3a (R&D, #5036-WN-010) at different concentrations for 16 hours at 37° C. and 5% CO₂ followed by subsequent luciferase measurement, to determine the Wnt-3a EC₅₀ for the HEK293 TOP cell line on the day of testing. The recombinant human Wnt-3a was thereby applied between 2500 and 5 ng/ml in two-fold dilution steps. Selective inhibitory activity for small molecules on the wildtype Wnt pathway was determined after parallel incubation of both (TOP and FOP) HEK293 reporter cell lines with a compound dilution series from 50 μM to 15 nM in steps of 3.16-fold dilutions in CAFTY buffer (130 mM NaCl, 5 mM KCl, 20 mM HEPES, 1 mM MgCl₂, 5 mM NaHCO₃, pH 7.4) containing 2 mM Ca²⁺ and 0.01% BSA.

The compounds were thereby serially prediluted in 100% DMSO and thereafter 50 fold into the CAFTY compound dilution buffer (described above). From this dilution 10 it were added in combination with the EC₅₀ concentration of recombinant Wnt3a to the cells in 30 it growth medium and incubated for 16 hours at 37° C. and 5% CO₂. Thereafter luciferase assay buffer (1:1 mixture of luciferase substrate buffer (20 mM Tricine, 2.67 mM MgSO₄, 0.1 mM EDTA, 4 mM DTT, 270 μM Coenzyme A, 470 μM Luciferin, 530 μM ATP, ph adjusted to pH 7.8 with a sufficient volume of 5M NaOH) and Triton buffer (30 ml Triton X-100, 115 ml glycerol, 308 mg Dithiothreitol, 4.45 g Na₂HPO₄ 2H₂O, 3.03 g TRIS HCl (CAS Number 1185-53-1), ad 1 l H₂0, pH 7.8) was added in an equal volume to determine luciferase expression as a measure of Wnt signaling activity in a luminometer. The Wnt inhibitory activity was determined as IC₅₀ of resulting dose response curves.

TABLE 3

| Example | HEK TOP OncoFlash | HEK FOP IC₅₀ |
|---|---|---|
| No | IC₅₀ [mol/L] | [mol/L] |
| 1 | 3.3E−6 | ≧5.0E−5 |
| 2 | 1.48E−6 | 5.0E−5 |
| 4 | 4.0E−7 | 8.6E−6 |
| 7 | 8.2E−7 | 1.0E−5 |
| 8 | 2.7E−7 | ≧5.0E−5 |
| 9 | 1.2E−7 | ≧5.0E−5 |
| 12 | 4.4E−7 | ≧5.0E−5 |
| 13 | 5.0E−7 | 1.2E−5 |
| 14 | 2.4E−7 | 7.0E−6 |
| 15 | 1.0E−7 | 4.0E−6 |
| 16 | 2.6E−8 | 7.9E−6 |
| 17 | 2.9E−7 | 3.3E−5 |
| 18 | 6.9E−7 | 1.9E−5 |
| 20 | 1.2E−7 | ≧5.0E−5 |
| 22 | 7.7E−8 | ≧5.0E−5 |
| 23 | 3.8E−8 | ≧5.0E−5 |
| 25 | 1.2E−7 | ≧5.0E−5 |
| 26 | 9.7E−7 | ≧5.0E−5 |
| 27 | 7.0E−7 | 9.3E−6 |
| 28 | 2.4E−6 | ≧5.0E−5 |
| 30 | 9.2E−7 | 9.2E−6 |
| 32 | 5.2E−7 | 1.7E−5 |
| 33 | 4.0E−7 | 9.0E−6 |
| 35 | 9.2E−7 | 2.9E−5 |
| 36 | 8.2E−7 | 9.9E−6 |
| 37 | 6.4E−7 | ≧5.0E−5 |
| 39 | 1.1E−6 | ≧5.0E−5 |
| 40 | 4.6E−8 | 1.2E−5 |
| 41 | 7.1E−7 | ≧5.0E−5 |
| 43 | 1.2E−6 | ≧5.0E−5 |
| 48 | 2.8E−6 | ≧5.0E−5 |
| 53 | 2.9E−7 | 6.2E−6 |
| 56 | 4.0E−6 | ≧5.0E−5 |
| 58 | 2.2E−6 | 3.5E−5 |
| 59 | 1.7E−6 | ≧5.0E−5 |
| 61 | 1.4E−7 | ≧5.0E−5 |
| 62 | 5.5E−8 | 8.0E−6 |
| 66 | 1.6E−6 | ≧5.0E−5 |
| 71 | 2.5E−6 | 2.5E−5 |
| 72 | 1.3E−6 | ≧5.0E−5 |
| 73 | 1.5E−6 | 3.2E−5 |
| 74 | 1.4E−7 | 8.3E−6 |
| 75 | 8.5E−7 | 4.0E−5 |
| 76 | 3.9E−8 | 1.1E−5 |
| 79 | 4.1E−6 | ≧5.0E−5 |
| 80 | 3.8E−6 | ≧5.0E−5 |
| 81 | 3.2E−7 | 9.8E−6 |
| 82 | 1.7E−6 | ≧5.0E−5 |
| 83 | 7.6E−7 | ≧5.0E−5 |
| 85 | 8.3E−7 | ≧5.0E−5 |
| 86 | 6.1E−7 | ≧5.0E−5 |
| 88 | 9.3E−8 | ≧5.0E−5 |
| 90 | 2.06E−8 | 6.1E−6 |
| 94 | 5.09E−9 | 7.6E−6 |
| 95 | 1.6E−8 | ≧5.0E−5 |
| 96 | 4.0E−8 | 9.6E−6 |
| 97 | 2.5E−7 | ≧5.0E−5 |
| 98 | 8.6E−8 | ≧5.0E−5 |
| 100 | 9.0E−8 | 1.9E−5 |
| 101 | 1.1E−8 | 1.7E−5 |
| 102 | 4.1E−8 | 5.8E−6 |
| 106 | 7.6E−8 | ≧5.0E−5 |
| 107 | 3.1E−7 | 3.6E−5 |
| 108 | 1.56E−8 | ≧5.0E−5 |
| 109 | 2.8E−7 | ≧5.0E−5 |
| 112 | 7.7E−8 | ≧5.0E−5 |
| 114 | 3.6E−8 | ≧5.0E−5 |
| 115 | 1.7E−7 | ≧5.0E−5 |
| 124 | 1.1E−7 | 1.1E−5 |
| 127 | 1.1E−7 | 3.1E−5 |

### QPCR Protocol

Real-time RT-PCR using a TaqMan fluorogenic detection system is a simple and sensitive assay for quantitative analysis of gene transcription. The TaqMan fluorogenic detection system can monitor PCR in real time using a dual-labeled fluorogenic hybridization probe (TaqMan probe) and a polymerase with 5′-3′ exonuclease activity.

Cells from different cancer cell lines (as HCT116, but not limited to) were grown at 500-1000 cells/well in 384 well cell culture plates. For cell lysis the cell medium was carefully removed. The cells were washed carefully once with 50 μL/well PBS. Then 9.75 μL/well cell lysis buffer (50 mM Tris Hcl pH 8.0, 40 mM NaCl, 1.5 mM MgCl₂, 0.5% IGEPAL CA 630, 50 mM Guanidium thiocyanate) and 0.25 μL RNASeOUT (40 U/μl, Invitrogen, 10777-019)) per well were added. The plate was incubated for 5 min at room temperature. Then 30 μL DNAse/RNAse-free water per well added and the lysates were mixed. For the One-Step RT-PCR 2 μL lysate (each) was transferred to a 384 well PCR plate. The PCR reaction was composed by 5 μL 2× One Step RT qPCR MasterMix Plus, 0.05 μL Euroscript RT/RNAse Inhibitor (50 U/μl, 20 U/μl) and 200 nM of the appropriate Primer/Hydrolysis Probe mix (primer sequences of forward, reverse and probe are given below for each analysed gene of interest or house keeping gene). 10 μL water were added per well. Seal the plate with an adhesive optical film. The RT-PCR protocol was setup with 30 min 48° C., then 10 min 95° C. followed by 50 cycles of 15 sec 95° C./1 min 60° C. and a cooling step of 40° C. for 30 sec using a Lightcycler L5440 from Roche. Relative expression was calculated using CP values from the gene of interest (e.g. AXIN2, but not limited to) and a house keeping gene (L32).

### Used Primers

L32 (forward primer: AAGTTCATCCGGCACCAGTC; reverse primer: TGGCCCTTGAATCTTCTACGA; probe: CCCAGAGGCATTGACAACAGGG)

AXIN2 (forward primer: AGGCCAGTGAGTTGGTTGTC; reverse primer: AGCTCTGAGCCTTCAGCATC; probe: TCTGTGGGGAAGAAATTCCATACCG)

### Sequence Listings

### SEQ ID NO

1 AAGTTCATCCGGCACCAGTC
2 TGGCCCTTGAATCTTCTACGA
3 CCCAGAGGCATTGACAACAGGG
4 AGGCCAGTGAGTTGGTTGTC
5 AGCTCTGAGCCTTCAGCATC
6 TCTGTGGGGAAGAAATTCCATACCG

1. A compound of general formula (I): 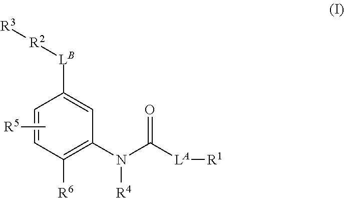
in which:
L^{A} represents a methylene or ethylene group, said methylene or ethylene group being optionally substituted, one or more times, identically or differently, with a substituent selected from:
hydroxy-, cyano-, C₁-C₃-alkoxy-, hydroxy-C₁-C₃-alkyl-, halo-C₁-C₃-alkoxy-, C₃-C₇-cycloalkyl-, 3- to 10-membered heterocycloalkyl-;
or, when two substituents are present at the same carbon atom, the two substituents, together with the carbon atom they are attached to, may form a
C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkoxy-;
L^{B} represents —N(H)—C(═O)— or —C(═O)—N(H)—;
R¹ represents a group selected from:
C₃-C₇-cycloalkyl-, C₄-C₇-cycloalkenyl-, 3- to 10-membered heterocycloalkyl-,
4- to 10-membered heterocycloalkenyl-, aryl-, heteroaryl-, —N(R⁷)—(C₁-C₆-alkyl), —N(R⁷)—C(═O)—O—(C₁-C₆-alkyl), —N(R⁷)R⁷;
wherein said C₃-C₇-cycloalkyl-, C₄-C₇-cycloalkenyl-, 3- to 10-membered heterocycloalkyl-, 4- to 10-membered heterocycloalkenyl-, aryl-, heteroaryl-, and —N(R⁷)—(C₁-C₆-alkyl) group is optionally substituted, one or more times, identically or differently, with a substituent selected from: halo-, hydroxy-, cyano-, C₁-C₃-alkyl-, C₁-C₃-alkoxy-,
halo-C₁-C₃-alkyl-, hydroxy-C₁-C₃-alkyl-, halo-C₁-C₃-alkoxy-,
C₃-C₇-cycloalkyl-, 3- to 10-membered heterocycloalkyl-,
—C(═O)R⁹, —C(═O)O—R⁹, —OC(═O)—R⁹,
—N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
—NR¹⁰R⁹, —C(═O)N(H)R⁹, —C(═O)NR¹⁰R⁹, R⁹—S—, R⁹—S(═O)—, R⁹—S(═O)₂—,
—N(H)S(═O)R⁹, —N(R¹⁰)S(═O)R⁹, —S(═O)N(H)R⁹, —S(═O)NR¹⁰R⁹,
—N(H)S(═O)₂R⁹, —N(R⁹)S(═O)₂R¹⁰, —S(═O)₂N(H)R⁹, —S(═O)₂NR¹⁰R⁹,
—S(═O)(═NR¹⁰)R⁹, —S(═O)(═NR¹⁰)R⁹, —N═S(═O)(R¹⁰)R⁹;
R² represents: 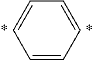
wherein “*” represents the point of attachment to R³ or L^{B}, respectively; wherein said group is optionally substituted, one or more times, identically or differently, with halo- or a C₁-C₃-alkyl-group;
R³ represents a phenyl-group,
said phenyl-group being optionally substituted, one or more times, identically or differently, with a substituent selected from: halo-, hydroxy-, cyano-, C₁-C₃-alkyl-, C₁-C₃-alkoxy-, halo-C₁-C₃-alkyl-, hydroxy-C₁-C₃-alkyl-, NH₂—C₁-C₃-alkyl-, halo-C₁-C₃-alkoxy-,
C₃-C₇-cycloalkyl-, 3- to 10-membered heterocycloalkyl-,
—C(═O)R⁹, —C(═O)O—R⁹, —OC(═O)—R⁹,
—N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹,
—NR¹⁰R⁹, —C(═O)N(H)R⁹, —C(═O)NR¹⁰R⁹, R⁹—S—, R⁹—S(═O)—, R⁹—S(═O)₂—,
—N(H)S(═O)R⁹, —N(R¹⁰)S(═O)R⁹, —S(═O)N(H)R⁹, —S(═O)NR¹⁰R⁹,
—N(H)S(═O)₂R⁹, —N(R⁹)S(═O)₂R¹⁰, —S(═O)₂N(H)R⁹, —S(═O)₂NR¹⁰R⁹,
—S(═O)(═NR¹⁰)R⁹, —S(═O)(═NR¹⁰)R⁹, —N═S(═O)(R¹⁰)R⁹;
or, when two substituents are present ortho to each other on the phenyl-group, said two substituents together form a bridge: *O(CH₂)₂O*, *O(CH₂)O*, *O—C(H)₂—C(H)₂*, *NH(C(═O))NH*, wherein * represent the points of attachment to the phenyl-group;
R⁴ represents a hydrogen atom or a group selected from:
C₁-C₆-alkyl-, C₃-C₄-alkenyl-, C₃-C₄-alkynyl-,
—(CH₂)ₘ—C₃-C₇-cycloalkyl, —(CH₂)ₘ—C₄-C₇-cycloalkenyl,
—(CH₂)ₘ-(3- to 10-membered heterocycloalkyl),
—(CH₂)ₘ-(4- to 10-membered heterocycloalkenyl),
—(CH₂)ₘ-aryl, —(CH₂)ₘ-heteroaryl;
R⁵ represents a hydrogen atom or a halogen atom or a group selected from:
cyano-, C₁-C₃-alkyl-, C₁-C₃-alkoxy-;
R⁶ represents a group selected from:
C₁-C₆ alkyl, C₂-C₆-alkenyl-, C₂-C₆-alkynyl-C₁-C₆-alkoxy-, C₃-C₆-cycloalkoxy-, halo-, hydroxy-, cyano-, aryl-, heteroaryl-, —N(R⁹)(R¹⁰), —C(═O)—O—R⁹, —C(═O)—N(R⁹)(R¹⁰), R⁹—S—, R⁹—S(═O)—, R⁹—S(═O)₂—;
said C₁-C₆-alkyl-, C₂-C₆-alkenyl-, C₂-C₆-alkynyl-, aryl-, heteroaryl- or C₁-C₆-alkoxy-group being optionally substituted, one or more times, identically or differently, with halo-, cyano-, nitro-, hydroxy-, C₁-C₃-alkyl-, C₁-C₃-alkoxy-, halo-C₁-C₃-alkoxy-,
hydroxy-C₁-C₃-alkoxy-, C₁-C₃-alkoxy-C₁-C₃-alkoxy-, C₃-C₇-cycloalkyl-, C₄-C₇-cycloalkenyl-, 3- to 10-membered heterocycloalkyl-, 4- to 10-membered heterocycloalkenyl-, aryl-, heteroaryl-, —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—, —N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰, —S(═O)₂N(H)R⁹, —S(═O)₂NR¹⁰R⁹, —S(═O)(═NR¹⁰)R⁹,
—S(═O)(═NR¹⁰)R⁹, —N═S(═O)(R¹⁰)R⁹;
R⁷ represents —H or C₁-C₃-alkyl-;
R⁹, R¹⁰, R¹¹
represent, independently from each other, —H, C₁-C₃-alkyl- or C₃-C₆-cycloalkyl-;
said C₁-C₃-alkyl-group being optionally substituted with C₁-C₃-alkoxy- or —N(R¹²)R¹³;
or
R⁹R¹⁰ together with the atom or the group of atoms they are attached to, form a 3- to 10-membered heterocycloalkyl- or 4- to 10-membered heterocycloalkenyl-group;
R¹², R¹³
represent, independently from each other, —H or C₁-C₃-alkyl-;
or
R¹², R¹³ together with the atom they are attached to, form a 3- to 10-membered heterocycloalkyl- or 4- to 10-membered heterocycloalkenyl-group;
m represents 0, 1, or 2;
or a tautomer, an N-oxide, a hydrate, a solvate, or a salt thereof, or a mixture of same.

2. A compound according to claim 1, wherein:
L^{A} represents a methylene or ethylene group, said methylene or ethylene group being optionally substituted, one or more times, identically or differently, with a substituent selected from:
halo-, hydroxy-, cyano-, C₁-C₃-alkoxy-, fluoro-C₁-C₃-alkyl-,
hydroxy-C₁-C₃-alkyl-, fluoro-C₁-C₃-alkoxy-, C₃-C₇-cycloalkyl-,
3- to 10-membered heterocycloalkyl-;
or, when two substituents are present at the same carbon atom, the two substituents, together with the carbon atom they are attached to, may form a
C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
C₃-C₇-cycloalkyl-, 3- to 10-membered heterocycloalkyl-, aryl-, heteroaryl-,
—N(R⁷)—(C₁-C₆-alkyl);
wherein each group is optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
C₃-C₇-cycloalkyl-, 3- to 10-membered heterocycloalkyl-,
—C(═O)R⁹, —C(═O)O—R⁹, —N(H)C(═O)R⁹, —N(R¹⁰)C(═O)R⁹, —N(H)C(═O)NR¹⁰R⁹,
—N(R¹¹)C(═O)NR¹⁰R⁹, —N(H)R⁹, —NR¹⁰R⁹, —C(═O)N(H)R⁹, —C(═O)NR¹⁰R⁹, R⁹—S(═O)₂—;
R² represents: 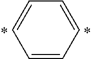
wherein “*” represents the point of attachment to R³ or L^{B}, respectively; wherein said group is optionally substituted, one or more times, identically or differently, with a C₁-C₃-alkyl-group;
R³ represents a phenyl-group,
said phenyl-group being optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
C₃-C₅-cycloalkyl-, 3- to 6-membered heterocycloalkyl-,
—C(═O)R⁹, —C(═O)O—R⁹, —N(H)C(═O)R⁹, —N(R¹⁰)C(═O)R⁹, —N(H)C(═O)NR¹⁰R⁹,
—N(R¹¹)C(═O)NR¹⁰R⁹, —N(H)R⁹, —NR¹⁰R⁹, —C(═O)N(H)R⁹, —C(═O)NR¹⁰R⁹;
R⁴ represents a hydrogen atom or a group selected from:
—C₁-C₆-alkyl-, —(CH₂)ₘ—C₃-C₇-cycloalkyl,
—(CH₂)ₘ-(3 to 10 membered heterocycloalkyl),
—(CH₂)ₘ-aryl, —(CH₂)ₘ-heteroaryl;
R⁵ represents a hydrogen atom or a halogen atom or a group selected from:
cyano-, C₁-C₃-alkyl-, C₁-C₃-alkoxy-;
R⁶ represents a group selected from:
C₁-C₆ alkyl, C₁-C₆-alkoxy-, halo-, hydroxy-, fluoro-C₁-C₆-alkyl-, fluoro-C₁-C₆-alkoxy-, phenyl-, 5- to 6-membered heteroaryl-, cyano-, —C(═O)—O—R⁹, —C(═O)—N(R⁹)(R¹⁰);
said C₁-C₆-alkyl- or C₁-C₆-alkoxy-group being optionally substituted, one or more times, identically or differently, with
halo-, cyano-, nitro-, hydroxy-, C₁-C₃-alkyl-, C₁-C₃-alkoxy-, halo-C₁-C₃-alkoxy-,
hydroxy-C₁-C₃-alkoxy-, C₁-C₃-alkoxy-C₁-C₃-alkoxy-,
C₃-C₇-cycloalkyl-, 3- to 10-membered heterocycloalkyl-, aryl-, heteroaryl-,
—C(═O)R⁹, —C(═O)O—R⁹, —OC(═O)—R⁹, —N(H)C(═O)R⁹, —N(R¹⁰)C(═O)R⁹,
—N(H)C(═O)NR¹⁰R⁹, —N(R¹¹)C(═O)NR¹⁰R⁹, —N(H)R⁹, —NR¹⁰R⁹,
—C(═O)N(H)R⁹, —C(═O)NR¹⁰R⁹;
R⁷ represents —H or C₁-C₃-alkyl-;
R⁹, R¹⁰, R¹¹
represent, independently from each other, —H or C₁-C₃-alkyl-;
or
R⁹R¹⁰ together with the atom or the group of atoms they are attached to, form a 3- to 10-membered heterocycloalkyl- or 4- to 10-membered heterocycloalkenyl-group;
m represents 0, 1- or 2;
or a tautomer, an N-oxide, a hydrate, a solvate, or a salt thereof, or a mixture of same.

3. A compound according to claim 1, wherein:
L^{A} represents a methylene group, said methylene group being optionally substituted, one or more times, identically or differently, with a substituent selected from:
cyano-, hydroxy-, C₁-C₃-alkoxy-, fluoro-C₁-C₃-alkyl-,
hydroxy-C₁-C₃-alkyl-, C₃-C₅-cycloalkyl-, 3- to 6-membered heterocycloalkyl-;
or, when two substituents are present at the same carbon atom, the two substituents, together with the carbon atom they are attached to, may form a
C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from:
3- to 10-membered heterocycloalkyl-, or 5- to 6-membered heteroaryl-,
wherein each group is optionally substituted, one or more times, identically or differently, with a substituent selected from: halo-, hydroxy-, cyano-, C₁-C₃-alkyl-, C₁-C₃-alkoxy-, fluoro-C₁-C₃-alkyl-, hydroxy-C₁-C₃-alkyl-, fluoro-C₁-C₃-alkoxy-,
C₃-C₅-cycloalkyl-, 3- to 6-membered heterocycloalkyl-,
—C(═O)R⁹, —C(═O)O—R⁹, —N(H)C(═O)R⁹, —N(R¹⁰)C(═O)R⁹, —N(H)C(═O)NR¹⁰R⁹,
—N(R¹¹)C(═O)NR¹⁰R⁹, —N(H)R⁹, —NR¹⁰R⁹, —C(═O)N(H)R⁹, —C(═O)NR¹⁰R⁹, R⁹—S(═O)₂—;
R² represents: 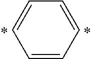
wherein “*” represents the point of attachment to R³ or L^{B}, respectively; wherein said group is optionally substituted, one or more times, identically or differently, with a C₁-C₃-alkyl-group;
R³ represents a phenyl-group,
said phenyl-group being optionally substituted, one or more times, identically or differently, with a substituent selected from: halo-, hydroxy-, cyano-, C₁-C₂-alkoxy-, fluoro-C₁-C₂-alkyl-, hydroxy-C₁-C₂-alkyl-, fluoro-C₁-C₂-alkoxy-,
—C(═O)R⁹, —C(═O)O—R⁹, —N(H)C(═O)R⁹, —N(H)R⁹, —NR¹⁰R⁹, —C(═O)N(H)R⁹, —C(═O)NR¹⁰R⁹;
R⁴ represents a hydrogen atom or group selected from:
C₁-C₆ alkyl, —(CH₂)ₘ—C₃-C₇-cycloalkyl, —(CH₂)ₘ-aryl;
R⁵ represents a hydrogen atom or a halogen atom or a group selected from:
cyano-, C₁-C₃-alkyl-, C₁-C₃-alkoxy-;
R⁶ represents a group selected from:
C₁-C₆ alkyl, C₁-C₆-alkoxy-, halo-, hydroxy-, fluoro-C₁-C₆-alkyl-, fluoro-C₁-C₆-alkoxy-, cyano-, —C(═O)—O—R⁹, —C(═O)—N(R⁹)(R¹⁰);
said C₁-C₆-alkyl-, or C₁-C₆-alkoxy-group being optionally substituted, one or more times, identically or differently, with
C₃-C₇-cycloalkyl-, 3- to 10-membered heterocycloalkyl-, aryl-, heteroaryl-,
—C(═O)R⁹, —C(═O)O—R⁹, —OC(═O)—R⁹, —N(H)C(═O)R⁹, —N(R¹⁰)C(═O)R⁹,
—N(H)C(═O)NR¹⁰R⁹, —N(R¹¹)C(═O)NR¹⁰R⁹, —N(H)R⁹, —NR¹⁰R⁹,
—C(═O)N(H)R⁹, —C(═O)NR¹⁰R⁹;
R⁹, R¹⁰, R¹¹
represent, independently from each other, —H or C₁-C₃-alkyl-;
m represents 0 or 1;
or a tautomer, an N-oxide, a hydrate, a solvate, or a salt thereof, or a mixture of same.

4. A compound according to claim 1, wherein:
L^{A} represents a methylene group, said methylene group being optionally substituted, one or more times, identically or differently, with a substituent selected from:
hydroxy-, C₁-C₃-alkyl-, C₁-C₃-alkoxy-, hydroxy-C₁-C₃-alkyl-,
or, when two substituents are present at the same carbon atom, the two substituents, together with the carbon atom they are attached to, may form a
C₃-C₆-cycloalkyl- or 3- to 6-membered heterocycloalkyl-ring; wherein said ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a morpholino group, which is attached to L^{A} via its nitrogen atom, and which may be optionally substituted one or two times, identically or differently, with C₁-C₃-alkyl-,
or two of said C₁-C₃-alkyl-groups together may form a C₁-C₃-alkylene group,
or
R¹ represents thiomorpholino, 4-cyclopropylpiperazino, 4-methylpiperazino, piperidino or pyrazol-1-yl group; said groups being attached to L^{A} via their ring nitrogen atom;
R² represents: 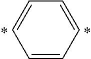
wherein “*” represents the point of attachment to R³ or L^{B}, respectively; wherein said group is optionally substituted, one or more times, identically or differently, with a C₁-C₃-alkyl-group;
R³ represents a phenyl-group,
said phenyl-group being optionally substituted, one or two times, identically or differently, with fluoro, chloro, —NH₂ or methoxy;
R⁴ represents hydrogen, C₁-C₃-alkyl- or benzyl-;
R⁵ represents hydrogen, fluoro or chloro;
R⁶ represents halo-, cyano-, C₁-C₄-alkyl-, fluoro-C₁-C₃-alkyl-, C₁-C₄-alkoxy- or fluoro-C₁-C₃-alkoxy-, —C(O)NR⁹R¹⁰ or a 5-membered heteroaryl-,
wherein said C₁-C₄-alkyl- and C₁-C₄-alkoxy group may be optionally substituted by one phenyl group;
or a tautomer, an N-oxide, a hydrate, a solvate, or a salt thereof, or a mixture of same.

5. A compound according to claim 1, wherein:
L^{A} represents methylene, said methylene group being optionally substituted one or two times, identically or differently, with C₁-C₃-alkyl-,
wherein, if said methylene is substituted with two C₁-C₃-alkyl-groups, these may, together with the carbon atom they are attached to, form a C₃-C₆-cycloalkyl-ring;
L^{B} represents —N(H)—C(═O)— or —C(═O)—N(H)—;
R¹ represents a morpholino group, which is attached to L^{A} via its nitrogen atom, and which may be optionally substituted one or two times, identically or differently, with C₁-C₃-alkyl-,
or two of said C₁-C₃-alkyl groups together may form a C₁-C₃-alkylene group;
R² represents: 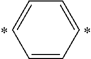
wherein “*” represents the point of attachment to R³ or L^{B}, respectively;
R³ represents a phenyl-group,
said phenyl-group being optionally substituted one or two times, identically or differently, with fluoro or methoxy;
R⁴ represents hydrogen;
R⁵ represents hydrogen;
R⁶ represents halogen, C₁-C₄-alkyl-, fluoro-C₁-C₃-alkyl-, C₁-C₄-alkoxy- or fluoro-C₁-C₃-alkoxy-;
or a tautomer, an N-oxide, a hydrate, a solvate, or a salt thereof, or a mixture of same.

6. A compound according to claim 1, wherein:
L^{A} represents —CH₂—, —CH(CH₃)—, or —C(CH₃)₂—;
L^{B} represents —N(H)—C(═O)— or —C(═O)—N(H)—;
R¹ represents a group selected from: 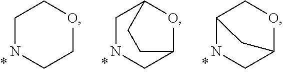
wherein “*” indicates the point of attachment to L^{A};
R² represents: 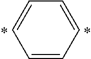
wherein “*” represents the point of attachment to R³ or L^{B}, respectively;
R³ represents a phenyl-group,
said phenyl-group being optionally substituted, one or two times, with fluoro;
R⁴ represents hydrogen;
R⁵ represents hydrogen;
R⁶ represents chloro, C₁-C₄-alkyl-, methoxy-, trifluoromethoxy- or trifluoromethyl-;
or a tautomer, an N-oxide, a hydrate, a solvate, or a salt thereof, or a mixture of same.

7. A compound according to claim 1, wherein:
L^{A} represents —CH₂— or —CH(CH₃)—;
L^{B} represents —N(H)—C(═O)— or —C(═O)—N(H)—;
R¹ represents a group selected from: 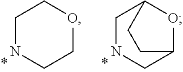
wherein “*” indicates the point of attachment to L^{A};
R² represents: 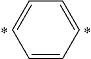
wherein “*” represents the point of attachment to R³ or L^{B}, respectively;
R³ represents a phenyl-group,
said phenyl-group being optionally substituted, one or two times, with fluoro;
R⁴ represents hydrogen;
R⁵ represents hydrogen;
R⁶ represents trifluoromethoxy or tert-butyl;
or a tautomer, an N-oxide, a hydrate, a solvate, or a salt thereof, or a mixture of same.

8. A compound according to claim 1, which is selected from the group consisting of:
N-(biphenyl-4-yl)-4-methoxy-3-[(morpholin-4-ylacetyl)amino]benzamide,
N-(biphenyl-4-yl)-4-methoxy-3-[(1H-pyrazol-1-ylacetyl)amino]benzamide,
N-(biphenyl-4-yl)-3-[(1H-pyrazol-1-ylacetyl)amino]-4-(trifluoromethyl)benzamide,
N-(biphenyl-4-yl)-3-{[2-methyl-2-(1H-pyrazol-1-yl)propanoyl]amino}-4-(trifluoromethyl)benzamide,
N-(biphenyl-4-yl)-2-chloro-4-methoxy-5-{[2-(morpholin-4-yl)propanoyl]amino}benzamide,
N-(biphenyl-4-yl)-2-chloro-4-methoxy-5-[(morpholin-4-ylacetyl)amino]benzamide,
N-(biphenyl-4-yl)-4-methoxy-3-{[(1S,4S)-2-oxa-5-azabicyclo[2.2.1]hept-5-ylacetyl]amino}benzamide,
N-(biphenyl-4-yl)-4-methoxy-3-[(8-oxa-3-azabicyclo[3.2.1]oct-3-ylacetyl)amino]benzamide,
N-(biphenyl-4-yl)-3-[(morpholin-4-ylacetyl)amino]-4-(trifluoromethyl)benzamide,
N-(biphenyl-4-yl)-3-[(8-oxa-3-azabicyclo[3.2.1]oct-3-ylacetyl)amino]-4-(trifluoromethyl)benzamide,
N-(biphenyl-4-yl)-3-{[(1S,4S)-2-oxa-5-azabicyclo[2.2.1]hept-5-ylacetyl]amino}-4-(trifluoromethyl)benzamide,
methyl 4-(biphenyl-4-ylcarbamoyl)-2-[(morpholin-4-ylacetyl)amino]benzoate,
N-(biphenyl-4-yl)-4-bromo-3-[(morpholin-4-ylacetyl)amino]benzamide,
N-(biphenyl-4-yl)-3-{[2-(morpholin-4-yl)propanoyl]amino}-4-(trifluoromethyl)benzamide,
N-(biphenyl-4-yl)-3-{[(2S)-2-(morpholin-4-yl)propanoyl]amino}-4-(trifluoromethyl)benzamide,
N-(biphenyl-4-yl)-3-{[(2R)-2-(morpholin-4-yl)propanoyl]amino}-4-(trifluoromethyl)benzamide,
N-(biphenyl-4-yl)-3-{[2-methyl-2-(morpholin-4-yl)propanoyl]amino}-4-(trifluoromethyl)benzamide,
N-(biphenyl-4-yl)-4-cyano-3-[(morpholin-4-ylacetyl)amino]benzamide,
N-(biphenyl-4-yl)-3-[(morpholin-4-ylacetyl)amino]-4-(2-thienyl)benzamide,
N-(biphenyl-4-yl)-4-(2-furyl)-3-[(morpholin-4-ylacetyl)amino]benzamide,
N⁴-(biphenyl-4-yl)-N¹,N¹-dimethyl-2-[(morpholin-4-ylacetyl)amino]terephthalamide,
N⁴-(biphenyl-4-yl)-N¹-methyl-2-[(morpholin-4-ylacetyl)amino]terephthalamide,
N-(biphenyl-4-yl)-3-[(morpholin-4-ylacetyl)amino]-4-(trifluoromethoxy)benzamide,
4-(benzyloxy)-N-(biphenyl-4-yl)-3-[(morpholin-4-ylacetyl)amino]benzamide,
N-(biphenyl-4-yl)-4-isopropoxy-3-[(morpholin-4-ylacetyl)amino]benzamide,
N-(biphenyl-4-yl)-4-ethoxy-3-[(morpholin-4-ylacetyl)amino]benzamide,
N-{4-methoxy-3-[(1H-pyrazol-1-ylacetyl)amino]phenyl}biphenyl-4-carboxamide,
N-(4-methoxy-3-{[2-methyl-2-(morpholin-4-yl)propanoyl]amino}phenyl)biphenyl-4-carboxamide,
N-{4-fluoro-3-[(morpholin-4-ylacetyl)amino]phenyl}biphenyl-4-carboxamide,
N-(4-fluoro-3-{[(2S)-2-(morpholin-4-yl)propanoyl]amino}phenyl)biphenyl-4-carboxamide,
N-(4-fluoro-3-{[(2R)-2-(morpholin-4-yl)propanoyl]amino}phenyl)biphenyl-4-carboxamide,
N-{4-methoxy-3-[(8-oxa-3-azabicyclo[3.2.1]oct-3-ylacetyl)amino]phenyl}biphenyl-4-carboxamide,
N-(4-methoxy-3-{[(1S,4S)-2-oxa-5-azabicyclo[2.2.1]hept-5-ylacetyl]amino}phenyl)biphenyl-4-carboxamide,
N-[3-({[(2R)-2-(hydroxymethyl)morpholin-4-yl]acetyl}amino)-4-methoxyphenyl]biphenyl-4-carboxamide,
N-(3-{[(4-cyclopropylpiperazin-1-yl)acetyl]amino}-4-methoxyphenyl)biphenyl-4-carboxamide,
N-{4-methoxy-3-[(1,4-oxazepan-4-ylacetyl)amino]phenyl}biphenyl-4-carboxamide,
N-{4-methoxy-3-[(thiomorpholin-4-ylacetyl)amino]phenyl}biphenyl-4-carboxamide,
N-(4-methoxy-3-{[(3-methoxypiperidin-1-yl)acetyl]amino}phenyl)biphenyl-4-carboxamide,
N-(4-methoxy-3-{[(4-methoxypiperidin-1-yl)acetyl]amino}phenyl)biphenyl-4-carboxamide,
N-[3-({[(3S)-3-hydroxypiperidin-1-yl]acetyl}amino)-4-methoxyphenyl]biphenyl-4-carboxamide,
N-(3-{[(2,2-dimethylmorpholin-4-yl)acetyl]amino}-4-methoxyphenyl)biphenyl-4-carboxamide,
N-(4-methoxy-3-{[N-(2-methoxyethyl)glycyl]amino}phenyl)biphenyl-4-carboxamide,
N-[3-({[(3R)-3-hydroxypyrrolidin-1-yl]acetyl}amino)-4-methoxyphenyl]biphenyl-4-carboxamide,
N-[3-({[(3R)-3-(2-hydroxyethyl)morpholin-4-yl]acetyl}amino)-4-methoxyphenyl]biphenyl-4-carboxamide,
N-(3-{[(4-hydroxypiperidin-1-yl)acetyl]amino}-4-methoxyphenyl)biphenyl-4-carboxamide,
N-{4-methoxy-3-[(1-oxa-6-azaspiro[3.4]oct-6-ylacetyl)amino]phenyl}biphenyl-4-carboxamide,
N-(4-methoxy-3-{[(4-methylpiperazin-1-yl)acetyl]amino}phenyl)biphenyl-4-carboxamide,
N-[4-methoxy-3-({[(3S)-3-methylmorpholin-4-yl]acetyl}amino)phenyl]biphenyl-4-carboxamide,
N-(4-methoxy-3-{[N-(2-methoxyethyl)-N-methylglycyl]amino}phenyl)biphenyl-4-carboxamide,
N-(3-{[(4-ethylpiperazin-1-yl)acetyl]amino}-4-methoxyphenyl)biphenyl-4-carboxamide,
N-[4-methoxy-3-({[4-(methylsulfonyl)piperazin-1-yl]acetyl}amino)phenyl]biphenyl-4-carboxamide,
N-{4-methoxy-3-[(morpholin-4-ylacetyl)amino]phenyl}biphenyl-4-carboxamide,
N-(4-methoxy-3-{[2-(morpholin-4-yl)propanoyl]amino}phenyl)biphenyl-4-carboxamide,
N-(4-methoxy-3-{[(2S)-2-(morpholin-4-yl)propanoyl]amino}phenyl)biphenyl-4-carboxamide,
N-(4-methoxy-3-{[(2R)-2-(morpholin-4-yl)propanoyl]amino}phenyl)biphenyl-4-carboxamide,
N-(4-methoxy-3-{[(2S)-2-(8-oxa-3-azabicyclo[3.2.1]oct-3-yl)propanoyl]amino}phenyl)biphenyl-4-carboxamide,
N-(4-methoxy-3-{[(2R)-2-(8-oxa-3-azabicyclo[3.2.1]oct-3-yl)propanoyl]amino}phenyl)biphenyl-4-carboxamide,
N-[3-{[2-(8-oxa-3-azabicyclo[3.2.1]oct-3-yl)propanoyl]amino}-4-(trifluoromethoxy)phenyl]biphenyl-4-carboxamide,
N-[3-{[(2S)-2-(8-oxa-3-azabicyclo[3.2.1]oct-3-yl)propanoyl]amino}-4-(trifluoromethoxy)phenyl]biphenyl-4-carboxamide,
N-[3-{[(2R)-2-(8-oxa-3-azabicyclo[3.2.1]oct-3-yl)propanoyl]amino}-4-(trifluoromethoxy)phenyl]biphenyl-4-carboxamide,
N-[3-{[2-(morpholin-4-yl)propanoyl]amino}-4-(trifluoromethoxy)phenyl]biphenyl-4-carboxamide,
N-[3-{[(2S)-2-(morpholin-4-yl)propanoyl]amino}-4-(trifluoromethoxy)phenyl]biphenyl-4-carboxamide,
N-[3-{[(2R)-2-(morpholin-4-yl)propanoyl]amino}-4-(trifluoromethoxy)phenyl]biphenyl-4-carboxamide,
N-{3-[benzyl(morpholin-4-ylacetyl)amino]-4-methoxyphenyl}biphenyl-4-carboxamide,
N-{4-methoxy-3-[methyl(morpholin-4-ylacetyl)amino]phenyl}biphenyl-4-carboxamide,
N-{3-[(morpholin-4-ylacetyl)amino]-4-(trifluoromethoxy)phenyl}biphenyl-4-carboxamide,
N-{4-tert-butyl-3-[(morpholin-4-ylacetyl)amino]phenyl}biphenyl-4-carboxamide,
N-{4-bromo-3-[(morpholin-4-ylacetyl)amino]phenyl}biphenyl-4-carboxamide,
N-{4-chloro-3-[(morpholin-4-ylacetyl)amino]phenyl}biphenyl-4-carboxamide,
N-{4-methyl-3-[(morpholin-4-ylacetyl)amino]phenyl}biphenyl-4-carboxamide,
N-{4-methoxy-3-[(morpholin-4-ylacetyl)amino]phenyl}-3′-methylbiphenyl-4-carboxamide,
3′-cyano-N-{4-methoxy-3-[(morpholin-4-ylacetyl)amino]phenyl}biphenyl-4-carboxamide,
3′-chloro-N-{4-methoxy-3-[(morpholin-4-ylacetyl)amino]phenyl}biphenyl-4-carboxamide,
3′-fluoro-N-{3-[(morpholin-4-ylacetyl)amino]-4-(trifluoromethoxy)phenyl}biphenyl-4-carboxamide,
4′-fluoro-N-{3-[(morpholin-4-ylacetyl)amino]-4-(trifluoromethoxy)phenyl}biphenyl-4-carboxamide,
4′-amino-N-{3-[(morpholin-4-ylacetyl)amino]-4-(trifluoromethoxy)phenyl}biphenyl-4-carboxamide,
methyl 4′-({4-methoxy-3-[(morpholin-4-ylacetyl)amino]phenyl}carbamoyl)biphenyl-3-carboxylate,
N-{4-methoxy-3-[(morpholin-4-ylacetyl)amino]phenyl}-3′-(trifluoromethyl)biphenyl-4-carboxamide,
methyl 4′-({4-methoxy-3-[(morpholin-4-ylacetyl)amino]phenyl}carbamoyl)biphenyl-4-carboxylate,
3′-methoxy-N-{4-methoxy-3-[(morpholin-4-ylacetyl)amino]phenyl}biphenyl-4-carboxamide,
3′-fluoro-N-{4-methoxy-3-[(morpholin-4-ylacetyl)amino]phenyl}biphenyl-4-carboxamide,
2′-fluoro-N-{4-methoxy-3-[(morpholin-4-ylacetyl)amino]phenyl}biphenyl-4-carboxamide,
4′-amino-N-{4-methoxy-3-[(morpholin-4-ylacetyl)amino]phenyl}biphenyl-4-carboxamide,
N-{4-hydroxy-3-[(morpholin-4-ylacetyl)amino]phenyl}biphenyl-4-carboxamide,
N-{4-ethoxy-3-[(morpholin-4-ylacetyl)amino]phenyl}biphenyl-4-carboxamide,
N-(biphenyl-4-yl)-4-[(2-methoxyethoxy)methyl]-3-[(morpholin-4-ylacetyl)amino]benzamide,
N-(biphenyl-4-yl)-4-[(3-methoxypropoxy)methyl]-3-[(morpholin-4-ylacetyl)amino]benzamide,
4-(benzyloxy)-N-(biphenyl-4-yl)-3-({[1-(morpholin-4-yl)cyclopropyl]carbonyl}amino)benzamide,
4-(3-aminopropoxy)-N-(biphenyl-4-yl)-3-({[1-(morpholin-4-yl)cyclopropyl]carbonyl}amino)benzamide hydrochloride (1:1),
4-(3-acetamidopropoxy)-N-(biphenyl-4-yl)-3-({[1-(morpholin-4-yl)cyclopropyl]carbonyl}amino)benzamide,
N-(biphenyl-4-yl)-4-(3-methoxypropoxy)-3-[(morpholin-4-ylacetyl)amino]benzamide,
N-(biphenyl-4-yl)-4-(2-methoxyethoxy)-3-[(morpholin-4-ylacetyl)amino]benzamide,
N-(biphenyl-4-yl)-4-(2-hydroxyethoxy)-3-[(morpholin-4-ylacetyl)amino]benzamide,
N-(biphenyl-4-yl)-3-({[1-(morpholin-4-yl)cyclopropyl]carbonyl}amino)-4-(trifluoromethoxy)benzamide,
N-(biphenyl-4-yl)-4-(methoxymethyl)-3-({[1-(morpholin-4-yl)cyclopropyl]carbonyl}amino)benzamide,
N-(biphenyl-4-yl)-4-(methoxymethyl)-3-({[1-(4-methylpiperazin-1-yl)cyclopropyl]carbonyl}amino)benzamide hydrochloride (1:1),
N-(biphenyl-4-yl)-4-chloro-3-({[1-(morpholin-4-yl)cyclopropyl]carbonyl}amino)benzamide,
N-(biphenyl-4-yl)-3-({[1-(morpholin-4-yl)cyclopropyl]carbonyl}amino)-4-(trifluoromethyl)benzamide,
N-(biphenyl-4-yl)-4-methoxy-3-{[2-(morpholin-4-yl)butanoyl]amino}benzamide,
N-(biphenyl-4-yl)-4-methoxy-3-({[1-(4-methylpiperazin-1-yl)cyclopropyl]carbonyl}amino)benzamide,
hydrochloride (1:1),
N-(biphenyl-4-yl)-4-methoxy-3-({[1-(morpholin-4-yl)cyclopropyl]carbonyl}amino)benzamide,
N⁴-(biphenyl-4-yl)-N¹-ethyl-2-[(morpholin-4-ylacetyl)amino]terephthalamide,
N⁴-(biphenyl-4-yl)-2-[(morpholin-4-ylacetyl)amino]-N¹-[3-(pyrrolidin-1-yl)propyl]terephthalamide,
N⁴-(biphenyl-4-yl)-N¹-[3-(dimethylamino)propyl]-2-[(morpholin-4-ylacetyl)amino]terephthalamide,
formic acid —N⁴-(biphenyl-4-yl)-N¹-[2-(dimethylamino)ethyl]-2-[(morpholin-4-ylacetyl)amino]terephthalamide (1:1),
N⁴-(biphenyl-4-yl)-N¹-(2-methoxyethyl)-2-[(morpholin-4-ylacetyl)amino]terephthalamide,
N⁴-(biphenyl-4-yl)-N¹-cyclopropyl-2-[(morpholin-4-ylacetyl)amino]terephthalamide,
N⁴-(biphenyl-4-yl)-N¹-(3-methoxypropyl)-2-[(morpholin-4-ylacetyl)amino]terephthalamide,
N-(biphenyl-4-yl)-4-(methylsulfanyl)-3-[(morpholin-4-ylacetyl)amino]benzamide,
N-(biphenyl-4-yl)-4-(methylsulfinyl)-3-[(morpholin-4-ylacetyl)amino]benzamide,
N-(biphenyl-4-yl)-4-(methylsulfonyl)-3-[(morpholin-4-ylacetyl)amino]benzamide,
N-(biphenyl-4-yl)-4-(cyclopropyloxy)-3-[(morpholin-4-ylacetyl)amino]benzamide,
N-(biphenyl-4-yl)-4-(cyclopropyloxy)-3-({[1-(morpholin-4-yl)cyclopropyl]carbonyl}amino)benzamide,
N-(biphenyl-4-yl)-4-(cyclopropyloxy)-3-({[1-(4-methylpiperazin-1-yl)cyclopropyl]carbonyl}amino)benzamide,
N-(biphenyl-4-yl)-4-(cyclopropyloxy)-3-({[1-(4-cyclopropylpiperazin-1-yl)cyclopropyl]carbonyl}amino)benzamide,
N⁴-(Biphenyl-4-yl)-2-[(morpholin-4-ylacetyl)amino]terephthalamide,
N-(biphenyl-4-yl)-4-(2-hydroxypropan-2-yl)-3-[(morpholin-4-ylacetyl)amino]benzamide,
4′-acetamido-N-{4-methoxy-3-[(morpholin-4-ylacetyl)amino]phenyl}biphenyl-4-carboxamide,
N-{4-methoxy-3-[(morpholin-4-ylacetyl)amino]phenyl}-4′-(methylamino)biphenyl-4-carboxamide,
4′-(aminomethyl)-N-{4-methoxy-3-[(morpholin-4-ylacetyl)amino]phenyl}biphenyl-4-carboxamide,
N-(biphenyl-4-yl)-4-(3-hydroxypropoxy)-3-[(morpholin-4-ylacetyl)amino]benzamide,
4-(2-amino-2-oxoethoxy)-N-(biphenyl-4-yl)-3-[(morpholin-4-ylacetyl)amino]benzamide,
4-methoxy-3-[(morpholin-4-ylacetyl)amino]-N-(2,3′,5′-trifluorobiphenyl-4-yl)benzamide,
N-(biphenyl-4-yl)-4-[(methylsulfonyl)methyl]-3-[(morpholin-4-ylacetyl)amino]benzamide,
N-(biphenyl-4-yl)-3-{[(4-methylpiperazin-1-yl)acetyl]amino}-4-[(methylsulfonyl)methyl]benzamide,
4-(3-acetamidopropoxy)-N-(biphenyl-4-yl)-3-[(morpholin-4-ylacetyl)amino]benzamide,
N-(biphenyl-4-yl)-3-[(morpholin-4-ylacetyl)amino]-4-(2,2,2-trifluoroethoxy)benzamide,
N-(biphenyl-4-yl)-4-(cyclopropyloxy)-3-[(8-oxa-3-azabicyclo[3.2.1]oct-3-ylacetyl)amino]benzamide,
tert-butyl[1-({5-[(biphenyl-4-ylcarbonyl)amino]-2-methoxyphenyl}carbamoyl)cyclopropyl]carbamate,
N-[3-{[N-(2-methoxyethyl)glycyl]amino}-4-(trifluoromethoxy)phenyl]biphenyl-4-carboxamide,
N-(biphenyl-4-yl)-4-methoxy-3-{[(2R*)-2-(morpholin-4-yl)butanoyl]amino}benzamide,
N-(biphenyl-4-yl)-4-methoxy-3-{[(2S*)-2-(morpholin-4-yl)butanoyl]amino}benzamide,
N-(biphenyl-4-yl)-4-methoxy-3-({[1-(4-methylpiperazin-1-yl)cyclopropyl]carbonyl}amino)benzamide,
N-(biphenyl-4-yl)-3-fluoro-4-methoxy-5-[(morpholin-4-ylacetyl)amino]benzamide,
N-{3-[(3,3,3-trifluoroalanyl)amino]-4-(trifluoromethoxy)phenyl}biphenyl-4-carboxamide,
N-(biphenyl-4-yl)-3-chloro-4-methoxy-5-[(morpholin-4-ylacetyl)amino]benzamide,
N-(biphenyl-4-yl)-4-methoxy-3-{[(2R)-3-methyl-2-(morpholin-4-yl)butanoyl]amino}benzamide,
N-(3-{[(4-fluorophenyl)acetyl]amino}-4-methoxyphenyl)biphenyl-4-carboxamide,
N-(biphenyl-4-yl)-3-{[2-methyl-3-(morpholin-4-yl)propanoyl]amino}-4-(trifluoromethyl)benzamide,
N-[3-{[N-(2-hydroxyethyl)glycyl]amino}-4-(trifluoromethoxy)phenyl]biphenyl-4-carboxamide,
N-(4-methoxy-3-{[3-(morpholin-4-yl)propanoyl]amino}phenyl)biphenyl-4-carboxamide,
N-(3-{[(3-fluorophenyl)acetyl]amino}-4-methoxyphenyl)biphenyl-4-carboxamide,
N-(4-methoxy-3-{[(3-methoxyphenyl)acetyl]amino}phenyl)biphenyl-4-carboxamide,
N-(4-methoxy-3-{[(4-methoxyphenyl)acetyl]amino}phenyl)biphenyl-4-carboxamide,
N-{3-[(cyclohexylacetyl)amino]-4-methoxyphenyl}biphenyl-4-carboxamide,
N-(biphenyl-4-yl)-4-(methoxymethyl)-3-{[(2R*)-2-(8-oxa-3-azabicyclo[3.2.1]oct-3-yl)propanoyl]amino}benzamide,
methyl 4-(biphenyl-4-ylcarbamoyl)-2-({[1-(morpholin-4-yl)cyclopropyl]carbonyl}amino)benzoate,
N-[3-({[1-(morpholin-4-yl)cyclopropyl]carbonyl}amino)-4-(trifluoromethoxy)phenyl]biphenyl-4-carboxamide,
N-(biphenyl-4-yl)-3-({[1-(4-cyclopropylpiperazin-1-yl)cyclopropyl]carbonyl}amino)-4-(methoxymethyl)benzamide,
N-[4-methoxy-3-({[1-(morpholin-4-yl)cyclopropyl]carbonyl}amino)phenyl]biphenyl-4-carboxamide,
N-(biphenyl-4-yl)-4-fluoro-3-({[1-(morpholin-4-yl)cyclopropyl]carbonyl}amino)benzamide,
N-(biphenyl-4-yl)-4-bromo-3-({[1-(morpholin-4-yl)cyclopropyl]carbonyl}amino)benzamide,
N-(biphenyl-4-yl)-4-methoxy-3-{[(2R)-2-(morpholin-4-yl)propanoyl]amino}benzamide,
N-(biphenyl-4-yl)-4-(methoxymethyl)-3-[(8-oxa-3-azabicyclo[3.2.1]oct-3-ylacetyl)amino]benzamide,
N4-(biphenyl-4-yl)-2-[(morpholin-4-ylacetyl)amino]-N1-(propan-2-yl)benzene-1,4-dicarboxamide,
N-(biphenyl-4-yl)-3-({[1-(4-cyclopropylpiperazin-1-yl)cyclopropyl]carbonyl}amino)-4-methoxybenzamide,
N-(biphenyl-4-yl)-4-(methoxymethyl)-3-{[2-(morpholin-4-yl)propanoyl]amino}benzamide,
N-(biphenyl-4-yl)-3-({[1-(dimethylamino)cyclopropyl]carbonyl}amino)-4-(methoxymethyl)benzamide,
N-(biphenyl-4-yl)-4-methyl-3-({[1-(morpholin-4-yl)cyclopropyl]carbonyl}amino)benzamide,
N-(biphenyl-4-yl)-4-[(3-methoxypropoxy)methyl]-3-{[2-(morpholin-4-yl)propanoyl]amino}benzamide,
N-[3-{[2-(morpholin-4-yl)propanoyl]amino}-4-(trifluoromethyl)phenyl]biphenyl-4-carboxamide,
N-(biphenyl-4-yl)-4-[(2-methoxyethoxy)methyl]-3-{[(2R*)-2-(morpholin-4-yl)propanoyl]amino}benzamide,
N-[4-methoxy-3-({[1-(4-methylpiperazin-1-yl)cyclopropyl]carbonyl}amino)phenyl]biphenyl-4-carboxamide hydrochloride (1:1),
N-(biphenyl-4-yl)-4-[(2-methoxyethoxy)methyl]-3-{[2-(morpholin-4-yl)propanoyl]amino}benzamide,
N-(biphenyl-4-yl)-4-(methoxymethyl)-3-[(morpholin-4-ylacetyl)amino]benzamide,
N-(biphenyl-4-yl)-4-[(3-methoxypropoxy)methyl]-3-{[(2R*)-2-(morpholin-4-yl)propanoyl]amino}benzamide,
N-(biphenyl-4-yl)-4-(methoxymethyl)-3-{[(2S*)-2-(8-oxa-3-azabicyclo[3.2.1]oct-3-yl)propanoyl]amino}benzamide,
N-(biphenyl-4-yl)-4-[(methylsulfonyl)methyl]-3-[(8-oxa-3-azabicyclo[3.2.1]oct-3-ylacetyl)amino]benzamide,
N-(biphenyl-4-yl)-4-(methoxymethyl)-3-{[(2R*)-2-(morpholin-4-yl)propanoyl]amino}benzamide,
N-(biphenyl-4-yl)-4-(methoxymethyl)-3-{[2-(8-oxa-3-azabicyclo[3.2.1]oct-3-yl)propanoyl]amino}benzamide,
N-[4-fluoro-3-({[1-(morpholin-4-yl)cyclopropyl]carbonyl}amino)phenyl]biphenyl-4-carboxamide,
N-[3-{[(2R*)-2-(morpholin-4-yl)propanoyl]amino}-4-(trifluoromethyl)phenyl]biphenyl-4-carboxamide,
N-(biphenyl-4-yl)-4-(methoxymethyl)-3-{[(2S*)-2-(morpholin-4-yl)propanoyl]amino}benzamide,
N-(biphenyl-4-yl)-4-(cyclopropyloxy)-3-{[(3-methoxypyrrolidin-1-yl)acetyl]amino}benzamide,
N-(biphenyl-4-yl)-4-(methoxymethyl)-3-{[(1S,4S)-2-oxa-5-azabicyclo[2.2.1]hept-5-ylacetyl]amino}benzamide,
N-(biphenyl-4-yl)-4-[(3-methoxypropoxy)methyl]-3-[(8-oxa-3-azabicyclo[3.2.1]oct-3-ylacetyl)amino]benzamide,
N-(biphenyl-4-yl)-4-[(2-methoxyethoxy)methyl]-3-{[(2S*)-2-(morpholin-4-yl)propanoyl]amino}benzamide,
N-[3-{[2-(8-oxa-3-azabicyclo[3.2.1]oct-3-yl)propanoyl]amino}-4-(trifluoromethyl)phenyl]biphenyl-4-carboxamide,
N-(biphenyl-4-yl)-4-[(3-methoxypropoxy)methyl]-3-{[(2S*)-2-(morpholin-4-yl)propanoyl]amino}benzamide,
N-[3-{[(2S*)-2-(morpholin-4-yl)propanoyl]amino}-4-(trifluoromethyl)phenyl]biphenyl-4-carboxamide,
N-(biphenyl-4-yl)-4-(cyclopropyloxy)-3-{[(1R,4R)-2-oxa-5-azabicyclo[2.2.1]hept-5-ylacetyl]amino}benzamide,
N-[3-{[(2R*)-2-(8-oxa-3-azabicyclo[3.2.1]oct-3-yl)propanoyl]amino}-4-(trifluoromethyl)phenyl]biphenyl-4-carboxamide,
N-{3-[(morpholin-4-ylacetyl)amino]-4-(trifluoromethyl)phenyl}biphenyl-4-carboxamide,
N-(biphenyl-4-yl)-4-(difluoromethoxy)-3-[(morpholin-4-ylacetyl)amino]benzamide,
N-[3-({[1-(4-cyclopropylpiperazin-1-yl)cyclopropyl]carbonyl}amino)-4-methoxyphenyl]biphenyl-4-carboxamide,
N-(biphenyl-4-yl)-4-(cyclopropyloxy)-3-{[(3-methoxyazetidin-1-yl)acetyl]amino}benzamide,
N-(biphenyl-4-yl)-4-(methoxymethyl)-3-[(1H-pyrazol-1-ylacetyl)amino]benzamide,
N-[3-({[1-(dimethylamino)cyclopropyl]carbonyl}amino)-4-(trifluoromethoxy)phenyl]biphenyl-4-carboxamide,
N-[3-({[1-(dimethylamino)cyclopropyl]carbonyl}amino)-4-methoxyphenyl]biphenyl-4-carboxamide,
N-(biphenyl-4-yl)-3-{[(1R,4R)-2-oxa-5-azabicyclo[2.2.1]hept-5-ylacetyl]amino}-4-(trifluoromethyl)benzamide,
N⁴-(biphenyl-4-yl)-2-[(morpholin-4-ylacetyl)amino]-N¹-[2-(pyrrolidin-1-yl)ethyl]terephthalamide
N-(biphenyl-4-yl)-4-(cyclopropyloxy)-3-{[(1S,4S)-2-oxa-5-azabicyclo[2.2.1]hept-5-ylacetyl]amino}benzamide,
N-(biphenyl-4-yl)-4-[(2-methoxyethoxy)methyl]-3-[(8-oxa-3-azabicyclo[3.2.1]oct-3-ylacetyl)amino]benzamide,
N-(biphenyl-4-yl)-4-(3-methyl-1,2,4-oxadiazol-5-yl)-3-[(morpholin-4-ylacetyl)amino]benzamide,
4′-hydroxy-N-{4-methoxy-3-[(morpholin-4-ylacetyl)amino]phenyl}biphenyl-4-carboxamide,
3,3′,5′-trifluoro-N-{4-methoxy-3-[(morpholin-4-ylacetyl)amino]phenyl}biphenyl-4-carboxamide,
N-[3-{[(2S*)-2-(8-oxa-3-azabicyclo[3.2.1]oct-3-yl)propanoyl]amino}-4-(trifluoromethyl)phenyl]biphenyl-4-carboxamide,
4′-(dimethylamino)-N-{4-methoxy-3-[(morpholin-4-ylacetyl)amino]phenyl}biphenyl-4-carboxamide,
3′,5′-difluoro-N-{4-methoxy-3-[(morpholin-4-ylacetyl)amino]phenyl}biphenyl-4-carboxamide,
N-(biphenyl-4-yl)-4-[3-(2-methoxyethyl)-1,2,4-oxadiazol-5-yl]-3-[(morpholin-4-ylacetyl)amino]benzamide,
N-(biphenyl-4-yl)-4-methoxy-3-{[(1R,4R)-2-oxa-5-azabicyclo[2.2.1]hept-5-ylacetyl]amino}benzamide,
N-(biphenyl-4-yl)-4-methoxy-3-{[(3-methoxypyrrolidin-1-yl)acetyl]amino}benzamide,
2-fluoro-N-{4-methoxy-3-[(morpholin-4-ylacetyl)amino]phenyl}biphenyl-4-carboxamide,
N-(biphenyl-4-yl)-4-(hydroxymethyl)-3-[(morpholin-4-ylacetyl)amino]benzamide,
N-(4-methoxy-3-{[(1R,4R)-2-oxa-5-azabicyclo[2.2.1]hept-5-ylacetyl]amino}phenyl)biphenyl-4-carboxamide,
N-(biphenyl-4-yl)-4-methoxy-3-({[1-(morpholin-4-yl)cyclobutyl]carbonyl}amino)benzamide,
N-(biphenyl-4-yl)-4-methoxy-3-{[(3-methoxyazetidin-1-yl)acetyl]amino}benzamide,
4-(2,3-dihydro-1-benzofuran-5-yl)-N-{4-methoxy-3-[(morpholin-4-ylacetyl)amino]phenyl}benzamide,
3′-amino-N-{4-methoxy-3-[(morpholin-4-ylacetyl)amino]phenyl}biphenyl-4-carboxamide,
N-(biphenyl-4-yl)-4-methoxy-3-[methyl(8-oxa-3-azabicyclo[3.2.1]oct-3-ylacetyl)amino]benzamide,
N-{4-(2-amino-2-oxoethoxy)-3-[(morpholin-4-ylacetyl)amino]phenyl}biphenyl-4-carboxamide,
N-[3-({[(2R,6S)-2,6-dimethylmorpholin-4-yl]acetyl}amino)-4-methoxyphenyl]biphenyl-4-carboxamide,
N-[3-({[(3S)-3-(2-hydroxyethyl)morpholin-4-yl]acetyl}amino)-4-methoxyphenyl]biphenyl-4-carboxamide,
N-[3-({[(2S)-2-(hydroxymethyl)morpholin-4-yl]acetyl}amino)-4-methoxyphenyl]biphenyl-4-carboxamide,
N-(biphenyl-4-yl)-3-chloro-4-methoxy-5-{[(4-methylpiperazin-1-yl)acetyl]amino}benzamide,
N-(biphenyl-4-yl)-3-fluoro-4-methoxy-5-{[(4-methylpiperazin-1-yl)acetyl]amino}benzamide,
N-(biphenyl-4-yl)-4-[2-(2-methoxyethoxy)ethoxy]-3-({[1-(morpholin-4-yl)cyclopropyl]carbonyl}amino)benzamide,
N-(biphenyl-4-yl)-4-(2-methoxyethoxy)-3-({[1-(morpholin-4-yl)cyclopropyl]carbonyl}amino)benzamide,
N-(biphenyl-4-yl)-4-(cyclopropylmethoxy)-3-{[(4-cyclopropylpiperazin-1-yl)acetyl]amino}benzamide,
N-(biphenyl-4-yl)-4-(cyclopropylmethoxy)-3-[(morpholin-4-ylacetyl)amino]benzamide,
4-[2-(2-methoxyethoxy)ethoxy]-N-(4′-methylbiphenyl-4-yl)-3-({[1-(morpholin-4-yl)cyclopropyl]carbonyl}amino)benzamide, and
N-(biphenyl-4-yl)-4-(cyclopropylmethoxy)-3-{[(4-methylpiperazin-1-yl)acetyl]amino}benzamide,
or a tautomer, an N-oxide, a hydrate, a solvate, or a salt thereof, or a mixture of same.

9. (canceled)

10. A pharmaceutical composition comprising a compound of general formula (I), or a stereoisomer, a tautomer, an N oxide, a hydrate, a solvate, or a pharmaceutically acceptable salt thereof, or a mixture of same, according to claim 1, and a pharmaceutically acceptable diluent or carrier.

11. (canceled)

12. (canceled)

13. A method for the treatment of a disease of uncontrolled cell growth, proliferation or survival, an inappropriate cellular immune response, or an inappropriate cellular inflammatory response, comprising administering to a patient in need thereof a therapeutically effective amount of a compound of general formula (I), or a stereoisomer, a tautomer, an N oxide, a hydrate, a solvate, or a pharmaceutically acceptable salt thereof, or a mixture of same.

14. An intermediate compound of general formula (VI): 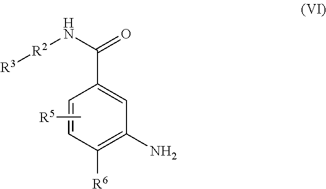
in which R², R³, R⁵, and R⁶ are as defined in claim 1.

15. An intermediate compound of general formula (XI): 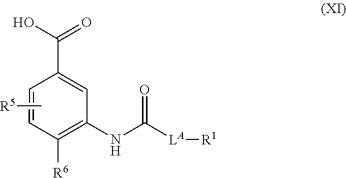
in which L^{A}, R¹, R⁵, and R⁶ are as defined in claim 1.

16. An intermediate compound of general formula (XIa): 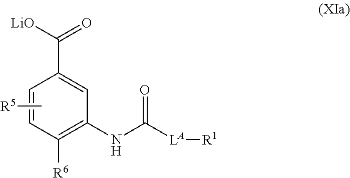
in which L^{A}, R¹, R⁵, and R⁶ are as defined in claim 1.

17. An intermediate compound of general formula (XVII): 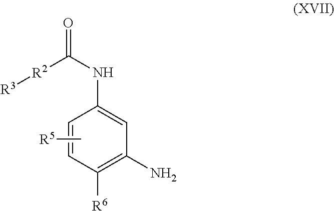
in which R², R³, R⁵, and R⁶ are as defined in claim 1.

18. An intermediate compound of general formula (XXII): 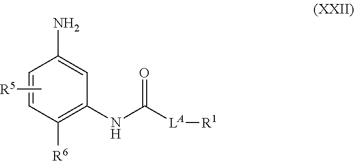
in which L^{A}, R¹, R⁵ and R⁶ are as defined in claim 1.

19. An intermediate compound of general formula (XXIV): 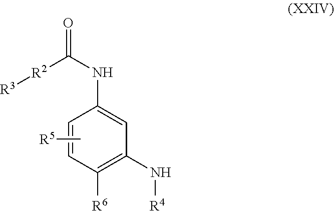
in which R², R³, R⁴, R⁵ and R⁶ are as defined in claim 1.

20. An intermediate compound of general formula (XXV): 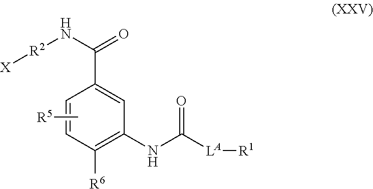
in which L^{A}, R¹, R², R⁵ and R⁶ are as defined in claim 1, and X represents chloro, bromo, iodo, trifluoromethylsulfonyloxy, or a boronic acid or an ester thereof.

21. The method according to claim 13, wherein the uncontrolled cell growth, proliferation or survival, inappropriate cellular immune response, or inappropriate cellular inflammatory response is mediated by the Wnt pathway.

22. The method according to claim 13, wherein the disease of uncontrolled cell growth, proliferation or survival, inappropriate cellular immune response, or inappropriate cellular inflammatory response is a haematological tumour, a solid tumour or metastases thereof.

23. The method according to claim 22, wherein the haematological tumour, solid tumour or metastases thereof is selected from leukaemias and myelodysplastic syndrome, malignant lymphomas, head and neck tumours, brain tumours and brain metastases, tumours of the thorax, non small cell and small cell lung tumours, gastrointestinal tumours, endocrine tumours, mammary and other gynaecological tumours, urological tumours, renal, bladder and prostate tumours, skin tumours, and sarcomas, and metastases thereof.


SEQUENCE LISTING (6 sequences)

SEQ ID NO: 1 - DNA, 20 bp, Unknown
  source 1..20: /mol_type=“unassigned DNA” /note=“L32 forward primer” /organism=“Unknown”
aagttcatccggcaccagtc

SEQ ID NO: 2 - DNA, 21 bp, Unknown
  source 1..21: /mol_type=“unassigned DNA” /note=“L32 revers primer” /organism=“Unknown”
tggcccttgaatcttctacga

SEQ ID NO: 3 - DNA, 22 bp, Unknown
  source 1..22: /mol_type=“unassigned DNA” /note=“L32 probe” /organism=“Unknown”
cccagaggcattgacaacaggg

SEQ ID NO: 4 - DNA, 20 bp, Unknown
  source 1..20: /mol_type=“unassigned DNA” /note=“AXIN2 forward primer” /organism=“Unknown”
aggccagtgagttggttgtc

SEQ ID NO: 5 - DNA, 20 bp, Unknown
  source 1..20: /mol_type=“unassigned DNA” /note=“AXIN2 revers primer” /organism=“Unknown”
agctctgagccttcagcatc

SEQ ID NO: 6 - DNA, 25 bp, Unknown
  source 1..25: /mol_type=“unassigned DNA” /note=“AXIN2 probe” /organism=“Unknown”
tctgtggggaagaaattccataccg